SEQUENCE LISTING (211 sequences)

SEQ ID NO: 1 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 2 - protein, 68 aa, synthetic construct
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 12: note = Any amino acid
  VARIANT 13: note = Any amino acid
  VARIANT 14: note = Any amino acid
  VARIANT 15: note = Any amino acid
  VARIANT 18: note = Any amino acid
  VARIANT 21: note = Any amino acid
  VARIANT 26: note = Any amino acid
  VARIANT 31: note = Any amino acid
  VARIANT 35: note = Any amino acid
  VARIANT 37: note = Any amino acid
  VARIANT 38: note = Any amino acid
  VARIANT 39: note = Any amino acid
  VARIANT 57: note = Any amino acid
  VARIANT 58: note = Any amino acid
  VARIANT 59: note = Any amino acid
  VARIANT 60: note = Any amino acid
  VARIANT 61: note = Any amino acid
  VARIANT 62: note = Any amino acid
  VARIANT 63: note = Any amino acid
  VARIANT 64: note = Any amino acid
  VARIANT 65: note = Any amino acid
  VARIANT 66: note = Any amino acid
  VARIANT 67: note = Any amino acid
MXXXXXFKYKGXXXXVDXSKXKKVWXVGKMXSFTXDXXXGKTGRGAVSEKDAPKELXXXXXXXXXXXK

SEQ ID NO: 3 - protein, 747 aa, synthetic construct
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGSMVTVKFKYKGEELEVDISKIKKVWRVGKMISFTYDDNGKTGRGAVSEKDAPKELLQMLEKSGKK

SEQ ID NO: 4 - DNA, 10 bp, synthetic construct
  source 1..10: mol_type = other DNA
tttttttttt

SEQ ID NO: 5 - protein, 747 aa, synthetic construct
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGSMVTVKFKYKGEEKEVDISKIKKVWRVGKMISFTYDDNGKTGRGAVSEKDAPKELLQMLEKSGKK

SEQ ID NO: 6 - protein, 726 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGMATVKFKYKGEEKEVDISKIKKVWRVGKMISFTYDEGGGKTGRGAVSEKDAPKELLQMLEKQKK

SEQ ID NO: 7 - protein, 671 aa, Moloney Murine Leukemia Virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 8 - protein, 726 aa, synthetic construct
MATVKFKYKGEEKEVDISKIKKVWRVGKMISFTYDEGGGKTGRGAVSEKDAPKELLQMLEKQKKGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 9 - RNA, 63 nt, synthetic construct
  source 1..63: mol_type = other RNA
acgaccgtcgtcatgtagcgtttgtcggagactcctagatcagatgtcctcctggctactgca

SEQ ID NO: 10 - DNA, 18 bp, synthetic construct
  source 1..18: mol_type = other DNA
cgactcactgacactcgc

SEQ ID NO: 11 - DNA, 30 bp, synthetic construct
  source 1..30: mol_type = other DNA
  misc_feature 1..27: note = DNA
  misc_feature 28..30: note = RNA
aagcagtggtatcaacgcagagtacatggg

SEQ ID NO: 12 - protein, 64 aa, unidentified
  source 1..64: note = Archaebacteria
MVTVKFKYKGEEKEVDISKIKKVWRVGKMISFTYDDNGKTGRGAVSEKDAPKELLQMLEKSGKK

SEQ ID NO: 13 - protein, 64 aa, unidentified
  source 1..64: note = Archaebacteria
MATVKFKYKGEEKEVDISKIKKVWRVGKMISFTYDEGGGKTGRGAVSEKDAPKELLQMLEKQKK

SEQ ID NO: 14 - protein, 661 aa, synthetic construct
MTWLSDFPQAWAETGGMGLAVRQAPLIIPLKATSTPVSIKQYPLSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSNARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 15 - protein, 5 aa, synthetic construct
HHHHH

SEQ ID NO: 16 - protein, 63 aa, unidentified
  source 1..63: note = Archaebacteria
VTVKFKYKGEEKEVDISKIKKVWRVGKMISFTYDDNGKTGRGAVSEKDAPKELLQMLEKSGKK

SEQ ID NO: 17 - protein, 63 aa, unidentified
  source 1..63: note = Archaebacteria
ATVKFKYKGEEKEVDISKIKKVWRVGKMISFTYDEGGGKTGRGAVSEKDAPKELLQMLEKQKK

SEQ ID NO: 18 - protein, 63 aa, unidentified
  source 1..63: note = Archaebacteria
VTVKFKYKGEELEVDISKIKKVWRVGKMISFTYDDNGKTGRGAVSEKDAPKELLQMLEKSGKK

SEQ ID NO: 19 - protein, 5 aa, synthetic construct
GGGGS

SEQ ID NO: 20 - protein, 728 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 21 - protein, 12 aa, synthetic construct
IENSSPNSRLIN

SEQ ID NO: 22 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 23 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 24 - protein, 649 aa, synthetic construct
MTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 25 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 26 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 27 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 28 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 29 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNHMAHQAARRAAITETPDTSTLP

SEQ ID NO: 30 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 31 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLP

SEQ ID NO: 32 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 33 - protein, 671 aa, synthetic construct
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 34 - protein, 671 aa, synthetic construct
TLNIEDEHRLHETSKEPDVSLGSTWLSDFPQAWAETGGVGLAVRQALLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 35 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 36 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 37 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 38 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 39 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 40 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPADRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 41 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 42 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 43 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 44 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 45 - protein, 649 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 46 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 47 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 48 - protein, 649 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 49 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 50 - protein, 649 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 51 - protein, 679 aa, synthetic construct
MGSSHHHHHHSSGLVPRGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 52 - protein, 679 aa, synthetic construct; identical to SEQ ID NO: 51

SEQ ID NO: 53 - protein, 661 aa, synthetic construct; identical to SEQ ID NO: 1

SEQ ID NO: 54 - protein, 662 aa, synthetic construct
GSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 55 - protein, 660 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 56 - protein, 6 aa, synthetic construct
HHHHHH

SEQ ID NO: 57 - protein, 11 aa, synthetic construct
SEEDEEKEEDG

SEQ ID NO: 58 - protein, 7 aa, synthetic construct
ENLYFQG

SEQ ID NO: 59 - protein, 5 aa, synthetic construct
DDDDK

SEQ ID NO: 60 - protein, 4 aa, synthetic construct
IEGR

SEQ ID NO: 61 - protein, 6 aa, synthetic construct
LVPRGS

SEQ ID NO: 62 - protein, 8 aa, synthetic construct
LEVLFQGP

SEQ ID NO: 63 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 64 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 65 - protein, 660 aa, synthetic construct
TWLSDFPQAWAETGGLGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 66 - DNA, 1980 bp, synthetic construct
  source 1..1980: mol_type = other DNA
acttggctgtctgatttccctcaggcgtgggccgaaacgggtggcctgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 67 - protein, 660 aa, synthetic construct
TWLSDFPQAWAETGGLGLAVRQAPLIIPLKATSTPVSIKQYPLSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 68 - DNA, 1980 bp, synthetic construct
  source 1..1980: mol_type = other DNA
acttggctgtctgatttccctcaggcgtgggccgaaacgggtggcctgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 69 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 70 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 71 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 72 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 73 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 74 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgtggaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 75 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGRAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 76 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaaattcttgggccgcgcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 77 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 78 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtctgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 79 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCRQGTRALLQTLGNLGYRASAKKAQICQKQVKYLGYLLKEGQRWLTEARKETVMGQPTPKTPRQLRKFLGRAGSCRLHIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 80 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgagcgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccgccagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaaattcttgggccgcgcgggtagctgccgcctgcatatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 81 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 82 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 83 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 84 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 85 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 86 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggcgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 87 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 88 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 89 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 90 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 91 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 92 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 93 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 94 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggcgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 95 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 96 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacggatggtagcagcctgttgcaagagggtcagcgtaaggccggtgccgcggtcaccaccgagactggtgtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcagggcgagatctatcgccgtcgcggctttctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgccgatcgagaatagcagcccgaacccgcgtctgatcaat

SEQ ID NO: 97 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 98 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 99 - protein, 660 aa, synthetic construct
TWLSDFPQAWAETGGVGLAVRQAPLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 100 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 101 - protein, 660 aa, synthetic construct
TWLSDFPQAWAETWGVGLAVRQALLIIPLKATSTPVSIKQYPMSQKARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 102 - DNA, 1980 bp, synthetic construct
  source 1..1980: mol_type = other DNA
acttggctgtctgatttccctcaggcgtgggccgaaacgtggggcgtgggtctggcagtgcgtcaggcactgctgattattccgctgaaagcgacgtcgaccccggtgagcatcaagcaatatccgatgtcccaaaaggcgcgcttaggtattaagccgcacattcagcgtctgctggatcaaggtattctggttccgtgtcgcagcccgtggaataccccgcttctcccggtgaagaaaccgggcacgagcgattaccgtccagtccaagacttgcgcgaagttaacaagcgcgttgaagatattcgcccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcaggcagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccggcggcaaagagatcaagaataaagacgaaattctggcgctgctgaaagcgctgttcctgccgaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 103 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLPIENSSPNSRLIN

SEQ ID NO: 104 - DNA, 1980 bp, synthetic construct
  source 1..1980: mol_type = other DNA
acttggctgtctgatttccctcaggcgtgggccgaaacgggtggcgtgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttgaagaagcgctgcgccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgagccaaccgctggtgatcaaagcaccgcatgcagtcgaggcccttgttaagcaaccggcagaaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccgatgagactgaagtgatttgggctaaagcattgcctgcgggtaccagcgcgcagcgtgccgagctgatcgcactgacccaagcgctgaaaatggctgagggtaagaaactgaatgtgtacacgaacagccgttatgcctttgcgaccgcccacattcacggcgagatctatcgccgtcgcggctggctgacgtccggcggcaaagagatcaagaataaagacgaaattctggcgctgctgaaagcgctgttcctgagcaaacgtctgtcgatcatccattgcccgggtcaccagaaaggccacagcgtggaggcgcgtggtaatcgcatggctcatcaggctgcgcgtcgcgccgcaattaccgaaaccccggacaccagcacgctgccgatcgagaatagcagcccgaacagccgtctgatcaat

SEQ ID NO: 105 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGAAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 106 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgcaaatttctgggtgcagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccgatggtagcagcctgctgcaggaaggtcagcgcaaagcaggtgcagcagtgaccaccgaaaccggtgtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatttatcgccgccgcggttttctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgccgattgaaaacagcagcccgaacccgcgcctgattaac

SEQ ID NO: 107 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGYSAEARGNRMAVQAARKAAITETPDTSTLLIENSSPNPRLIN

SEQ ID NO: 108 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggttatagcgcagaagcacgcggtaaccgcatggcagtgcaggcagcacgcaaagcagcaattaccgaaaccccggataccagcaccctgctgattgaaaacagcagcccgaacccgcgcctgattaac

SEQ ID NO: 109 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 110 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 111 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 112 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 113 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLPIENSSPNPRLIN

SEQ ID NO: 114 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccgatggtagcagcctgctgcaggaaggtcagcgcaaagcaggtgcagcagtgaccaccgaaaccggtgtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatttatcgccgccgcggttttctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgccgattgaaaacagcagcccgaacccgcgcctgattaac

SEQ ID NO: 115 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 116 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 117 - protein, 660 aa, synthetic construct
TWLSDFPQAWAETGGVGLAVRQAPLIIPLKATSTPVSIKQYPMSQRARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 118 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 119 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTSDYRPVQDLREVNKRVEDIRPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 120 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccagcgattatcgcccggtgcaggatctgcgcgaagtgaacaaacgcgtggaagatattcgcccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 121 - protein, 660 aa, synthetic construct
TWLSDFPQAWAETGGVGLAVRQAPLIIPLKATSTPVSIKQYPMSQRARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 122 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagcgcgcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 123 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSVEARGNRMAHQAARRAAITETPDTSTLPIENSSPNSRLIN

SEQ ID NO: 124 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 125 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTNSRYAFATAHIQGEIYRRRGFLTSKGKEIKNKDEILALLKALFLPKRLSIIHCPGHQKGYSAEARVNRMADQAARKAAITETPDTSTLPIENSSPNSRLIN

SEQ ID NO: 126 - DNA, 1980 bp, synthetic construct
  source 1..1980: mol_type = other DNA
acctggctgagcgattttccgcaagcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgaagaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgttaaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtactctgtttaactggggtccggatcagcagaaagcatatcaggaaattaaacaggcactgctgaccgcaccggcactgggtctgccggatctgaccaaaccttttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggttgcagcaattgcagtgctgaccaaagatgcaggtaaactgaccatgggtcagcctctggtgattggtgcaccgcatgcagtggaagcactggtgaaacagccggcaggtcgctggctgagcaaagcacgcatgacccattatcaggcactgttactggataccgatcgcgtgcagtttggtccggtggtggcactgaacccggcaaccctgctgccgctgccggaagaaggtctgcagcataactgcctggatattctggcagaagcacatggtacccgcccggatctgactgatcagccgctgcctgatgcagatcatacctggtataccaacggtagcagcctgttacaggaaggtcagcgtaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcagggtgaaatctatcgccgccgcggtttcctgaccagcaaaggtaaagaaattaaaaacaaagatgaaattctggcactgctgaaagcactgtttctgccgaaacgcctgagcattattcattgcccgggtcatcagaaaggttatagcgcagaagcacgcgtgaaccgcatggcagatcaggcagcacgcaaagcagcaattaccgaaaccccggataccagcaccctgccgattgaaaacagcagcccgaacagccgcctgattaac

SEQ ID NO: 127 - protein, 660 aa, synthetic construct
TWLSDFPQAWAETGGFGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARDNRMAHQAARRAAITETPDTSTLPIENSSPNSRLIN

SEQ ID NO: 128 - DNA, 1980 bp, synthetic construct
  source 1..1980: mol_type = other DNA
acctggctgagcgattttccgcaggcatgggcagaaaccggtggttttggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcgataaccgcatggcacatcaggcagcacgccgcgcagcaattaccgaaaccccggataccagcaccctgccgattgaaaacagcagcccgaacagccgcctgattaac

SEQ ID NO: 129 - protein, 660 aa, synthetic construct
TWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEVKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIQGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 130 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaagtgaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcagggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 131 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLIN

SEQ ID NO: 132 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaac

SEQ ID NO: 133 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 134 - DNA, 1980 bp, synthetic construct
  source 1..1980: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgagccagccgctggtgattaaagcaccgcatgcagtggaagcactggtgaaacagccggcagaacgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 135 - protein, 631 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYGGGGSVTVKFKYKGEELEVDISKIKKVWRVGKMISFTYDDNGKTGRGAVSEKDAPKELLQMLEKSGKK

SEQ ID NO: 136 - DNA, 1893 bp, synthetic construct
  source 1..1893: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 137 - protein, 543 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKK

SEQ ID NO: 138 - DNA, 1629 bp, synthetic construct
  source 1..1629: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatgggacgggaggaggcggagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 139 - protein, 560 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPGGGGSVTVKFKYKGEELEVDISKIKKVWRVGKMISFTYDDNGKTGRGAVSEKDAPKELLQMLEKSGKK

SEQ ID NO: 140 - DNA, 1680 bp, synthetic construct
  source 1..1680: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 141 - protein, 660 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTSDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFVEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 142 - DNA, 1980 bp, synthetic construct
  source 1..1980: mol_type = other DNA
acctggctgagcgattttccgcaagcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccagcgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttgtggaagcactgcgccgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgttaaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtactctgtttaactggggtccggatcagcagaaagcatatcaggaaattaaacaggcactgctgaccgcaccggcactgggtctgccggatctgaccaaaccttttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggttgcagcaattgcagtgctgaccaaagatgcaggtaaactgaccatgggtcagcctctggtgattggtgcaccgcatgcagtggaagcattagtgaaacagccggcaggtcgctggctgagcaaagcacgcatgacccattatcaggcactgttactggataccgatcgcgtgcagtttggtccggtggtggcactgaacccggcaaccctgctgccgctgccggaagaaggtctgcagcataactgcctggatattctggcagaagcacatggtacccgcccggatctgactgatcagccgctgcctgatgcagatcatacctggtataccaacggtagcagcctgttacaggaaggtcagcgtaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcatggtgaaatctatcgccgccgcggttggctgaccagcaaaggtaaagaaattaaaaacaaagatgagatcctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaac

SEQ ID NO: 143 - protein, 660 aa, synthetic construct
TWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 144 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgatgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 145 - protein, 660 aa, synthetic construct; identical to SEQ ID NO: 55

SEQ ID NO: 146 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggcgcaccgcatgcagtcgaggcccttgttaagcaaccggcaggaagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcgcacggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatccattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 147 - protein, 660 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 148 - DNA, 1980 bp, synthetic construct
  source 1..1980: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggtgcaccgcatgcagtcgaggcccttgttaagcaaccgccggatagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggcggtgggcactcgcccggatctgacggaccagccgctgccggacgccgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatctattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaat

SEQ ID NO: 149 - protein, 728 aa, synthetic construct
TWLSDFPQAWAETGGVGLAVRQAPLIIPLKATSTPVSIKQYPISQKARLGIKPHIQRLLDQGILVPCRSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEVKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIQGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 150 - DNA, 2184 bp, synthetic construct
  source 1..2184: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgattagccagaaagcacgcctgggtattaaaccgcatattcagcgcctgctggatcagggtattctggtgccgtgccgcagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaagtgaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccaccgaaaccgaagtgatttgggcaaaagcactgccggcaggtaccagcgcacagcgcgcagaactgattgcactgacccaggcactgaaaatggcagaaggtaaaaaactgaacgtgtataccgatagccgctatgcatttgcaaccgcacatattcagggtgaaatttatcgccgccgcggttggctgaccagcaaaggtaaagaaattaaaaacaaagatgaaattctggcactgctgaaagcactgtttctgccgaaacgcctgagcattattcattgcccgggtcatcagaaaggtcatagcgcagaagcacgcggtaaccgcatggcagatcaggcagcacgcaaagcagcaattaccgaaaccccggataccagcaccctgctgattgaaaacagcagcccgaacagccgcctgattaacggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 151 - protein, 728 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLPKRLSIIHCPGHQKGHSVEARGNRMAHQAARRAAITETPDTSTLPIENSSPNSRLINGGGGSVTVKFKYKGEELEVDISKIKKVWRVGKMISFTYDDNGKTGRGAVSEKDAPKELLQMLEKSGKK

SEQ ID NO: 152 - DNA, 2184 bp, synthetic construct
  source 1..2184: mol_type = other DNA
acctggctgagcgattttccgcaggcatgggcagaaaccggtggtgtgggtctggcagtgcgccaggcaccgctgattattccgctgaaagcaaccagcaccccggtgagcattaaacagtatccgatgagccagaaagcacgcctgggtattaaaccgcatattcagcgcctgctggatcagggtattctggtgccgtgccagagcccgtggaacaccccgctgctgccggtgaaaaaaccgggtaccaacgattatcgcccggtgcaggatctgcgcgaagtgaacaaacgcgtggaagatattcatccgaccgtgccgaacccgtataacctgctgagcggtccgccgccgagccatcagtggtataccgtgctggatctgaaagatgcatttttttgcctgcgcctgcatccgaccagccagccgctgtttgcatttgaatggcgcgatccggaaatgggtattagcggtcagctgacctggacccgcctgccgcagggttttaaaaacagcccgaccctgtttaacgaagcactgcatcgcgatctggcagattttcgcattcagcatccggatctgattctgctgcagtatgtggatgatctgctgctggcagcaaccagcgaactggattgccagcagggtacccgcgcactgctgcagaccctgggtaacctgggttatcgcgcaagcgcaaaaaaagcacagatttgccagaaacaggtgaaatatctgggttatctgctgaaagaaggtcagcgctggctgaccgaagcacgcaaagaaaccgtgatgggtcagccgaccccgaaaaccccgcgccagctgcgccgctttctgggtaaagcaggtttttgccgcctgtttattccgggttttgcagaaatggcagcaccgctgtatccgctgaccaaaccgggtaccctgtttaactggggtccggatcagcagaaagcatatcaggaaattaaacaggcactgctgaccgcaccggcactgggtctgccggatctgaccaaaccgtttgaactgtttgtggatgaaaaacagggttatgcaaaaggtgtgctgacccagaaactgggtccgtggcgccgcccggtggcatatctgagcaaaaaactggatccggtggcagcaggttggccgccgtgcctgcgcatggtggcagcaattgcagtgctgaccaaagatgcaggtaaactgaccatgggtcagccgctggtgattggtgcaccgcatgcagtggaagcactggtgaaacagccggcaggtcgctggctgagcaaagcacgcatgacccattatcaggcactgctgctggataccgatcgcgtgcagtttggtccggtggtggcactgaacccggcaaccctgctgccgctgccggaagaaggtctgcagcataactgcctggatattctggcagaagcacatggtacccgcccggatctgaccgatcagccgctgccggatgcagatcatacctggtataccaacggtagcagcctgctgcaggaaggtcagcgcaaagcaggtgcagcagtgaccgatgaaaccgaagtgatttgggcaaaagcactgccggcaggtaccagcgcacagcgcgcagaactgattgcactgacccaggcactgaaaatggcagaaggtaaaaaactgaacgtgtataccaacagccgctatgcatttgcaaccgcacatattcatggtgaaatttatcgccgccgcggttggctgaccagcggtggtaaagaaattaaaaacaaagatgaaattctggcactgctgaaagcactgtttctgccgaaacgcctgagcattattcattgcccgggtcatcagaaaggtcatagcgtggaagcacgcggtaaccgcatggcacatcaggcagcacgccgcgcagcaattaccgaaaccccggataccagcaccctgccgattgaaaacagcagcccgaacagccgcctgattaacggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 153 - protein, 728 aa, synthetic construct
TWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 154 - DNA, 2184 bp, synthetic construct
  source 1..2184: mol_type = other DNA
acctggctgtctgactttccgcaggcttgggcagagaccggggggatgggtttagcggtacggcaggcaccactgattataccgctgaaagcaacatcgactcctgtctcgataaagcaataccctatgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 155 - protein, 728 aa, synthetic construct; identical to SEQ ID NO: 20

SEQ ID NO: 156 - DNA, 2184 bp, synthetic construct
  source 1..2184: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 157 - protein, 728 aa, synthetic construct
TWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 158 - DNA, 2184 bp, synthetic construct
  source 1..2184: mol_type = other DNA
acctggctgtctgactttccgcaggcttgggcagagaccgggggggtgggtttagcggtacggcaggcaccactgattataccgctgaaagcaacatcgactcctgtctcgataaagcaataccctatgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaataaggatgaaattctggcacttctgaaagcgttatttctgcctaaacgcttaagtatcatacactgtcctggtcaccaaaaaggtcatagtgctgaagcgcgcgggaaccgcatggccgatcaggccgcccgtaaagctgcaattaccgaaacccccgatacatctacgctgctgattgaaaactctagcccaaatagcagactgattaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 159 - protein, 574 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLGGGGSVTVKFKYKGEELEVDISKIKKVWRVGKMISFTYDDNGKTGRGAVSEKDAPKELLQMLEKSGKK

SEQ ID NO: 160 - DNA, 1722 bp, synthetic construct
  source 1..1722: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 161 - protein, 579 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRGGGGSVTVKFKYKGEELEVDISKIKKVWRVGKMISFTYDDNGKTGRGAVSEKDAPKELLQMLEKSGKK

SEQ ID NO: 162 - DNA, 1737 bp, synthetic construct
  source 1..1737: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 163 - protein, 605 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRGGGGSVTVKFKYKGEELEVDISKIKKVWRVGKMISFTYDDNGKTGRGAVSEKDAPKELLQMLEKSGKK

SEQ ID NO: 164 - DNA, 1815 bp, synthetic construct
  source 1..1815: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 165 - skipped

SEQ ID NO: 166 - protein, 658 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNGGGGSVTVKFKYKGEELEVDISKIKKVWRVGKMISFTYDDNGKTGRGAVSEKDAPKELLQMLEKSGKK

SEQ ID NO: 167 - DNA, 1974 bp, synthetic construct
  source 1..1974: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaagaaataaaaaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 168 - protein, 654 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKGGGGSVTVKFKYKGEELEVDISKIKKVWRVGKMISFTYDDNGKTGRGAVSEKDAPKELLQMLEKSGKK

SEQ ID NO: 169 - DNA, 1962 bp, synthetic construct
  source 1..1962: mol_type = other DNA
acctggctgtctgactttccgcaggcttgggcagagaccggggggatgggtttagcggtacggcaggcaccactgattataccgctgaaagcaacatcgactcctgtctcgataaagcaataccctctgtcccaaaaggctcgcttaggcattaagccacatatccagcgtctgctagatcaaggtattctcgttccttgtcagtctccgtggaataccccgttgctgccagttaagaaaccgggcacaaacgactatcgcccggttcaggacctgcgagaggtaaacaaacgtgtggaagatatccatccgacagtgcccaacccctacaatctgttgtctggtcctcctccaagccaccagtggtacacagtcctggacttaaaagatgcattcttctgcctccgtctgcatccaactagccaacctctgttcgcgtttgaatggcgggacccggaaatgggaattagcggtcagctcacctggacaagactcccgcaaggattcaagaactccccgactctgtttaatgaagcgctgcaccgcgacctggccgactttagaatacagcacccagatcttatccttctgcagtacgtcgatgatcttttgctggccgcaacaagcgagctggattgtcaacaggggacgcgtgctcttttacagaccctgggtaatctcggttaccgcgcgagcgcaaaaaaagcccaaatatgtcaaaaacaggtaaagtatctgggatacctgcttaaagagggtcagcgatggctcactgaggcacgtaaagagaccgttatgggacagccgacaccaaaaacaccgcgacaactgcggcggtttctgggaaaagcgggtttttgtcgattatttatccctgggtttgccgaaatggcggcacctttatatccactcactaagccaggcacactgtttaattggggaccggatcaacagaaggcgtaccaggagataaaacaagcgctgttaaccgcccccgctttaggtctgcctgacctgactaagccgttcgaactgtttgtagatgaaaaacaaggttacgcgaaaggtgttttaactcagaaacttgggccatggcgtcgtcctgtcgcgtatctctctaaaaaacttgacccggttgcggctggctggcctccatgcctgagaatggtggcagctatagcagtcctgactaaagatgccggtaaattgaccatgggtcagccacttgtgatcggtgctccccacgcggtggaggcgttggttaaacagcctgccgggcgttggttgtcgaaagctcgcatgacacattatcaggccttattacttgatacggacagagttcagtttggaccggtagtcgccctgaacccggccacgttacttccgctgccagaagaaggtttacagcataattgtctcgatatccttgccgaggctcatggaacccgcccagaccttactgaccaaccacttcccgatgctgaccacacctggtataccaatggttcatctctgctacaggagggtcaacgcaaagcgggtgcagcagttaccactgaaactgaggttatttgggcgaaagcgctgccagcaggaacgagcgctcagagagccgagttaattgcgttaacacaggcgctgaagatggctgaagggaaaaaactgaacgtctataccgacagccgctatgcatttgctacagcccatattcatggagaaatttatcgccggcgaggttggctcacgtcgaaaggtaaaggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 170 - protein, 728 aa, synthetic construct
TWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLINGGGGSVTVKFKYKGEELEVDISKIKKVWRVGKMISFTYDDNGKTGRGAVSEKDAPKELLQMLEKSGKK

SEQ ID NO: 171 - DNA, 2184 bp, synthetic construct
  source 1..2184: mol_type = other DNA
acttggctgtctgatttccctcaggcgtgggccgaaacgggtggcatgggtctggcagtgcgtcaggcaccgctgattattccgctgaaagcgacgtcgaccccggtgagcatcaagcaatatccgctgtcccaaaaggcgcgcttaggtattaagccgcacattcagcgtctgctggatcaaggtattctggttccgtgtcagagcccgtggaataccccgcttctcccggtgaagaaaccgggcacgaacgattaccgtccagtccaagacttgcgcgaagttaacaagcgcgttgaagatattcacccgaccgtcccgaacccgtacaatctgctgagcggtccgccgccaagccaccaatggtacaccgtgctggatctgaaagatgctttcttctgtctgcgtctgcacccaaccagccagcctctgtttgcatttgagtggcgtgaccctgagatgggtattagcggccagctgacgtggacccgcctgccgcaaggttttaagaattcccctacgctgtttaacgaagcgctgcaccgtgacctggcggatttccgtatccagcacccggacctgatcttgctgcagtacgttgatgacctgttgctggcggcgacgagcgagctggattgccaacagggcacccgtgcgctgttgcagaccttgggtaacctgggttatcgcgctagcgcgaagaaagcgcagatttgccaaaaacaagttaagtatctgggctacctgttaaaggaaggccaacgttggctgaccgaagcccgcaaagaaactgtcatgggtcagccgaccccgaaaacgccacgccaactgcgtaggttcttgggcaaagcgggtttctgccgcctgttcatcccgggctttgccgaaatggcagccccgctgtatccgttgaccaagccgggcaccctgttcaactggggtccggaccagcagaaagcgtaccaagaaattaaacaagcactgctgacggcaccggcgctgggtctgccggacctgaccaagccgtttgagctgttcgtggatgagaagcaaggttacgcgaagggcgtgttgacccagaaattgggtccgtggcgtcgtccggttgcatacctgtccaagaaactggacccggttgctgctggttggccgccttgcctgcgcatggttgccgctatcgcggtgctgactaaagacgcgggtaagctgacgatgggtcaaccgctggtgatcggtgcaccgcatgcagtcgaggcccttgttaagcaaccgccggatagatggctgagcaaggcgcgtatgacgcattaccaggcactgctgttggacaccgatcgtgtgcagtttggcccggtcgttgcgctcaacccggcgaccctgctgccgctcccggaagaaggcttgcagcacaactgtttggacatcctggcagaggctgtgggaacccgcccagaccttactgaccaaccacttcccgatgctgatcatacctggtatacgaatggtagcagcctgttgcaagagggtcagcgtaaggccggtgccgcggtcaccaccgagactgaagtgatttgggctaaagcattgcctgcgggtaccagcgcgcagcgtgccgagctgatcgcactgacccaagcgctgaaaatggctgagggtaagaaactgaatgtgtacacggatagccgttatgcctttgcgaccgcccacattcacggcgagatctatcgccgtcgcggctggctgacgtccaaaggcaaagagatcaagaataaagacgaaattctggcgctgctgaaagcgctgttcctgccgaaacgtctgtcgatcatctattgcccgggtcaccagaaaggccacagcgcagaggcgcgtggtaatcgcatggctgaccaggctgcgcgtaaagccgcaattaccgaaaccccggacaccagcacgctgctgatcgagaatagcagcccgaacagccgtctgatcaatggtggaggtgggtcagtaacagtaaaatttaaatataaaggtgaagagttggaggttgatatttcaaagataaaaaaagtgtggagagtcggtaaaatgataagttttacgtatgatgacaacgggaaaaccggtcggggcgccgtgtccgaaaaagacgctccgaaggaactactccaaatgcttgagaaaagtggaaaaaaa

SEQ ID NO: 172 - protein, 671 aa, synthetic construct; identical to SEQ ID NO: 7

SEQ ID NO: 173 - RNA, 1315 nt, unidentified
  source 1..1315: mol_type = unassigned RNA
agagggaaagcagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 174 - DNA, 29 bp, synthetic construct
  source 1..29: mol_type = other DNA
  modified_base 1: mod_base = OTHER; note = 5' 6-fluorescein thymidine
tggttgagcacagggtactttattgatgg

SEQ ID NO: 175 - DNA, 30 bp, synthetic construct; identical to SEQ ID NO: 11
  source 1..30: mol_type = other DNA
  misc_feature 1..27: note = DNA
  misc_feature 28..30: note = RNA

SEQ ID NO: 176 - DNA, 29 bp, synthetic construct
  source 1..29: mol_type = other DNA
tggttgagcacagggtactttattgatgg

SEQ ID NO: 177 - protein, 5 aa, synthetic construct
HHHHH

SEQ ID NO: 178 - protein, 671 aa, Moloney Murine Leukemia Virus; identical to SEQ ID NO: 7

SEQ ID NO: 179 - protein, 661 aa, synthetic construct; identical to SEQ ID NO: 1

SEQ ID NO: 180 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 181 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 182 - protein, 661 aa, synthetic construct
MTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 183 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 184 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 185 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 186 - protein, 671 aa, synthetic construct; identical to SEQ ID NO: 28

SEQ ID NO: 187 - protein, 671 aa, synthetic construct; identical to SEQ ID NO: 29

SEQ ID NO: 188 - protein, 671 aa, synthetic construct; identical to SEQ ID NO: 30

SEQ ID NO: 189 - protein, 671 aa, synthetic construct; identical to SEQ ID NO: 31

SEQ ID NO: 190 - protein, 671 aa, synthetic construct; identical to SEQ ID NO: 32

SEQ ID NO: 191 - protein, 671 aa, synthetic construct; identical to SEQ ID NO: 33

SEQ ID NO: 192 - protein, 671 aa, synthetic construct; identical to SEQ ID NO: 34

SEQ ID NO: 193 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 194 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 195 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 196 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 197 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 198 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPADRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 199 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 200 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 201 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLLIENSSPNSRLIN

SEQ ID NO: 202 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 203 - protein, 661 aa, synthetic construct
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 204 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLLIENSSPNSRLIN

SEQ ID NO: 205 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLLIENSSPNSRLIN

SEQ ID NO: 206 - protein, 661 aa, synthetic construct
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 207 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 208 - protein, 661 aa, synthetic construct
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSPNSRLIN

SEQ ID NO: 209 - protein, 60 aa, synthetic construct
  VARIANT 1..20: note = This region may encompass 1-20 residues
  VARIANT 21..40: note = This region may encompass 1-20 residues
  VARIANT 41..60: note = This region may encompass 1-20 residues
GGGGGGGGGGGGGGGGGGGGSSSSSSSSSSSSSSSSSSSSGGGGGGGGGGGGGGGGGGGG

SEQ ID NO: 210 - protein, 4 aa, synthetic construct
CCCC

SEQ ID NO: 211 - RNA, 11 nt, synthetic construct
  source 1..11: mol_type = other RNA
aaaaaaaaaaa

(19) United States
(12) Patent Application Publication
(10) Pub. No.: US 2024/0368567 A1
(43) Pub. Date: Nov. 7, 2024

(54) RECOMBINANT REVERSE TRANSCRIPTASE VARIANTS FOR IMPROVED PERFORMANCE

(71) Applicant: 10x Genomics, Inc., Pleasanton, CA (US)

(72) Inventors: Derek Hunter VALLEJO, San Francisco, CA (US); Sonya A. CLARK, Pleasanton, CA (US); Yufeng QIAN, Union City, CA (US); Lorita BOGHOSPOR, Lathrop, CA (US); Shankar SHASTRY, San Francisco, CA (US)

(73) Assignee: 10x Genomics, Inc., Pleasanton, CA (US)

(21) Appl. No.: 18/744,254

(22) Filed: Jun. 14, 2024

Related U.S. Application Data
(63) Continuation of application No. PCT/US2022/053174, filed on Dec. 16, 2022.
(60) Provisional application No. 63/290,329, filed on Dec. 16, 2021.
(60) Provisional application No. 63/421,919, filed on Nov. 2, 2022.

(30) Foreign Application Priority Data
Apr. 29, 2022 (WO) PCT/US2022/027024
Jun. 13, 2022 (WO) PCT/US2022/033199

(51) Int. Cl. C12N 9/12; C12N 9/10

(52) U.S. Cl. CPC C12N 9/1276; C12N 9/1096; C12Y 207/07049

(57) ABSTRACT

The disclosure provides recombinant reverse transcriptases comprising one or more DNA binding domains conjugated to an engineered reversed transcriptase that have been modified to exhibit one or more altered reverse transcriptase related activities such as but not limited to altered template switching efficiency, altered transcription efficiency or both. The disclosure further provides compositions and kits comprising the recombinant reverse transcriptase enzymes and methods of producing, amplifying or sequencing nucleic acid molecules using these fusion reverse transcriptase enzymes.

### CROSS-REFERENCE TO RELATED PATENT APPLICATIONS

This application is a bypass Continuation of International Patent Application No. PCT/US2022/053174, filed Dec. 16, 2022, which claims priority from U.S. Provisional Application No. 63/290,329, filed Dec. 16, 2021, and U.S. Provisional Application No. 63/421,919, filed Nov. 2, 2022; this application also claims foreign priority from International Application PCT/US2022/027024, filed Apr. 29, 2022, and International Application No. PCT/US2022/033199, filed Jun. 13, 2022, the contents of each application are incorporated herein by reference in their entireties.

### SEQUENCE LISTING

The present application contains a Sequence Listing which is hereby incorporated by reference in its entirety. Said Sequence Listing xml file was created on Jan. 25, 2023, is 384 bytes in size, and is named 131488_0214_SL.xml.

### FIELD OF THE INVENTION

The present invention relates to the field of protein engineering, particularly development of recombinant reverse transcriptase variants that exhibit one or more improved properties of interest.

### BACKGROUND

One of the major challenges in cDNA synthesis reactions is interference in cDNA synthesis from RNA secondary structures. While a higher reaction temperature can remove secondary structure from the template RNA, elevated temperatures typically lead to lower reverse-transcriptase (RT) enzyme activity without the use of an efficient, thermostable RT enzyme. Wild-type (WT) Moloney Murine Leukemia Virus (MMLV) reverse-transcriptase is an RT enzyme that is typically inactivated at higher temperatures. RT enzyme activity can also be reduced by inhibitors, such as inhibitors that might be present in cell lysates, associated reagents and fixation reagents. Low volume reactions can also negatively impact wild-type (WT) MMLV reverse-transcriptase activity. Specific residues of MMLV have been linked to thermostability. For example, M39V, M66L, E69K, E302R, T306K, W313F, L/K435G, and N454K sites have been shown to improve thermostability, see Arezi et al (2009) Nucleic Acids Res. 37(2):473-481, U.S. Pat. No. 7,078,208, and Baranauskas et al 2012 Prot. Engineering 25(10): 657-668, which are hereby incorporated by reference in their entireties.

A wide variety of different applications of single cell processing and analysis methods and systems are known in the art, including analysis of specific individual cells, analysis of different cell types within populations of differing cell types, analysis and characterization of large populations of cells for environmental, human health, epidemiological forensic, or any of a wide variety of different applications. However, reverse transcription of mRNA from a single cell can be inhibited when the reaction volume is less than about 1 nL. Overcoming this reaction volume effect has been a challenge.

RT enzymes were initially found in retroviruses such as Moloney murine leukemia virus (MMLV)). It is now clear that RTs are present in other microorganisms, including transposable elements, where RTs are responsible for converting an RNA genome of these organisms into DNA to facilitate the integration of the microorganisms into a host's chromosome. Generally, RTs are mesophilic enzymes that function best at moderate temperatures ranging from 20° C. to 45° C. The mesophilic nature of RTs is problematic for in vitro amplification reactions because RNAs tend to adopt stable secondary structures at lower temperatures resulting in inefficient reverse transcription reactions at these low to moderate temperatures. In addition to the RNA secondary structures, RT reactions and amplification reactions also fail because biological samples from which nucleic acids are extracted often contain additional compounds that are inhibitory to reverse transcription and/or amplification reactions. This inhibition is particularly problematic when the volume of an amplification reaction is very small (e.g., nanoliter), such as in single cell profiling reactions and additional methods where small reaction volumes are preferential.

Accordingly, there is a need for improved reverse transcriptases with improved properties, particularly for use in small reaction volumes, such as improved efficiency, processivity, thermoreactivity, and/or thermostability. The present disclosure addresses this need.

### SUMMARY

One aspect of the present disclosure provides an engineered fusion reverse transcriptase comprising: (a) at least one DNA binding domain selected from a DNA binding domain from an archaeal DNA binding protein or a single-stranded DNA binding domain; and (b) an engineered reverse transcriptase having an amino acid sequence variation that is: (i) at least about 90% identical to SEQ ID NO: 1 or 143; (ii) 90-99.99% identical to SEQ ID NO: 1 or 143, 92-99.99% identical to SEQ ID NO: 1 or 143, 93-99.99% identical to SEQ ID NO: 1 or 143, 94-99.99% identical to SEQ ID NO: 1 or 143, 95-99.99% identical to SEQ ID NO: 1 or 143, 96-99.99% identical to SEQ ID NO: 1 or 143, 97-99.99% identical to SEQ ID NO: 1 or 143, or 98-99.99% identical to SEQ ID NO: 1 or 143; or (iii) 90%, 91%, 92%, 93%, 94%, 95%, 96%, 97%, 98%, 99% or 99.5% identical to SEQ ID NO: 1 or 143.

In another aspect the present disclosure provides a recombinant reverse transcriptase variant comprising an amino acid sequence variation that is: (i) at least about 90% identical to SEQ ID NO: 1 or 143; (ii) 90-99.99% identical to SEQ ID NO: 1 or 143, 92-99.99% identical to SEQ ID NO: 1 or 143, 93-99.99% identical to SEQ ID NO: 1 or 143, 94-99.99% identical to SEQ ID NO: 1 or 143, 95-99.99% identical to SEQ ID NO: 1 or 143, 96-99.99% identical to SEQ ID NO: 1 or 143, 97-99.99% identical to SEQ ID NO: 1 or 143, or 98-99.99% identical to SEQ ID NO: 1 or 143; or (iii) 90%, 91%, 92%, 93%, 94%, 95%, 96%, 97%, 98%, 99% or 99.5% identical to SEQ ID NO: 1 or 143

In some embodiments, the amino acid variation(s) are at any one position or combination thereof as identified in an alignment of SEQ ID NO: 1 or 143 to any one of the RT polypeptide sequences in Table 4, Table 5, or Table 6.

In non-limiting embodiments, the recombinant fusion RT or recombinant RT is any one of the sequences disclosed in Table 4, Table 5 or Table 6. In non-limiting embodiments, the engineered RT variants of the invention comprise a M39V, M66I, Q91R, I347V, H594Q, or a combination thereof in the RT backbone of SEQ ID NO: 143 or SEQ ID NO: 7. In non-limiting embodiments, the engineered RT variant comprises: M39V, M66I, Q91R, 1347V, and H594Q (SEQ ID NO: 129, SOLD 034).

In some embodiments, the engineered RT variant comprises: M39V, M66I, Q91R, I347V, H594Q, or a combination thereof, and optionally M39V, M66I, Q91R, I347V, H594Q, or the combination thereof (substituted) in the RT sequence of SEQ ID NO: 143 (SEQ ID NO: 129, SOLD 034).

In some embodiments, the engineered RT variant comprises: M39V, T542D, D583N, E607G, A644V, D653H, K658R, L671P, or a combination thereof, and optionally M39V, T542D, D583N, E607G, A644V, D653H, K658R, L671P, or the combination thereof substituted in the RT sequence of SEQ ID NO: 143 (or SEQ ID NO: 7) (SEQ ID NO: 111, SOLD 025).

In non-limiting embodiments, the engineered RT variants of the invention comprise M39V, T542D, D583N, E607G, A644V, D653H, K658R, L671P, or a combination thereof in the RT backbone of SEQ ID NO: 143 or SEQ ID NO: 7. In some embodiments, 42B comprises E607K. In non-limiting embodiments, the engineered RT variant comprises: M39V, T542D, D583N, E607G, A644V, D653H, K658R, and L671P (SEQ ID NO: 111, SOLD 025).

In non-limiting embodiments, the recombinant RT is any one of the RTs listed in Table 5. In non-limiting embodiments, the recombinant RT is any one of 42B_V, 42B_L (SEQ ID NO: 145), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), and SOLD 035 (SEQ ID NO: 131). In non-limiting embodiments, the recombinant fusion RT is any one of the RTs listed in Table 5, fused to Sto7. In non-limiting embodiments, the recombinant fusion RT is any one of 42B (SEQ ID NO: 1, 143, or 179), 42B_L (SEQ ID NO: 145), 42B_V, SOLD 025 (SEQ ID NO: 111), SOLD 031 (SEQ ID NO: 123), SOLD 033 (SEQ ID NO: 127), SOLD 034 (SEQ ID NO: 129), fused to Sto7.

In some embodiments, the engineered fusion reverse transcriptase exhibits an altered reverse transcriptase-related activity as compared to a reverse transcriptase having the amino acid sequence set forth in SEQ ID NO: 1. In that embodiment, the altered reverse transcriptase-related activity is selected from increased template switching (TS) efficiency, higher end-to-end template jumping/switching, increased processivity efficiency, increased binding affinity, increased transcription efficiency, increased chemical tolerance, improved ability to yield mitochondrial unique molecular identifier (UMI) counts, improved ability to yield ribosomal unique molecular identifier (UMI) counts, longer shelf life, higher strand displacement, increased thermostability, improved thermoreactivity, or any combination thereof.

In some embodiments, the altered reverse transcriptase-related activity comprises at least two or more of increased template switching (TS) efficiency, increased processivity efficiency, increased binding affinity, increased transcription efficiency, increased chemical tolerance, improved ability to yield mitochondrial unique molecular identifier (UMI) counts, longer shelf life, higher strand displacement, higher end-to-end template jumping, or improved ability to yield ribosomal unique molecular identifier (UMI) counts.

In some embodiments, the altered reverse transcriptase-related activity is an increased TS efficiency as compared to the TS efficiency of a reverse transcriptase having the amino acid sequence set forth in SEQ ID NO: 1.

In some embodiments, the increased TS efficiency is: (a) from 0.1× to 10×, from 1× to 10×, from 0.25× to 7.5×, from 0.5× to 5×, or from 1× to 4× greater than the template switching activity of a reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the template switching activity of a reverse transcriptase having the amino acid sequence set forth in SEQ ID NO: 1; or (c) at least 0.5× greater than the TS efficiency exhibited by an engineered reverse transcriptase having the amino acid sequence set forth in SEQ ID NO:1.

In some embodiments, the altered reverse transcriptase-related activity is an increased processivity efficiency during reverse transcription as compared to the processivity efficiency during reverse transcription of a reverse transcriptase having the amino acid sequence set forth in SEQ ID NO: 1. In that embodiment, the increased processivity efficiency during reverse transcription is: (a) from 0.1× to 10×, from 1× to 10×, from 0.25× to 7.5×, from 0.5× to 5×, or from 1× to 4× greater than the processivity efficiency during reverse transcription exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the processivity efficiency during reverse transcription exhibited by an engineered reverse transcriptase having the amino acid sequence set forth in SEQ ID NO: 1; or (c) at least 0.5× greater than the processivity efficiency during reverse transcription exhibited by an engineered reverse transcriptase having the amino acid sequence set forth in SEQ ID NO: 1.

In some embodiments, the altered reverse transcriptase-related activity is an increased binding affinity during reverse transcription as compared to the binding affinity during reverse transcription of a reverse transcriptase having the amino acid sequence set forth in SEQ ID NO: 1. In that embodiment, the increased DNA binding affinity during reverse transcription is: (a) from 0.1× to 10×, from 1× to 10×, from 0.25× to 7.5×, from 0.5× to 5×, or from 1× to 4× greater than the DNA binding affinity during reverse transcription exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the DNA binding affinity during reverse transcription exhibited by an engineered reverse transcriptase having the amino acid sequence set forth in SEQ ID NO: 1; or (c) at least 0.5× greater than the DNA binding affinity during reverse transcription exhibited by an engineered reverse transcriptase having the amino acid sequence set forth in SEQ ID NO: 1.

In some embodiments, the altered reverse transcriptase-related activity is an increased transcription efficiency during reverse transcription as compared to the transcription efficiency during reverse transcription of a reverse transcriptase having the amino acid sequence set forth in SEQ ID NO: 1. In that embodiment, the increased transcription efficiency during reverse transcription is: (a) from 0.1× to 10×, from 1× to 10×, from 0.25× to 7.5×, from 0.5× to 5×, or from 1× to 4× greater than the transcription efficiency during reverse transcription exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the transcription efficiency during reverse transcription exhibited by an engineered reverse transcriptase having the amino acid sequence set forth in SEQ ID NO: 1; or (c) at least 0.5× greater than the transcription efficiency during reverse transcription exhibited by an engineered reverse transcriptase having the amino acid sequence set forth in SEQ ID NO: 1.

In some embodiments, the altered reverse transcriptase-related activity is an increased chemical tolerance during reverse transcription as compared to the chemical tolerance during reverse transcription of a reverse transcriptase having the amino acid sequence set forth in SEQ ID NO: 1. In that embodiment, the increased chemical tolerance during reverse transcription is: (a) from 0.1× to 10×, from 1× to 10×, from 0.25× to 7.5×, from 0.5× to 5×, or from 1× to 4× greater than the chemical tolerance during reverse transcription exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the chemical tolerance during reverse transcription exhibited by an engineered reverse transcriptase having the amino acid sequence set forth in SEQ ID NO: 1; or (c) at least 0.5× greater than the chemical tolerance during reverse transcription exhibited by an engineered reverse transcriptase having the amino acid sequence set forth in SEQ ID NO: 1.

In some embodiments, the altered reverse transcriptase-related activity is an improved ability to yield mitochondrial UMI counts as compared to a reverse transcriptase having the amino acid sequence set forth in SEQ ID NO: 1. In that embodiment, the improved ability to yield mitochondrial UMI counts is: (a) from 0.1× to 10×, from 1× to 10×, from 0.25× to 7.5×, from 0.5× to 5×, or from 1× to 4× greater than the ability to yield mitochondrial UMI counts exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the ability to yield mitochondrial UMI counts exhibited by an engineered reverse transcriptase having the amino acid sequence set forth in SEQ ID NO: 1; or (c) at least 0.5× greater than the ability to yield mitochondrial UMI counts exhibited by an engineered reverse transcriptase having the amino acid sequence set forth in SEQ ID NO: 1.

In some embodiments, the altered reverse transcriptase-related activity is an improved thermostability as compared to a reverse transcriptase having the amino acid sequence set forth in SEQ ID NO: 1. In that embodiment, the improved thermostability is: (a) from 0.1× to 10×, from 1× to 10×, from 0.25× to 7.5×, from 0.5× to 5×, or from 1× to 4× greater than the thermostability exhibited by an engineered reverse transcriptase having the amino acid sequence set forth in SEQ ID NO: 1; (b) at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or 10× greater than the thermostability exhibited by an engineered reverse transcriptase having the amino acid sequence set forth in SEQ ID NO: 1; or (c) at least 0.5× greater than the thermostability exhibited by an engineered reverse transcriptase having the amino acid sequence set forth in SEQ ID NO: 1. In some embodiments, the engineered fusion reverse transcriptase comprises the amino acid sequence of SEQ ID NO: 20 or SEQ ID NO: 111, SEQ ID NO: 129.

In some embodiments, the engineered reverse transcriptase comprises the combination of the following amino acid substitutions in SEQ ID NO:7: (a) E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or (b) E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P.

In some embodiments, the engineered reverse transcriptase comprises an amino acid sequence that is at least about 90% identical to (a) an amino acid sequence selected from: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and SEQ ID NO: 55; (b) SEQ ID NOs: 180-208; (c) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (d) an amino acid sequence listed in Table 5.

In some embodiments, the amino acid sequence of the engineered reverse transcriptase comprises E69K, L139P, D200N, E302R, T306K, W313F, T330P, N454K, H503V, D524N, L603W, E607K, and H634Y.

In some embodiments, the amino acid sequence of the engineered reverse transcriptase comprises a combination of mutations selected from: (a) M66L and L435G; (b) M39V, M66L, and L435K; (c) M39V and L435K; (d) M66L, L435G, P448A and D449G; (e) M39V, M66L, L435G, P448A and D449G; (f) M66L.

In some embodiments, the amino acid sequence of the engineered reverse transcriptase comprises E69K, L139P, D200N, E302R, T306K, W313F, T330P, L435G, P448A, D449G, N454K, D524N, L603W, and E607K; and comprises a combination of mutations selected from (a) M66L; (b) M66L and H503V; (c) M66L and H634Y; and (d) M66L, H503V, and H634Y.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P, and comprises a second combination of mutations selected from: (a) D524N, T542D, P627S, A644V, D653H, K658R mutation, and wherein the D200 mutation is a D200N mutation, the D449 mutation is a D449G, the L603 mutation is an L603W, and the E607 mutation is an E607G mutation; (b) D524N, T542D, A644V, D653H, an R650H and K658R, and wherein the D200 mutation is a D200N mutation, the D449 mutation is a D449E mutation, the L603 mutation is an L603W mutation, and the E607 mutation is an E607G mutation; (c) E545G, D583N, and H594Q, and wherein the D200 mutation is a D200N mutation, the D449 mutation is a D449G mutation, the L603 mutation is an L603F mutation, and the E607 mutation is an E607K mutation; (d) D524N, T542D, A644V, D653H, and K658R, wherein the D200 mutation is a D200N mutation, the D449 mutation is a D449E mutation, the L603 mutation is an L603W mutation, the E607 mutation is an E607G mutation; (e) H204R, D524N, T542D, P627S, D583N, A644V, D653H and K658R, wherein the D200 mutation is a D200E mutation, the D449 mutation is a D449G mutation, the L603 mutation is an L603W mutation, the E607 mutation is an E607G mutation; (f) H204R, E545G, D583N, and H594Q, wherein the D200 mutation is a D200E mutation, the D449 mutation is a D449G mutation, the L603 mutation is an L603F mutation, the E607 mutation is an E607K mutation; or (g) P47L, D524N, T542D, D583N, P627S, A644V, D653H, and K658R, wherein the D200 mutation is a D200N mutation, the D449 mutation is a D449G mutation, the L603 mutation is an L603W mutation, the E607 mutation is an E607G mutation.

In some embodiments, the at least one DNA binding domain is located at the C-terminus or at the N-terminus of the engineered fusion reverse transcriptase. In some embodiments, the DNA binding domain is: (a) an archaeal DNA binding domain from a protein selected from Sto7d, Sso7d, Sis7b, Sis7a, Ssh7b, Sto7, Aho7C, Aho7B, Aho7A, Mcu7, Mse7, Sac7e, or Sac7d; (b) Stod7; or (c) Stod7d. In some embodiments, the amino acid sequence of the DNA binding domain comprises a DNA binding domain consensus motif set forth in SEQ ID NO:2.

In some embodiments, the DNA binding domain comprises: (a) an amino acid sequence selected from SEQ ID NO: 12, 13, 16, 17, or 18; or (b) an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 12, 13, 16, 17, or 18. In some embodiments, the DNA binding domain comprises the amino acid sequence of SEQ ID NO: 12, 13, 16, 17, or 18.

In some embodiments, the DNA binding domain is a single-stranded DNA binding domain. In some embodiments, the DNA binding domain exhibits reduced RNAase activity. In that embodiment, the amino acid sequence of the DNA binding domain has been altered to reduce RNAase activity. In another embodiment, the DNA binding domain comprises a mutation selected from a K13 mutation, a K13L mutation, a D36 mutation, an N37 mutation, a V2 mutation, a D36L mutation, an insertion, or a combination thereof. In one embodiment, the DNA binding domain comprises the amino acid sequence of SEQ ID NO: 18. In some embodiments, the amino acid sequence of the engineered fusion reverse transcriptase comprises a Sto7 DNA binding domain at the C-terminus or N-terminus of the engineered fusion reverse transcriptase.

In some embodiments of the engineered fusion reverse transcriptase described herein: (a) the DNA binding domain comprises an amino acid sequence selected from SEQ ID NO: 12, 13, 16, 17, or 18; (b) the engineered reverse transcriptase comprises (i) an amino acid sequence selected from: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55; (ii) SEQ ID NOs: 180-208; (iii) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (iv) an amino acid sequence listed in Table 5; and (c) the DNA binding domain is located at the C-terminus or N-terminus of the engineered fusion reverse transcriptase.

In some embodiments, the amino acid sequence of the engineered fusion reverse transcriptase comprises: (a) an amino acid sequence selected from SEQ ID NO: 3, SEQ ID NO: 5, SEQ ID NO: 6, SEQ ID NO: 8, SEQ ID NO: 20, SEQ ID NOs: 135, 137, 139, 153, 155, 161, 163, 166, 168, 170; or an amino acid sequence listed in Table 5 or (b) an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, SEQ ID NOs: 135, 137, 139, 153, 155, 161, 163, 166, 168, 170, or an amino acid sequence listed in Table 5.

In some embodiments, the amino acid sequence of the engineered reverse transcriptase comprises at least one mutation selected from an M39V mutation or an M66L mutation, wherein the mutation is indexed to an amino acid sequence set forth in SEQ ID NO:7. In some embodiments, the engineered fusion reverse transcriptase comprises at least two DNA binding domains. In some embodiments, at least one DNA binding domain is located at the N-terminus of the engineered fusion reverse transcriptase and at least one DNA binding domain is located at the C-terminus of the engineered fusion reverse transcriptase. In some embodiments, the at least two DNA binding domains are both located at the C-terminus or N-terminus of the engineered fusion reverse transcriptase.

In some embodiments of the engineered fusion reverse transcriptase described herein: (a) the DNA binding fusion domain located at the N-terminus is Sso7d DNA binding domain and the DNA binding domain located at the C-terminus of is Sso7d DNA binding domain; or (b) the DNA binding domain located at the N-terminus is Sto7 DNA binding domain and the DNA binding domain located at the C-terminus is Sto7 DNA binding domain; (c) the DNA binding domain located at the N-terminus is Ss07d DNA binding domain and the DNA binding domain located at the C-terminus is Sto7 DNA binding domain; or (d) the DNA binding domain located at the N-terminus is Sto7 DNA binding domain and the DNA binding domain located at the C-terminus is Ss07d DNA binding domain

In some embodiments, the engineered fusion reverse transcriptase comprises: (a) a Sso7d DNA binding domain located at the N-terminus and a Sto7 DNA domain located at the C-terminus of the amino acid sequence; (b) a Sto7 DNA binding domain located at the N-terminus and Ss07d DNA binding domain located at the C-terminus.

In some embodiments, the engineered reverse transcriptase: (a) has an amino acid sequence at least about 95% identical to SEQ ID NO: 1, and (b) comprises at least one mutation indexed to SEQ ID NO:7 selected from: an M17 mutation; an A32 mutation, a M44 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation, a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, an N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, or an H638 mutation.

In some embodiments, the engineered reverse transcriptase is at least about 95% identical to SEQ ID NO: 1, and the amino acid sequence of the engineered reverse transcriptase comprises a second combination of mutations indexed to SEQ ID NO: 7 selected from: (a) an E69K mutation, an E302R mutation, a T306K mutation, a W313F mutation, an L435G mutation, and an N454K mutation, and comprising at least one mutation selected from an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation or an H638G mutation; (b) an L139P mutation, a D200N mutation, a T330P mutation, an L603W mutation, and an E607K mutation, and comprising at least one mutation selected from: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation or an H638G mutation; (c) an A32V mutation, an L72R mutation, a D200C mutation, a G248C mutation, an E286R mutation, an E302R mutation, a W388R mutation, and an L435G mutation; or (d) a Y344L mutation and an I347L mutation.

In some embodiments, the DNA binding domain comprises: (a) an amino acid sequence selected from SEQ ID NO: 12, 13, 16, 17, or 18; or (b) an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 12, 13, 16, 17, or 18.

Another aspect of the present disclosure provides engineered reverse transcriptases. In some embodiments, the engineered reverse transcriptase has an amino acid sequence that is at least 95% identical to SEQ ID NO:179 or SEQ ID NO: 143, and the amino acid sequence of the engineered reverse transcriptase comprises a combination of mutations indexed to SEQ ID NO:7 selected from the group comprising: (a) an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (b) an M66L mutation, an E69K mutation, an L139P mutation, a D200N mutation, an E302R mutation, a T306K mutation, a W313F mutation, a T330P mutation, a N454K mutation, a D524N mutation, an H503V mutation, an L603W mutation, an E607K mutation, and an H634Y mutation, and at least one mutation selected from the group comprising an L435G mutation, an L435K mutation, an M39V mutation, a P448A mutation and a D449G mutation; (c) an M39V mutation, an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; and (d) an M39V mutation, an E69K mutation, an L139P mutation, a D200 mutation, an E302R mutation, a T306K mutation, a W313F mutation, a T330P mutation, an L435K mutation, a G429S mutation, a P448A mutation, a D449 mutation, an N454K mutation, an L603 mutation, an E607 mutation and an L671P mutation, where the D200 mutation is selected from the group consisting of D200N and D200E, the D449 mutation is selected from the group consisting of D449G and D449E, the L603 mutation is selected from the group consisting of L603W and L603F, and the E607 mutation is selected from the group consisting of E607G and E607K; and the amino acid sequence of the engineered reverse transcriptase further comprises at least one mutation selected from the group comprising P47L, H204R, D524N, T542D, E545G, D583N, H594Q, P627S, A644V, R650H, D653H, K658R, L671P, and S679P.

In some embodiments, the amino acid sequence of the engineered reverse transcriptase comprises an amino acid sequence selected from the group of amino acid sequences set forth in SEQ ID NO:180, SEQ ID NO:181, SEQ ID NO: 182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, and SEQ ID NO:192.

Another aspect of the present disclosure provides an engineered reverse transcriptase (RT) comprising a combination of mutations selected from: (a) E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or (b) E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P. In some embodiments, the mutations are introduced in SEQ ID NO: 7 or SEQ ID NO: 178.

In some embodiments, the engineered reverse transcriptase (RT) comprises a combination of mutations selected from: (a) E69K, L139P, E302R, T306K, W313F, T330P, N454K; and one or more of M39V, P47L, Q91R, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or (b) E69K, L139P, D200N, E302R, T306K, W313F, T330P, L435G, P448A, D449G, N454K, D524N, L603W, and E607K and one or more of M39V, P47L, M66L, Q91R, F155Y, H204R, G429S, H503V, T542D, E545G, D583N, H594Q, P627S, H634Y, H638G, A644V, D653H, K658R and L671P, where the mutations are introduced in SEQ ID NO: 7 or SEQ ID NO: 178.

Another aspect of the present disclosure provides an engineered reverse transcriptase (RT) comprising combination of mutations selected from E69K, L139P, D200N, E302R, T306K, W313F, T330P, L435G, P448A, D449G, N454K, D524N, L603W, and E607G and one or more of M39V, P47L, M66L, Q91R, F155Y, H204R, G429S, H503V, T542D, E545G, D583N, H594Q, P627S, H634Y, H638G, A644V, D653H, K658R and L671P, wherein the mutations are introduced in SEQ ID NO: 7 or SEQ ID NO: 178.

Another aspect of the present disclosure provides an engineered reverse transcriptase (RT) comprising the amino acid sequence of SEQ ID NO: 1, 179, or 143, and further comprising a combination of mutations selected from: (a) E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or (b) E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P.

Another aspect of the present disclosure provides an engineered reverse transcriptase comprising: an engineered reverse transcriptase having an amino acid sequence that is at least 90% identical to SEQ ID NO: 1, 143, or 179 and wherein the amino acid sequence of said engineered reverse transcriptase comprises a combination of mutations indexed to SEQ ID NO: 7 or 178 selected from the group comprising: (a) an E69K mutation, an L139P mutation, a D200N mutation, an E302R mutation, a T306K mutation, a W313F mutation, a T330P mutation, a N454K mutation, a D524N mutation, an L603W mutation, and an E607K mutation; (b) an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (c) an M66L mutation, an E69K mutation, an L139P mutation, a D200N mutation, an E302R mutation, a T306K mutation, a W313F mutation, a T330P mutation, a N454K mutation, a D524N mutation, an L603W mutation, and an E607K mutation, and at least one mutation selected from the group comprising an L435G mutation, an L435K mutation, an M39V mutation, a P448A mutation and a D449G mutation; (d) an M66L mutation, an E69K mutation, an L139P mutation, a D200N mutation, an E302R mutation, a T306K mutation, a W313F mutation, a T330P mutation, a N454K mutation, a D524N mutation, an L603W mutation, and an E607K mutation, and at least one mutation selected from the group comprising an L435G mutation and an L435K mutation; (e) an M66L mutation, an E69K mutation, an L139P mutation, a D200N mutation, an E302R mutation, a T306K mutation, a W313F mutation, a T330P mutation, a N454K mutation, a D524N mutation, an L603W mutation, and an E607K mutation, and at least one mutation selected from the group comprising an L435G mutation, an L435K mutation, and an M39V mutation; (f) an M66L mutation, an E69K mutation, an L139P mutation, a D200N mutation, an E302R mutation, a T306K mutation, a W313F mutation, a T330P mutation, a N454K mutation, a D524N mutation, an L603W mutation, and an E607K mutation, and at least one mutation selected from the group comprising an L435G mutation, an L435K mutation, and a P448A mutation; (g) an M66L mutation, an E69K mutation, an L139P mutation, a D200N mutation, an E302R mutation, a T306K mutation, a W313F mutation, a T330P mutation, a N454K mutation, a D524N mutation, an L603W mutation, and an E607K mutation, and at least one mutation selected from the group comprising an L435G mutation, an L435K mutation, an M39V mutation, a D449G mutation; (h) an M66L mutation, an E69K mutation, an L139P mutation, a D200N mutation, an E302R mutation, a T306K mutation, a W313F mutation, a T330P mutation, a N454K mutation, a D524N mutation, an H503V mutation, an L603W mutation, an E607K mutation, and an H634Y mutation, and at least one mutation selected from the group comprising an L435G mutation, an L435K mutation, an M39V mutation, a P448A mutation and a D449G mutation; (i) an M39V mutation, an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (j) an M39V mutation, an E69K mutation, an L139P mutation, a D200 mutation, an E302R mutation, a T306K mutation, a W313F mutation, a T330P mutation, an L435K mutation, a G429S mutation, a P448A mutation, a D449 mutation, an N454K mutation, an L603 mutation, an E607 mutation and an L671P mutation, wherein said D200 mutation is selected from the group consisting of D200N and D200E, wherein said D449 mutation is selected from the group consisting of D449G an D449E, wherein said L603 mutation is selected from the group consisting of L603W and L603F, wherein said E607 mutation is selected from the group consisting of E607G and E607K, and further comprising at least one mutation selected from the group consisting of P47L, H204R, D524N, T542D, E545G, D583N, H594Q, P627S, A644V, R650H, D653H, K658R, L671P, and S679P; (k) an E69K mutation, an L139P mutation, a D200N mutation, an E302R mutation, a T306K mutation, a W313F mutation, a T330P mutation, an L435G mutation, a N454K mutation, a D524N mutation, an L603W mutation, and an E607K mutation; and (1) an E69K mutation, an L139P mutation, a D200N mutation, an E302R mutation, a T306K mutation, a W313F mutation, a T330P mutation, an L435G mutation, a N454K mutation, a D524N mutation, an L603W mutation, and an E607K mutation and at least one mutation selected from the group comprising an M39V mutation, an M66L mutation, an F155 mutation, a P448 mutation, a D449 mutation, an H503 mutation, an H634 mutation, and an H638 mutation.

In some embodiments of the engineered reverse transcriptase described herein, the amino acid sequence of said engineered reverse transcriptase comprises a combination of mutations selected from the group consisting of: an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation and further comprising a second combination of mutations selected from the group consisting of: (a) an M66L mutation and an L435G mutation; (b) an M39V mutation, an M66L mutation, and an L435K mutation; (c) an M39V mutation and an L435K mutation; (d) an M66L mutation, an L435G mutation, a P448A mutation, and a D449G mutation; and (e) an M39V mutation, an M66L mutation, an L435G mutation, a P448A mutation and a D449G mutation.

In some embodiments, the amino acid sequence of said engineered reverse transcriptase comprises a combination of mutations selected from the group consisting of: an M39V mutation, an E69K mutation, an L139P mutation, a D200 mutation, an E302R mutation, a T306K mutation, a W313F mutation, a T330P mutation, a G429S mutation, a P448A mutation, a D449 mutation, an L435K mutation, a N454K mutation, an L603 mutation, an E607 mutation, and an L671P mutation and further comprising a second combination of mutations selected from the group consisting of: (a) a D524N mutation, a T542D mutation, a P627S mutation, an A644V mutation, a D653H mutation, a K658R mutation, a S679P mutation, and wherein said D200 mutation is a D200N mutation, said D449 mutation is a D449G, said L603 mutation is an L603W, and said E607 mutation is an E607G mutation; (b) a D524N mutation, a T542D mutation, an A644V mutation, a D653H mutation, an R650 mutation and a K658R mutation, and wherein said D200 mutation is a D200N mutation, said D449 mutation is a D449E mutation, said L603 mutation is an L603W mutation, and said E607 mutation is an E607G mutation; (c) an E545G mutation, a D583N mutation, an H594Q mutation, and an S679P mutation, and wherein said D200 mutation is a D200N mutation, said D449 mutation is a D449G mutation, said L603 mutation is an L603F mutation, said E607 mutation is an E607K mutation; (d) a D524N mutation, a T542D mutation, an A644V mutation, a D653H mutation, and a K658R mutation, wherein said D200 mutation is a D200N mutation, said D449 mutation is a D449E mutation, said L603 mutation is an L603W mutation, said E607 mutation is an E607G mutation; (e) an H204R mutation, a D524N mutation, a T542D mutation, a D583N mutation, an A644V mutation, a D653H mutation, and a K658R mutation, wherein said D200 mutation is a D200E mutation, said D449 mutation is a D449G mutation, said L603 mutation is an L603W mutation, said E607 mutation is an E607G mutation, and a P627S mutation; (f) an H204R mutation, an E454G mutation, a D583N mutation, an H594Q mutation, and an S679P mutation, wherein said D200 mutation is a D200E mutation, said D449 mutation is a D449G mutation, said L603 mutation is an L603F mutation, said E607 mutation is an E607K mutation; and (g) a P47 mutation, a D524N mutation, a T542D mutation, a D583N mutation, an A644V mutation, a D653H mutation, a K658R mutation and an S679P mutation, wherein said P47 mutation is a P47L mutation, D200 mutation is a D200N mutation, said D449 mutation is a D449G mutation, said L603 mutation is an L603W mutation, said E607 mutation is an E607G mutation, and a P627S mutation.

In some embodiments, the engineered reverse transcriptase comprises an amino acid sequence that is at least 90% identical to an amino acid sequence of SEQ ID NO: 180-208, or comprises an amino acid sequence of SEQ ID NO: 180-208.

Another aspect of the present disclosure provides an engineered reverse transcriptase having an amino acid sequence that is at least 95% identical to SEQ ID NO:1, 7, 179. In that embodiment, the amino acid sequence of said engineered reverse transcriptase comprises a combination of mutations indexed to SEQ ID NO:7 or 178 selected from the group comprising: (a) the combination of variants consisting of a T542D mutation, a D583N mutation, an E607G mutation, an A644V mutation, a D653H mutation and a K658R mutation; and (b) the combination of variants consisting of an E545G mutation, a D583N mutation, an H594Q mutation, an L603F mutation and a S679P mutation.

In some embodiments of the engineered reverse transcriptase described herein, the amino acid sequence of the engineered reverse transcriptase comprises E69K, L139P, D200N, E302R, T306K, W313F, T330P, N454K, H503V, D524N, L603W, E607K, and H634Y.

In some embodiments of the engineered reverse transcriptase described herein, the amino acid sequence of the engineered reverse transcriptase comprises a combination of mutations selected from: (a) M66L and L435G; (b) M39V, M66L, and L435K; (c) M39V and L435K; (d) M66L, L435G, P448A and D449G; (e) M39V, M66L, L435G, P448A and D449G; or (f) M66L.

In some embodiments, the amino acid sequence of the engineered reverse transcriptase comprises E69K, L139P, D200N, E302R, T306K, W313F, T330P, L435G, P448A, D449G, N454K, D524N, L603W, and E607K; and comprises a combination of mutations selected from (a) M66L; (b) M66L and H503V; (c) M66L and H634Y; or (d) M66L, H503V, and H634Y.

In some embodiments of the engineered reverse transcriptase described herein, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P, and comprises a second combination of mutations selected from: (a) D524N, T542D, P627S, A644V, D653H, K658R mutation, and wherein the D200 mutation is a D200N mutation, the D449 mutation is a D449G, the L603 mutation is an L603W, and the E607 mutation is an E607G mutation; (b) D524N, T542D, A644V, D653H, an R650H and K658R, and wherein the D200 mutation is a D200N mutation, the D449 mutation is a D449E mutation, the L603 mutation is an L603W mutation, and the E607 mutation is an E607G mutation; (c) E545G, D583N, and H594Q, and wherein the D200 mutation is a D200N mutation, the D449 mutation is a D449G mutation, the L603 mutation is an L603F mutation, and the E607 mutation is an E607K mutation; (d) D524N, T542D, A644V, D653H, and K658R, wherein the D200 mutation is a D200N mutation, the D449 mutation is a D449E mutation, the L603 mutation is an L603W mutation, the E607 mutation is an E607G mutation; (e) H204R, D524N, T542D, P627S, D583N, A644V, D653H and K658R, wherein the D200 mutation is a D200E mutation, the D449 mutation is a D449G mutation, the L603 mutation is an L603W mutation, the E607 mutation is an E607G mutation; (f) H204R, E545G, D583N, and H594Q, wherein the D200 mutation is a D200E mutation, the D449 mutation is a D449G mutation, the L603 mutation is an L603F mutation, the E607 mutation is an E607K mutation; or (g) P47L, D524N, T542D, D583N, P627S, A644V, D653H, and K658R, wherein the D200 mutation is a D200N mutation, the D449 mutation is a D449G mutation, the L603 mutation is an L603W mutation, the E607 mutation is an E607G mutation.

Another aspect of the present disclosure provides an engineered fusion RT or an engineered RT comprising an amino acid sequence that is at least about 90%, at least about 92%, at least about 95%, at least about 97%, at least about 98%, or at least about 99% identical to: (a) an amino acid sequence to an amino acid sequence of an RT disclosed in Table 4, Table 5 or Table 6; or (b) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (c) SEQ ID NOs: 180-208.

Another aspect of the present disclosure provides an engineered fusion RT or an engineered RT comprising: (a) an amino acid sequence of an RT disclosed in Table 4, Table 5 or Table 6; or (b) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (c) SEQ ID NOs: 180-208.

In some embodiments, an engineered reverse transcriptase of the present disclosure exhibits an altered reverse transcriptase related activity as compared to a reverse transcriptase having the amino acid sequence set forth in SEQ ID NO:179. In some embodiments, the altered reverse transcriptase related activity is selected from the group of reverse transcriptase related activities comprising processivity, template switching efficiency, binding affinity and transcription efficiency. In one embodiment, the altered reverse transcriptase related activity is an altered template switching (TS) efficiency as compared to the template switching efficiency of a reverse transcriptase having the amino acid sequence set forth in SEQ ID NO:179. In one embodiment, the altered reverse transcriptase related activity is an altered transcription efficiency as compared to the transcription efficiency of a reverse transcriptase having the amino acid sequence set forth in SEQ ID NO:179. In one embodiment, the altered reverse transcriptase related activity is an altered transcription efficiency and an altered template switching efficiency as compared to a reverse transcriptase having the amino acid sequence set forth in SEQ ID NO:179. In one embodiment, the altered reverse transcriptase related activity is an increased transcription efficiency as compared to a reverse transcriptase having the amino acid sequence set forth in SEQ ID NO:179. In one embodiment, the altered reverse transcriptase related activity is an increased transcription efficiency and an increased template switching efficiency as compared to a reverse transcriptase having the amino acid sequence set forth in SEQ ID NO:179. In one embodiment, the altered reverse transcriptase related activity is an altered processivity as compared to the processivity of a reverse transcriptase having the amino acid sequence set forth in SEQ ID NO:179. In one embodiment, the altered reverse transcriptase related activity is an altered ability to yield mitochondrial UMI counts as compared to a reverse transcriptase having the amino acid sequence set forth in SEQ ID NO:179. In one embodiment, the reverse transcriptase related activity is an altered ability to yield ribosomal UMI counts as compared to a reverse transcriptase having the amino acid sequence set forth in SEQ ID NO:179.

In some embodiments, the amino acid sequence of an engineered reverse transcriptase of the present application comprises a combination of mutations selected from the group consisting of an E69K mutation, an L139P mutation, a D200N mutation, an E302R mutation, a T306K mutation, a W313F mutation, a T330P mutation, an N454K mutation, an H503V mutation, a D524N mutation, an L603 mutation, an E607K mutation and an H634Y mutation, and further comprising a second combination of mutations selected from the group consisting of (a) an M66L mutation and an L435G mutation, (b) an M39V mutation, an M66L mutation and an L435K mutation, (c) an M39V mutation and an L435K mutation, (d) an M66L mutation, an L435G mutation, a P448 mutation, and a D448 mutation, and (e) an M39V mutation, an M66L mutation, an L435G mutation, a P448A mutation and a D449G mutation.

In some embodiments, the amino acid sequence of an engineered reverse transcriptase of the present disclosure comprises a combination of mutations selected from the group consisting of an M39V mutation, an E69K mutation, an L139P mutation, a D200 mutation, an E302R mutation, a T306K mutation, a W313F mutation, a T330P mutation, a G429S mutation a P448A mutation, a D449 mutation, an L435K mutation, a N454K mutation, an L603 mutation, an E607 mutation, and an L671P mutation; and further comprises a second combination of mutations selected from the group consisting of: (a) a D524N mutation, a T542D mutation, a P627S mutation, an A644V mutation, a D653H mutation, a K658R mutation, a S679P mutation, and wherein said D200 mutation is a D200N mutation, said D449 mutation is a D449G, said L603 mutation is an L603W, and said E607 mutation is an E607G mutation; (b) a D524N mutation, a T542D mutation, an A644V mutation, a D653H mutation, an R650 mutation and a K658R mutation, and where the D200 mutation is a D200N mutation, the D449 mutation is a D449E mutation, the L603 mutation is an L603W mutation, and the E607 mutation is an E607G mutation; (c) an E545G mutation, a D583N mutation, an H594Q mutation, and an S679P mutation, and where the D200 mutation is a D200N mutation, the D449 mutation is a D449G mutation, the L603 mutation is an L603F mutation, and the E607 mutation is an E607K mutation; (d) a D524N mutation, a T542D mutation, an A644V mutation, a D653H mutation, and a K658R mutation, where the D200 mutation is a D200N mutation, the D449 mutation is a D449E mutation, the L603 mutation is an L603W mutation, and the E607 mutation is an E607G mutation; (e) an H204R mutation, a D524N mutation, a T542D mutation, a D583N mutation, an A644V mutation, a D653H mutation, and a K658R mutation, where the D200 mutation is a D200E mutation, the D449 mutation is a D449G mutation, the L603 mutation is an L603W mutation, and the E607 mutation is an E607G mutation, and a P627S mutation; (f) an H204R mutation, an E454G mutation, a D583N mutation, an H594Q mutation, and an S679P mutation, where the D200 mutation is a D200E mutation, the D449 mutation is a D449G mutation, the L603 mutation is an L603F mutation, and the E607 mutation is an E607K mutation; and (g) a P47 mutation, a D524N mutation, a T542D mutation, a D583N mutation, an A644V mutation, a D653H mutation, a K658R mutation and an S679P mutation, where the P47 mutation is a P47L mutation, the D200 mutation is a D200N mutation, the D449 mutation is a D449G mutation, the L603 mutation is an L603W mutation, the E607 mutation is an E607G mutation, and a P627S mutation.

In another aspect, the present disclosure provides an engineered reverse transcriptase where the amino acid sequence of the engineered reverse transcriptase comprises an amino acid sequence selected from the group of amino acid sequences set forth in SEQ ID NO:180, SEQ ID NO:181, SEQ ID NO: 182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, and SEQ ID NO:192. In some embodiments, the engineered reverse transcriptase of the present disclosure exhibits an altered reverse transcriptase related activity as compared to a reverse transcriptase having the amino acid sequence set forth in SEQ ID NO:179.

In some embodiment, the altered reverse transcriptase related activity is selected from the group of reverse transcriptase related activities comprising processivity, template switching efficiency, binding affinity and transcription efficiency. In one embodiment, the altered reverse transcriptase related activity is an altered template switching (TS) efficiency as compared to the template switching efficiency of a reverse transcriptase having the amino acid sequence set forth in SEQ ID NO:179. In one embodiment, the altered reverse transcriptase related activity is an altered transcription efficiency as compared to the transcription efficiency of a reverse transcriptase having the amino acid sequence set forth in SEQ ID NO:179. In one embodiment, the altered reverse transcriptase related activity is an altered transcription efficiency and an altered template switching efficiency as compared to a reverse transcriptase having the amino acid sequence set forth in SEQ ID NO:179. In an embodiment, the altered reverse transcriptase related activity is an increased transcription efficiency as compared to a reverse transcriptase having the amino acid sequence set forth in SEQ ID NO:179. In one embodiment, the altered reverse transcriptase related activity is an increased transcription efficiency and an increased template switching efficiency as compared to a reverse transcriptase having the amino acid sequence set forth in SEQ ID NO:179. In one embodiment, the altered reverse transcriptase related activity is an altered processivity as compared to the processivity of a reverse transcriptase having the amino acid sequence set forth in SEQ ID NO:179. In one embodiment, the altered reverse transcriptase related activity is an altered ability to yield mitochondrial UMI counts as compared to a reverse transcriptase having the amino acid sequence set forth in SEQ ID NO:179. In some embodiments, the reverse transcriptase related activity is an altered ability to yield ribosomal UMI counts as compared to a reverse transcriptase having the amino acid sequence set forth in SEQ ID NO:179.

Another aspect of the present disclosure provides an engineered reverse transcriptase having an amino acid sequence that is at least 95% identical to SEQ ID NO:179, where the amino acid sequence of the engineered reverse transcriptase comprises a combination of mutations indexed to SEQ ID NO:178 selected from the group comprising: (a) the combination of variants consisting of a T542D mutation, a D583N mutation, an E607G mutation, an A644V mutation, a D653H mutation and a K658R mutation; and (b) the combination of variants consisting of an E545G mutation, a D583N mutation, an H594Q mutation, an L603F mutation and a S679P mutation. In some embodiments, the engineered reverse transcriptase exhibits an altered reverse transcriptase related activity as compared to a reverse transcriptase having the amino acid sequence set forth in SEQ ID NO:179. In some embodiments, the altered reverse transcriptase related activity is selected from the group of reverse transcriptase related activities comprising an RNAase H activity, processivity, template switching efficiency, binding affinity and transcription efficiency.

Another aspect of the present disclosure provides an isolated nucleic acid molecule encoding: (a) an engineered reverse transcriptase described herein; (b) a DNA binding domain described herein; and/or (c) an engineered fusion reverse transcriptase described herein. In some embodiments, the isolated nucleic acid molecule comprises a sequence selected from SEQ ID NO: 136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:167, SEQ ID NO:169, or SEQ ID NO: 171; or a nucleic acid sequence of Table 5.

Another aspect of the present disclosure provides an expression vector comprising an isolated nucleic acid described herein.

Another aspect of the present disclosure provides a host cell transfected with an expression vector described herein or an isolated nucleic acid described herein.

Another aspect of the present disclosure provides a method for performing a reverse transcription reaction for generating a nucleic acid product from an RNA template using an engineered fusion reverse transcriptase or an engineered reverse transcriptase described herein.

In some embodiments of the method, (a) the engineered fusion reverse transcriptase comprises a DNA binding domain comprising an amino acid sequence selected from SEQ ID NO: 12, 13, 16, 17, or 18; (b) the engineered reverse transcriptase comprises: (i) an amino acid sequence selected from: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55; or (ii) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (iii) an amino acid sequence disclosed in Table 5 or 6.

In some embodiments, the amino acid sequence of the engineered fusion reverse transcriptase comprises: (a) an amino acid sequence selected from SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170; or (b) an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170.

In some embodiments, the engineered RT or the engineered fusion RT comprises M39V, M66I, Q91R, I347V, H594Q, or a combination thereof, and optionally M39V, M66I, Q91R, I347V, H594Q, or the combination thereof (substituted) in the RT sequence of SEQ ID NO: 143 (SEQ ID NO: 129, SOLD 034). IN some embodiments, the engineered RT or the engineered fusion RT comprises M39V, T542D, D583N, E607G, A644V, D653H, K658R, L671P, or a combination thereof (substituted) in the RT sequence of SEQ ID NO: 143 (or SEQ ID NO: 7).

In some embodiments, the engineered RT or the engineered fusion RT comprises M39V, T542D, D583N, E607G, A644V, D653H, K658R, L671P, or a combination thereof, and optionally M39V, T542D, D583N, E607G, A644V, D653H, K658R, L671P, or the combination thereof substituted in the RT sequence of SEQ ID NO: 143 (or SEQ ID NO: 7) (SEQ ID NO: 111, SOLD 025). In some embodiments, the engineered RT or the engineered fusion RT comprises a M39V, M66I, Q91R, I347V, H594Q, or a combination thereof in the RT backbone of SEQ ID NO: 143 or SEQ ID NO: 7.

In some embodiment, the engineered fusion RT or the engineered RT comprises an amino acid sequence that is at least about 90%, at least about 92%, at least about 95%, at least about 97%, at least about 98%, or at least about 99% identical to an amino acid sequence to: (a) an amino acid sequence of an RT disclosed in Table 4, Table 5 or Table 6; (b) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (c) SEQ ID NOs: 180-208.

In some embodiments, the engineered fusion RT or the engineered RT comprising: (a) an amino acid sequence of an RT disclosed in Table 4, Table 5 or Table 6; or (b) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (c) SEQ ID NOs: 180-208.

In some embodiments, the engineered fusion reverse transcriptase of any one of claims 12-18, having the amino acid sequence of SEQ ID NO: 111, SEQ ID NO: 129, or SEQ ID NO: 20.

In some embodiments, the engineered fusion reverse transcriptase comprises an M39 mutation, a K47 mutation, an L435 mutation, a D449 mutation, a D524 mutation, an E607 mutation, a D653 mutation and an L671 mutation in SEQ ID NO:7. In some embodiments, the engineered fusion reverse transcriptase comprises a mutation selected from a K13 mutation, a K13L mutation, a D36 mutation, an N37 mutation, a V2 mutation, a D36L mutation, an insertion, or a combination thereof.

In some embodiments, the engineered fusion reverse transcriptase comprises: (a) an amino acid sequence selected from SEQ ID NO:3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170; or (b) an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170.

In some embodiments, the amino acid sequence of the engineered fusion reverse transcriptase comprises a second combination of mutations indexed to SEQ ID NO:7 consisting of: an E69K mutation, an E302R mutation, a T306K mutation, a W313F mutation, an L435G mutation, and an N454K mutation, and comprising at least one mutation selected from an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation.

In some embodiments, the amino acid sequence of the engineered fusion reverse transcriptase comprises a second combination of mutations indexed to SEQ ID NO:7 consisting of: an L139P mutation, a D200N mutation, a T330P mutation, an L603W mutation, and an E607K mutation, and comprising at least one mutation selected from: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation.

In some embodiments, the amino acid sequence of the engineered reverse transcriptase comprises a second combination of mutations indexed to SEQ ID NO:7 consisting of: an A32V mutation, an L72R mutation, a D200C mutation, a G248C mutation, an E286R mutation, an E302R mutation, a W388R mutation, and an L435G mutation. In some embodiments, the amino acid sequence of the engineered reverse transcriptase comprises a second combination of mutations selected from a Y344L mutation or an I347L mutation of SEQ ID NO: 7.

In another aspect, the invention provides methods for using any of the RTs of the invention in methods comprising contacting the engineered fusion reverse transcriptase with a nucleic acid template under suitable conditions to produce a polymerized nucleic acid product, wherein the nucleic acid template is an RNA, a DNA, or a nucleic acid comprising an unnatural nucleotide.

In some embodiments, the methods can be carried out in a partition comprising a single cell or single nucleus. In some embodiments, the method can be carried out in a bulk reaction.

In non-limiting embodiments of the methods, the recombinant fusion RT or recombinant RT is any one of the sequences disclosed in Table 4, Table 5 or Table 6.

Another aspect of the present disclosure provides a method of using the engineered fusion reverse transcriptase or the engineered reverse transcriptase described herein, the method comprising contacting the engineered fusion reverse transcriptase with a nucleic acid template under suitable conditions to produce a polymerized nucleic acid product, wherein the nucleic acid template is an RNA, a DNA, or a nucleic acid comprising an unnatural nucleotide.

Another aspect of the present disclosure provides a nucleic acid extension method comprising: (a) contacting a target nucleic acid molecule with an engineered fusion reverse transcriptase or an engineered reverse transcriptase and a plurality of nucleic acid barcoded molecules comprising a barcode sequence, and (b) incubating the target nucleic acid, the engineered fusion reverse transcriptase or the engineered reverse transcriptase and barcoded molecules under conditions in which the barcoded molecules are extended by the engineered fusion reverse transcriptase. In some embodiments, the engineered fusion reverse transcriptase or the engineered reverse transcriptase comprises the amino acid sequence of an engineered fusion transcriptase described herein.

Another aspect of the present disclosure provides a recombinant reverse transcriptase (RT) fusion protein comprising: a RT polypeptide fused to a DNA binding domain. In some embodiments, the RT polypeptide and the DNA binding domain are separated by an amino acid linker. In that embodiment, the DNA binding domain is fused to the C-terminus of the RT polypeptide.

In some embodiments, the RT polypeptide comprises of the amino acid sequence of any one of the RT polypeptide amino acid sequences listed in Table 4, table 5 or Table 6. In some embodiments, the DNA binding domain is from any one of the DNA binding proteins Sto7d, Sso7d, Sis7b, Sis7a, Ssh7b, Sto7, Aho7C, Aho7B, Aho7A, Mcu7, Mse7, Sac7e, Sac7d, Stod7; or Stod7d.

In some embodiments, the linker is a G(n)S(m)G(p) linker, where n=0, 1, 2, 3, 4, 5, 6, 7, 8, 9, 10 11, 12, 13, 14, 15, 16, 17, 18, 19 or 20, m=0, 1, 2, 3, 4, 5, 6, 7, 8, 9, 10 11, 12, 13, 14, 15, 16, 17, 18, 19 or 20, p=0, 1, 2, 3, 4, 5, 6, 7, 8, 9, 10 11, 12, 13, 14, 15, 16, 17, 18, 19 or 20, and n, m, and p are selected independently.

In some embodiments, the DNA binding domain is Sto7 or a truncation thereof. In some embodiments, the RT polypeptide is 42B L (SEQ ID NO: 145), 50A+G (SEQ ID NO: 147), or an RT polypeptide set forth in SEQ ID NO: 143 or SEQ ID NO: 172.

Another aspect of the present disclosure provides a recombinant RT fusion protein comprising, consisting essentially of, or consisting of SEQ ID NO: 20, SEQ ID NO: 135, SEQ ID NO: 137, SEQ ID NO: 139, SEQ ID NO: 153, SEQ ID NO: 155, SEQ ID NO: 157, SEQ ID NO: 159, SEQ ID NO: 161, SEQ ID NO: 163, SEQ ID NO: 166, SEQ ID NO: 168, or SEQ ID NO: 170.

In some embodiments of the recombinant RT fusion protein described herein, the recombinant RT fusion protein exhibits increased template switching (TS) efficiency, increased processivity efficiency, increased binding affinity, increased transcription efficiency, increased chemical tolerance, improved ability to yield mitochondrial unique molecular identity (UMI) counts, improved ability to yield ribosomal unique molecular identity (UMI) counts, longer shelf life, higher strand displacement, higher end-to-end template jumping, or any combination thereof.

In some embodiments, the recombinant RT fusion protein comprises at least two or more of increased template switching (TS) efficiency, increased processivity efficiency, increased binding affinity, increased transcription efficiency, increased chemical tolerance, improved ability to yield mitochondrial unique molecular identity (UMI) counts, longer shelf life, higher strand displacement, higher end-to-end template jumping, or improved ability to yield ribosomal unique molecular identity (UMI) counts.

Another aspect of the present disclosure provides an isolated nucleic acid molecule encoding a recombinant fusion RT protein described herein. In some embodiments, the nucleic acid molecule comprises a sequence selected from SEQ ID NO: 136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:167, SEQ ID NO:169, or SEQ ID NO: 171 or a nucleic acid sequence of Table 5.

Another aspect of the present disclosure provides a method for performing a reverse transcription reaction for generating a nucleic acid product from an RNA template using the recombinant RT fusion protein described herein.

Another aspect of the present disclosure provides a method of using any one of the recombinant RT fusion proteins or engineered RT proteins described herein, the method comprising contacting the recombinant RT fusion protein or an engineered RT protein with a nucleic acid template under suitable conditions to produce a polymerized nucleic acid product. In some embodiments, the nucleic acid template is an RNA, a DNA, or a nucleic acid comprising an unnatural nucleotide.

Another aspect of the present disclosure provides a composition comprising (a) a recombinant fusion RT protein described herein; or (b) an engineered reverse transcriptase described herein, or (c) an isolated nucleic acid described herein; or (d) an expression vector described herein; or (e) a host cell described herein; and (f) a buffer. In non-limiting embodiments, the buffer includes reagents suitable for carrying out an RT reaction.

Another aspect of the present disclosure provides a kit comprising: (a) a recombinant RT fusion protein described herein; or (b) an engineered reverse transcriptase described herein; or (c) the isolated nucleic acid described herein; or (d) an expression vector described herein; or (e) a host cell described herein; (f) a composition described herein; and (g) instructions.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

Section headings, numerical and/or alphabetical listings, e.g., (a), (b), (i) etc., are presented merely for ease of reading the disclosure, including the specification and claims. The use of headings in the disclosure, including the specification or claims does not require the steps or elements be performed in alphabetical or numerical order or the order in which they are presented.

### BRIEF DESCRIPTION OF THE DRAWINGS

FIG. 1 provides a schematic of an exemplary capillary electrophoresis (CE) validation assay process. 5′-end labeled DNA primers are hybridized to RNA templates at room temperature (approx. 25° C.). Poly rG-labeled template switching oligonucleotides (rG-TSO) are added to the reaction mixture. The temperature is raised to 53° C. and first strand cDNA synthesis, the addition of a poly-C tail (tailing), template switching and TSO extension occur. Samples are then transferred to a Genetic Analyzer for analysis.

FIG. 2 provides an exemplary trace of a CE assay output following the process from FIG. 1. Product size was calibrated with synthetically sized controls for the primer alone size, a full-length extension of the primer length, and a full-length extension of the primer plus the switching oligo (TSO). Product length is indicated on the x-axis, fluorescent signal intensity is indicated on the y-axis.

FIG. 3 provides an exemplary trace of a capillary electrophoresis (CE) assay output for an RT enzyme control (containing a commercially prepared engineered reverse transcriptase; enzyme mix C, bottom) and an engineered reverse transcriptase having the amino acid sequence set forth in SEQ ID NO:14 (a transcription positive, template switching null engineered reverse transcriptase (AR)), top. See for example PCT/US20/64323 regarding the engineered reverse transcriptase having the amino acid sequence set forth in SEQ ID NO:14. Product length is indicated on the x-axis; fluorescent signal intensity is indicated on the y-axis. Peaks associated with the full-length product, the full-length product plus tail and the full-length product plus tail and template switching are indicated. The trace indicates the control RT reaction (enzyme mix C) yields full sized template switched products. The trace indicates reactions with an engineered reverse transcriptase enzyme having the amino acid sequence set forth in SEQ ID NO:14 (AR) yield full length transcription products, however a full-length template switched product peak is not significantly present.

FIG. 4 provides an exemplary trace of a CE assay output for control enzyme mix C and the length parameters associated with various reaction products as used for transcription efficiency and template switching efficiency calculations. Reads less than 45 nucleotides are considered incomplete (section 1). Reads including the full length and the full length plus the tail are considered the elongation and tailing phase (section 2). Reads longer than the full length plus the tail and shorter than the full length plus tail and template switching are considered incomplete template switching products (incomplete TSO, section 3). Reads having the full length plus tail and template switching length are considered template switched (TSO, section 4). Transcription efficiency is the sum of the area under the curve for section 2, section 3 and section 4 divided by the total area under the curve. Template switching efficiency is the area under the curve of the template switched (section 4) divided by the sum of the area under curve for section 2, section 3 and section 4.

FIG. 5 provides a chart summarizing the percent of valid barcodes (y axis) in reads obtained for a control Enzyme Mix C, a reverse transcriptase having the amino acid sequence set forth in SEQ ID NO: 1, an engineered reverse transcriptase having the amino acid sequence set forth in SEQ ID NO: 6 and an engineered reverse transcriptase having the amino acid sequence set forth in SEQ ID NO: 8, as assayed using a GEM-X assay.

FIG. 6 provides a chart summarizing the percent of reads confidently mapped to the transcriptome (y axis) obtained for a control Enzyme Mix C, a reverse transcriptase having the amino acid sequence set forth in SEQ ID NO: 1, an engineered reverse transcriptase having the amino acid sequence set forth in SEQ ID NO: 6, and an engineered reverse transcriptase having the amino acid sequence set forth in SEQ ID NO: 8 as assayed using a GEM-X assay.

FIG. 7 provides a chart summarizing the median genes per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO: 8 as assayed using a GEM-X assay.

FIG. 8 provides a chart summarizing the median UMI counts per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO: 8 as assayed using a GEM-X assay.

FIG. 9 provides a chart summarizing the fraction of ribosomal protein UMI counts per cell (y axis) Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO: 8 as assayed using a GEM-X assay.

FIG. 10 provides a chart summarizing the fraction of mitochondrial UMI counts per cell (y axis) obtained for a control Enzyme Mix C, a reverse transcriptase having the amino acid sequence set forth in SEQ ID NO:1, an engineered reverse transcriptase having the amino acid sequence set forth in SEQ ID NO:6, and an engineered reverse transcriptase having the amino acid sequence set forth in SEQ ID NO: 8 as assayed using a GEM-X assay.

FIG. 11 provides a summary of results obtained when assessing a variety of engineered reverse transcriptases for transcription efficiency and template switching efficiency. The template switching efficiency of a fusion variant having the amino acid sequence set forth in SEQ ID NO: 8 is greater than the template switching efficiency of enzymes having an amino acid sequence set forth in SEQ ID NO:1 or SEQ ID NO:6. Y-axis is the % of generated nucleic acid product.

FIG. 12 provides a summary of results obtained from an experiment evaluating template switching ability of an enzyme having the amino acid sequence set forth in SEQ ID NO:1 and an engineered reverse transcriptase having the amino acid sequence set forth in SEQ ID NO: 5. The template switching efficiency of the engineered reverse transcriptase having the amino acid sequence set forth in SEQ ID NO:5 is significantly increased compared to the template switching efficiency of an engineered reverse transcriptase having the amino acid sequence set forth in SEQ ID NO:1.

FIGS. 13A-B provide a bar graph (FIG. 13A) and a quantification (FIG. 13B) illustrating the enhanced sensitivity of a 5′ single cell assay using an engineered reverse transcriptase or an engineered fusion reverse transcriptase comprising sto-7 described herein as shown by the median genes identified per cell (Median genes/cell (20 k) or the median UMIs identified per cell (median UMIs/cell (20 k). Specifically, an engineered reverse transcriptase of the present disclosure significantly improved the RT sensitivity when compared to a reverse transcriptase set forth in SEQ ID NO: 1; and an engineered fusion reverse transcriptase comprising a Sto7 binding domain further significantly increased the gain in sensitivity of the engineered reverse transcriptase described herein.

FIG. 14 provides a bar graph illustrating that an engineered fusion protein comprising Sto7 significantly enhanced the number of genes detected in the assay when compared to unfused engineered reverse transcriptase or a reverse transcriptase set forth in SEQ ID NO: 1.

FIG. 15 shows a CLUSTAL O (1.2.4) multiple protein alignment report of the wild-type (WT) and engineered Moloney Murine Leukemia Virus reverse-transcriptases (MMLV RT). The sequence alignment illustrates the difference between an engineered MMLV RT variant (SEQ ID NO:1, 143 or 179) and the wt MMLV (SEQ ID NO: 7 or 178; GenBank Seq ID NP_955591.1 p80RT (ebi.ac.uk/Tools/msa/clustalo/)). The MMLV RT variant of SEQ ID NO: 1, 143, or 179 is an embodiment of an RT enzyme found in enzyme mix C (EMC) and was used as a control in the Examples disclosed herein and FIGS. 5-9, 13, 14, 27-30, 32-37.

FIGS. 16A-B show bar graphs summarizing the results obtained from CE analysis of 8 reverse transcriptase variants. RT variants are indicated on the x-axis. The amino acid sequences of variant 1, variant 2, variant 3, variant 4, variant 5, variant 6 and variant 8 are set forth in SEQ ID NOS: 186, 187, 188, 189, 190, 191, and 192, respectively. The y-axes indicate the fraction of full-length product (FIG. 16A) and the fraction of template switched product (FIG. 16B). FIG. 16A shows the full-length product obtained from the indicated variants; and further demonstrates that the amount of full-length product, an indicator of transcription efficiency, obtained from the variants having the amino acid sequences set forth in SEQ ID NOS: 186, 187, 188, 189, 190, 191, and 192 is greater than the amount of full-length product obtained from an engineered reverse transcriptase having the amino acid sequence set forth in SEQ ID NO:1 or 179. FIG. 16B shows the template switching efficiency of the indicated variants; and further demonstrates that the template switching efficiency of the variants having the amino acid sequences set forth in SEQ ID NOS: 186, 187, 188, 189, 190, 191, and 192 is greater than the template switching efficiency of an engineered reverse transcriptase having the amino acid sequence set forth in SEQ ID NO:1 or 179.

FIG. 17 shows a bar chart comparing the transcription efficiency and template switching efficiency of multiple engineered reverse transcriptases in CE assays. Bars indicating the transcription efficiency are indicated on the left for each enzyme tested; bars indicating the template switching efficiency are indicated on the right for each enzyme tested. The percent product is indicated on the y axis; the enzyme tested is indicated on the x axis. SEQ ID NO:1 or 179 refers to an engineered reverse transcriptase having the amino acid sequence set forth in SEQ ID NO:1 or 179. Results from the indicated engineered reverse transcriptase are provided.

FIG. 18 shows a table comparing the transcription efficiency, template switching efficiency and fraction of product (plus TSO) of multiple engineered reverse transcriptases compared to the variant having the amino acid sequence set forth in SEQ ID NO: 1 or 179 in CE assays performed with a GAPDH template. All indicated variants showed similar levels of full length product formation indicative of the transcription efficiency. Template switching efficiency and target product formation are improved in variants with mutations L435G and M66L. The improvement increases slightly with the variants in combination.

FIG. 19 shows a bar graph summarizing the cDNA yields obtained from engineered reverse transcriptases having an amino acid sequence set forth in the indicated SEQ ID NO in single cell experiments. The single cell experiments were performed in the 3′ and 5′ configurations. Results from the 3′ configuration are shown as the left bar for each enzyme, and results from the 5′ configuration are shown as the right bar for each enzyme. Yields for variants with a M66L mutation and/or the M39V mutation exceed the yield obtained from a reverse transcriptase having the amino acid sequence set forth in SEQ ID NO:1 or 179 in the 3′ experiments. These results are comparable to the results from tests of total product yield on the GAPDH template. Surprisingly, the yields for the single cell 5′ configuration differ from expectations based on the total product yield on the GAPDH template.

FIGS. 20A-C show tables summarizing metrics of the 3′ single cell experiments; FIG. 20A provides 20 k read metrics, FIG. 20B provides 50 kilo read metrics and FIG. 20C provides reads mapped to the transcriptome. The amino acid sequences of the indicated engineered reverse transcriptases are provided in the indicated SEQ ID NO. The percent indicates the percent change from the results obtained with an engineered reverse transcriptase having the amino acid sequence set forth in SEQ ID NO:1 or 179. All variants with a M66L mutation showed improved sensitivity at 50 kilo reads per cell (krpc) but the extent of the improvement depends on the context.

FIGS. 21A-B show tables summarizing additional metrics related to results obtained from the indicated engineered reverse transcriptase in single cell 3′ experiments. Most of the variants yielded metrics within parity for valid UMI's, valid barcodes, ribosomal UMI's, mitochondrial UMI's, transcript coverage (FIG. 21A), and reads with any poly A sequence, reads with any switch oligo sequence and reads with primer or homopolymer sequence (FIG. 21B). However, when the libraries produced by most variants with the M66L mutation in combination with either P448A, D449G and/or M39V were evaluated for reads mapped to the transcriptome, there was a decrease in reads mapped to the transcriptome. Surprisingly, the variant having the amino acid sequence set forth in SEQ ID NO: 180 (2), which has the M66L alteration exhibited improved template switching efficiency and maintains levels of reads mapped to the transcriptome close to that obtained with the engineered reverse transcriptase having the amino acid sequence set forth in SEQ ID NO:1 or 179.

FIGS. 22A-B show tables summarizing metrics of the 5′ single cell experiments, including 20 k read metrics, 50K read metrics and reads mapped to the transcriptome. The engineered reverse transcriptase variants have the amino acid sequences provided in the indicated SEQ ID NO. The percent indicates the percent change from the results obtained with an engineered reverse transcriptase having the amino acid sequence set forth in SEQ ID NO:1 or 179. In particular, an engineered reverse transcriptase having the amino acid sequence set forth in SEQ ID NO:180 (2) showed a significant improvement in sensitivity. Engineered reverse transcriptases with the M66L alteration, P448A, D449G and/or M39V suffered mapping loss.

FIGS. 23A-B show tables summarizing additional metrics related to results obtained from the indicated engineered reverse transcriptase in single cell 5′ experiments. Most of the variants yielded metrics within parity for valid UMI's, valid barcodes, ribosomal UMI's, mitochondrial UMI's, transcript coverage (FIG. 23A), reads with any poly A sequence, reads with any switch oligo sequence and reads with primer or homopolymer sequence (FIG. 23B). However, when the libraries produced by most variants with the M66L mutation in combination with either P448A, D449G and/or M39V were evaluated for reads mapped to the transcriptome, there was a decrease in reads mapped to the transcriptome. Surprisingly the variant having the amino acid sequence set forth in SEQ ID NO: 180 (2), which has the M66L alteration exhibits improved template switching efficiency and the levels of reads mapped to the transcriptome is impacted less than when other engineered reverse transcriptases are used.

FIGS. 24A-B show tables summarizing metrics obtained from engineered reverse transcriptases having the amino acid sequence set forth in the indicated SEQ ID NO. The engineered reverse transcriptases were evaluated with human and mouse peripheral blood monocytes in 5′ and 3′ chemistries. The percent change is as compared to a commercially available variant MMLV reverse transcriptase. The change in median genes and median UMI's queried at 20,000 reads per cell and the change in reads mapped to the transcriptome and reads mapped to exons are shown. A commercially available engineered reverse transcriptase was used as the control. The amino acid sequences of the engineered reverse transcriptases are set forth in SEQ ID NO: 180 (2), SEQ ID NO: 185 (7), SEQ ID NO: 195 (24) and SEQ ID NO: 196 (25). Improvements in both the 5′ and 3′ chemistries are more pronounced in the mouse PBMC's than in the human PBMCs. Note the significant improvements exhibited by the engineered reverse transcriptase having the amino acid sequence set forth in SEQ ID NO: 180 (2). It is also noted that the reads mapped to the transcriptome or the exon obtained with an engineered reverse transcriptase having the amino acid sequence set forth in SEQ ID NO: 180 (2) decreased as compared to a commercially available engineered reverse transcriptase.

FIGS. 25A-C show feature scatter plots and an overlaid TSNE plot by enzyme obtained from an engineered reverse transcriptase having the amino acid sequence set forth in the indicated SEQ ID NO in experiments using the 5′ chemistry in human PBMCs (FIG. 25A) and in mouse PBMCs (C57BL/6 cells; FIG. 25B). FIGS. 25A-B show that the engineered reverse transcriptase having the amino acid sequence set forth in SEQ ID NO: 180 (2) exhibited tight correlation in both human and mouse samples. The correlation exhibited by an engineered reverse transcriptase having the amino acid sequence set forth in SEQ ID NO: 180 was tighter than the correlation exhibited by an engineered reverse transcriptase having the amino acid sequence set forth in SEQ ID NO:185 (7). The engineered reverse transcriptase having the amino acid sequence set forth in SEQ ID NO:185 (7) exhibited a tighter correlation in mouse cells than in human cells in 5′ and 3′ chemistries (3′ data not shown). FIG. 25C shows an overlaid TSNE plot by enzyme. The engineered reverse transcriptase having the amino acid sequence set forth in SEQ ID NO: 180 (2) and a commercially available engineered reverse transcriptase show homogeneity in cell populations compared to the engineered reverse transcriptase having the amino acid sequence set forth in SEQ ID NO:185 (7).

FIGS. 26A-B show tables summarizing immune profiling results obtained from the indicated engineered reverse transcriptase as compared to a commercially available engineered reverse transcriptase. FIG. 26 shows immunoprofiling based on TCR Improvements as a percent change and FIG. 26B shows immunoprofiling based on Ig improvement as a percent change. The median TRA and TRB UMI's are shown. The median TRA UMIs and median TRB UMIs obtained with an engineered reverse transcriptase having the amino acid sequence set forth in SEQ ID NO:180 (2) were greater than those obtained with a commercially available engineered reverse transcriptase in both human PBMCs and mouse PBMCs. Engineered reverse transcriptases previously shown to exhibit IG sensitivity exhibited a comparable or improved IG sensitivity (as compared to previous ATP results). In mouse PBMC's, the median IGH UMIs, median IGK UMIs and median IGL UMIs obtained with enzymes having the amino acid sequence set forth in SEQ ID NO: 180 (2), SEQ ID NO: 195 (24) or SEQ ID NO: 196 (25) were greater than those obtained with a commercially available engineered reverse transcriptase (right chart). The results obtained with an engineered reverse transcriptase having the amino acid sequence set forth in SEQ ID NO: 180 (2) were substantially higher than those obtained with engineered reverse transcriptases having the amino acid sequence set forth in SEQ ID NO: 195 (24) or SEQ ID NO: 196 (25). The improvement shown with mouse PBMCs were similar to the results observed with GEX in FIG. 24.

FIGS. 27A-D show the performance of one RT variant (50A+G; SEQ ID NO: 147) in a Single Cell 5′ (SC-5′) gene expression assay when compared to a control MMLV variant (42B); and show the superiority of the 50A+G as an improved RT for single cell assays. FIG. 27A shows a performance comparison of the control MMLV variant and 50A+G summarizing median genes and UMIs per cell at 20 k and 50 k raw-reads per cell (rrpc); and illustrating that 50A+G enhanced the median genes per cell by about 4.54% at 20 k rrpc or 13.1% at 50 k rrpc, while the median UMIs per cell was enhanced by 13.50% at 50 k rrpc when compared to the control MMLV variant. FIG. 27B shows a bar graph illustrating the performance of 50A+G at maximum normalization depth. FIGS. 27C-D show saturation curves for the control MMLV (42B) and 50A+G demonstrating the median genes (FIG. 27C) and counts/cell (FIG. 27D) as a function of read depth; and further demonstrating a clear benefit of using the 50A+G variant in the SC-5′assay as read depth increases.

FIGS. 28A-B show the performance of various RT variants in a Single Cell 5′ (SC-5′) gene expression assay at 20 k raw-reads per cell (rrpc). Median genes and UMIs/cell at 20 k rrpc show the enhanced performance, based on sensitivity gain of the novel variants when compared to the control MMLV variant (42B). SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131).

FIGS. 29A-B show the performance of various RT variants in a Single Cell 5′ (SC-5′) gene expression assay at 50 k raw-reads per cell (rrpc). Median genes and UMIs/cell at 50 k rrpc show the enhanced performance, based on sensitivity gain, of the novel variants when compared to the control MMLV variant (42B). SOLD 025 (SEQ ID NO: 111), SOLD 031 (SEQ ID NO: 123), SOLD 033 (SEQ ID NO: 127), SOLD 034 (SEQ ID NO: 129), and SOLD 035 (SEQ ID NO: 131).

FIGS. 30A-B show bar graphs illustrating the performance of various novel RT variants at maximum normalization depth. Median genes and UMIs/cell, at maximum normalization depth, show the enhanced performance, based on sensitivity gain, of the novel variants when compared to the control MMLV variant (42B). SOLD 025 (SEQ ID NO: 111), SOLD 031 (SEQ ID NO: 123), SOLD 033 (SEQ ID NO: 127), SOLD 034 (SEQ ID NO: 129), and SOLD 035 (SEQ ID NO: 131).

FIGS. 31A-C show scatter plots illustrating differential gene expression of some top performing novel RT variants (SOLD 025 (SEQ ID NO: 111), SOLD 031 (SEQ ID NO: 123), and SOLD 034 (SEQ ID NO: 129)) and gene expression correlation between 42B and some top preforming novel RT variants.

FIGS. 32A-C show volcano plots illustrating the number of differentially expressed genes between 42B and the same top preforming novel RT variants (SOLD 034 (SEQ ID NO: 129)) of FIGS. 31A-C.

FIGS. 33A-B show the performance of an engineered fusion reverse transcriptase comprising sto-7 described herein in a Single Cell 5′ (SC-5′) gene expression assay at 20 k raw-reads per cell (rrpc). Median genes and UMIs/cell at 20 k rrpc show the enhanced performance of the engineered fusion reverse transcriptase comprising sto-7, based on sensitivity gain of the sto-7 fusion RT when compared to the control MMLV variant (42B) and two additional 42B variants.

FIGS. 34A-B show the performance of an engineered fusion reverse transcriptase comprising sto-7 described herein in a Single Cell 5′ (SC-5′) gene expression assay at 50 k raw-reads per cell (rrpc). Median genes and UMIs/cell at 50 k rrpc show the enhanced performance of the engineered fusion reverse transcriptase comprising sto-7, based on sensitivity gain of the sto-7 fusion RT when compared to the control MMLV variant (42B) and two additional 42B variants.

FIGS. 34A-B show the performance of engineered fusion reverse transcriptases comprising sto-7 in a Single Cell 5′ (SC-5′) gene expression assay at maximum normalization depth. Median genes and UMIs/cell at maximum normalization depth show the enhanced performance of the engineered fusion reverse transcriptase comprising sto-7, based on sensitivity gain of the sto-7 fusion RT, when compared to the control MMLV variant (42B) and two additional 42B variants.

FIGS. 36A-C show scatter plots illustrating differential gene expression of engineered fusion reverse transcriptases comprising sto-7 and gene expression correlation between 42B and its variants (42B L and 42B V) and the corresponding engineered fusion reverse transcriptases comprising sto-7.

FIGS. 37A-C show volcano plots illustrating the number of differentially expressed genes of the variants tested in FIGS. 36A-C and showing that differential gene expression was more pronounced with Sto7 fusions on 42B L and 42B V.

FIGS. 38A-C show aggregated metrics graphs illustrating the performance comparison of the impact of the Sto7 fusion domain across three reverse transcriptase backbones (42B, 42B L and 42B V) based on median genes and UMIs/cell at maximum normalization depth (FIG. 38A), gene expression correlation (FIG. 38B), and differential gene expression (FIG. 38C). Comparing performance among 42B, 42B L, and 42B V backbones with and without the Sto7 fusion showed a clear performance benefit (assay sensitivity enhancement) associated with the Sto7 fusion.

FIG. 39 shows the performance of various RT variants in a Single Cell 3′ (SC-35′) gene expression assay at 50 k raw-reads per cell (rrpc). Median genes and UMIs/cell at 50 k rrpc show the enhanced performance, based on sensitivity gain, of the novel variants when compared to the control MMLV variant (enzyme mix B or enzyme mix C), SOLD 025 (SEQ ID NO: 111), and SOLD 034 (SEQ ID NO: 129).

FIGS. 40A-D show the performance of various engineered RT variants (e.g., SOLD 025 (SEQ ID NO: 111), and SOLD 034 (SEQ ID NO: 129)) in a Single Cell 3′ (SC-3′) gene expression assay when compared to a control MMLV variants (enzyme mix B and enzyme mix C); and show the superiority of SOLD 25 and SOLD 34 as improved RTs for single cell assays. FIGS. 40A-B show saturation curves for the control MMLV and the engineered RT demonstrating the median genes (FIG. 40A) and counts/cell (FIG. 40B) as a function of read depth; and further demonstrating a clear benefit of using the engineered RT variants in the SC-3′assay as read depth increases. FIGS. 40C-D show bar graphs demonstrating the performance comparison summarizing median genes and UMIs per cell at maximum normalization depth; and illustrating the superior performance of the RT variants at maximum normalization depth.

FIGS. 41A-C show scatter plots illustrating the differential gene expression of some top performing novel RT variants (SOLD 025 (SEQ ID NO: 111), and SOLD 034 (SEQ ID NO: 129)) based on a Single Cell 3′ (SC-3′) gene expression assay and gene expression correlation between a control RT (Enzyme Mix C), SOP and some top preforming novel RT variants.

FIGS. 42A-C show volcano plots illustrating the number of differentially expressed genes between SOP, the control RT (Enzyme Mix C) and top preforming novel RT variants of FIGS. 41A-C.

FIG. 43 shows a schematic diagram of a generalized capture probe used in spatial transcriptomics and single cell transcriptomic analyses, exemplary applications in addition to general reverse transcription reactions where the engineered thermostable reverse transcriptase of the invention could be used to extend a capture probe using a captured target nucleic acid as a template, thereby generating a cDNA product.

### DETAILED DESCRIPTION

### I. Overview

A challenge in cDNA synthesis reactions is interference from RNA secondary structures. While a higher reaction temperature can remove secondary structure from the template RNA, elevated temperatures typically lead to lower reverse-transcriptase (RT) enzyme activity if the enzyme is not nascently thermostable. Additionally, RT enzyme activity can be reduced by inhibitors, such as those which might be found in cell lysates and associated reagents. Wild-type (WT) Moloney Murine Leukemia Virus (MMLV) reverse-transcriptase is an RT enzyme that is typically inactivated at higher temperatures. Several commercially available mutant MMLV RT enzymes have been generated that exhibit improved thermostability, fidelity, substrate affinity, and/or reduced terminal deoxynucleotidyltransferase activity. However, while these variant MMLV RT may function well in routine amplification reactions, these variants are not optimal for reverse transcription of mRNA when using high throughput amplification reaction assays (e.g., spatial arrays and single cell transcriptomics assays) and the like. This is because high throughput amplification reaction assays require reaction volumes that are usually less than about 1 nanoliter. In addition, sample processing chemicals can negatively impact the function and activity of wild-type and available MMLV variants.

In a first aspect, the present disclosure is directed to an engineered fusion reverse transcriptase comprising: (a) at least one DNA binding domain selected from a DNA binding domain from an archaeal DNA binding protein or a single-stranded DNA binding domain; and (b) an engineered reverse transcriptase having an amino acid sequence that is at least about 90% identical to SEQ ID NO: 1. In another aspect, the engineered fusion reverse transcriptase exhibits an altered reverse transcriptase-related activity as compared to a reverse transcriptase having the amino acid sequence set forth in SEQ ID NO: 1.

In another aspect, the disclosure is directed to an engineered fusion reverse transcriptase designated MMLV-Sto7 (K13L) (SEQ ID NO:20). The linker in this fusion is depicted in SEQ ID No: 19. The Sto-7 sequence (or DNA binding protein) is shown in SEQ ID NO:18. SEQ ID NO: 55 sets forth the amino acid sequence of the engineered RT (MMLV variant). Non-limiting embodiments of additional engineered Sto7 fusion RTs are shown in Table 5, e.g., SEQ ID NOs: 3, and 5.

The DNA binding domain enhances the enzymatic activity of the engineered reverse transcriptase. For example, the addition of the DNA binding domain can enhance the template switching (TS) efficiency, higher end-to-end template jumping/switching, processivity efficiency, binding affinity, transcription efficiency, chemical tolerance, ability to yield mitochondrial unique molecular identifier (UMI) counts, ability to yield ribosomal unique molecular identifier (UMI) counts, shelf life, higher strand displacement, increased thermostability, improved thermoreactivity, and any combination thereof, for the engineered (i.e., recombinant) reverse transcriptase when compared to WIT MMLV or known MMLV variants.

TS efficiency: Small RNAs (<200 nucleotides) are for the most part non-coding regulatory elements and play a key role in gene expression. Small RNAs regulate gene expression in plants, animals, and many fungi-including several roles in development, proliferation, differentiation, immune reaction, apoptosis, tumorigenesis and adaptation to stress. Given their importance in regulation, miRNAs are candidates as biomarkers for several human diseases. Thus, developing accurate and reproducible ways to study these and other small RNAs is necessary to further decipher their biological consequences.

The main sources of bias in a typical library preparation workflow are the enzymatic ligations that introduce 5′ and 3′ sequencing adaptors to single-stranded templates. Template switching permits ligation-free incorporation of the 5′ adapter during reverse transcription. Template switching-based methods depend upon the natural tendency of MMLV-type reverse transcriptases to add nontemplated nucleotides at the 3′ end of the emerging cDNA strand. These nontemplated additions serve as an anchoring unit for annealing complementary nucleotides in a provided template switching oligonucleotide (TSO); upon reaching the cDNA-TSO cross-junction, the reverse transcriptase effectively switches templates, continuing cDNA synthesis out of the TSO sequence. By incorporating the 5′ adapter sequence into the TSO, and using polyadenylation to prime reverse transcription, ligation steps can be avoided altogether. For applications where the total RNA input is limited, such as single-cell RNA sequencing, template switching offers a critical advantage as it reduces the number of steps and sample loss during library preparation. Thus, the engineered fusion reverse transcriptase described herein, exhibiting improved TS efficiency, is highly desirable.

Higher end-to-end template jumping or switching: End-to-end template jumping or switching refers to the ability of a reverse transcriptase to template-switch from the 5′ end of one template to the 3′ end of another. Improved end-to-end template jumping or switching can result in an improved process efficiency. Thus, the engineered fusion reverse transcriptase described herein, exhibiting improved or higher end-to-end template jumping or switching, is highly desirable.

The processivity of a reverse transcriptase refers to the number of nucleotides incorporated in a single binding event of the enzyme. Therefore, a highly processive reverse transcriptase can synthesize longer cDNA strands in a shorter reaction time. Some engineered MMLV reverse transcriptases can add as many as 1,500 nucleotides in a single binding event, which represents a processivity that is about 65 times greater than that of wild-type MMLV reverse transcriptase. Enzyme processivity is also associated with its affinity for the template. As such, reverse transcriptases with high processivity are resistant to common inhibitors that may have carried over from the RNA sources. Examples of reverse transcriptase inhibitors include heparin and bile salts from blood and stool, humic acid and polyphenols from soil and plants, and formalin and paraffin from formalin-fixed, paraffin-embedded (FFPE) samples. These inhibitors often remain bound to RNA and/or reduce polymerization activity, and highly processive reverse transcriptases are better able to overcome such inhibition.

Highly processive reverse transcriptases also perform better with RNA samples of low quality and quantity. This attribute makes highly processive reverse transcriptases ideal for RNA isolated from plant and animal tissues as well as clinical research samples, which tend to be degraded due to processing and RNase-rich environments. Likewise, these enzymes are a good choice for experiments when limited amounts of RNA are available. Thus, the engineered fusion reverse transcriptase described herein, exhibiting improved processivity and/or processivity efficiency, is highly desirable.

DNA binding affinity: To initiate reverse transcription, reverse transcriptases require a short DNA oligonucleotide called a primer to bind to its complementary sequences on the RNA template and serve as a starting point for synthesis of a new strand. Improved binding affinity results in a more efficient process, particularly when limited amounts of RNA are available. Thus, the engineered fusion reverse transcriptase described herein, exhibiting improved DNA binding affinity, is highly desirable.

Transcription efficiency: The RNA-to-cDNA conversion step in transcriptomics experiments is widely recognized as inefficient and variable. This issue is particularly significant for transcriptomics at the single cell level, which is preferable due to greater recognition of sample heterogeneity. Transcriptomics measurements almost invariably include a reverse transcription (RT) step, where RNA transcripts are used as templates to generate cDNA transcripts for quantification. This significantly complicates data interpretation as techniques are not directly measuring RNA transcript number, and results are therefore dependent on the efficiency of the RNA to cDNA conversion. Thus, the engineered fusion reverse transcriptase described herein, exhibiting improved transcription efficiency, is highly desirable.

Chemical tolerance: Reverse transcriptases function in an environment that may include processing chemicals, such as cell fixation chemicals or processing reagents, which can negatively impact the function and activity of the enzyme. Thus, the engineered fusion reverse transcriptase described herein, exhibiting improved chemical tolerance, is highly desirable.

Ability to yield mitochondrial and/or ribosomal unique molecular identifier (UMI) counts: Unique molecular identifier (UMI) counting is a gene expression quantification scheme used in single-cell RNA-sequencing (scRNA-seq) analysis. Single-cell RNA-sequencing (scRNA-seq) technology provides transcriptome profiles of individual cells, enabling the dissection of the heterogeneity of different cell populations and tissues. The paucity of starting material for reverse transcription remains an inherent limitation of scRNA-seq protocols and contributes to the relatively low rate at which messenger RNA (mRNA) molecules in individual cells are converted to cDNA molecules that can be captured and sequenced. The miniscule quantity of transcripts captured from a single cell requires cDNA amplification for library construction; this inevitably results in large amplification bias. To mitigate this bias, some scRNA-seq protocols employ an additional step in which individual transcripts are barcoded with unique molecular identifiers (UMIs) before amplification, resulting in a more accurate quantification of the transcript count. UMIs incorporate a unique barcode onto each molecule within a given sample library. By incorporating individual barcodes on each original DNA fragment, variant alleles present in the original sample (true variants) can be distinguished from errors introduced during library preparation, target enrichment, or sequencing. Thus, the engineered fusion reverse transcriptase described herein, exhibiting an improved ability to yield mitochontrial and/or ribosomal UMI counts, is highly desirable.

Shelf life and/or stability: In another aspect of the disclosure, the engineered fusion reverse transcriptase described herein, exhibit improved stability and/or shelf life. A longer period of stability, and/or shelf life, is desirable as it can result in more efficient processes.

Higher strand displacement: Strand displacement is the process through which two strands with partial or full complementarity hybridize to each other, displacing one or more pre-hybridized strands in the process. Reverse transcriptase first transcribes a complementary strand of DNA to make an RNA:DNA hybrid. Next, reverse transcriptase or RNase H degrades the RNA strand of the hybrid. The single-stranded DNA is then used as a template for synthesizing double-stranded DNA (cDNA). Thus, reverse transcriptase (RT) catalyzes the conversion of RNA into an integration-competent double-stranded DNA, with a variety of enzymatic activities that include the ability to displace a non-template strand concomitantly with polymerization. RT are capable of efficiently unwinding duplexes in the template during polymerization. This strand displacement synthesis activity by RT is required for the polymerization on the highly structured RNA and the removal of RNA fragments which cannot be cleaved by the enzymes RNase H activity. In addition, strand displacement synthesis on a DNA duplex is particularly important to complete the plus- and minus-strands by polymerizing on the long terminal repeats. As such, an RT with a higher strand displacement property is more efficient. Accordingly, the engineered fusion reverse transcriptase described herein, exhibiting an improved strand displacement property, is highly desirable.

Thermostability and or thermoreactivity: The ability of a reverse transcriptase to withstand high temperatures is an important aspect of cDNA synthesis. Elevated reaction temperatures help denature RNA with strong secondary structures and/or high GC content, allowing reverse transcriptases to read through the sequence. As a result, reverse transcription at higher temperatures enables full-length cDNA synthesis and higher yields, which leads to better representation of an RNA population by the cDNAs. With gene-specific primers in one-step RT-PCR, reverse transcription at higher temperatures enhances specificity of the primers' binding to the target. This strategy enables increased yield and reduced background in subsequent PCR, making reverse transcriptases with high thermostability desirable for cDNA synthesis. Thus, the engineered fusion reverse transcriptase described herein, exhibiting improved thermostability, is highly desirable.

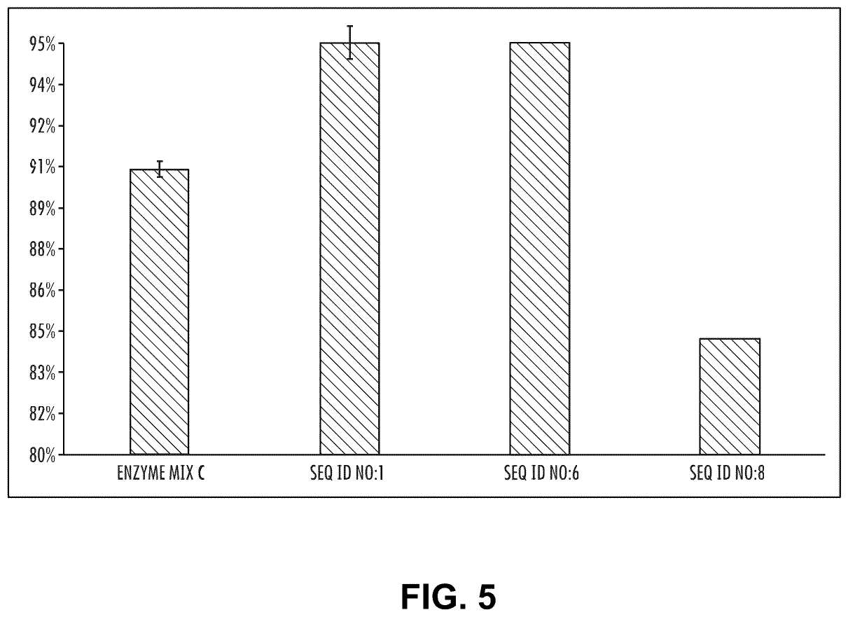

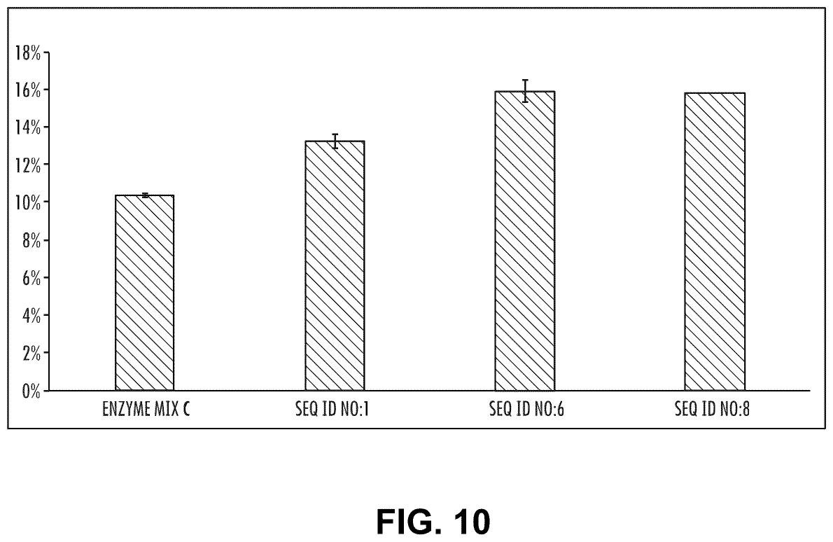

Experimental results: As detailed in Examples 3, 4, and 5, the fusion RTs of the present disclosure exhibited increased UMI counts (Example 3; FIGS. 5-10). In particular, FIG. 10 shows that the fusion MMLV enzymes provided products that yielded higher fraction mitochondrial UMI counts as compared to the control. Improved template switching efficiency is detailed in Example 5, where the tested fusion RT showed enhanced template switching efficiency as compared to the MMLV variant of SEQ ID NO: 1.

As disclosed herein, in one aspect the disclosure encompasses an engineered fusion reverse transcriptase comprising at least one DNA binding domain of SEQ ID NO: 18 and an engineered reverse transcriptase of any one of SEQ ID NO: 1, 14, and 22-55 showed improved processivity, template switching efficiency, DNA binding affinity, and/or transcription efficiency when compared to an unconjugated reverse transcriptase, a wild-type MMLV reverse transcriptase, or a variant MMLV. In a non-limiting embodiment, the engineered fusion RT is 42B L Sto7K13L (shown in Table 4 as SEQ ID: 20).

To improve the biophysical properties of the engineered reverse transcriptase disclosed herein, the engineered reverse transcriptase is engineered as a recombinant fusion protein comprising at least one DNA binding domain derived from an archaeal DNA binding protein, such as for example, Sto7d, Sso7d, Sis7b, Sis7a, Ssh7b, Sto7, Aho7C, Aho7B, Aho7A, Mcu7, Mse7, Sac7e, and Sac7d; and an engineered reverse transcriptase disclosed herein.

Another aspect of the present disclosure provides an engineered fusion reverse transcriptase comprising: (a) an engineered reverse transcriptase comprising, consisting essentially of, or consisting of (i) an amino acid sequence that is at least about 90% identical to SEQ ID NO: 1; (ii) 90-99.99% identical to SEQ ID NO: 1, 92-99.99% identical to SEQ ID NO: 1, 93-99.99% identical to SEQ ID NO: 1, 94-99.99% identical to SEQ ID NO: 1, 95-99.99% identical to SEQ ID NO: 1, 96-99.99% identical to SEQ ID NO: 1, 97-99.99% identical to SEQ ID NO: 1, 98-99.99% identical to SEQ ID NO: 1; or (iii) 90%, 91%, 92%, 93%, 94%, 95%, 96%, 97%, 98%, 99% or 99.5% identical to SEQ ID NO: 1, wherein the amino acid variation(s) are at any one position or combination thereof as identified in an alignment of SEQ ID NO: 1 to any one of the RT polypeptide sequences in Table 4, Table 5, or Table 6; and (b) a DNA binding domain from an archaeal protein selected from Sto7d, Sso7d, Sis7b, Sis7a, Ssh7b, Sto7, Aho7C, Aho7B, Aho7A, Mcu7, Mse7, Sac7e, and Sac7d; or (c) a DNA binding domain having at least 90%, at least about 92%, at least about 95%, at least about 97%, at least about 98%, or at least about 99% sequence identity to SEQ ID NO: 12, 13, 16, 17, and 18.

Another aspect of the present disclosure provides an engineered fusion reverse transcriptase comprising: (a) an engineered reverse transcriptase comprising, consisting essentially of, or consisting of (i) an amino acid sequence consisting essentially of consisting of: SEQ ID NO: 14, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55; (ii) SEQ ID NOs: 180-208; (iii) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; (iv) or any of the RT polypeptide sequences listed in Table 4, Table 5 or Table 6; and (b) a DNA binding domain from an archaeal protein selected from Sto7d, Sso7d, Sis7b, Sis7a, Ssh7b, Sto7, Aho7C, Aho7B, Aho7A, Mcu7, Mse7, Sac7e, and Sac7d; or (c) a DNA binding domain having at least 90%, at least about 92%, at least about 95%, at least about 97%, at least about 98%, or at least about 99% sequence identity to SEQ ID NO: 12, 13, 16, 17, and 18.

Another aspect of the present disclosure provides an engineered fusion reverse transcriptase comprising an amino acid sequence selected from SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170; or an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, or 170.

Another aspect of the present disclosure provides an isolated nucleic acid molecule encoding an engineered reverse transcriptase described herein; a DNA binding domain described herein; or an engineered fusion reverse transcriptase described herein. In some embodiments, the isolated nucleic acid molecule comprises a sequence selected from SEQ ID NO: 136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:167, SEQ ID NO:169, or SEQ ID NO: 171; or a nucleic acid sequence of Table 5.

Another aspect of the present disclosure provides an expression vector comprising the isolated nucleic acid. Another aspect of the present disclosure provides a host cell transfected with the expression vector or the isolated nucleic acid described herein.

Another aspect of the present disclosure provides methods for performing a reverse transcription reaction for generating a nucleic acid product from an RNA template using an engineered fusion reverse transcriptase described herein; or a nucleic acid extension method comprising an engineered fusion reverse transcriptase or an engineered reverse transcriptase described herein.

Another aspect of the present disclosure provides methods of using the engineered fusion reverse transcriptase described herein in an amplification reaction and/or high throughput amplification reaction assays (e.g. spatial arrays and single cell transcriptomics assays).

Any of the engineered RT enzymes of the present disclosure, including without limitation any of the enzymes comprising the amino acid sequence and/or nucleic acid sequences shown in Table 4 or Table 5 could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of mutliomic extensions or applications.

### II. Engineered Fusion Reverse Transcriptases

Reverse transcriptases or reverse transcription (RT) enzymes are RNA-dependent DNA polymerases, typically used to create a copy of an RNA sequence thereby generating a cDNA molecule. Reverse transcription is initiated by hybridization of a priming sequence to an RNA molecule which is extended by a reverse transcription enzyme in a template directed fashion. A reverse transcription enzyme adds a plurality of non-template nucleotides to a nucleotide strand, thereby producing complementary deoxyribonucleic acid (cDNA) molecules. The resultant cDNA can then be dehybridized from the template RNA molecule in any number of ways as known in the art.

Engineered and/or recombinant are used interchangeably with respect to reverse transcriptase (RT) variant and/or fusion RT.

One aspect of the present disclosure provides an engineered fusion reverse transcriptase comprising at least one DNA binding domain and an engineered reverse transcriptase. The at least one DNA binding domain and the engineered reverse transcriptase of the engineered fusion reverse transcriptase may be immediately adjacent to each other or separated by a linker region/linker. The DNA binding domain may be selected from the DNA binding domains of an archaeal DNA binding protein and/or single-stranded DNA binding domains. A DNA binding domain may be N-terminal to the engineered reverse transcriptase, C-terminal to the engineered reverse transcriptase, at the C-terminus of the engineered fusion reverse transcriptase, or at the N-terminus of the engineered fusion reverse transcriptase. When the engineered fusion reverse transcriptase comprises at least two DNA binding domains, the DNA binding domains may be at the same terminus or at different termini. The at least two DNA binding domains may be the same DNA binding domains or may be different DNA binding domains. A non-limiting embodiment comprising a linker GGGGS (SEQ ID NO: 19) between the RT sequence and Sto7 sequence as shown in SEQ ID NO: 20 (Table 4). Any suitable linker, including without limitation any variation of G(n)S(m)G(p) linker, where n=0, 1, 2, 3, 4, 5, 6, 7, 8, 9, 10 11, 12, 13, 14, 15, 16, 17, 18, 19 or 20, m=0, 1, 2, 3, 4, 5, 6, 7, 8, 9, 10 11, 12, 13, 14, 15, 16, 17, 18, 19 or 20, p=0, 1, 2, 3, 4, 5, 6, 7, 8, 9, 10 11, 12, 13, 14, 15, 16, 17, 18, 19 or 20, and n, m, and p are selected independently, could be inserted between the RT polypeptide and the DNA binding protein.

For example, (1) the DNA binding domains located at the N-terminus and the C-terminus can both be a Sso7d DNA binding domain; (2) the DNA binding domain located at the N-terminus and the C-terminus can both be a Sto7 DNA binding domain; (3) the DNA binding domain located at the N-terminus can be a Ss07d DNA binding domain and the DNA binding domain located at the C-terminus can be s Sto7 DNA binding domain; or (4) the DNA binding domain located at the N-terminus can be a Sto7 DNA binding domain and the DNA binding domain located at the C-terminus can be a Ss07d DNA binding domain.

Accordingly, in some embodiments the engineered fusion reverse transcriptase comprises a Ss07d DNA binding domain located at the N-terminus and a Sto7 DNA binding domain located at the C-terminus of the amino acid sequence. In another embodiment, the engineered fusion reverse transcriptase comprises a Sto7 DNA binding domain located at the N-terminus and an Ss07d DNA binding domain located at the C-terminus of the amino acid sequence.

In some embodiments, the DNA binding domain located at the N-terminus can be selected from a Sto7d, Sso7d, Sis7b, Sis7a, Ssh7b, Sto7, Aho7C, Aho7B, Aho7A, Mcu7, Mse7, Sac7e, or Sac7d DNA binding domain and the DNA binding domain located at the C-terminus can be selected from a Sto7d, Sso7d, Sis7b, Sis7a, Ssh7b, Sto7, Aho7C, Aho7B, Aho7A, Mcu7, Mse7, Sac7e, or Sac7d DNA binding domain. In some embodiments, the DNA binding domain located at the N-terminus can be a Sso7d DNA binding domain and the DNA binding domain located at the C-terminus of can be a Sto7d, Sso7d, Sis7b, Sis7a, Ssh7b, Sto7, Aho7C, Aho7B, Aho7A, Mcu7, Mse7, Sac7e, or Sac7d DNA binding domain.

In some embodiments, the DNA binding domain located at the N-terminus can be a Sto7d DNA binding domain and the DNA binding domain located at the C-terminus of can be a Sto7d, Sso7d, Sis7b, Sis7a, Ssh7b, Sto7, Aho7C, Aho7B, Aho7A, Mcu7, Mse7, Sac7e, or Sac7d DNA binding domain.

In some embodiments, the engineered fusion reverse transcriptase described herein comprises a DNA binding domain comprising an amino acid sequence selected from SEQ ID NO: 12, 13, 16, 17, or 18; and an engineered reverse transcriptase comprising an amino acid sequence selected from: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NOs: 180-208, SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or an amino acid sequence listed in Table 5. In this embodiment, the DNA binding domain can be located at the C-terminus or N-terminus of the engineered fusion reverse transcriptase.

### A. DNA Binding Domains

A DNA binding domain is a protein, or a defined region of a protein, that binds to a nucleic acid in a sequence-independent matter. For example, binding of the protein to DNA does not exhibit any preference for a particular sequence. The DNA binding domain may be single or double stranded. The nucleic acid binding domain can comprise a single stranded DNA binding protein; a double stranded DNA binding protein; a single stranded RNA binding protein; a double stranded RNA binding protein; a continuous RNA-DNA hybrid binding protein; or a discontinuous RNA-DNA hybrid binding protein.

The nucleic acid binding domain can help stabilize the interaction between the RNA template and the DNA primer during reverse transcription. For example, the nucleic acid binding domain can enhance the efficiency and/or processivity of the engineered thermostable enzyme during reverse transcription. Suitable DNA binding domains of the present disclosure can be identical to or substantially identical to a known DNA binding protein over a comparison window of about 25 amino acids, about 50 to about 100 amino acids, any value in-between these two parameters of 25 and 100 amino acids (e.g., about 55 to about 75 amino acids), or over the length of the entire protein. The sequence can be compared and aligned for maximum correspondence over a comparison window, or designated region as measured using one of the described comparison algorithms or by manual alignment and visual inspection. For purposes of this disclosure, percent amino acid identity is determined by the default parameters of BLAST and or CLUSTAL W.

DNA binding domain (DBD) proteins or polypeptides are capable of binding DNA. DNA binding domains may include, but are not limited to, one or more DNA binding domains from an archaeal DNA binding protein, single-stranded DNA binding domains and/or 7 kDa DNA binding domains. One or more DNA binding domains described herein can be obtained from archaebacterial proteins and may include, but not limited to, Sto7, Sso7d, Sis7b, Sis7a, Ssh7b, Sto7, Aho7C, Aho7B, Aho7A, Mcu7, Mse7, Sac7e, and Sac7d.

The DNA binding domain may be from Sto7, or Sto7d. The DNA binding domain may be from a Sso7d, Sso7d like or Sso7d nucleic acid binding domain. Sso7d and Sso7d-like proteins, Sac7d and Sac7d-like proteins (e.g., Sac7a, Sac7b, Sac7d, and Sac7e) are small (about 7,000 kd MW), basic chromosomal proteins from the hyperthermophilic archaebacteria Sulfolobus solfataricus and S. acidocaldarius, respectively. These proteins are lysine-rich and have high thermal, acid and chemical stability. They bind DNA in a sequence-independent manner and when bound, increase the melting temperature (TM) of DNA by up to 40° C. These proteins and their homologs are typically believed to be involved in stabilizing genomic DNA at elevated temperatures. Suitable Sso7d-like DNA binding domains for use in the present disclosure can be modified based on their sequence homology to Sso7d. In some embodiments, the DNA binding domain is derived Sulfolobus solfataricus Sso7d and/or comprises the amino acid sequence set forth in SEQ ID NO:13. In some embodiments, the engineered fusion reverse transcriptase comprises a Sulfolobus solfataricus Sso7d DNA binding domain comprising for example the amino acid sequence of SEQ ID NO: 6 or 8.

In some embodiments, the DNA binding domain may comprise an archaeal DNA binding domain consensus motif having the amino acid sequence set forth in SEQ ID NO: 2. Sto7 is a DBD from Sulfolobus tokadaii. The Sto7 amino acid sequence is set forth in SEQ ID NO:12. 7 kDa DBD may include, but are not limited to, DBDs approximately 7 kDa, Sto7 and Sso7d. In some embodiments, the DNA binding domain can comprise a mutation selected from a K13 mutation, a K13L mutation, a D36 mutation, a D36E mutation, a E36 mutation, a E36D mutation, an N37 mutation, an N37G mutation, an G37 mutation, an G37N mutation, a V2 mutation, a V2A mutation, a D36L mutation, an insertion, a glycine insertion at for example position 38, a deletion, a deletion of a glycine at for example position 38 in SEQ ID NO: 12 or 13 or a combination thereof. The DNA binding domain can comprise an amino acid sequence selected from SEQ ID NO: 12, 13, 16, 17, or 18; or an amino acid sequence having at least 90%, at least about 92%, at least about 95%, at least about 97%, at least about 98%, or at least about 99% sequence identity to SEQ ID NO: 12, 13, 16, 17, or 18. In some embodiments, the DNA binding domain comprises the amino acid sequence of SEQ ID NO: 18.

The DNA binding domain can be a single-stranded DNA binding domain. Single-stranded DNA binding domains preferentially bind single-stranded DNA. DBD may comprise one or more site specific alterations including, but not limited to a K13 alteration, such as a K13L alteration. Such alterations may alter one or more aspects of DNA binding. In some embodiments, the K13L mutation is an RNAse silencing mutation on Sto7. In some embodiments, a DNA binding domain comprising a K13L mutation comprises SEQ ID NO: 18. The alteration may be an increase or decrease in an aspect of DNA binding. Furthermore, it is recognized that an alteration that increases one aspect of DNA binding may alter a different aspect of DNA binding. The alteration of a different aspect of DNA binding may be an increase or a decrease. The DNA binding domain can also exhibit reduced RNAase activity. Alternatively, the amino acid sequence of any DNA binding domain described herein can be altered to reduce RNAase activity.

### B. Reverse Transcriptases

Reverse transcriptases or reverse transcription enzymes are known in the art to perform a reverse transcription reaction. As used herein, “Reverse transcriptase” and “reverse transcription enzyme” are synonymous. Reverse transcription is initiated by hybridization of a priming sequence to an RNA molecule which is extended by an engineered reverse transcription enzyme in a template directed fashion. A reverse transcription enzyme adds a plurality of non-template oligonucleotides to a nucleotide strand. The reverse transcription reaction can produce single stranded complementary deoxyribonucleic acid (cDNA) molecules each having a molecular tag on a 5′ end thereof, followed by amplification of cDNA to produce a double stranded DNA having the molecular tag on the 5′ end and a 3′ end of the double stranded DNA. As used herein, the term “wild-type” refers to a gene or gene product that has the characteristics of that gene or gene product when isolated from a naturally occurring source. For example, the amino acid sequence set forth in SEQ ID NO: 7 is a wild-type MMLV amino acid sequence.

An engineered fusion reverse transcriptase may exhibit one or more reverse transcriptase related activities including but not limited to, an RNA-dependent DNA polymerase activity, an RNAse H activity, a DNA-dependent DNA polymerase activity, an RNA binding activity, a DNA binding activity, a polymerase activity, a primer extension activity, a strand-displacement activity, a helicase activity, a strand transfer activity, a template binding activity, transcription template switching, transcription efficiencies, template switching efficiencies, processivity efficiencies, incorporation efficiencies, fidelity efficiencies, polymerization efficiencies, altered specificity, altered non-templated base addition, altered thermostability, altered tailing, altered adapter binding, binding efficiencies, ability to yield unique molecular identifiers (UMI), ability to yield median UMI, transcription efficiency, template switching efficiency, processivity, incorporation efficiency, Kd, distribution, fidelity, polymerization efficiency, Km, specificity, non-templated base addition, thermostability, tailing, adapter binding, binding efficiency, binding affinity (Km/Kcat), Vmax and ability to yield median UMI/cell and altered binding affinities.

A change in any activity may increase, decrease or have no effect on a different reverse-transcriptase related activity. In addition, a change in one activity may alter multiple properties of a reverse transcriptase. When multiple properties are affected, the properties may be altered similarly or differently. Methods of evaluating reverse transcriptase related activities are known in the art. A change in a reverse transcriptase related activity may alter one or more of the following results including but not limited to the yield of unique molecular identifiers (UMI), the median UMI obtained, the yield of mitochondrial UMI counts, and/or the yield of ribosomal UMI counts. A change or alteration in the yield of UMI the median UMI obtained, the yield of mitochondrial UMI counts, and/or the yield of ribosomal UMI counts may indicate one or more altered reverse transcriptase related activities.

In some embodiments, the fusion domain may occur at the N-terminus or C-terminus of the variant engineered reverse transcriptase amino acid sequence. Further, an engineered reverse transcription enzyme may comprise a DBD fusion domain at the N-terminus and C-terminus of the reverse transcriptase amino acid sequence. In some embodiments, a DBD fusion domain occurs at the actual N-terminus or C-terminus of the entire polypeptide. In some embodiments, a DBD fusion domain occurs at the N-terminus or C-terminus of the engineered reverse transcriptase amino acid sequence and is internal to an additional affinity tag. The amino acid sequence of a DNA binding domain consensus motif is set forth in SEQ ID NO:2.

DNA binding involves multiple aspects or properties related to an enzyme's ability to interact with and bind to a DNA molecule. DNA binding related properties may include, but are not limited to, processivity, clamping, off rate and on rate kinetics, template switching and RNase activity.

In various embodiments, the amino acid sequence of the engineered reverse transcriptase comprises a Sto7 DNA binding domain at the C-terminus. In various embodiments, the amino acid sequence of the engineered reverse transcriptase comprises an Ss07d DNA binding domain at the N-terminus or an Ss07d DNA binding domain at the C-terminus, or vice versa.

In some embodiments, engineered reverse transcription enzymes, engineered reverse transcriptases, engineered fusion reverse transcriptases described herein may comprise an affinity tag at the N-terminus or at a C-terminus of the amino acid sequence. In some instances, the affinity tag may include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, said affinity tag is at least 5 histidine amino acids (SEQ ID NO: 177).

In some embodiments, an engineered reverse transcriptase and/or an engineered fusion reverse transcriptase described herein can comprise a protease cleavage sequence. In that embodiment, cleavage by a protease results in cleavage of the affinity tag from the engineered reverse transcription enzyme. In some instances, the protease cleavage sequence is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase. In some instances, the protease cleavage sequence is a thrombin cleavage sequence.

### C. Reverse Transcriptase Variants

One aspect of the present disclosure provides an engineered fusion reverse transcription enzyme comprising at least one DNA binding domain and an engineered reverse transcriptase comprising an amino acid sequence that is (i) at least 90% identical to SEQ ID NO: 1, (ii) 90-99.99% identical to SEQ ID NO: 1, 92-99.99% identical to SEQ ID NO: 1, 93-99.99% identical to SEQ ID NO: 1, 94-99.99% identical to SEQ ID NO: 1, 95-99.99% identical to SEQ ID NO: 1, 96-99.99% identical to SEQ ID NO: 1, 97-99.99% identical to SEQ ID NO: 1, 98-99.99% identical to SEQ ID NO: 1; or (iii) 90%, 91%, 92%, 93%, 94%, 95%, 96%, 97%, 98%, 99% or 99.5% identical to SEQ ID NO: 1, wherein the amino acid variation(s) are at any one position or combination thereof as identified in an alignment of SEQ ID NO: 1 to any one of the RT polypeptide sequences in Table 4, Table 5, or Table 6.

Another aspect of the present disclosure provides an engineered fusion reverse transcription enzyme comprising at least one DNA binding domain and an engineered reverse transcriptase comprising the amino acid sequence set forth in SEQ ID NO: 7. The engineered reverse transcriptase can exhibit an altered reverse transcriptase activity as compared to a reverse transcriptase having the amino acid sequence set forth in SEQ ID NO: 1 or 7.

The engineered reverse transcriptase of the present disclosure is a variant MMLV reverse-transcriptase having one or more mutations. Specifically, an engineered reverse transcriptase described herein comprises a combination of mutations in the amino acid sequence of either the wild-type MMLV (SEQ ID NO 7 or 178) or in a MMLV variant (SEQ ID NO: 1, 143 or 179).

As used herein, a “Mutation” refers to a change introduced into a parental or wild type DNA sequence that changes the amino acid sequence encoded by the DNA, including, but not limited to, substitutions, insertions, deletions, point mutations, mutation of multiple nucleotides or amino acids, transposition, inversion, frame shift, nonsense mutations, truncations or other forms of aberration that differentiate the polynucleotide or protein sequence from that of a wild-type sequence of a gene or gene product. The consequences of a mutation include, but are not limited to, the creation of a new character, property, function, or trait not found in the protein encoded by the parental DNA, including, but not limited to, N terminal truncation, C terminal truncation or chemical modification. A “mutation” also includes an N- or C-terminal extension. In some embodiments, the mutations disclosed herein are substitutions.

In particular, the present disclosure relates to mutant or modified reverse transcriptases that comprise one or more (e.g., one, two, three, four, five, ten, twelve, fifteen, twenty, etc.) amino acid changes. These amino acid changes render the reverse transcriptase more efficient for nucleic acid synthesis (e.g., single cell profiling assay) requiring very small volume, as compared to an unmutated or an unmodified reverse transcriptase. As will be appreciated by those skilled in the art, one or more of the amino acids identified may be deleted and/or replaced with one or a number of amino acid residues. In a preferred aspect, any one or more of the amino acids may be substituted with any one or more amino acid residues such as Ala, Arg, Asn, Asp, Cys, GIn, GIu, GIy, His, He, Leu, Lys, Met, Phe, Pro, Ser, Thr, Trp, Tyr, and/or Val.

In some embodiments, the engineered reverse transcriptase described herein comprises the amino acid sequence of SEQ ID NO:7, and comprises a combination of mutations selected from E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R or L671P. The engineered reverse transcriptase described herein can also comprise the amino acid sequence of SEQ ID NO:7, and comprises a combination of mutations selected from E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R or L671P.

In some embodiments, the engineered reverse transcriptase comprises an amino acid sequence that is at least 90% identical to an amino acid sequence selected from: SEQ ID NO: 14, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55; or SEQ ID NOs: 180-208; SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or (d) an amino acid sequence listed in Table 4, 5, or 6.

The amino acid sequence of the engineered reverse transcriptase can also comprise E69K, L139P, D200N, E302R, T306K, W313F, T330P, N454K, H503V, D524N, L603W, E607K, and H634Y. In some embodiments, the amino acid sequence of the engineered reverse transcriptase comprises a combination of mutations selected from: M66L and L435G; M39V, M66L, and L435K; M39V and L435K; M66L, L435G, P448A and D449G; M39V, M66L, L435G, P448A and D449G; or M66L.

In some embodiments, the amino acid sequence of the engineered reverse transcriptase comprises E69K, L139P, D200N, E302R, T306K, W313F, T330P, L435G, P448A, D449G, N454K, D524N, L603W, and E607K; and comprises a combination of mutations selected from M66L; M66L and H503V; M66L and H634Y; and M66L, H503V, or H634Y.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises a second combination of mutations selected from t D524N, T542D, P627S, A644V, D653H, K658R mutation, and wherein the D200 mutation is a D200N mutation, the D449 mutation is a D449G, the L603 mutation is an L603W, or the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises D524N, T542D, A644V, D653H, an R650H and K658R, and wherein the D200 mutation is a D200N mutation, the D449 mutation is a D449E mutation, the L603 mutation is an L603W mutation, and the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises E545G, D583N, and H594Q, and wherein the D200 mutation is a D200N mutation, the D449 mutation is a D449G mutation, the L603 mutation is an L603F mutation, and the E607 mutation is an E607K mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises D524N, T542D, A644V, D653H, and K658R, wherein the D200 mutation is a D200N mutation, the D449 mutation is a D449E mutation, the L603 mutation is an L603W mutation, the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises H204R, D524N, T542D, P627S, D583N, A644V, D653H and K658R, wherein the D200 mutation is a D200E mutation, the D449 mutation is a D449G mutation, the L603 mutation is an L603W mutation, the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises H204R, E545G, D583N, and H594Q, wherein the D200 mutation is a D200E mutation, the D449 mutation is a D449G mutation, the L603 mutation is an L603F mutation, the E607 mutation is an E607K mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and comprises P47L, D524N, T542D, D583N, P627S, A644V, D653H, and K658R, wherein the D200 mutation is a D200N mutation, the D449 mutation is a D449G mutation, the L603 mutation is an L603W mutation, the E607 mutation is an E607G mutation.

In some embodiments, the engineered reverse transcription enzyme comprises an amino acid sequence that is at least 95% identical to SEQ ID NO: 1 and the amino acid sequence of the engineered reverse transcriptase comprises at least one mutation indexed to SEQ ID NO:7 selected from a M17 mutation; an A32 mutation, a M44 mutation, a M39 mutation, a K47 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435 mutation, an L435G mutation, an L435K mutation, a P448 mutation, a D449 mutation, an R450 mutation, a n N454 mutation, an A480 mutation, an H481 mutation, a N502 mutation, an A502 mutation, an H503 mutation, a D524 mutation, an H572 mutation, a W581 mutation, a D583 mutation, a K585 mutation, an H594 mutation, an L603 mutation, an E607 mutation, an H612 mutation, a P614 mutation, a G615 mutation, an H634 mutation, a P636 mutation, a G637 mutation, an H638 mutation, a D653 mutation, or an L671 mutation, or a combination thereof, and in some embodiments further including a DBD sequence.

In some embodiments, the engineered reverse transcription enzyme comprises an amino acid sequence that is at least 95% identical to SEQ ID NO: 1 and the amino acid sequence of the engineered reverse transcriptase comprises an M39 mutation, a K47 mutation, an L435 mutation, a D449 mutation, a D524 mutation, an E607 mutation, a D653 mutation, and an L671 mutation as indexed to SEQ ID NO:7 and comprising at least one mutation indexed to SEQ ID NO:7 selected from a M17 mutation; an A32 mutation, a M44 mutation, a M39V mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, a n N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, or an H638 mutation, or a combination thereof, and optionally further including a DBD sequence.

In other embodiments, the engineered fusion reverse transcription enzyme exhibits an altered reverse transcriptase related activity when compared to a reverse transcriptase having the amino acid sequence set forth in SEQ ID NO: 1.

In some embodiments, an engineered reverse transcriptase comprises an amino acid sequence that is at least 95% identical to SEQ ID NO: 1. In other embodiments, the engineered reverse transcriptase exhibits an altered reverse transcriptase related activity as compared to a reverse transcriptase having the amino acid sequence set forth in SEQ ID NO: 1. In additional embodiments, the engineered reverse transcriptase comprises a combination of mutations indexed to SEQ ID NO:7 selected from i) an E69K mutation, an E302R mutation, a T306K mutation, a W313F mutation, a L435G mutation, or an N454K mutation, and comprising at least one mutation selected from an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; ii) an L139P mutation, a D200N mutation, a T330P mutation, an L603W mutation, or an E607K mutation, and comprising at least one mutation selected from: an M39V mutation, an M66L mutation an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation or an H638G mutation; iii) an A32V mutation, an L72R mutation, a D200C mutation, a G248C mutation, an E286R mutation, an E302R mutation, a W388R mutation, and an L435G mutation; or iv) a Y344L mutation and an I347L mutation.

In some embodiments, the engineered reverse transcription enzyme comprises an amino acid sequence that is at least 95% identical to a reverse transcriptase having the amino acid sequence set forth in SEQ ID NO:1. In some embodiments, the engineered reverse transcription enzyme comprises an amino acid sequence that is at least 95% identical to SEQ ID NO: 1 and has at least one mutation selected from the group comprising, consisting or consisting essentially of an M39V mutation, a P47L mutation, M66L mutation, an E69K mutation, an L139P mutation, a D200N mutation, an H204R mutation, an E302R mutation, a T306K mutation, a W313F mutation, a T330P mutation, an L435G mutation, a G429S mutation, an L435K mutation, a P448A mutation, a D449G mutation, a N454K mutation, an H503V mutation, a D524N mutation, a T542 mutation, an E545G mutation, a D583N mutation, an H594Q mutation, an L603W mutation, an E607K mutation, a P627S mutation, an H634Y mutation, an A644V mutation, an R650H mutation, a D653H mutation, a K658R mutation, an L671P mutation, or an S679P mutation; and the engineered reverse transcription enzyme exhibits an altered reverse transcriptase related activity.

In some embodiments, the application provides an engineered reverse transcriptase comprising an amino acid sequence that is at least 95% identical to SEQ ID NO:1 and the amino acid sequence of said engineered reverse transcriptase comprises a combination of mutations indexed to SEQ ID NO:7 or 178 selected from the group comprising (a) an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (b) an M66L mutation, an E69K mutation, an L139P mutation, a D200N mutation, an E302R mutation, a T306K mutation, a W313F mutation, a T330P mutation, a N454K mutation, a D524N mutation, an H503V mutation, an L603W mutation, an E607K mutation, and an H634Y mutation, and at least one mutation selected from the group comprising an L435G mutation, an L435K mutation, an M39V mutation, a P448A mutation and a D449G mutation; (c) an M39V mutation, an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; and (d) an M39V mutation, an E69K mutation, an L139P mutation, a D200 mutation, an E302R mutation, a T306K mutation, a W313F mutation, a T330P mutation, an L435K mutation, a G429S mutation, a P448A mutation, a D449 mutation, an N454K mutation, an L603 mutation, an E607 mutation and an L671P mutation, wherein said D200 mutation is selected from the group consisting of D200N and D200E, wherein said D449 mutation is selected from the group consisting of D449G an D449E, wherein said L603 mutation is selected from the group consisting of L603W and L603F, wherein said E607 mutation is selected from the group consisting of E607G and E607K, and further comprising at least one mutation selected from the group consisting of P47L, H204R, D524N, T542D, E545G, D583N, H594Q, P627S, A644V, R650H, D653H, K658R, L671P, and S679P.

In some embodiments an engineered reverse transcriptase of the present application has an amino acid sequence that is at least 95% identical to SEQ ID NO:1 and the amino acid sequence of said engineered reverse transcriptase comprises a combination of mutations indexed to SEQ ID NO:7 or 178; and the amino acid sequence of said engineered reverse transcriptase comprises a combination of mutations selected from the group consisting of: an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation and further comprising a second combination of mutations selected from the group consisting of: (a) an M66L mutation and an L534G mutation, (b) an M39V mutation, an M66L mutation and an L435K mutation, (c) an M39V mutation and an L435K mutation, (d) an M66L mutation, an L435G mutation, a P448 mutation, and D449G mutation, and (e) an M39V mutation, an M66L mutation, an L435G mutation, a P448 mutation and a D449G mutation.

In some embodiments an engineered reverse transcriptase of the present application has an amino acid sequence that is at least 95% identical to SEQ ID NO:1 and the amino acid sequence of the engineered reverse transcriptase comprises a combination of mutations selected from the group consisting of: an M39V mutation, an E69K mutation, an L139P mutation, a D200 mutation, an E302R mutation, a T306K mutation, a W313F mutation, a T330P mutation, a G429S mutation a P448A mutation, a D449 mutation, an L435K mutation, a N454K mutation, an L603 mutation, an E607 mutation, and an L671P mutation and further comprising a second combination of mutations selected from the group consisting of: (a) a D524N mutation, a T542D mutation, an A644V mutation, a D653H mutation, a K658R mutation, a S679P mutation, and wherein said D200 mutation is a D200N mutation, said D449 mutation is a D449G, said L603 mutation is an L603W, said E607 mutation is an E607G mutation, and a P627S mutation; (b) a D524N mutation, a T542D mutation, an A644V mutation, a D653H mutation, an R650 mutation and a K658R mutation, and wherein said D200 mutation is a D200N mutation, said D449 mutation is a D449E mutation, said L603 mutation is an L603W mutation, and said E607 mutation is an E607G mutation; (c) an E545G mutation, a D583N mutation, an H594Q mutation, and an S679P mutation, and wherein said D200 mutation is a D200N mutation, said D449 mutation is a D449G mutation, said L603 mutation is an L603F mutation, said E607 mutation is an E607K mutation; (d) a D524N mutation, a T542D mutation, an A644V mutation, a D653H mutation, and a K658R mutation, and wherein said D200 mutation is a D200N mutation, said D449 mutation is a D449E mutation, said L603 mutation is an L603W mutation, said E607 mutation is an E607G mutation; (e) an H204R mutation, a D524N mutation, a T542D mutation, a D583N mutation, an A644V mutation, a D653H mutation, and a K658R mutation, wherein said D200 mutation is a D200E mutation, said D449 mutation is a D449G mutation, said L603 mutation is an L603W mutation, said E607 mutation is an E607G mutation, and a P627S mutation, (f) an H204R mutation, an E454G mutation, a D583N mutation, an H594Q mutation, and an S679P mutation, wherein said D200 mutation is a D200E mutation, said D449 mutation is a D449G mutation, said L603 mutation is an L603F mutation, said E607 mutation is an E607K mutation; and (g) a P47 mutation, a D524N mutation, a T542D mutation, a D583N mutation, an A644V mutation, a D653H mutation, a K658R mutation and an S679P mutation, wherein said P47 mutation is a P47L mutation, D200 mutation is a D200N mutation, said D449 mutation is a D449G mutation, said L603 mutation is an L603W mutation, said E607 mutation is an E607G mutation, and a P627S mutation.

In some embodiments an engineered reverse transcriptase of the present disclosure has an amino acid sequence set forth in Table 6 or set forth in the group comprising SEQ ID NO:180, SEQ ID NO:181, SEQ ID NO:182, SEQ ID NO:183, SEQ ID NO:184, SEQ ID NO: 185, SEQ ID NO: 186, SEQ ID NO:187, SEQ ID NO:188, SEQ ID NO:189, SEQ ID NO:190, SEQ ID NO:191, and SEQ ID NO:192.

A variant may comprise a first combination of mutations or alterations and may comprise an additional or second combination of mutations.

A first combination of mutations or alterations may include, but is not limited to, a combination set forth herein: a M39 mutation, a K47 mutation, an L435 mutation, a D449 mutation, a D524 mutation, an E607 mutation, a D653 mutation and an L671 mutation; an M39V mutation, a K47 mutation, an L435K mutation, a D449G mutation, a D524N mutation, an E607 mutation, a D653 mutation and an L671 mutation; an M39 mutation, an M66 mutation, an E302 mutation, a T306 mutation, an L435 mutation, a D449 mutation, a D524 mutation, an E607 mutation, a D653 mutation and an L671 mutation; an M39 mutation, an M66 mutation, an E302 (K or R) mutation, a T306 (R or K) mutation, an L435 (K or G), a D449 mutation, a D524 mutation, an E607 (G or K) mutation, a D653 mutation, and an L671 mutation; and an M39V mutation, an M66 mutation, an E302 (K or R) mutation, a T306 (R or K) mutation, an L435 (K or G), a D449G mutation, a D524N mutation, an E607 (G or K) mutation, a D653 mutation, and an L671 mutation.

The second combination of mutations in a first engineered reverse transcriptase may comprise either a different set of mutations or a partially different second set of mutations as in a second engineered reverse transcriptase. A second combination of mutations or alterations may include but is not limited to (a) one or more mutations selected from an M17 mutation; an A32 mutation, a M44 mutation, a P51 mutation, an M66 mutation, an S67 mutation, an E69 mutation, a L72 mutation, a W94 mutation, a K103 mutation, an R110 mutation, a P117 mutation, an L139 mutation, an F155 mutation, an N178 mutation, an E179 mutation, a T197 mutation, a D200 mutation, an E201 mutation, an H204 mutation, a Q221 mutation, a V223 mutation, a V238 mutation, a G248 mutation, a T265 mutation, an E268 mutation, an R279 mutation, an R280 mutation, a K284 mutation, a T287 mutation, a F291 mutation, an E302 mutation, an E302K mutation, an E302R mutation, a T306 mutation, a T306R mutation, a T306K mutation, a P308 mutation, an F309 mutation, a W313 mutation, a T330 mutation, a Y344 mutation, an 1347 mutation, a C387 mutation, a W388 mutation, an R389 mutation, a C409 mutation, an R411 mutation, a G413 mutation, an A426 mutation, a G427 mutation, an L435G mutation, an L435K mutation, a P448 mutation, a D449G mutation, an R450 mutation, an N454 mutation, an A480 mutation, an H481 mutation, a N502 mutation, an A502 mutation, an H503 mutation, a D524N mutation, an H572 mutation, a W581 mutation, a D583 mutation, a K585 mutation, an H594 mutation, an L603 mutation, an H612 mutation, a P614 mutation, a G615 mutation, an H634 mutation, a P636 mutation, a G637 mutation, or an H638 mutation; (b) an E69K mutation, an E302R mutation, a T306K mutation, a W313F mutation, an L435G mutation, and an N454K mutation, and comprising at least one mutation selected from the group consisting of an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation and an H638G mutation; (c) an L139P mutation, a D200N mutation, a T330P mutation, an L603W mutation, and an E607K mutation, and comprising at least one mutation selected from the group consisting of: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation and an H638G mutation; (d) an A32V mutation, an L72R mutation, a D200C mutation, a G248C mutation, an E286R mutation, an E302R mutation, a W388R mutation, or an L435G mutation; and (e) a Y344L mutation and an I347L mutation. It is recognized that the second combination of mutations may comprise a group of mutations as described herein and one or more additional mutations.

In non-limiting embodiments, the engineered RT variants of the invention comprise a M39V, M66I, Q91R, I347V, H594Q, or a combination thereof in the RT backbone of SEQ ID NO: 143 or SEQ ID NO: 7. In non-limiting embodiments, the engineered RT variant comprises: M39V, M66I, Q91R, I347V, and H594Q (SEQ ID NO: 129, SOLD 034). In non-limiting embodiments, the engineered RT variants of the invention comprise M39V, T542D, D583N, E607G (42B is E607K), A644V, D653H, K658R, L671P, or a combination thereof in the RT backbone of SEQ ID NO: 143 or SEQ ID NO: 7. In non-limiting embodiments, the engineered RT variant comprises: M39V, T542D, D583N, E607G (42B is E607K), A644V, D653H, K658R, and L671P (SEQ ID NO: 111, SOLD 025).

In some embodiments, the engineered RT variant comprises: M39V, M66I, Q91R, I347V, H594Q, or a combination thereof, and optionally M39V, M66I, Q91R, I347V, H594Q, or the combination thereof (substituted) in the RT sequence of SEQ ID NO: 143 (SEQ ID NO: 129, SOLD 034).

In some embodiments, the engineered RT variant comprises: M39V, T542D, D583N, E607G, A644V, D653H, K658R, L671P, or a combination thereof, and optionally M39V, T542D, D583N, E607G, A644V, D653H, K658R, L671P, or the combination thereof substituted in the RT sequence of SEQ ID NO: 143 (or SEQ ID NO: 7) (SEQ ID NO: 111, SOLD 025).

### D. Enhanced Reverse Transcriptase Activity

The engineered reverse transcriptase of the present disclosure is a variant MMLV reverse-transcriptase with increased or enhanced reverse transcriptase activity. The term “increased” reverse transcriptase activity refers to the level of reverse transcriptase activity of a variant (e.g., mutant reverse transcriptase enzyme (e.g., MMLV variants disclosed herein) as compared to its wild-type form (e.g., wt MMLV or MMLV having the amino acid of SEQ ID NO: 7) or a known variant (e.g., MMLV having the amino acid of SEQ ID NO: 1). A mutant enzyme is said to have an “increased” reverse transcriptase activity if the level of its reverse transcriptase activity (as measured by methods described herein or known in the art) is at least 10% or more than its wild-type or a known variant. For example, the variant can have at least 10%, 15%, 20%, 25%, 30%, 40%, 50%, 60%, 70%, 80%, 90%, 100% more or at least 2-fold, 3-fold, 4-fold, 5-fold, or 10-fold or more activity than the wild-type or known variant.

The engineered fusion reverse transcription enzyme variants of the present disclosure unexpectedly provide an altered or improved reverse transcriptase activity, such as but not limited to, improved template switching (TS) efficiency, higher end-to-end template jumping/switching, improved processivity efficiency, improved binding affinity, improved transcription efficiency, improved chemical tolerance, improved ability to yield mitochondrial unique molecular identifier (UMI) counts, improved ability to yield ribosomal unique molecular identifier (UMI) counts, improved shelf life, higher strand displacement, increased thermostability, improved thermoreactivity, and any combination thereof. An engineered reverse transcription enzyme of the current application may exhibit an altered base-biased template switching activity such as an increased base-biased template switching activity, decreased base-biased template switching activity or an altered base-bias to the template switching activity.

The engineered reverse transcription enzyme variants of the present disclosure unexpectedly provided an altered reverse transcriptase activity, such as but not limited to, improved thermal stability, processive reverse transcription, non-templated base addition, binding affinity, and template switching ability. An engineered reverse transcription enzyme of the current application may exhibit an altered base-biased template switching activity such as an increased base-biased template switching activity, decreased base-biased template switching activity or an altered base-bias to the template switching activity. An engineered reverse transcriptase variant may exhibit enhanced template switching with a 5′-G cap on the substrate. Furthermore, an engineered reverse transcription enzyme variants described herein may also exhibit unexpectedly higher resistance to cell lysate (i.e., are less inhibited by cell lysate) than that exhibited by an enzyme having the amino acid sequence set forth in SEQ ID NO:1. Lastly, an engineered reverse transcription enzyme variants of the present disclosure may have an unexpectedly greater ability to capture full-length transcripts (e.g., in T-cell receptor paired transcriptional profiling), as compared to that exhibited by an enzyme having the amino acid sequence set forth in SEQ ID NO:1.

It is recognized that mutation of one or more residues may alter a first reverse transcriptase activity differently than a second reverse transcriptase activity. Further it is recognized that a different combination of mutations, such as different sites or residue changes may alter a reverse transcriptase activity similarly or differently. The variants that can template switch in the 5′ assay share the following alterations: E69K, E302R, T306K, W313F, L/K435G, and N454K. These variants may further comprise additional alterations that may affect one or more reverse transcriptase related activities. M39V and M66L may improve template switching. Without being limited by mechanism, variants comprising a M39V or a M66L mutation that do not exhibit altered performance in the 5′ GEM assay may exhibit an altered processivity, an altered k_{d} or both. K/L435 mutants may improve thermostability in the presence of primer template. In the absence of primer template K/L435 variants may exhibit a thermal denaturation profile similar to that of the wild-type protein. K/L435, P448 and D449 are residues in the connection domain; altering these residues may result in increased conformational flexibility. Additionally, the connection domain is thought to impact the conformational flexibility of the RNAase H domain. H503 and H634 occur within the RNAase H domain. The H503V and H634Y variants may impact primer-template contacting, processivity or both primer-template contacting and processivity.

Some variants share the following alterations: (a) the combination of variants consisting of a T542D mutation, a D583N mutation, an E607G mutation, an A644V mutation, a D653H mutation, and a K658R mutation. Some variants share the following alterations: (b) the combination of variants consisting of an E545G mutation, a D583N mutation, an H594Q mutation, an L603F mutation and a S679P mutation. These variants may further comprise additional alterations that may affect one or more reverse transcriptase related activities. The combination of variants consisting of a T542D mutation, a D583N mutation, an E607G mutation, an A644V mutation, a D653H mutation, and a K658R mutation and the combination of variants consisting of an E545G mutation, a D583N mutation, an H594Q mutation, an L603F mutation and a S679P mutation may exhibit an altered RNAse H activity.

### 1. RNase H Activity

In some embodiments, the engineered reverse transcriptase enzyme is engineered to have reduced and/or abolished RNase activity. RNase H activity refers to endoribonuclease degradation of the RNA of a DNA-RNA hybrid to produce 5′ phosphate terminated oligonucleotides that are 2-9 bases in length. RNase H activity does not include degradation of single-stranded nucleic acids, duplex DNA or double-stranded RNA. Removal of the RNase H activity of reverse transcriptase can eliminate the problem of RNA degradation of the RNA template and improve the efficiency of reverse transcription.

In some embodiments, the engineered reverse transcriptases or the engineered fusion reverse transcriptases of the present disclosure can have a reduced or substantially reduced RNase H activity. The reduction or substantial reduction or complete removal of the RNase H activity of a reverse transcriptase (e.g., MMLV) can prevent the degradation of an RNA template before the initiation of the RT reaction, thereby improving the efficiency of reverse transcription.

In some embodiments, the engineered reverse transcriptases or the engineered fusion reverse transcriptases of the present disclosure substantially lacks RNase H activity. In that embodiment, t the engineered reverse transcriptases or the engineered fusion reverse transcriptases of the present disclosure can have less than 10%, 5%, 1%, 0.5%, or 0.1% of the RNAse H activity of a wild type enzyme or a variant having the amino acid of SEQ ID NO: 1. In some embodiments, t the engineered reverse transcriptases or the engineered fusion reverse transcriptases of the present disclosure lacks RNase H activity. In that embodiment, the engineered reverse transcriptases or the engineered fusion reverse transcriptases of the present disclosure have undetectable RNase H activity or have an RNase H activity that is less than about 1%, 0.5%, or 0.1% of the RNase H activity of a wild type enzyme or a variant comprising the amino acid of SEQ ID NO: 1.

As used herein, the term “reduced RNase H activity” means that the enzyme has less than 50%, e.g., less than 40%, 30%, or less than 25%, 20%, more preferably less than 15%, less than 10%, or less than 7.5%, and most preferably less than 5% or less than 2%, of the RNase H activity of the corresponding wild type enzyme or a variant comprising the amino acid of SEQ ID NO: 1. The RNase H activity of an enzyme may be determined by assays known in the art.

In some embodiments, the engineered reverse transcription enzyme engineered to have reduced and/or abolished RNase H activity comprises a D524 mutation in SEQ ID NO: 1 or 7.

In some aspects, the amino acid sequence of the DNA binding domain portion of the fusion polypeptide has an alteration that impacts RNAase activity. Alterations to the amino acid sequence that may alter RNAase activity include, but are not limited to, a K13 mutation, a K13L mutation, a D36 mutation, and a D36L mutation. The amino acid sequence of an engineered fusion reverse transcriptase comprises a Sto7 DNA binding domain at the C-terminus of the polypeptide, where the DNA binding domain comprises a K13 mutation as provided in SEQ ID NO: 3. In some embodiments, the K13L mutation in Sto7 is a RNAse silencing mutation.

### 2. Template Switching Oligonucleotides

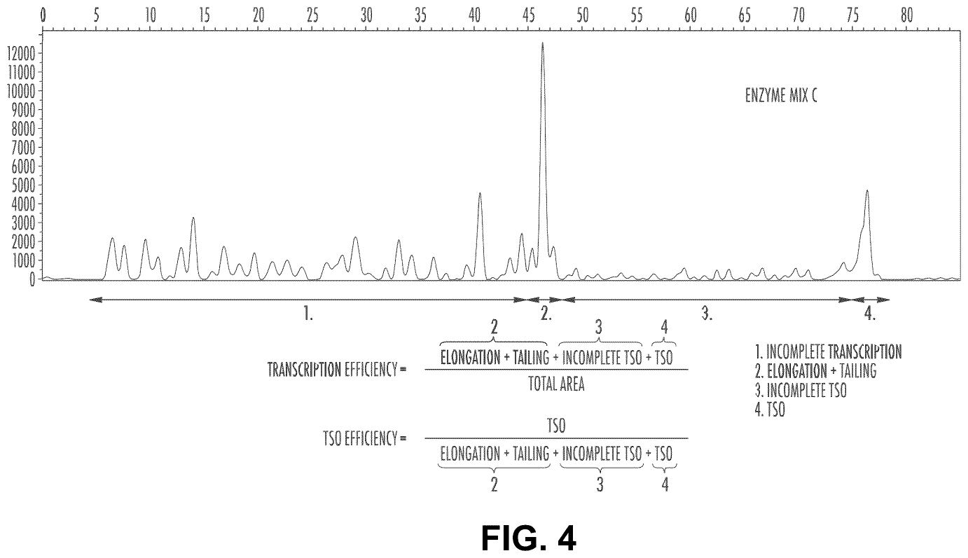

Transcription efficiency for a reverse transcription enzyme may be calculated as the sum of the area under the curve for the elongation and tailing (2), incomplete template switching (TSO) (3) and complete template switching (TSO) (4) regions over the total area under the curve for all products (FIG. 4). Transcription efficiency reflects all those products for which transcription was successfully completed. Template switching oligonucleotide efficiency may be calculated as the area under the curve for the complete template switching region (4) over the total area under the curve for all products including elongation and tailing (2), incomplete TSO (3) and complete TSO (4) (FIG. 4). An engineered reverse transcriptase or an engineered fusion reverse transcriptase may have an increased transcription efficiency, an increased TSO efficiency or both an increased transcription efficiency and an increased TSO efficiency.

For both transcription efficiency and template switching efficiency, lengths less than 45 nucleotides are considered incomplete (1). Lengths including the full length and the full length plus the tail are considered the elongation and tailing phase (2). Lengths longer than the full length plus the tail and shorter than the full length plus tail and template switching are considered incomplete template switching products (incomplete TSO, 3). Lengths having the full length plus tail and template switching size are considered template switched (TSO, 4).

Template switching oligonucleotides (also referred to herein as “switch oligos” or “switch oligonucleotides”) may be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as a template to further extend the cDNA.

Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxylnosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination. Suitable lengths of a switch oligo are known in the art. See for example U.S. patent application Ser. No. 15/975,516 herein incorporated by reference in its entirety.

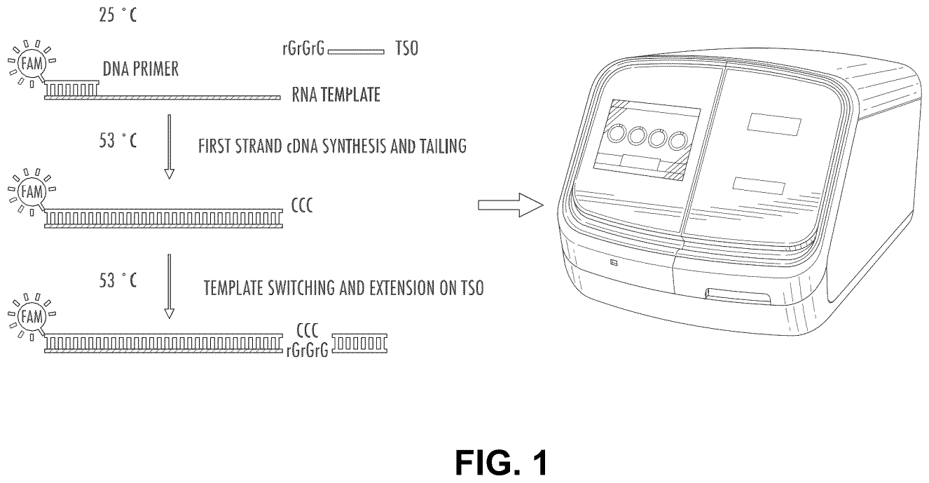

The general overview of template switching can be seen in FIG. 1. A primer can be hybridized to a RNA template, wherein the primer is extended by reverse transcription using a reverse transcriptase, thereby generating a first strand cDNA molecule. A polyC sequence can be added to the cDNA by a terminal transferase enzyme. A template switching oligonucleotide comprising a complementary polyG sequence to the polyC sequence added to the first strand cDNA, is added to the reaction, the polyG-TSO oligonucleotide hybridizes via complementarity to the polyC, and the reverse transcriptase can use that TSO sequence as a template for further extension. In the Examples, experiments for determining the efficiency of template switching are assayed on a capillary electrophoresis system such as a SeqStudio CE analyzer (ThermoFisher).

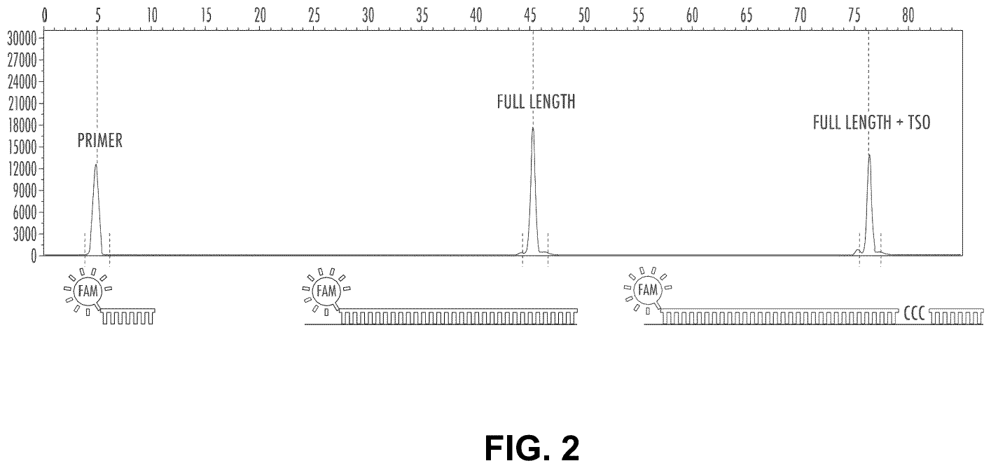

Results from a CE assay, using fluorescently labelled polynucleotides, is exemplified in FIG. 2. With fluorescence on the Y axis and the nucleotide length on the X axis, a FAM labelled primer of 5 nt is shown, a FAM labelled first strand cDNA product of 45 nt is shown and a TSO extended first strand cDNA of approximately 75 nucleotides (nt) is exemplified. FIG. 2 exemplifies this workflow showing experimental results using an RT enzyme known to have the ability to extend a polyG-TSO (enzyme C, or SEQ ID NO:1) compared to an RT that is not expected to extend a polyG-TSO (AR). On the top capillary electrophoretic graph, the full length cDNA product and a full length cDNA product with a TSO tail (tailing) are only approximately 1 nucleotide (nt) different; no polyG TSO extension was generated. Conversely, using enzyme mix C that include SEQ ID NO: 1 RT, the full length cDNA product and the full length cDNA product with efficient polyG-TSO extension were both generated.

In some embodiments, an engineered reverse transcription enzyme of the current application may exhibit an altered base-biased template switching activity such as an increased base-biased template switching activity, decreased base-biased template switching activity or an altered base-bias to the template switching activity. An engineered reverse transcriptase variant of the present disclosure may exhibit enhanced template switching with a 5′-G cap on the nucleic acid. Furthermore, engineered reverse transcription enzyme variants described herein may also exhibit unexpectedly higher tolerance to inhibitory compositions which might be present in cell lysates (i.e., are less inhibited by cell lysates) than that exhibited by an enzyme having the amino acid sequence set forth in SEQ ID NO: 1.

It is recognized that a different combination of mutations, such as different sites or residue changes may alter a reverse transcriptase activity similarly or differently. The variants that can template switch in the 5′ assay share the following alterations relative to SEQ ID NO: 7, E69K, E302R, T306K, W313F, K435G, and N454K. These variants may comprise additional alterations that may affect one or more reverse transcriptase related activities. Relative to SEQ ID NO: 7, M39V and M66L may improve template switching. Without being limited by mechanism, variants comprising a M39V or a M66L mutation that do not exhibit altered performance in the 5′ GEM single cell assay may exhibit an altered processivity, an altered K_{D} or both. Relative to SEQ ID NO: 7, K435 mutants may improve thermostability in the presence of primer template. In the absence of primer template K435 variants may exhibit a thermal denaturation profile similar to that of the wild-type protein. Relative to SEQ ID NO: 7, K435, P448 and D449 are residues in the connection domain; it was found that altering these residues may result in increased conformational flexibility.

An altered template switching efficiency may be an increased template switching efficiency or a decreased template switching efficiency as compared to the template switching efficiency of a reverse transcriptase having the amino acid sequence set forth in SEQ ID NO:1. Altered template switching efficiency may be at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9× or at least 10× greater than the template switching activity of a reverse transcriptase having the amino acid sequence set forth in SEQ ID NO:1. Altered template switching efficiency may range from 0.1× greater to 10× greater than the template switching activity of a reverse transcriptase having the amino acid sequence set forth in SEQ ID NO:1, from 0.25× greater to 7.5× greater than the template switching activity of a reverse transcriptase having the amino acid sequence set forth in SEQ ID NO:1, from 0.5× greater to 5× greater than the template switching activity of a reverse transcriptase having the amino acid sequence set forth in SEQ ID NO:1, or from 1× greater to 4× greater than the template switching activity of a reverse transcriptase having the amino acid sequence set forth in SEQ ID NO:1.

### 3. Transcription Efficiency

In some embodiments, the engineered reverse transcriptase or engineered fusion reverse transcriptases disclosed herein exhibits enhanced transcription efficiency when compared to the transcription efficiency of a reverse transcriptase having the amino acid sequence set forth in SEQ ID NO:1 or SEQ ID NO: 7. As noted herein, the conversion of mRNA into cDNA by reverse transcriptase-mediated reverse transcription is an essential step in single cell profiling and gene expression analyses. However, the use of unmodified reverse transcriptase to catalyze reverse transcription is inefficient for all the reasons disclosed herein. The engineered reverse transcriptases or engineered fusion reverse transcriptases of the disclosure are preferably modified or mutated such that the transcription efficiency of the engineered enzyme is increased or enhanced.

Further, engineered reverse transcription enzyme variants or engineered fusion reverse transcription enzyme variants of the present disclosure may have an unexpectedly greater ability to associate or bind to full-length transcripts (e.g., in T-cell receptor paired transcriptional profiling), as compared to that exhibited by an enzyme having the amino acid sequence set forth in SEQ ID NO:1.

It is recognized that salt concentration, the concentration of a cell fixation chemical and/or the concentration of a process reagent in a reverse transcriptase reaction may impact function of a reverse transcriptase. For example, “chemical tolerance” is intended that an engineered fusion reverse transcription enzyme of the current application may exhibit a reverse transcriptase related activity in either an expanded salt concentration range or in the presence of an increased concentration of a cell fixation chemical or process reagent, or in both an expanded salt concentration range and in the presence of an increased concentration of a cell fixation chemical or process reagent, as compared to the reverse transcriptase related activity of an enzyme having the amino acid sequence set forth in SEQ ID NO: 1.

An altered transcription efficiency may be an increased transcription efficiency or a decreased transcription efficiency as compared to the transcription efficiency of a reverse transcriptase having the amino acid sequence set forth in SEQ ID NO: 1. Altered transcription efficiency may be at least 0.1×, 0.2×, 0.3×, 0.4×, 0.5×, 0.6×, 0.7×, 0.8×, 0.9×, 1×, 1.5×, 2×, 2.5×, 3×, 3.5×, 4×, 4.5×, 5×, 5.5×, 6×, 6.5×, 7×, 7.5×, 8×, 8.5×, 9×, 10×, 15×, 20×, 25× or at least 30× greater than the transcription efficiency of a reverse transcriptase having the amino acid sequence set forth in SEQ ID NO: 1.

Transcription efficiency may be calculated as the sum of the area under the curve for the elongation, elongation plus tail, incomplete template switching (TSO) and complete template switching (TSO) regions over the total area under the curve for all products (see FIG. 4). Transcription efficiency reflects all those products for which transcription was successfully completed. Template switching oligonucleotide efficiency may be calculated as the area under the curve for the complete template switching region over the total area under the curve for all full-length products (see FIG. 4). An engineered reverse transcriptase may have an increased transcription efficiency, an increased TSO efficiency or both an increased transcription efficiency and an increased TSO efficiency.

### 4. Processivity

In some embodiments, the engineered reverse transcriptase enzyme or engineered fusion reverse transcriptase enzyme described herein possesses one or more of the following characteristics when compared to a wild-type polymerase and/or reverse transcriptase: increased thermostability; increased thermoreactivity; increased resistance to reverse transcriptase inhibitors; increased ability to reverse transcribe difficult templates; increased speed; increased processivity; increased specificity; enhanced polymerization activity; increased sensitivity, or any combination thereof.

Processivity is defined as the ability of a polymerase or reverse transcriptase to carry out continuous nucleic acid synthesis on a template nucleic acid without frequent dissociation. It can be measured by the average number of nucleotides incorporated by a polymerase on a single association/disassociation event. DNA polymerase or reverse transcriptase alone produces short DNA product strand per binding event. Most DNA polymerases or reverse transcriptases are intrinsically low-processivity enzymes. The low processivity of DNA polymerase or reverse transcriptase alone is insufficient for the timely replication of a large genome.

In some embodiments, the polymerization activity of the engineered reverse transcriptase enzyme as described herein is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type reverse transcriptase.

In some embodiments, the engineered e reverse transcriptase enzyme or engineered fusion reverse transcriptase enzyme reverse transcribes a RNA molecule having at least about 100, at least about 200, at least about 300, at least about 400, at least about 500, at least about 600, at least about 700, at least about 800, at least about 900, or at least about 1000 nucleotides. In another embodiment, the engineered reverse transcriptase enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In another embodiment, the engineered reverse transcriptase enzyme reverse transcribes a RNA molecule that is at least about 7 kb or at least about 8 kb.

In some embodiments, the increase in thermoreactivity, resistance to reverse transcriptase inhibitors, ability to reverse transcribe difficult templates, speed, processivity, specificity, or sensitivity of the engineered reverse transcriptase enzyme as described herein has is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the enhanced reverse transcriptase activity is an increased binding affinity and template switching efficiency as compared to a reverse transcriptase having the amino acid sequence set forth in SEQ ID NO:1. In some embodiments, the enhanced reverse transcriptase activity is an enhanced processivity as compared to the processivity of a reverse transcriptase having the amino acid sequence set forth in SEQ ID NO: 1.

Processivity relates to a reverse transcriptase's ability to remain associated with the template while incorporating nucleotides. Measurements of processivity may include but are not limited to the number of nucleotides incorporated in a single binding event of a reverse transcriptase molecule. Processivity also relates to the affinity of the enzyme for the substrate; thus, an enzyme with increased processivity may be more resistant to the presence of an inhibitor.

### III. Nucleic Acids and Expression Vectors

One aspect of the present disclosure provides an isolated nucleic acid molecule encoding the engineered reverse transcriptase, the engineered fusion reverse transcriptase or a derivatives thereof as described herein. In some embodiments, the engineered fusion reverse transcriptase is encoded by a nucleic acid set forth herein or readily derived in light of polypeptide information provided herein (e.g., SEQ ID NO: 1, 3, 4-8, 12-14, 16-18, 20, and 22-55) and known in the art. In some embodiments, the isolated nucleic acid molecule comprises a sequence selected from SEQ ID NO: 136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:167, SEQ ID NO:169, or SEQ ID NO: 171; or a nucleic acid sequence of Table 5.

The engineered fusion reverse transcriptases, the engineered reverse transcriptases, or the DNA binding domains need not be encoded by any specific nucleic acid exemplified herein. For example, redundancy in the genetic code allows for variations in nucleotide codon sequences that nevertheless encode the same amino acid. Accordingly, engineered polymerases of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type polymerase nucleic acids may be isolated from naturally occurring sources to be used as starting material to generate novel polymerases. Generally, the nomenclature and the laboratory procedures in recombinant DNA technology described below are those well known and commonly employed in the art. Standard techniques for cloning, DNA and RNA isolation, amplification and purification are known. Generally enzymatic reactions involving DNA ligase, DNA polymerase, restriction endonucleases are the like are performed according to the manufacturer's specifications. These techniques and various other techniques are generally performed according to Sambrook & Russell, Molecular Cloning-A Laboratory Manual, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of polymerase nucleic acids may be accomplished by a variety of techniques. The polymerase nucleic acids of the present invention can be generated from the wild type sequences. The wild type sequences are altered to create modified sequences. Wild type polymerases can be modified to create the polymerases claimed in the present application using methods that are well known in the art. Exemplary modification methods are site-directed mutagenesis, point mismatch repair, or oligonucleotide-directed mutagenesis.

Another aspect of the present disclosure provides an expression vector comprising the isolated nucleic acid encoding the engineered reverse transcriptase or derivatives thereof as described herein. A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. Synthesis of heterologous proteins in yeast is well known and described in the literature. There are many expression systems for producing the polymerase polypeptides of the present invention that are well known to those of ordinary skill in the art.

Another aspect of the present disclosure provides a host cell transfected with the expression vector comprising the isolated nucleic acid encoding the engineered reverse transcriptase as described herein. Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Once expressed, the engineered reverse transcriptase or a derivative thereof can be purified according to standard procedures of the art, including ammonium sulfate precipitation, affinity purification columns, column chromatography, gel electrophoresis and the like. Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate purification of the engineered reverse transcriptase or a derivative thereof, the nucleic acids that encode the engineered reverse transcriptase or derivatives thereof can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the disclosure, and corresponding detection systems are known to those of skill in the art as described herein, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used (6His-tag, his-tag), although one can use more or less than six. Suitable metal chelate affinity ligands that can serve as the binding moiety for a polyhistidine tag include nitrilo-tri-acetic acid (NTA).

One of skill in the art would recognize that after biological expression or purification, the engineered reverse transcriptase or derivatives thereof may possess a conformation substantially different than the native conformations of the constituent polypeptides. In this case, it may be necessary or desirable to denature and reduce the engineered reverse transcriptase or a derivative thereof and cause the engineered reverse transcriptase or a derivative thereof to re-fold into the preferred conformation. Methods of reducing and denaturing proteins and inducing re-folding are well known to those of skill in the art.

### IV. Compositions and Reaction Mixtures

The present disclosure further provides compositions comprising a variety of components in various combinations needed for nucleic acid amplification. In some embodiments of the present disclosure, the compositions are formulated by admixing one or more engineered reverse transcriptase enzymes, engineered fusion reverse transcriptase enzymes, or derivatives thereof of the present disclosure in a buffered salt solution. One or more DNA polymerases and/or one or more nucleotides, and/or one or more primers may optionally be added to create the compositions of the invention. These compositions can be used in the methods disclosed herein to produce, analyze, quantitate and otherwise manipulate nucleic acid molecules (e.g., using reverse transcription or one-step RT-PCR procedures).

In some embodiments, the engineered reverse transcriptase or the engineered fusion reverse transcriptase disclosed herein are provided at working concentrations (e.g., 1×) in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C. As used herein, the term “working concentration” means the concentration of an enzyme that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present invention is preferably distilled, deionized and sterile filtered (through a 0.1-0.2 micrometer filter), and is free of contamination by DNase and RNase enzymes. Such water is available commercially, for example from Life Technologies (Carlsbad, Calif.) or may be made as needed according to methods well known to those skilled in the art.

### V. Methods for Using Engineered Fusion Reverse Transcriptases

The engineered reverse transcriptases of the present application may be used in any application in which a reverse transcriptase with the indicated altered activity is desired. Methods of using reverse transcriptases are known in the art; one skilled in the art may select any of the engineered reverse transcriptases disclosed herein. In some embodiments, a reverse transcription reaction introduces a bar code. In some embodiments, the barcoding reaction is an enzymatic reaction. In some embodiments, the barcoding reaction is a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by lysing the cell. In some embodiments, the RNA molecules are messenger RNA (mRNA).

### A. Amplification Methods

One aspect of the present disclosure provides a method for performing a reverse transcription reaction for generating a nucleic acid product from an RNA template using an engineered fusion reverse transcriptase described herein. The engineered reverse transcriptases of the present application may be used in any application in which a reverse transcriptase with the indicated altered activity is desired. Methods of using reverse transcriptases are known in the art; one skilled in the art may select any of the engineered reverse transcriptases disclosed herein.

In some embodiments, the engineered fusion reverse transcriptase comprises an M39 mutation, a K47 mutation, an L435 mutation, a D449 mutation, a D524 mutation, an E607 mutation, a D653 mutation and an L671 mutation in SEQ ID NO:7. In some embodiments, the engineered fusion reverse transcriptase comprises a mutation selected from a K13 mutation, a K13L mutation, a D36 mutation, an N37 mutation, a V2 mutation, a D36L mutation, an insertion, and a combination thereof.

In some embodiments, the engineered fusion reverse transcriptase comprises: an amino acid sequence selected from SEQ ID NO:3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170; or an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, or SEQ ID NOs: 135, 137, 139, 153, 155, 161, 163, 166, 168, 170.

In some embodiments, the engineered reverse transcriptases or the engineered fusion reverse transcriptases, or derivatives thereof of the present disclosure are used in reverse transcription reactions, such as RT-PCR, or other known reactions in the art where nucleic acids, for example RNA molecules, are reverse transcribed using a reverse transcriptase.

Another aspect of the present disclosure provides a method of using the engineered fusion reverse transcriptase or the engineered fusion reverse transcriptase described herein comprising contacting the engineered fusion reverse transcriptase with a nucleic acid template under suitable conditions to produce a polymerized nucleic acid product. In some embodiments, the nucleic acid template is an RNA, a DNA, or a nucleic acid comprising an unnatural nucleotide.

The engineered reverse transcriptase, the engineered fusion reverse transcriptase or a derivative thereof as described herein may be used to make nucleic acid molecules from one or more templates. Such methods can comprise mixing one or more nucleic acid templates (e.g., RNA, such as non-coding RNA (ncRNA), messenger RNA (mRNA), micro RNA (miRNA), and small interfering RNA (siRNA) molecules) with one or more of the reverse transcriptases of the disclosure and incubating the mixture under conditions sufficient to generate one or more nucleic acid molecules complementary to all or a portion of the one or more nucleic acid templates. Other methods of cDNA synthesis which may advantageously use the present disclosure will be readily apparent to one of ordinary skill in the art.

In some embodiments, the method of using the engineered reverse transcriptase, the engineered fusion reverse transcriptase or a derivative thereof as described herein comprises the amplification of one or more nucleic acid molecules comprising mixing one or more nucleic acid templates with one of the engineered reverse transcriptase enzymes or a derivative thereof of the disclosure, and incubating the mixture under conditions sufficient to amplify one or more nucleic acid molecules complementary to all or a portion of the one or more nucleic acid templates. In one embodiment, the method may comprise the use of one or more DNA polymerases and may be employed as in standard reverse transcription-polymerase chain reaction (RT-PCR) reactions.

In some embodiments, the method of using the engineered reverse transcriptase, the engineered fusion reverse transcriptase or a derivative thereof as described herein may be one-step (e.g., one-step RT-PCR) or two-step (e.g., two-step RT-PCR) reactions. In one embodiment, the one-step RT-PCR type reactions may be accomplished in one tube thereby lowering the possibility of contamination. Such one-step reactions comprise (a) mixing a nucleic acid template (e.g., mRNA) with one or more engineered reverse transcriptase enzymes or derivatives thereof of the present disclosure and one or more polymerases and (b) incubating the mixture under conditions sufficient to amplify a nucleic acid molecule complementary to all or a portion of the template.

In another embodiment, a two-step RT-PCR reaction may be accomplished in two separate steps. Such a method comprises (a) mixing a nucleic acid template (e.g., mRNA) with a engineered reverse transcriptase enzyme or a derivative thereof of the present disclosure, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 kb in length), a combination of DNA polymerases and the engineered reverse transcriptase enzyme or a derivative thereof of the present disclosure may be used.

Amplification methods which may be used in accordance with the present invention (using one or more engineered reverse transcriptase enzymes or derivatives thereof of the present disclosure) include PCR, Isothermal Amplification, Strand Displacement Amplification (SDA), and Nucleic Acid Sequence-Based Amplification (NASBA); as well as more complex PCR-based nucleic acid fingerprinting techniques such as Random Amplified Polymorphic DNA (RAPD) analysis, Arbitrarily Primed PCR (AP-PCR) DNA Amplification Fingerprinting (DAF); microsatellite PCR; Directed Amplification of Minisatellite-region DNA (DAVID); digital droplet PCT (ddPCR) and Amplification Fragment Length Polymorphism (AFLP) analysis. In some embodiments, the engineered reverse transcriptase disclosed herein may be used in methods of amplifying or sequencing a nucleic acid molecule comprising one or more polymerase chain reactions (PCRs), such as any of the PCR-based methods described above.

Methods of producing an engineered reverse transcriptase, an engineered fusion reverse transcriptase or a derivative thereof of the present disclosure are known to those of skill in the art of molecular biology or molecular genetics. For example, nucleic acids encoding the wild type polymerase or nucleic acid binding domains can be generated using routine techniques in the field of recombinant genetics.

### B. Nucleic Acid Sample Processing

Another aspect of the present disclosure provides a nucleic acid extension method comprising contacting a target nucleic acid molecule with an engineered fusion reverse transcriptase or an engineered reverse transcriptase and a plurality of nucleic acid barcoded molecules comprising a barcode sequence, and incubating the target nucleic acid, the engineered fusion reverse transcriptase and barcoded molecules under conditions in which the barcoded molecules are extended by the engineered fusion reverse transcriptase. In some embodiments, the engineered fusion reverse transcriptase comprises the amino acid sequence of an engineered fusion transcriptase described herein or a derivatives thereof. The target nucleic acid hybridizes to one of the plurality of barcoded molecules and the hybridized barcoded molecule is extended by the engineered reverse transcriptase described herein.

The novel engineered reverse transcriptase variants and/or engineered fusion reverse transcriptase variants described herein can be used to generate a Single Cell 3′ (SC-3′) and/or 5′ (SC-5′) gene expression libraries. The SC-3′ and SC-5′ assays are similar but capture different ends of the polyadenylated transcript in the final library. Both solutions use polydT primer for reverse transcription (Tables 1-2). In the SC-3′ assay, the polydT sequence is located on the gel bead oligo. In the SC-5′ assay, the polydT is supplied as an RT primer. A template switching oligo (TSO) is used in both assays to reverse transcribe the full-length transcript.

After amplifying the cDNA, transcripts are randomly fragmented under conditions that favor 300-400 bp length fragments. Downstream of fragmentation, only transcripts containing both (1) a 10× Barcode and (2) an Illumina Read 2 adaptor, which is ligated on to the cDNA after fragmentation, will be amplified during the Sample Index PCR. This results in final 10× libraries that either represent the 3′ end of the transcript (as the 10× Barcode is adjacent to the polyA tail on the 3′ end of the transcript) or the 5′ end of the transcript (as the 10× Barcode is adjacent to the TSO and the 5′ end of the transcript). See e.g., kb.10xgenomics.com/hc/en-us/articles/360000939852-What-is-the-difference-between-Single-Cell-3-and-5-Gene-Expression-libraries-.

### 1. RNA Template

In some embodiments, the nucleic acid is a ribonucleic acid (RNA) molecule; and the engineered reverse transcriptase enzyme reverse transcribes the RNA molecule thereby generating a first strand cDNA.

In some embodiments, a reverse transcription reaction introduces a barcode. In some embodiments, the barcoding reaction is an enzymatic reaction. In some embodiments, the barcoding reaction is a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by lysing the cell. In some embodiments, the RNA molecules are released from the cell by permeabilizing the cell, or a tissue which comprises a plurality of the same and/or different cell types. In some embodiments, the RNA molecules are messenger RNA (mRNA).

In some embodiments, a reverse transcription reaction of the engineered reverse transcriptase, the engineered fusion reverse transcriptase or derivative thereof e of the present disclosure is initiated at the point of hybridization of the capture sequences to the RNA molecules, with the capture probe being extended by the engineered reverse transcriptase enzyme of the present disclosure in a template directed fashion using the hybridized mRNA as a template. In some embodiments, the reverse transcription reaction produces single stranded cDNA molecules each having a molecular tag and barcode associated with the cDNA, followed by amplification of cDNA to produce a double stranded cDNA that includes the sequences of the barcoded molecules.

In some embodiments, the plurality of nucleic acid barcoded molecules comprise an oligo(dT) sequence. In that embodiment, the engineered reverse transcriptase enzyme reverse transcribes the mRNA molecule into a complementary DNA molecule using the mRNA hybridized to the oligo(dT) sequence of the nucleic acid barcoded molecules as a template, and the nucleic acid binding domain binds and stabilizes the mRNA-oligo(dT) hybrid during the reverse transcription. Following reverse transcription, the engineered reverse transcriptase enzyme as described herein further amplifies the complementary DNA molecule comprising the barcode sequence, thereby generating an amplified DNA product comprising the barcode sequence, molecular tag sequence, or complements thereof.

In some embodiments of the nucleic acid extension method described herein, the method comprises a second nucleic acid molecule comprising an oligo(dT) sequence. In that embodiment, the plurality of nucleic acid barcoded molecules comprise an oligo(dT) sequence; and the nucleic acid binding domain of the engineered reverse transcriptase enzyme binds and stabilizes the mRNA-Oligo(dT) hybrid, while the polymerase domain of the engineered reverse transcriptase enzyme reverse transcribes the mRNA molecule using the second nucleic acid molecule comprising the oligo(dT) sequence, thereby generating a complementary DNA molecule. In this embodiment, the engineered reverse transcriptase enzyme further amplifies the complementary DNA molecule, thereby generating an amplified DNA product comprising a barcode sequence.

In some embodiments, the nucleic acid extension method comprises a cell, a population of cells, or a tissue and the template nucleic acid molecule is from the cell, population of cells or the tissue.

In some embodiments, the molecular tags are coupled to priming sequences and the barcoding reaction is initiated by hybridization of the priming sequences to the RNA molecules. In some embodiments, each priming sequence comprises a random N-mer sequence. In some embodiments, the random N-mer sequence is complementary to a 3′ sequence of a ribonucleic acid molecule of the cell. In some embodiments, the random N-mer sequence comprises a poly-dT sequence having a length of at least 5 bases. In some embodiments, the random N-mer sequence comprises a poly-dT sequence having a length of at least 10 bases (SEQ ID NO: 4).

In some embodiments, the barcoding reaction is performed by extending the priming sequences in a template directed fashion using reagents for reverse transcription. In some embodiments, the reagents for reverse transcription comprise a reverse transcription enzyme, a buffer and a mixture of nucleotides. In some embodiments, the reverse transcription enzyme adds a plurality of non-template oligonucleotides upon reverse transcription of a ribonucleic acid molecule. In some embodiments, the reverse transcription enzyme is an engineered fusion reverse transcription enzyme as disclosed herein.

In some embodiments, the barcoding reaction produces single stranded complementary deoxyribonucleic acid (cDNA) molecules each having a molecular tag from said molecular tags on a 5′ end thereof, followed by amplification of cDNA to produce a double stranded cDNA having the molecular tag on the 5′ end and a 3′ end of the double stranded cDNA.

In some embodiments, a molecular tag which comprises a barcode plus additional functional sequences, or only additional functional sequences, is further included into a cDNA molecule generated during a reverse transcription reaction. In some embodiments, the reagents for reverse transcription comprise a reverse transcription enzyme, a buffer and a mixture of nucleotides. In some embodiments, the reverse transcription enzyme adds a plurality of non-template oligonucleotides upon reverse transcription of a ribonucleic acid molecule from the nucleic acid molecules. In some embodiments, the reverse transcription enzyme is an engineered reverse transcription enzyme as disclosed herein.

In one aspect, the present disclosure provides methods that utilize the engineered reverse transcriptases or the engineered fusion reverse transcriptases described herein for nucleic acid sample processing. In one embodiment, the method comprises contacting a template ribonucleic acid (RNA) molecule with an engineered reverse transcriptase to reverse transcribe the RNA molecule to a complementary DNA (cDNA) molecule. The contacting step may be in the presence of a plurality of nucleic acid barcode molecules, wherein each nucleic acid barcode molecule comprises a barcode sequence. The nucleic acid barcode molecule may comprise a sequence configured to couple to a template RNA molecule. Suitable sequences include, without limitation, an oligo(dT) sequence, a random N-mer primer, or a target-specific primer. The nucleic acid barcode molecule may comprise a template switching sequence.

In other embodiments, the RNA molecule is a messenger RNA (mRNA) molecule. In one embodiment, contacting step provides conditions suitable to allow the engineered reverse transcriptase to (i) transcribe the mRNA molecule into the cDNA molecule with the oligo(dT) sequence and/or (ii) perform a template switching reaction, thereby generating the cDNA molecule which comprises the barcode sequence, or a derivative thereof. In another embodiment, the contacting step may occur in (i) a partition having a reaction volume (as further described herein and see e.g., U.S. Pat. Nos. 10,400,280 and 10,323,278, each of which is incorporated herein by reference in its entirety), (ii) in a bulk reaction where the reaction components (e.g., template RNA and engineered reverse transcriptase) are in solution, or (iii) on a nucleic acid array (see e.g., U.S. Pat. Nos. 10,480,022 and 10,030,261 as well as WO/2020/047005 and WO/2020/047010, each of which is incorporated herein by reference in its entirety). Further, the reverse transcription reaction may occur in a tissue (in situ reverse transcription), on a template that is associated with a sequence on a substrate such as practiced in spatial transcriptomics, or further in a RT-PCR or other reverse transcription reaction in vitro on a purified target, partially purified target or unpurified target as found for example in a cellular lysate.

Examples of assays involving nucleic acid sample processing may include, but are not limited to, single-cell transcription profiling, single-cell sequence analysis, immune profiling of individual T and B cells, single-cell chromatin accessibility analysis (e.g. ATAC seq analysis), single cell processing and analysis, paired single cell TCR sequencing, paired TCRα and TCRβ. These exemplary assays may be carried out using commercially available systems for encapsulating biological samples, gel beads, barcodes, and/or other compounds/materials in droplets, such as The Chromium System (10× Genomics, Pleasanton CA USA). Engineered reverse transcriptases may be used in methods of profiling a T-Cell receptor (TCR).

In various embodiments, the poly-dT sequence may be extended in a reverse transcription reaction using the mRNA as a template to produce a cDNA transcript complementary to the mRNA and also includes sequence of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product can be ligated to additional functional sequences, and further amplified (e.g., via PCR). The functional sequences may include a sequencer specific flow cell attachment sequence such as but not limited to, a P7 sequence for Illumina sequencing systems, as well as functional sequence, which may include a sequencing primer binding site, e.g., for a R2 primer for Illumina sequencing systems, as well as functional sequence, which may include a sample index, e.g., an i7 sample index sequence for Illumina sequencing systems.

Although described in terms of specific sequence references used for certain sequencing systems, e.g., Illumina systems, it will be understood that the reference to these sequences is for illustration purposes only, and the methods described herein may be configured for use with other sequencing systems incorporating specific priming, attachment, index, or other operational sequences used in those systems, e.g., systems available from Ion Torrent, Oxford Nanopore, Genia, Pacific Biosciences, Complete Genomics, and the like.

### 2. Volume

As described herein, wild-type and variants MMLV RT are not optimal for reverse transcription of mRNA when using high throughput amplification reaction assays (e.g. spatial array and single cell transcriptomics assay) and the like. This is because high throughput amplification reaction assays require reaction volumes that are usually less than about 1 nanoliter. Accordingly, the present disclosure provides novel engineered reverse transcriptase enzymes that function efficiently in high throughput amplification reaction assays that require reaction volumes of less than about 1 nanoliter.

In some embodiments, the method comprises providing a reaction volume which comprises an engineered reverse transcriptase and a template ribonucleic acid (RNA) molecule. In one other embodiment, the contacting occurs in a reaction volume, which may be less than 1 nanoliter, less than 750 picoliters, or less than 500 picoliters. In other embodiments, the reaction volume is present in a partition, such as a droplet or well (including a microwell or a nanowell).

In some embodiments, the engineered reverse transcriptase, the engineered fusion reverse transcriptase or derivatives thereof as described herein are used in a reaction volume less than about 1 nanoliter (nL). In some embodiments, the engineered reverse transcriptase, the engineered fusion reverse transcriptase or derivatives thereof as described herein are used in a reaction volume that is less than about 500 picoliter (pL). In some embodiments, the reaction volume is contained within a partition. In some embodiments, the reaction volume is contained within a droplet. In some embodiments, the reaction volume is contained within a droplet in an emulsion. In some embodiments, the reaction volume is contained within a droplet emulsion having a reaction volume of less than about 1 nL. In some embodiments, the reaction volume is contained within a droplet emulsion having a reaction volume of less than about 500 pL.

In some embodiments, the reaction volume is contained within a well. In some embodiments, the reaction volume is contained within a well having a reaction volume less than about 1 nL. In some embodiments, the reaction volume is contained within a well. In some embodiments, the reaction volume is contained within a well having a reaction volume less than about 500 pL. In some embodiments, the reaction volume is contained within a well in an array of wells having an extracted nucleic acid molecule, and the template nucleic acid molecule is the extracted nucleic acid molecule. In some embodiments, the reaction volume is contained within a well in an array of wells having a cell comprising a template nucleic acid molecule, and where the template nucleic acid molecule is released from the cell.

In another embodiment, a method comprises providing a reaction volume, which comprises an engineered fusion reverse transcriptase and a template ribonucleic acid (RNA) molecule and is considered a “low volume reaction”. The reaction volume may comprise a plurality of nucleic acid barcode molecules, wherein each nucleic acid barcode molecule comprises a barcode sequence. In an embodiment, the contacting occurs in a reaction volume, a low volume reaction, which may be less than 1 nanoliter, less than 750 picoliters, or less than 500 picoliters. In other embodiments, the reaction volume is present in a partition, such as a droplet or well (including a microwell or a nanowell).

### 3. Unique Molecular Identifier (UMI)

In some embodiments, the barcoding reaction produces single stranded complementary deoxyribonucleic acid (cDNA) molecules each having a molecular tag on a 5′ end thereof, followed by amplification of the cDNA to produce a double stranded DNA having the molecular tag on the 5′ end and a 3′ end of the double stranded DNA.

In some embodiments, the molecular tags (e.g., barcode oligonucleotides) include unique molecular identifiers (UMIs). In some embodiments, the UMIs are oligonucleotides. In some embodiments, the molecular tags are coupled to priming sequences. In some embodiments, each of the priming sequences comprises a random N-mer sequence. In some embodiments, the random N-mer sequence is complementary to a 3′ sequence of the RNA molecules. In some embodiments, the priming sequence comprises a poly-dT sequence having a length of at least 5 bases. In some embodiments, the priming sequence comprises a poly-dT sequence having a length of at least 10 bases (SEQ ID NO: 4). In some embodiments, the priming sequence comprises a poly-dT sequence having a length of at least 5 bases, at least 6 bases, at least 7 bases, at least 8 bases, at least 9 bases, at least 10 bases.

Unique molecular identifiers (UMIs), e.g., in the form of nucleic acid sequences are assigned or associated with individual cells or populations of cells, in order to tag or label the cell's components (and as a result, its characteristics). These unique molecular identifiers may be used to attribute the cell's components and characteristics to an individual cell or group of cells, additionally to be used as a method for counting the individual cells or groups of cells by their incorporation.

In some aspects, the unique molecular identifiers are provided in the form of nucleic acid molecules (e.g., oligonucleotides) that comprise nucleic acid barcode sequences that may be attached to or otherwise associated with the nucleic acid contents of individual cell, or to other components of the cell, and particularly to fragments of those nucleic acids. The nucleic acid molecule can, and do have differing barcode sequences, or at least represent a large number of different barcode sequences across all of the partitions in a given analysis. In some aspects only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

Moreover, when a population of barcodes is partitioned, the resulting population of partitions can also include a diverse barcode library that may include at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some embodiments, the enhanced reverse transcriptase activity of the engineered reverse transcriptase disclosed herein is an enhanced ability to yield mitochondrial UMI counts as compared to a reverse transcriptase having the amino acid sequence set forth in SEQ ID NO:1 or 15. In some embodiments, the enhanced reverse transcriptase activity is an enhanced ability to yield increased ribosomal UMI counts as compared to a reverse transcriptase having the amino acid sequence set forth in SEQ ID NO:1 or 15. Read counting and UMI counting are the principal gene expression quantification schemes used in single-cell RNA-sequencing (scRNA-seq) analysis, as such with increased ribosomal UMI counts sensitivity and accuracy increases for a scRNA-seq assay in determining transcriptome profiles for any given cell, group of cells or tissues. Numerous metrics can be used for quality control of single-cell RNA-sequencing, including percent of reads mapping to ribosomal genes, percent of reads mapping to mitochondrial genes, total number of UMIs detected, or number of features to which 50% of the reads map.

Beneficially, even following any subsequent amplification of the contents of a given partition, the number of different UMIs can be indicative of the quantity of mRNA originating from a given partition, and thus from the cell. As noted above, the transcripts can be amplified, purified and sequenced to identify the sequence of the cDNA transcript of the mRNA, as well as to sequence the barcode segment and the UMI segment. While a poly-dT primer sequence is described, other targeted or random primer sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

It is recognized that certain reverse transcriptase enzymes may increase UMI reads from genes of a desired length or length of interest. The desired length of genes may be selected from lengths comprising less than 500 nucleotides, between 500 and 1000 nucleotides, between 1000 and 1500 nucleotides and greater than 1500 nucleotides. It is recognized that a reverse transcriptase may preferentially increase UMI reads from genes of one length range. It is recognized that an engineered reverse transcriptase may perform similarly, differently or comparably in a 3′-reverse transcription assay or a 5′-reverse transcription assay. It is similarly recognized that an engineered reverse transcriptase may preferentially increase UMI reads from a length of genes in a 3′-reverse transcription assay than in a 5′-reverse transcription assay.

### 4. Gel Bead

The engineered reverse transcriptases or the engineered fusion reverse transcriptases of the present disclosure may be suitable for use in methods in which a cell can be co-partitioned along with a nucleic acid barcode molecule bearing bead. The nucleic acid barcode molecules can be released from the bead in the partition. By way of example, in the context of analyzing sample RNA, the poly-dT (poly-deoxythymine, also referred to as oligo (dT)) segment of one of the released nucleic acid molecules can hybridize to the poly-A tail of a mRNA molecule. Reverse transcription results in a cDNA transcript of the mRNA, but that transcript includes each of the sequence segments of the nucleic acid molecule. Without being limited by mechanism, because the nucleic acid molecule comprises an anchoring sequence, it may be more likely hybridize to and prime reverse transcription at the sequence end of the poly-A tail of the mRNA. Within any given partition, all of the cDNA transcripts of the individual mRNA molecules may include a common barcode sequence segment. However, the transcripts made from the different mRNA molecules within a given partition may vary at the unique UMI segment.

Beneficially, even following any subsequent amplification of the contents of a given partition, the number of different UMIs can be indicative of the quantity of mRNA originating from a given partition, and thus from the cell. As noted above, the transcripts can be amplified, cleaned up and sequenced to identify the sequence of the cDNA transcript of the mRNA, as well as to sequence the barcode segment and the UMI segment. While a poly-dT primer sequence is described, other targeted or random priming sequences may also be used in priming the reverse transcription reaction.

In some embodiments of the nucleic acid extension method described herein, the plurality of nucleic acid barcoded molecules are attached to a support (e.g. a particle, a slide, a chip, a bead, etc.). In one embodiment, the support is selected from an array, a bead, a gel bead, a microparticle, and a polymer. In some embodiments, the nucleic acid barcoded molecules attached to a support comprise molecular tags (UMIs), primer sequences, capture sequences, cleavage sequences, or additional functional sequences. In some embodiments, the support is a gel bead. In that embodiment, the nucleic acid barcoded molecules are releasably attached to the gel bead. In some embodiments, the gel bead comprises a polyacrylamide polymer.

In some embodiments, a cross-section of the gel bead is less than about 100 μm. In some embodiments, a cross-section of a gel bead is less than about 60 μm. In some embodiments, a cross-section of a gel bead is less than about 50 μm. In some embodiments, a cross-section of a gel bead is less than about 40 μm. In some embodiments, a cross-section of a gel bead is less than about 100 μm, less than about 99 μm, less than about 98 μm, less than about 97 μm, less than about 96 μm, less than about 95 μm, less than about 94 μm, less than about 93 am, less than about 92 μm, less than about 91 μm, less than about 90 μm, less than about 89 μm, less than about 88 μm, less than about 87 μm, less than about 86 μm, less than about 85 μm, less than about 84 μm, less than about 83 μm, less than about 82 μm, less than about 81 μm, less than about 80 μm, less than about 79 μm, less than about 78 μm, less than about 77 μm, less than about 76 μm, less than about 75 μm, less than about 74 μm, less than about 73 μm, less than about 72 μm, less than about 71 μm, less than about 70 μm, less than about 69 μm, less than about 68 μm, less than about 67 μm, less than about 66 μm, less than about 65 μm, less than about 64 μm, less than about 63 μm, less than about 62 μm, less than about 61 μm, or less than about 60 μm.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can comprise a unique molecular identifier (UMI). In some cases, the primer can comprise an R1 sequence for use in Illumina sequencing workflows. In some cases, the primer can comprise an R2 sequence for use in Illumina sequencing workflows. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference. However, the present invention is not limited as to a composition of any nucleic acid molecule or derivative thereof, or any particular sequencing platform and these characterizations serve as examples only which may be useful in a reverse transcription workflow.

In operation, a cell can be co-partitioned along with a barcode bearing bead. The barcoded nucleic acid molecules affixed to a bead can be released from the bead in the partition. By way of example, in the context of analyzing sample RNA, the poly-dT (poly-deoxythymine, also referred to as oligo (dT)) segment of one of the released nucleic acid molecules can hybridize to (e.g., capture)_the poly-A tail of a mRNA molecule. Reverse transcription may result in a cDNA transcript of the mRNA which cDNA transcript also includes each of the sequence segments of the nucleic acid molecule. Because the nucleic acid molecule comprises additional functional sequences (e.g., capture domains, primer domains, UMIs, barcodes, etc.), it can hybridize to and prime reverse transcription of the mRNA using the hybridized mRNA as a template. Within any given partition, all of the cDNA transcripts of the individual mRNA molecules may include a common barcode sequence. However, the transcripts made from the different mRNA molecules within a given partition may vary with respect to unique molecular identifying sequences (e.g., UMIs). Beneficially, following any subsequent amplification of the contents of a given partition, the number of different UMIs can be indicative of the quantity of mRNA originating from a given partition, and thus from the cell. As noted above, the transcripts can be amplified and sequenced to identify the sequence of the original mRNA captured template, as well as the sequence of the associated barcode and UMI. While a poly-dT capture sequence is described, other targeted or random capture sequences may also be used in capture or hybridize to a template for initiating the reverse transcription reaction.

In various embodiments, the poly-dT segment may be extended in a reverse transcription reaction using the mRNA as a template to produce a cDNA transcript complementary to the mRNA and also includes sequence segments of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence segment. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product may be sheared, ligated to additional functional sequences, and further amplified (e.g., via PCR).

Any of the engineered RT enzymes of the present disclosure, including without limitation any of the enzymes comprising the amino acid sequence and/or nucleic acid sequences shown in Table 4, Table 5, or Table 6, could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of mutliomic extensions or applications.

### C. Immunoprofiling

Engineered reverse transcriptases may be used in methods of a T-Cell receptor (TCR) and a B-cell receptor (BRC) profiling.

In some embodiments, an engineered reverse transcriptase is used in methods including but not limited to processing of a TCR from an individual T cell(s) or groups of T cell(s), determining the nucleotide sequence of the TCR(s) of T cell(s), and obtaining TCR repertoire profile. In some methods, a nucleic acid barcode sequence is appended to a nucleic acid molecule encoding for a TCR (e.g. a molecule derived from a T cell containing a nucleic acid sequence encoding for a TCR, such as a TCRa and/or a TCRb mRNA) resulting in a barcoded nucleic acid molecule comprising a sequence corresponding to a nucleic acid sequence of the TCR (e.g. comprises a V(D)J region of a TCR gene or a reverse complement thereof) and a sequence corresponding to the barcode sequence (which in some instances is the reverse complement of the barcode sequence present in the nucleic acid barcode molecule). A barcoded nucleic acid molecule may serve as a template, such as a template polynucleotide, that can be further processed (e.g. amplified) and sequenced to obtain the target nucleic acid sequence. For example, a barcoded nucleic acid molecule may be further processed (e.g. amplified) and sequenced to obtain the nucleic acid sequence of the TCR.

TCR is a molecule found on the surface of T cells. Typically binding of the TCR by an antigenic molecule results in cell activation and response. The TCR is a heterodimer composed of two different protein chains. In many T cells, these two proteins are alpha (α) and beta (β) chains. In a smaller percentage of T cells, these two proteins are gamma (γ) and delta (δ) chains. The ratio of TCRs comprised of α/β chains versus γ/δ chains may change during a diseased state such as cancer, tumor, infectious disease, inflammatory disease or autoimmune disease. Engagement of the TCR with a peptide-MHC activates a T cell through a series of biochemical events mediated by associated enzymes, co-receptors, specialized adaptor molecules, and activated or released transcription factors.

Each of the two chains of a TCR contains multiple copies of gene segments—a variable ‘V’ gene segment, a diversity ‘D’ segment and a joining ‘J’ segment. The TCR alpha chain is generated by recombination of V and J segments, while the beta chain is generated by recombination of V, D and J segments. Similarly, generation of the TCR gamma chain involves recombination of V and J segments. Generation of the TCR delta chain occurs by recombination of V, D and J gene segments. The intersection of these specific regions (V and J for the alpha or gamma chain, or V, D, J for the beta or delta chain) corresponds to the CDR3 region involved in antigen-MHC recognition. Complementarity determining regions (e.g. CDR1, CDR2 and CDR3) or hypervariable regions are sequences in the variable domains of antigen receptors (e.g. T cell receptor and immunoglobulin) that can complement an antigen. Most of the diversity of CDRs is found in CDR3, with the diversity being generated by somatic recombination events during the development of T lymphocytes. CDR3, which is encoded by the junctional region between the V and J or D and J genes, is highly variable. CDR3 is often used as a region of interest to determine T cell clonotypes, a unique nucleotide sequence that arises during the gene rearrangement process, as it is highly unlikely that two T cells will express the same CDR3 nucleotide sequence unless they are derived from the same clonally expanded T cell. Because an active TCR consists of paired chains within single T cells, determination of the active paired chains within single T cells, determination of the active paired chains requires the sequencing of single T cells. TCR gene sequences may include, but are not limited to, sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes) and T cell receptor delta constant genes (TRDC genes).

### VI. Kits

One aspect of the present disclosure provides a kit comprising the engineered fusion reverse transcriptase enzyme, the engineered reverse transcriptase enzyme, the DNA binding domains or a derivative thereof as described herein. In some embodiments, the kit comprises one or more of a vector, a nucleotide, a buffer, a composition, a salt, and/or instructions. In another embodiment, a kit may comprise an engineered fusion reverse transcriptase enzyme or a derivative thereof for use in reverse transcription or amplification of a nucleic acid molecule. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The engineered reverse transcriptase enzyme or a derivative thereof, reaction buffer, and dNTPs may be provided separately or may be provided together in a master mix solution. When the engineered reverse transcriptase enzyme or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein.

The instructions for using any of the methods are generally recorded on a suitable recording medium (e.g. printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit, but means for obtaining the instructions from a remote source, e.g. via the internet, may be provided. For example, a kit that includes a web address where the instructions may be viewed and/or from which the instructions may be downloaded. As with the instructions, this means for obtaining the instructions is recorded on a suitable substrate.

Kits according to this aspect of the present disclosure comprise a carrier means, such as a box, carton, tube or the like, having in close confinement therein one or more container means, such as vials, tubes, ampoules, bottles and the like, wherein a first container means contains one or more of the engineered reverse transcriptase enzymes or derivatives thereof of the present disclosure having reverse transcriptase activity. When more than one polypeptide having reverse transcriptase activity is used, they may be in a single container as mixtures of two or more engineered reverse transcriptase enzymes or derivatives thereof, or in separate containers. The kits of the disclosure can also comprise (in the same or separate containers) one or more DNA polymerases, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5a, DH10B, HB101, Top 10, and other K-12 strains as well as E. coli B and E. coli W strains).

In a specific aspect of the present disclosure, the kits of the disclosure (e.g., reverse transcription and amplification kits) can include one or more components (in mixtures or separately) including one or more engineered reverse transcriptase enzymes or derivative thereof having reverse transcriptase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc). Such kits can comprise one or more DNA polymerases.

### VII. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. “A and/or B” is used herein to include all of the following alternatives: “A”, “B”, “A or B”, and “A and B”. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “About” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

Numeric ranges are inclusive of the numbers defining the range. The term about is used herein to mean plus or minus ten percent (10%) of a value. For example, “about 100” refers to any number between 90 and 110.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, the term “Analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “Adaptor(s),” “Adapter(s)” and “Tag(s)” may be used synonymously. An adaptor or tag can be coupled to a polynucleotide sequence to be “tagged” by any approach, including ligation, hybridization, or other approaches.

As used herein, the term “Barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

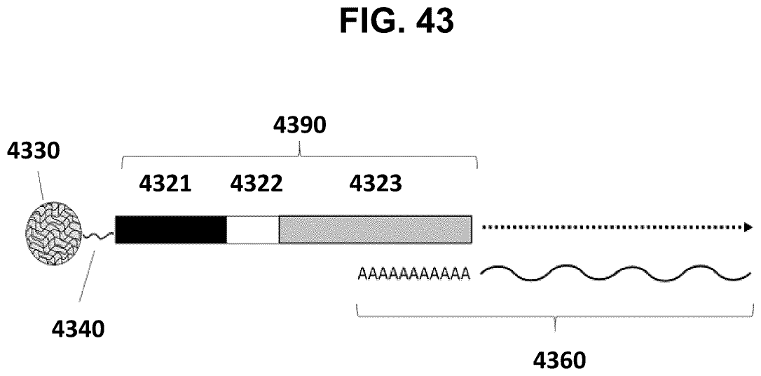

Barcoded nucleic acids may be generated (e.g., via a nucleic acid reaction, such as nucleic acid extension or ligation) from the constructs described in FIG. 43. For example, capture handle sequence may then be hybridized to complementary sequence, such as capture sequence 4323 to generate (e.g., via a nucleic acid reaction, such as nucleic acid extension or ligation) a barcoded nucleic acid molecule comprising cell (e.g., partition specific) barcode sequence 4322 (or a reverse complement thereof) and reporter barcode sequence 4322 (or a reverse complement thereof). In some embodiments capture handle sequence 4323 comprises a sequence complementary to a template switching oligonucleotide on the capture sequence 4323. In some embodiments, the nucleic acid barcoded molecule 4390 (e.g., partition-specific barcoded molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. In some instances, analysis of an analyte (e.g. a nucleic acid, a polypeptide, a carbohydrate, a lipid, a glycan, a glycan motif, a metabolite, a protein, etc.) comprises a workflow as generally depicted in FIG. 43. A nucleic acid barcoded molecule 4390 (e.g. partition specific barcoded molecule) may be co-partitioned with the one or more analytes. In some instances, nucleic acid barcoded molecule 4390 is attached to a support 4330 (e.g., a bead, such as a gel bead), such as those described elsewhere herein. For example, nucleic acid barcoded molecule 4390 may be attached to support 4330 via a releasable linkage 4340 (e.g., comprising a labile bond), such as those described elsewhere herein. Nucleic acid barcoded molecule 4390 may comprise a functional sequence 4321 and optionally comprise other additional sequences, for example, a barcode sequence 4322 (e.g., common barcode, partition-specific barcode, or other functional sequences described elsewhere herein), and/or a UMI sequence (not shown). The nucleic acid barcoded molecule 4390 may comprise a capture sequence 4323 that may be complementary to another nucleic acid sequence, such that it may hybridize to a particular sequence, e.g., capture handle sequence 4323.

For example, capture sequence 4323 may comprise a poly-T sequence and may be used to hybridize to mRNA. Referring to FIG. 43, in some embodiments, nucleic acid barcoded molecule 4390 comprises capture sequence 4323 complementary to a sequence of RNA molecule 4360 from a cell. In some instances, capture sequence 4323 comprises a sequence specific for an RNA molecule. Capture sequence 4323 may comprise a known or targeted sequence or a random sequence. In some instances, a nucleic acid extension reaction may be performed, thereby generating a barcoded nucleic acid product comprising capture sequence 4323, the functional sequence 4321, barcode sequence 4322, any other functional sequence, and a sequence corresponding to the RNA molecule 4360.

In another example, capture sequence 4323 may be complementary to an overhang sequence or an adapter sequence that has been appended to an analyte. Any suitable agent may degrade beads. Suitable agents may include, but are not limited to, changes in temperature, changes in pH, reduction, oxidation and exposure to water or other aqueous solutions.

In some instances, a cell that is bound to labelling agent which is conjugated to oligonucleotide and support 4330 (e.g., a bead, such as a gel bead) comprising nucleic acid barcoded molecule 4390 is partitioned into a partition amongst a plurality of partitions (e.g., a droplet of a droplet emulsion or a well of a microwell array).

The term “Bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “Efficiency” in the context of a nucleic acid modifying enzyme of this invention refers to the ability of the enzyme to perform its catalytic function under specific reaction conditions. Typically, “efficiency” as defined herein is indicated by the amount of product generated under given reaction conditions.

As used herein, the term “Enhances” in the context of an enzyme refers to improving the activity of the enzyme, i.e., increasing the amount of product per unit enzyme per unit time.

As used herein, the term “Fidelity” refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

As used herein, the term “% homology,” which is used interchangeably with the term “% identity,” refers to the level of nucleic acid or amino acid sequence identity between the nucleic acid sequence that encodes any one of the inventive polypeptides (e.g., variant reverse transcriptases) or the inventive polypeptide's amino acid sequence, when aligned using a sequence alignment program.

As used herein, the term “Identical” in the context of two nucleic acids or polypeptide sequences refers to the residues in the two sequences that are the same when aligned for maximum correspondence, as measured using a sequence comparison algorithms. Sequence comparison algorithms are know to those skill in the art. See. E.g., ebi.ac.uk/Tools/msa/clustalo/.

As used herein, the term “Inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity).

As used herein, the term “Low volume reaction” means a reaction volume less than 1 nanoliter, less than 750 picoliters, or less than 500 picoliters.

The term “Molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

As used herein, the term “mutation” or “mutant” or “variant” indicates a change or changes introduced in a wild type DNA sequence or a wild type amino acid sequence. Examples of mutations or variants include, but are not limited to, substitutions, insertions, deletions, and point mutations. Mutations can be made either at the nucleic acid level or at the amino acid level.

As used herein, the term “Operably linked” or “conjugated” or “fusion” means that, in relation to the recombinant thermostable polymerase enzyme sequence there are one or more sequences at the N or C terminus that, when transcribed and translated, create additional polypeptides in association with the enzyme amino acid sequence, thereby created a conjugation or fusion of one or more polypeptides from one expression vector.

The term “Partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “Partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), wherein each partition maintains separation of its own content from the contents of other partitions are known in the art. See for example US 2020/0032335, herein incorporated by reference in its entirety. The partition can be a droplet in an emulsion. A partition may comprise one or more other partitions.

A “plurality of nucleic acid barcoded molecules” may comprise at least about 500 nucleic acid barcoded molecules, at least about 1,000 nucleic acid barcoded molecules, at least about 5,000 nucleic acid barcoded molecules, at least about 10,000 nucleic acid barcoded molecules, at least about 50,000 nucleic acid barcoded molecules, at least about 100,000 nucleic acid barcoded molecules, at least about 500,000 nucleic acid barcoded molecules, at least about 1,000,000 barcoded molecules, at least about 5,000,000 nucleic acid barcoded molecules, at least about 10,000,000 nucleic acid barcoded molecules, at least about 100,000,000 nucleic acid barcoded molecules, at least about 1,000,000,000 nucleic acid barcoded molecules. In some cases, a plurality of nucleic acid barcoded molecules comprise a partition-specific barcode sequence.

Each of the plurality of nucleic acid barcoded molecules may include an identifier sequence separate from the partition-specific barcode sequence, where the identifier sequence is different for each nucleic acid partition-specific barcoded molecule of the plurality of nucleic acid partition specific barcoded molecules. In some cases, such an identifier sequence is a unique molecular identifier (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, each of the plurality of nucleic acid barcoded molecules can comprise the partition specific barcode sequence and the bead can be from plurality of beads, such as a population of barcoded beads. Each of the partition specific barcode sequences can be different from partition specific barcode sequences of nucleic acid barcoded molecules of other beads of the plurality of beads. Where this is the case, a population of barcoded beads, with each bead comprising a different partition specific barcode sequence can be analyzed.

As used herein, the term “Processivity” refers to the ability of a reverse transcriptase to continuously extend a primer without disassociating from the nucleic acid template. The length of a template a reverse transcriptase or polymerase is capable of replicating can also be used to describe the processivity of that reverse transcriptase or polymerase. In some embodiments, “Processivity” refers to the ability of a polymerase to remain bound to the template or substrate and perform DNA synthesis. Processivity is measured by the number of catalytic events that take place per binding event.

As used herein, “Purified” means that a molecule is present in a sample at a concentration of at least 95% by weight, or at least 98% by weight of the sample in which it is contained.

As used herein, the term “Reverse transcriptase activity,” “reverse transcription activity,” or “reverse transcription” indicates the capability of an enzyme to synthesize a DNA strand (that is, complementary DNA or cDNA) using RNA as a template. Reverse transcriptase activity may be measured by incubating an enzyme in the presence of an RNA template and deoxynucleotides, in the presence of an appropriate buffer, under appropriate conditions, for example as described in the Example below. Methods for measuring RT activity are provided in the example below and also are well known in the art. Bosworth, et al., Nature 1989, 341:167-168.

As used herein, the term “recombinant RT” comprises the engineered RT fusion protein described herein or the engineered RT variant described herein.

As used herein, the term “Reverse transcriptase (RT)” is used in its broadest sense to refer to any enzyme that exhibits reverse transcription activity as measured by methods disclosed herein or known in the art. A “reverse transcriptase” of the present invention, therefore, includes reverse transcriptases from retroviruses, other viruses, as well as a DNA polymerase exhibiting reverse transcriptase activity, such as Tth DNA polymerase, Taq DNA polymerase, Tne DNA polymerase, Tma DNA polymerase, etc. RT from retroviruses include, but are not limited to, Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, and as described in U.S. Patent Application 2003/0198944 (hereby incorporated by reference in its entirety). For review, see e.g. Levin, 1997, Cell, 88:5-8; Brosius et al. 5 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

The term “Sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from blood, plasma, serum, urine, saliva, mucosal excretions, sputum, stool and tears.

The term “Subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

As used herein, the term “Sequencing,”, generally refers to methods and technologies for determining the sequence of nucleotide bases in one or more polynucleotides. Any method of sequencing known in the art may be used to evaluate the products of a reaction performed by an engineered reverse transcriptase of the current application. Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term “Thermoreactivity” or “Thermoreactive” refers to the ability of a reverse transcriptase to exhibit enzyme activity at elevated temperatures.

As used herein, “Thermostability” or “thermostable” refers to the ability of a reverse transcriptase to withstand exposure to elevated temperatures, but not necessarily show activity at such elevated temperatures. In some embodiments, thermostable reverse transcriptase or polymerase refers to any enzyme that catalyzes polynucleotide synthesis by addition of nucleotide units to a nucleotide chain using DNA or RNA as a template and has an optimal activity at a temperature above 53° C.

As used herein, the terms “Unique molecular identifier”, “Unique molecular identifying sequence”, “UMI” and “UMI sequence” are used synonymously. Individual barcoded molecules may comprise a common barcode sequence such as a partition specific sequence or a spatial array where every capture probe has a unique barcode sequence.

By “Binding sequence” is intended a nucleic acid sequence capable of binding to an analyte.

As used herein, the term “Variant” means a protein which is derived from a precursor protein (such as the native protein, for example MMLV native protein as set forth in SEQ ID NO:7) by addition of one or more amino acids to either or both the C- and N-terminal end, substitution of one or more amino acids at one or a number of different sites in the amino acid sequence, deletion of one or more amino acids at either or both ends of the protein or at one or more sites in the amino acid sequence, or addition of a fusion domain. SEQ ID NO:1 is a variant of MMLV. The preparation of an enzyme variant is preferably achieved by modifying a DNA sequence which encodes for the wild-type protein, transformation of that DNA sequence into a suitable host, and expression of the modified DNA sequence to form the derivative enzyme. A variant reverse transcriptase of the invention includes a protein comprising altered amino acid sequences in comparison with a precursor enzyme amino acid sequence wherein the variant reverse transcriptase retains the characteristic enzymatic nature of the precursor enzyme but which may have altered properties in some specific aspect. For example, an engineered reverse transcriptase variant may have an altered pH optimum or increased temperature stability but may retain its characteristic transcriptase activity.

A “Variant” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to a polypeptide sequence when optimally aligned for comparison. Percent identity may pertain to the percent identity of the DNA binding domain or the engineered reverse transcriptase portion of an engineered fusion reverse transcriptase. As used herein, a variant residue position is described in relation to the wild-type or precursor amino acid sequence set forth in SEQ ID NO:7; the amino acid position is indexed to SEQ ID NO:7. A fusion variant comprises at least one fusion domain selected from DNA binding domains described elsewhere herein.

As used herein, a protein having a certain percent (e.g., at least 90%, at least 91%, at least 92%, at least 93%, at least 94%, at least 95%, at least 96%, at least 97%, at least 98%, or at least 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, CA), GCG Pileup, FASTA (Pearson et al. (1988) Proc. Natl Acad. ScL USA 85:2444-2448), and BLAST (BLAST Manual, Altschul et al., Nat'l Cent. Biotechnol. Inf., Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Other sequence alignment software programs that find use are the TFASTA Data Searching Program available in the Sequence Software Package Version 6.0 (Genetics Computer Group, University of Wisconsin, Madison, WI and CLC Main Workbench (Qiagen) Version 20.0. The present disclosure is not limited to the software being used to align two or more sequences.

As used herein, the term “Wild-type” or “Wt” refers to a gene or gene product that has the characteristics of that gene or gene product when isolated from a naturally occurring source. The amino acid sequence set forth in SEQ ID NO:7 is a wt Murine Moloney Leukemia Virus (MMLV) sequence (Genbank NP_955591.1 p80 RT).

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

### EXAMPLES

It will be understood that the reference to the below examples is for illustration purposes only and do not limit the scope of the claims. Each aspect, embodiment, or feature of the invention may be combined with any other aspect, embodiment, or feature the invention unless clearly indicated to the contrary. Unless defined otherwise, all technical and scientific terms used herein have the meaning commonly understood by a person skilled in the art to which this invention belongs.

### Example 1: Capillary Electrophoresis Assay Validation

Reverse transcription and sequencing reactions were prepared. The reaction volume was 50 μl and reactions contained 5′-end labeled FAM Reverse Transcriptase primer 2, RT Reagent B (Chromium Next GEM Single Cell Reagent, 10× Genomics), RNA template (RNA Temp 2), template switching oligo 1 (TSO1), and the indicated engineered reverse transcriptase.

TABLE 1

| | | |
|---|---|---|
| Reagent | Stock | Final |

A: Capillary Electrophoresis Assay Reactants

| | | |
|---|---|---|
| RT Reagent B | 2.66x | 1.00X |

| | | | | |
|---|---|---|---|---|
| FAM.RT.Primer2 | 100.00 | uM | 0.50 | uM |
| RNA.Temp2.CE | 84.4 | uM | 0.50 | uM |
| TSO1.Oligo | 91.20 | uM | 5.00 | uM |
| Enzyme | 15.40 | uM | 0.50 | uM |

| | | |
|---|---|---|
| Water | — | — |

B: Capillary Electrophoresis Assay Reactants-GAPDH template

| | | |
|---|---|---|
| GEM-U Reagent | 4.00 x | 1.00 x |

| | | | | |
|---|---|---|---|---|
| GAPDH Primer | 10.00 | uM | 25 | nM |
| GAPDH | 5.6 | uM | 50 | nM |
| TSO1.Oligo | 93.00 | uM | 12.00 | uM |

| | | | |
|---|---|---|---|
| Enzyme | Variable uM | 0.8 | uM |

| | | |
|---|---|---|
| Water | — | — |

Experimental workflow replicated that of the Chromium Single Cell Gene Expression 5′ kit (10× Genomics, Inc), except the reverse transcriptase was altered for a particular reaction. Stock concentrations and final concentrations in the reactions are shown in Tables 1A-B. Variations of the assay stock concentrations and final concentrations in the reactions shown in Table 2 were used. The reactions included stoichiometrically equal amounts of enzyme and template for single turnover conditions.

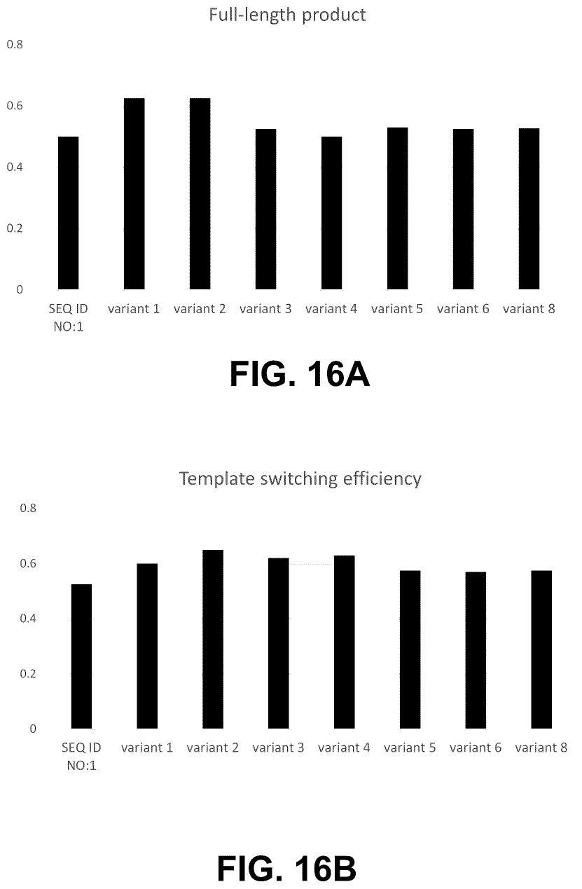

Additionally, reverse transcription and sequencing reactions were prepared using GAPDH as a template. The reaction volume was 50 μl; reactions contained 5′-end labeled GAPDH Primer (SEQ ID NO: 174), GEM-U reagent, RNA template (GAPDH template; (SEQ ID NO: 173), template switching oligo 1 (TSO1; (SEQ ID NO: 175), and the indicated engineered reverse transcriptase. Stock concentrations and final concentrations in the reactions are shown in Table 1B. The reactions included stoichiometrically equal amounts of enzyme and template for single turnover conditions. Reactants were incubated at 53° C. for 45 minutes, then diluted 1:20 in HiDi formamide. The formamide mixture was heated to 95° C. for 5 mins, then chilled on ice for 2 mins. Samples were loaded on the CE, the DS-33 dye set was selected and long fragment analysis was performed using the GS1200LIZ size standard. The GEM-U reagent approximates the formulation of the actual reagent mixture in a GEM assay when the contents of the Z1 and Z2 channels are mixed. Results from one such experiment are shown in FIG. 16.

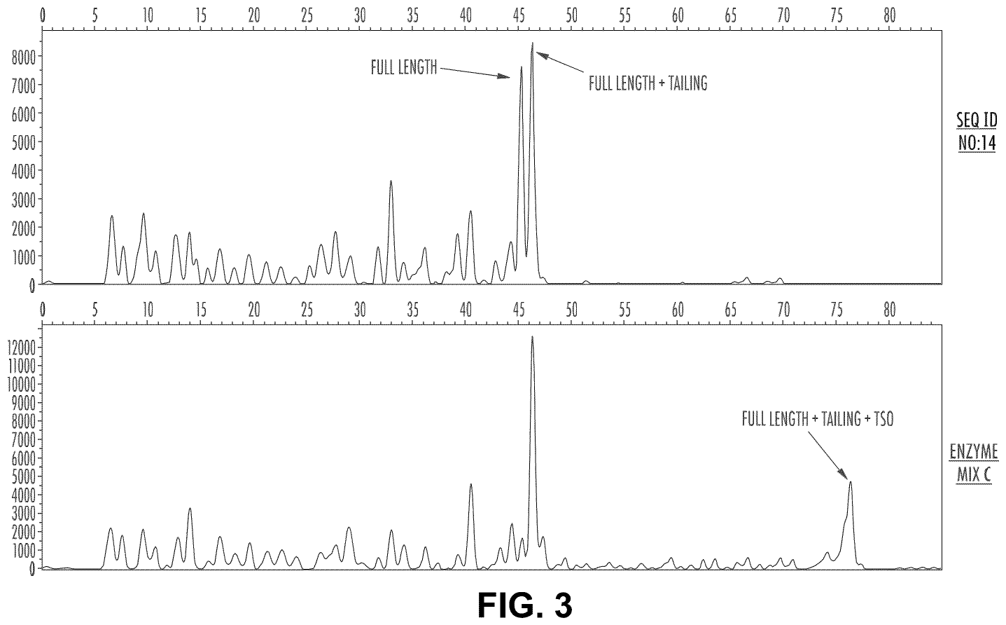

Reactants were incubated at 53° C. for one hour, then diluted 1:40 in water and then 1:20 in HiDi formamide. The formamide mixture was heated to 95° C. for 5 mins, then chilled on ice for 2 mins. Samples were loaded on a Seqstudio (Thermofisher) and fragment analysis by capillary electrophoresis was carried out with the appropriate dye channels and size standards. The assay was validated with synthetically sized oligonucleotides (FIG. 2) and with a transcription positive, template switching null engineered reverse transcriptase (SEQ ID NO:14) and a transcription positive, template switching positive reverse transcriptase (Enzyme Mix C, FIG. 3). The GEM-U reagent approximates the formulation of the actual reagent mixture in a GEM assay when the contents of the Z₁ and Z₂ channels are mixed. Capillary Electrophoresis Assay Reactants are disclosed in Table 1A, Capillary Electrophoresis Assay template, Primer and TSO sequences (SEQ ID NOS: 9-11, respectively in order of appearance) are shown in Table 2A.

Reverse transcription and sequencing reactions were also prepared using GAPDH as a template. The reaction volume was 50 μl; reactions contained 5′-end labeled GAPDH primer (SEQ ID NO: 174), GEM-U reagent, RNA template (GAPDH template; SEQ ID NO: 175), template switching oligo 1 (TSO1), and the indicated engineered reverse transcriptase. Stock concentrations and final concentrations in the reactions are shown in Table 2B. The reactions included stoichiometrically equal amounts of enzyme and template for single turnover conditions. Reactants were incubated at 53° C. for 45 minutes, then diluted 1:20 in HiDi formamide. The formamide mixture was heated to 95° C. for 5 mins, then chilled on ice for 2 mins. Samples were loaded on the CE, the DS-33 dye set was selected and long fragment analysis was performed using the GS1200LIZ size standard. The GEM-U reagent approximates the formulation of the actual reagent mixture in a GEM assay when the contents of the Z₁ and Z₂ channels are mixed.

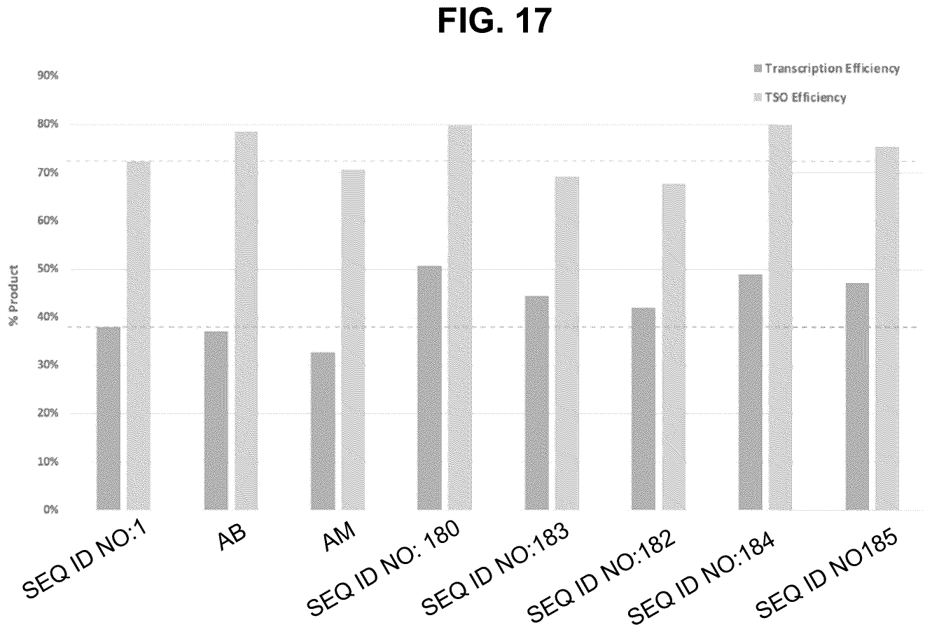

Results from one such experiment are shown in FIG. 17. Variants having the amino acid sequence set forth in SEQ ID NO: 1 or 179, SEQ ID NO: 180, SEQ ID NO: 182, SEQ ID NO: 183, SEQ ID NO: 184 and SEQ ID NO: 185 exhibited transcription efficiencies at or above about 40%. Variants AB and AM exhibited transcription efficiencies below 40%. Variants SEQ ID NO: 1 or 179, AB, SEQ ID NO: 180, SEQ ID NO: 184 and SEQ ID NO: 185 exhibited template switching efficiencies above 70%. Variants AM, SEQ ID NO: 182 and SEQ ID NO: 183 exhibited template switching efficiencies below that exhibited by a variant having the amino acid sequence set forth in SEQ ID NO: 1 or 179.

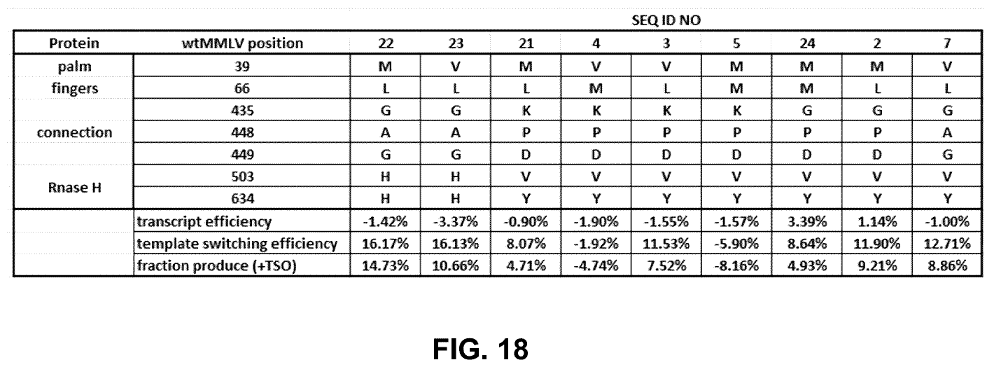

FIG. 18 summarizes results from one series of experiments where various engineered reverse transcriptases were evaluated as described herein. As shown in FIG. 18, variants having the amino acid sequence set forth in the indicated SEQ ID NO: SEQ ID NO:183 (shown as 5 in the table) vs SEQ ID NO:185 (shown as 7 in the table) or SEQ ID NO:195 (shown as 24 in the table); SEQ ID NO:185 (shown as 7 in the table) or SEQ ID NO:195 (24) vs SEQ ID NO:180 (2); and SEQ ID NO:183 (5) vs SEQ ID NO:180 (2). M39V improves template switching (variant having the amino acid sequence set forth in SEQ ID NO:182 (shown as 4 in the table) vs SEQ ID NO:183 (5)) but does little in combination with M66L. In addition, variants having the amino acid sequence set forth SEQ ID NO: 180 (shown as 2) vs SEQ ID NO: 185 (7), and SEQ ID NO: 193 (22) vs SEQ ID NO: 194 (23). P448A, D449G, H503V, and H634Y alterations appear neutral in this context.

In particular, the reaction volume was 50 μl; reactions contained 5′-end labeled GAPDH primer, GEM-U reagent, RNA template (GAPDH template), template switching oligo 1 (TSO1), and the indicated engineered reverse transcriptase(s). The final concentrations in the reactions are shown in Table 2B. The reaction buffer was SOP for SC-5′ and the reaction time was 45 minutes.

Tables 2A-B show Capillary Electrophoresis (CE) Assay Reactants and Template, Primer and TSO sequences (SEQ ID NOS:173, 175, 176, respectively in order of appearance.)

TABLE 2
A: Capillary Electrophoresis Assay Template, Primer and TSO sequences

| Reagent | Stock | | Final | | Volume | | | |
|---|---|---|---|---|---|---|---|---|
| RT Reagent B (2000165) | 4.00 x | | 1.00 x | | 9.54 | ul | 76.34 | ul |
| FAM.RT.Primer2 (Variable) | 100.00 | uM | 0.5000 | uM | 0.250 | ul | 2.000 | ul |
| RNA.Temp2.CE (Variable) | 84.40 | uM | 1.00 | uM | 0.59 | ul | 4.74 | ul |
| TSO1.Oligo (Variable) | 1000.00 | uM | 64.00 | uM | 3.20 | ul | 25.60 | ul |
| DTT | 1000.00 | mM | 20.00 | mM | 1.00 | ul | 8.00 | ul |
| Gel Bead Buffer (2000018) | 1.00 x | | 0.24x | | 11.83 | ul | 94.66 | ul |
| Polyacylamide Solution | 10% | | 0.50% | | 2.50 | ul | 20.00 | ul |
| (2000052) | | | | | | | | |
| Enzyme | 15.40 | uM | 0.50 | uM | 1.62 | ul | 12.99 | ul |
| Water | | | | | 19.46 | ul | 155.68 | ul |

| | | | | | |
|---|---|---|---|---|---|
| Total | — | 50.00 | ul | 400.00 | ul |

| B: Capillary Electrophoresis Assay GAPDH Template, Primer and TSO sequences | | |
|---|---|---|
| Reagent | Stock | Final |
| GEM-U Reagent | 4.00 x | 1.00 x |

| | | | | |
|---|---|---|---|---|
| GAPDH Primer | 10.00 | uM | 25 | nm |
| GAPDH | 5.00 | uM | 50 | nM |
| TSO1.Oligo | 93.00 | uM | 12.00 | uM |
| Enzyme | 15.40 | uM | 1.31 | uM |

| | | |
|---|---|---|
| Water | — | — |

### Example 2. Construction, Cloning and Expression of Engineered Reverse Transcriptases

Several mutants were constructed using a Q5 mutagenesis kit (NEB) with mutagenic primers per manufacturing instructions. Linearized products were circularized by KLD treatment (kinase, ligase, DpN1) and cloned. Several mutants were synthesized as whole plasmids and furnished by Twist Biosciences, South San Francisco CA.

Briefly, a vector comprising the Ss07d sequence was obtained from Integrated DNA Technologies (IDT, Coralville, IA). Cloning was performed using a Gibson Assembly kit from New England Biolabs (NEB, Ipswitch, ME). Q5 polymerase was used to generate Gibson vectors. Amplification conditions were an initial denaturation at 95° C. for 2.5 minutes, 30 cycles of denature (95° C., 30 sec), a 45 sec gradient annealing and extension at 72° C. for 6 minutes, 35 sec, followed by a final extension at 72° C. for 2 minutes. Amplification reactions with multiple annealing gradient temperatures (65.2° C., 67° C., 68.5° C. and 69.6° C.) were performed.

Amplification products were evaluated on a 1.2% agarose E-Gel using SYBR-Safe. Products were pooled prior to clean-up. Cloning and expression were performed in the Acella cell line from EdgeBio (San Jose, CA). Cells were selected on LB-Kanamycin plates. Ss07d N-terminal and C-terminal fusions to an engineered reverse transcriptase having the amino acid sequence set forth in SEQ ID NO:1 were obtained by screening of bacterial colonies. The sequences of the fusion proteins were confirmed.

An Ss07d N-terminal fusion protein of the amino acid sequence set forth in SEQ ID NO: 8 was generated; and an Ss07d C-terminal fusion protein of the amino acid sequence set forth in SEQ ID NO: 6 was generated. In some aspects the Sso7d fusion proteins are produced with an N-terminal 6× HisTag and thrombin cleavage site. The 6× HisTag is used for purification purposes and removed by thrombin cleavage.

### Example 3: 5′ GEM-X Analysis of N-Terminal and C-Terminal Fusion Protein

Experiments were carried out as found in the manufacturer's instructions for the Chromium Single Cell 5′ Gene Expression Assay kit (10× Genomics). Table 3 details the reverse transcriptases and the fusion variants that were generated in Example 3.

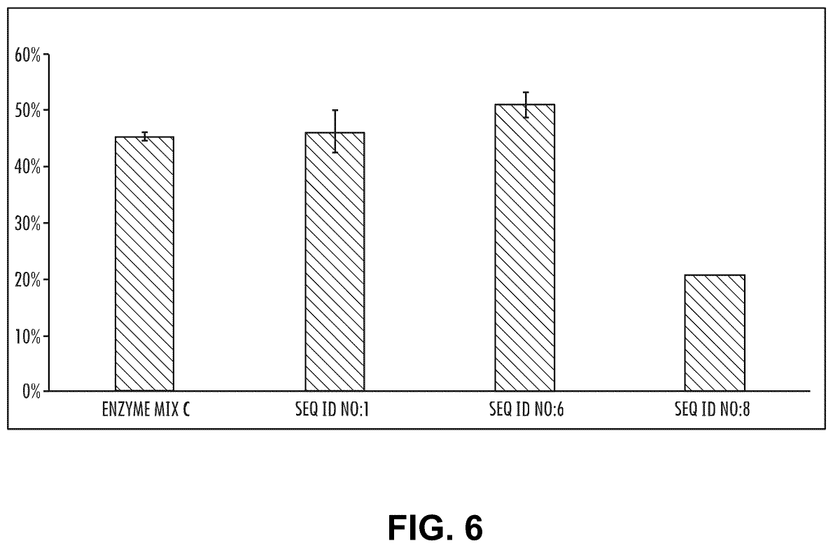

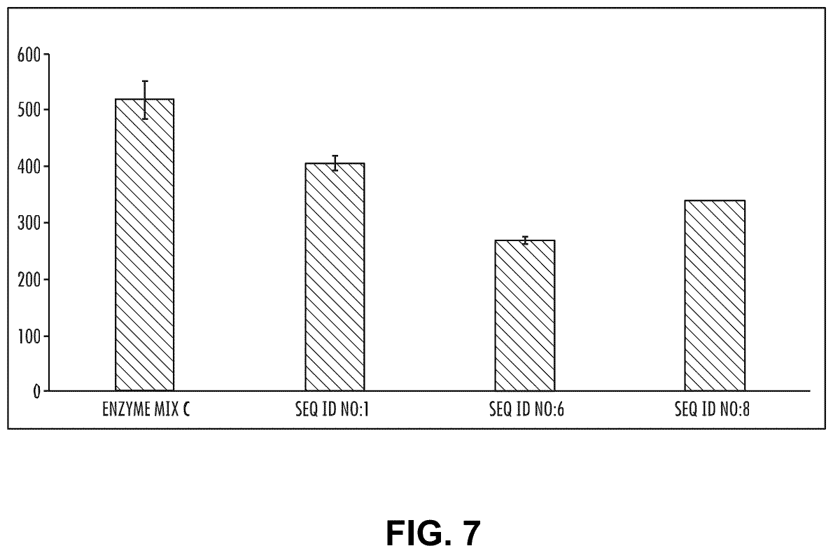

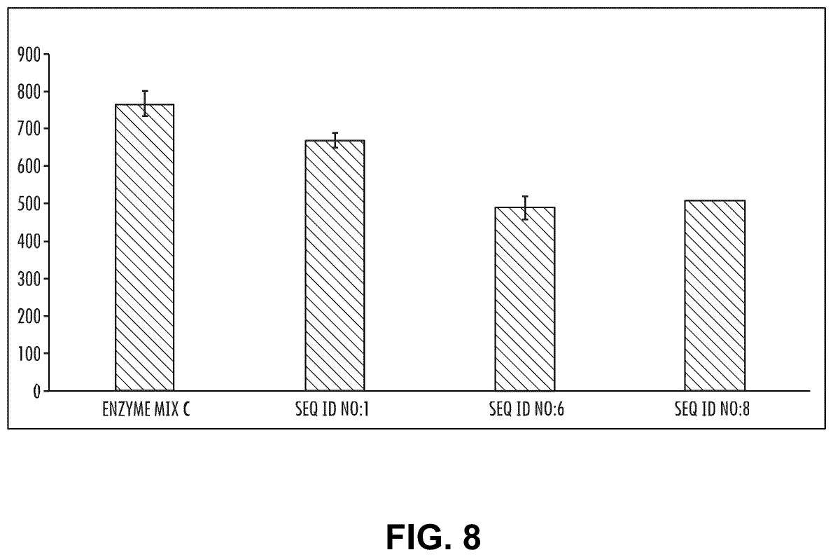

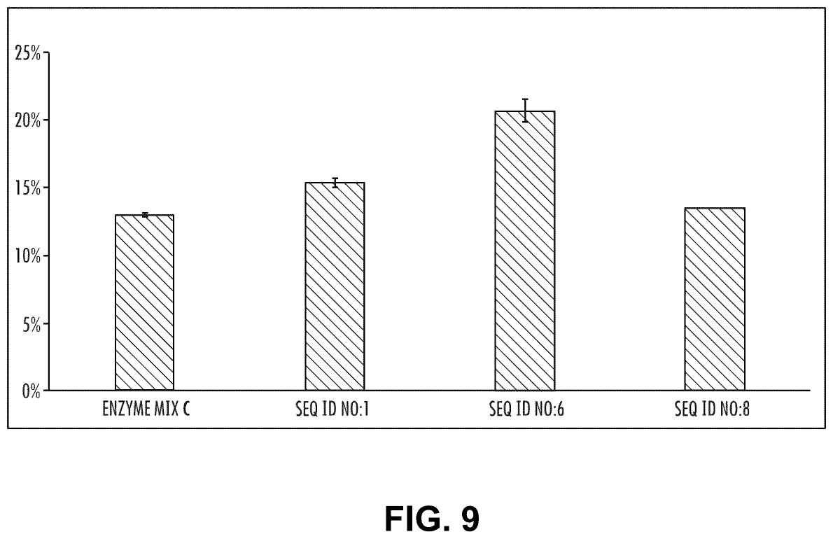

Exemplary results can be seen in FIGS. 5-10. FIG. 5 data demonstrates the increased percentage of valid barcodes read upon sequencing of products generated using one of four different RT enzyme configurations. Both SEQ ID NO: 1 and 6 demonstrated enhanced ability to incorporate barcodes into a nucleic acid product upon reverse transcription compared to the control Enzyme mix C. Conversely, SEQ ID NO: 8 was less efficient than the control enzyme mix. The same type of pattern is seen in FIG. 6 (mapped reads to transcriptome) and FIG. 9 (fraction of ribosomal protein UMI counts). FIGS. 7 and 8 reveal a different pattern of efficiency, where, while transcription products were produced by all enzymes tested, the control yielded more transcription products that results in more genes per cell and higher median UMI counts per cell, respectively, compared to SEQ ID NOS: 1, 6 or 8. FIG. 10 shows that the three variants and fusion MMLV enzymes provided products that yielded higher fraction mitochondrial UMI counts compared to the Enzyme mix C control.

As such, the data demonstrate that the SEQ ID NOs: 1, 6 and 8 were comparable to the control reverse transcriptase in a variety of experiments, and in many cases were equivalent or exceeded the activity of the control reverse transcriptase.

TABLE 3
SEQ ID NOs of MMLV enzymes used
and fusion variants generated

| SEQ | | | DNA BINDING | DBD NAME/ |
|---|---|---|---|---|
| ID NO | MMLV | 6HIS | DOMAIN (DBD) | LOCATION |
| 1 | Variant | No | No | NA |
| 2 | NA | NA | Yes | Archeal consensus |
| | | | | sequence |
| 3 | Fusion variant | Yes | SEQ ID NO: 12 | Sto7; C-terminus |
| 5 | Fusion variant | Yes | SEQ ID NO: 12 | Sto7; C-terminus |
| 6 | Fusion variant | No | SEQ ID NO: 13 | Sso7d; C-terminus |
| 7 | Wild type | No | No | NA |
| 8 | Fusion variant | No | SEQ ID NO: 13 | Sso7d; N-terminus |

### Example 4: Transcription Efficiency and Template Switching Efficiency Analysis

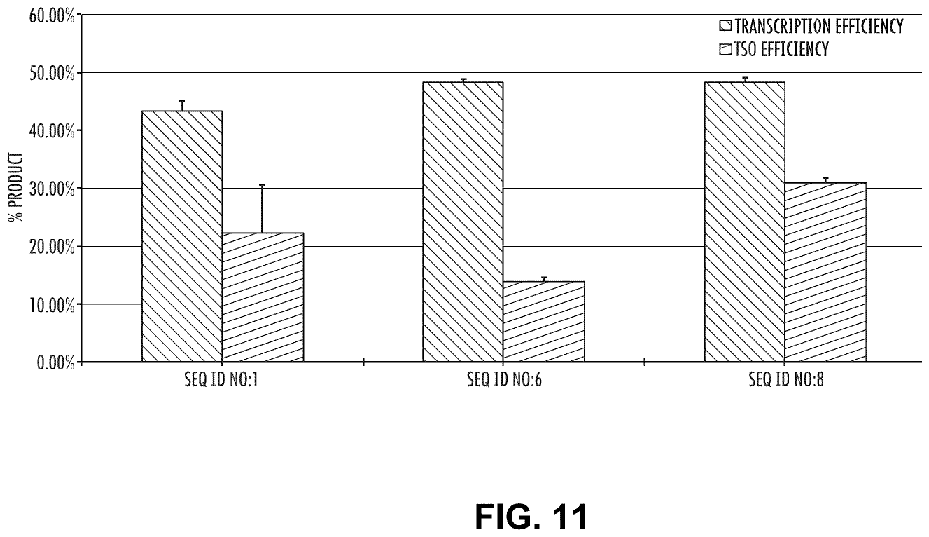

Capillary electrophoretic reactions were performed generally as described above in the previous examples, using a variety of reverse transcriptases and engineered reverse transcriptases as found in Table 3. The transcription efficiency and template switching efficiency as a percent product were determined via calculations as shown in FIG. 4. FIG. 11 shows the results of an exemplary set of experiments for determining transcription efficiency and template switching efficiency of control reverse transcriptase enzyme (SEQ ID NO: 1) and two engineered fusion reverse transcriptase enzymes described herein (SEQ ID NO: 6 and SEQ ID NO: 8). The transcription efficiencies of the clones was found to be comparable. However, the TSO efficiency was shown to be variable from one clone to the next.

### Example 5: Template Switching Efficiency Analysis

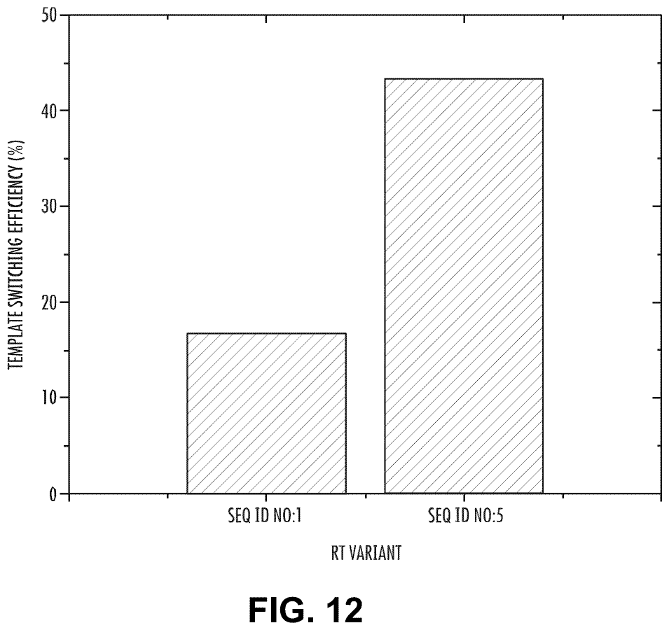

A reverse transcriptase having the amino acid sequence set forth in SEQ ID NO:1 and an engineered reverse transcriptase comprising an amino acid sequence set forth in SEQ ID NO:5 were evaluated for template switching efficiency. Results from one such series of experiments are shown in FIG. 12, where the RT from SEQ ID NO: 5 showed enhanced TSO comparative to the MMLV variant of SEQ ID NO: 1.

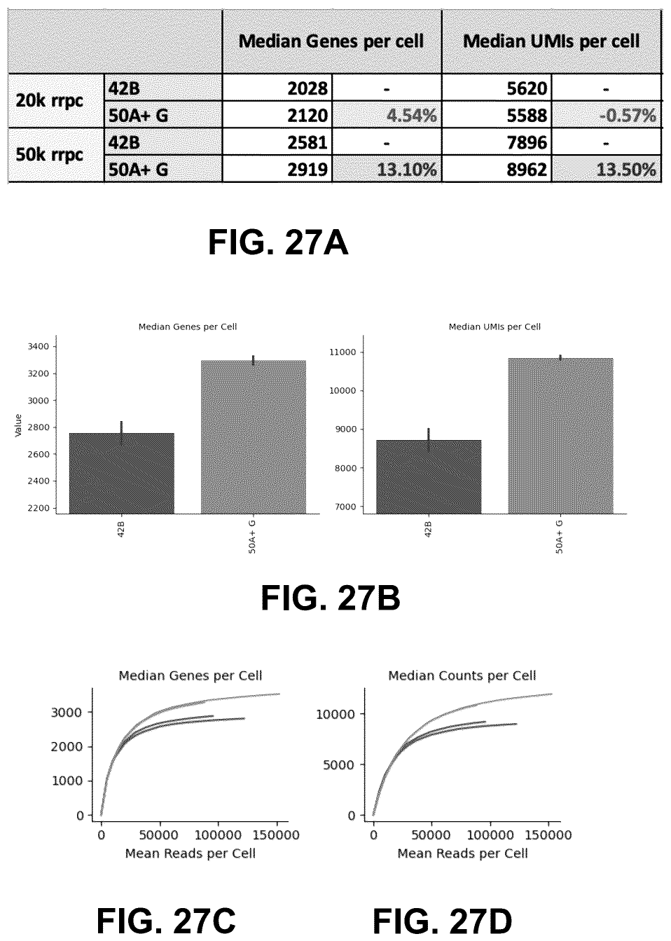

Another RT variant, 50A+G showed 23.22% (Median genes/cell) and 36.49% (median UMIs/cell) enhancement over 42B alone. The performance of 50A+G (SEQ ID NO: 147) in a Single Cell 5′ (SC-5′) gene expression assay when compared to a control MMLV variant (SEQ ID NO: 1 or 179; 42B) is shown in FIGS. 27A-D. FIGS. 27A-D show the superiority of the 50A+G as an improved RT for single cell assays. A performance comparison of the control MMLV variant and 50A+G shows that 50A+G enhanced the median genes per cell by about 4.54% at 20 k rrpc or 13.1% at 50 k rrpc, while the median UMIs per cell was enhanced by 13.50% at 50 k rrpc when compared to the control MMLV variant. FIG. 27B shows the enhanced performance of 50A+G at maximum normalization depth. FIGS. 27C-D demonstrate a clear benefit of using the 50A+G variant in the SC-5′assay as the read depth increases. 50A+G show Saturation curves for the control MMLV (42B) and 50A+G demonstrating the median genes (FIG. 27C) and counts/cell (FIG. 27D) as a function of read depth were higher using the 50A+G variant when compared to the control MMLV variant.

To determine the properties of the engineered RTs described herein, these RT enzymes were analyzed in 5′ gene expression assay. All RT enzymes were used at 1.31 uM concentration, and all purified by the same method. RT enzymes tested included 42B (SEQ ID NO: 1), 50A+G (Table 5; SEQ ID NO: 147), 42B_L (Table 5; SEQ ID NO: 145), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), and SOLD 035 (SEQ ID NO: 131).

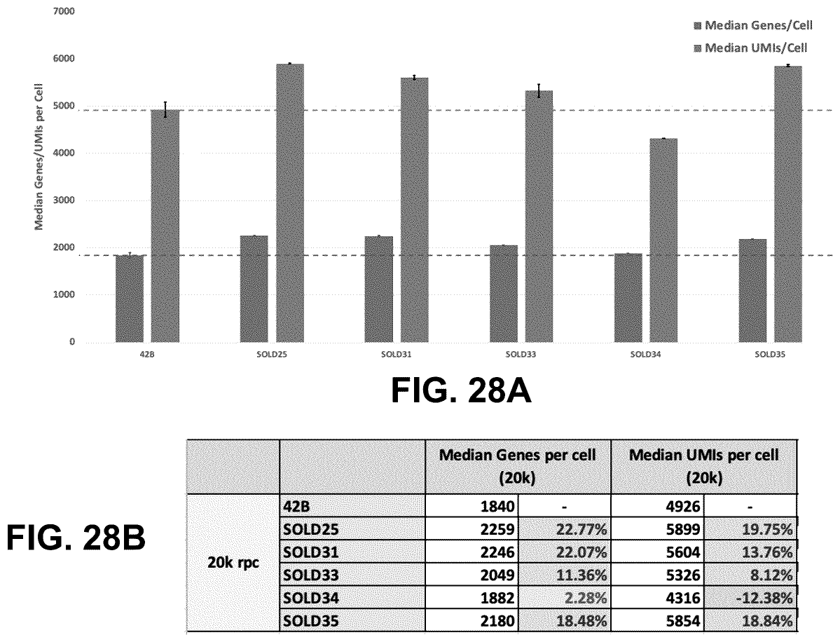

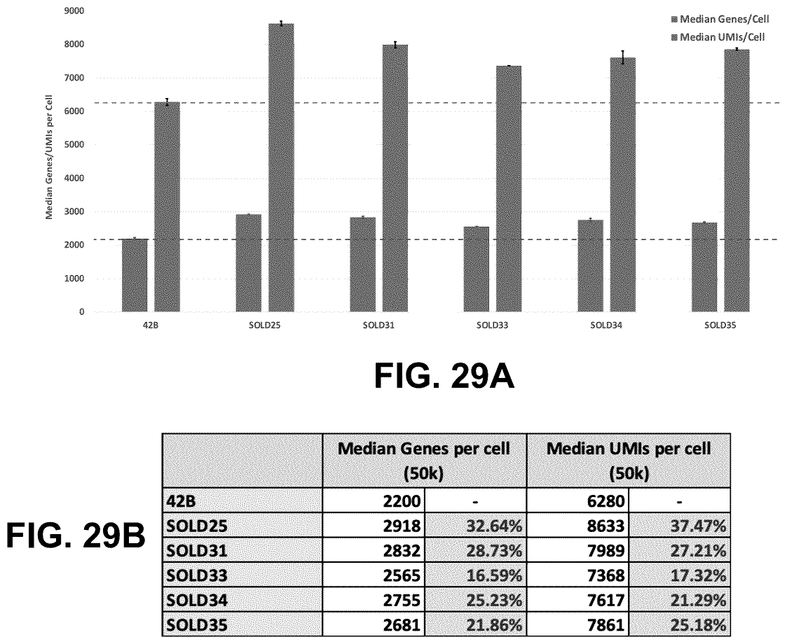

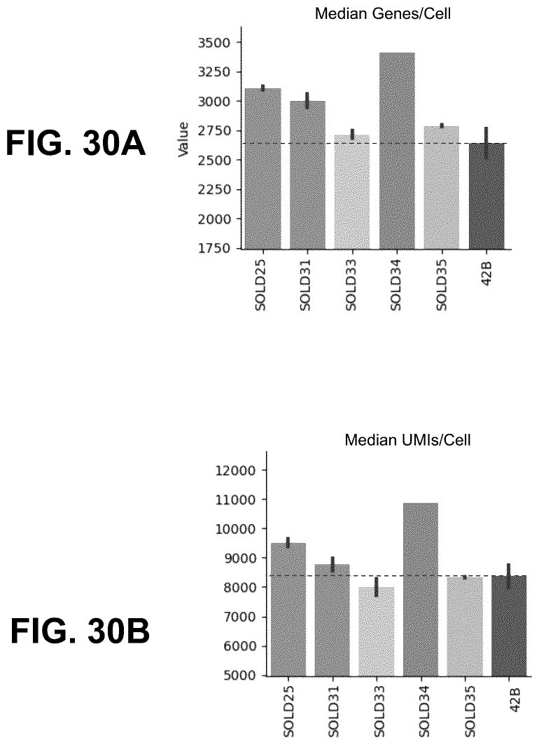

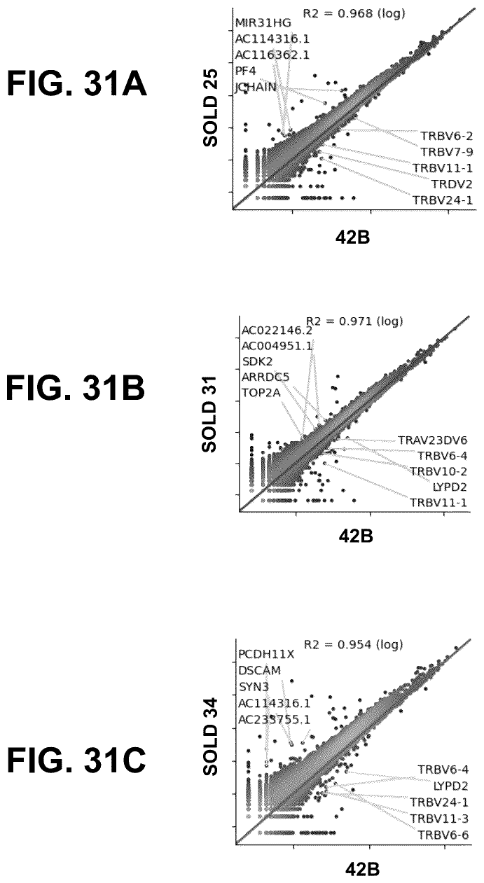

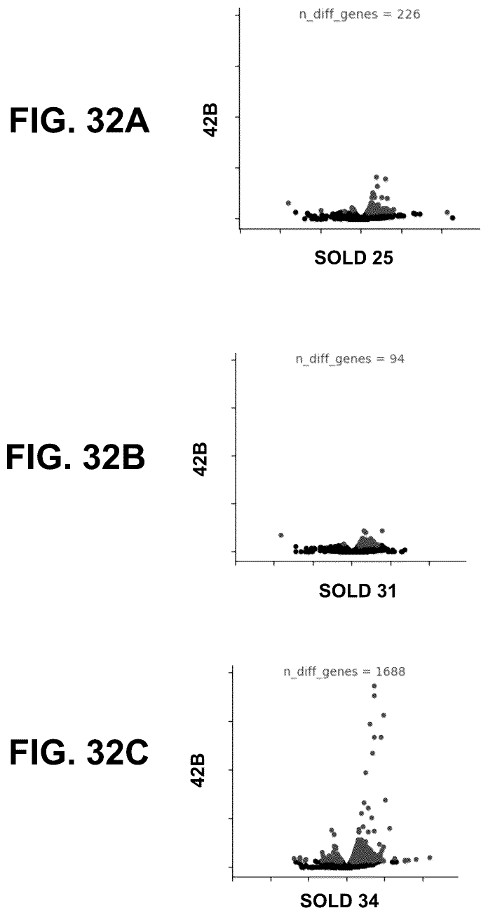

Several RTs variants show sensitivity gains relative to 42B at 20 k rrpc (FIGS. 28A-B). SOLD 34 performance surpassed the performance of the control MMLV RT (42B), SOLD 025, SOLD 031, SOLD 033, and SOLD 035, at maximum normalization depth. SOLD 25 benefits from library quality and mapping metrics on par with 42B, and can realize significant sensitivity gains at lower reads depths (FIGS. 28-30). A good correlation in differential gene expression (e.g., gene calling) between of some top performing RT variants (SOLD 025, SOLD 031, and SOLD 034) relative to the control MMLV (42B) was observed (FIGS. 31-32). FIGS. 31-32 show significant levels of differential gene expression with SOLD 034 and SOLD 025, and differential gene expression for SOLD 031.

### Example 6: Analysis of 42B L Sto7 Fusion in 5′ Single Cell Assay

This example shows that an engineered fusion reverse transcriptase described herein (e.g., 42B-L-Sot7 fusion) significantly improved the sensitivity of a 5′ single cell assay.

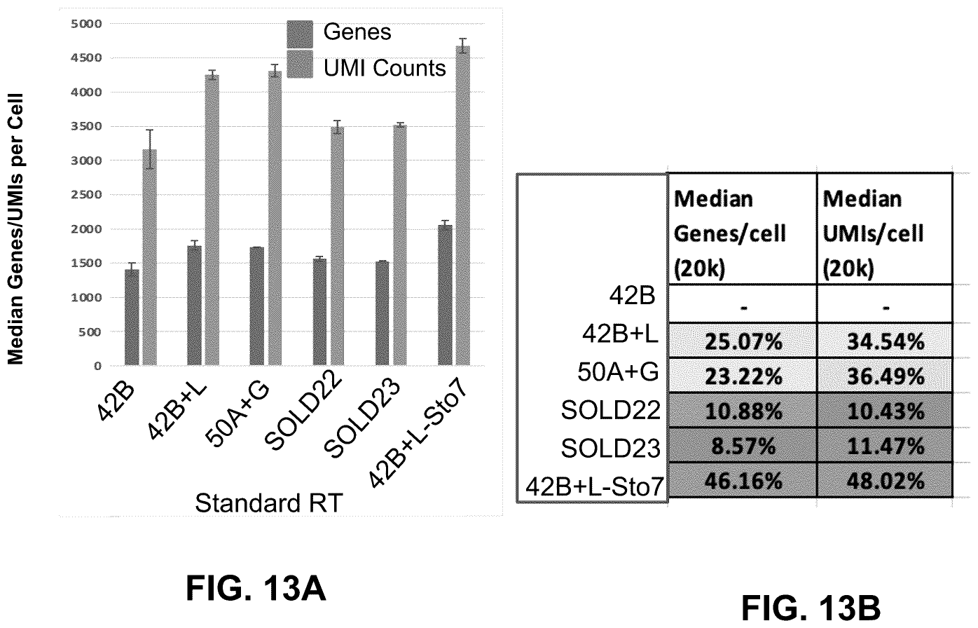

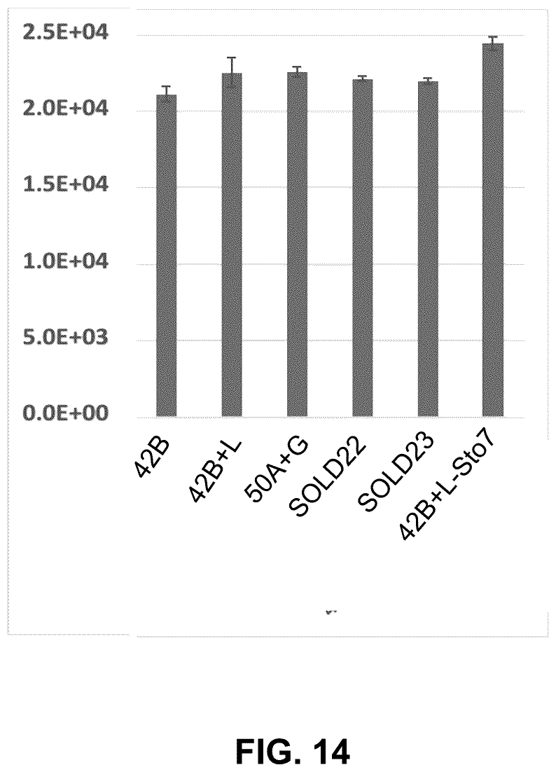

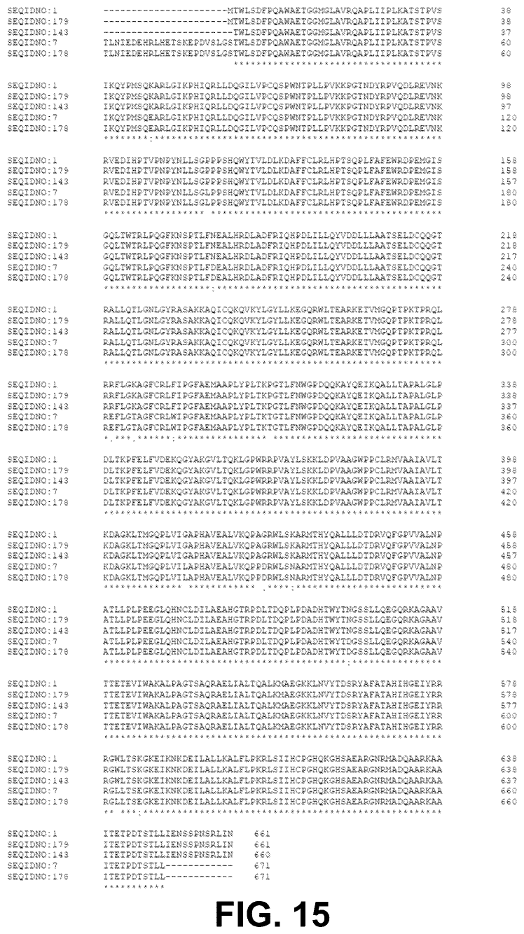

To determine the effectiveness of the engineered RT and/or engineered fusion RT described herein, these RT enzymes were analyzed in 5′ gene expression assay. Analysis of 42B L Sto7 fusion in 5′ single cell assay was performed and compared to an assay using 42B, or 42B L RT. All RT enzymes were used at 1.31 uM concentration, and all purified by the same method. RT enzymes tested included 42B (SEQ ID NO: 1), 50A+G (Table 5; SEQ ID NO: 147), 42B_L (Table 5; SEQ ID NO: 145), SOLD 022 (SEQ ID NO: 105), SOLD 023 (SEQ ID NO: 107), SOLD 025 (SEQ ID NO: 111), SOLD 031 (SEQ ID NO: 123), SOLD 033 (SEQ ID NO: 127), SOLD 034 (SEQ ID NO: 129), SOLD 035 (SEQ ID NO: 131), and 42BL_Sto7K13L (SEQ ID NO: 20; SEQ ID NO: 153). The K13L mutation in Sto7 (SEQ ID NO: 18) is a RNAse silencing mutation on Sto7. As shown in FIGS. 13A-B, 42B_L improved the sensitivity significantly over a known RT enzyme variant (42B or SEQ ID NO: 1 or 143). 42B_L (M66L) showed 25.07% (Median genes/cell) and 34.54% (median UMIs/cell) enhancement over 42B alone.

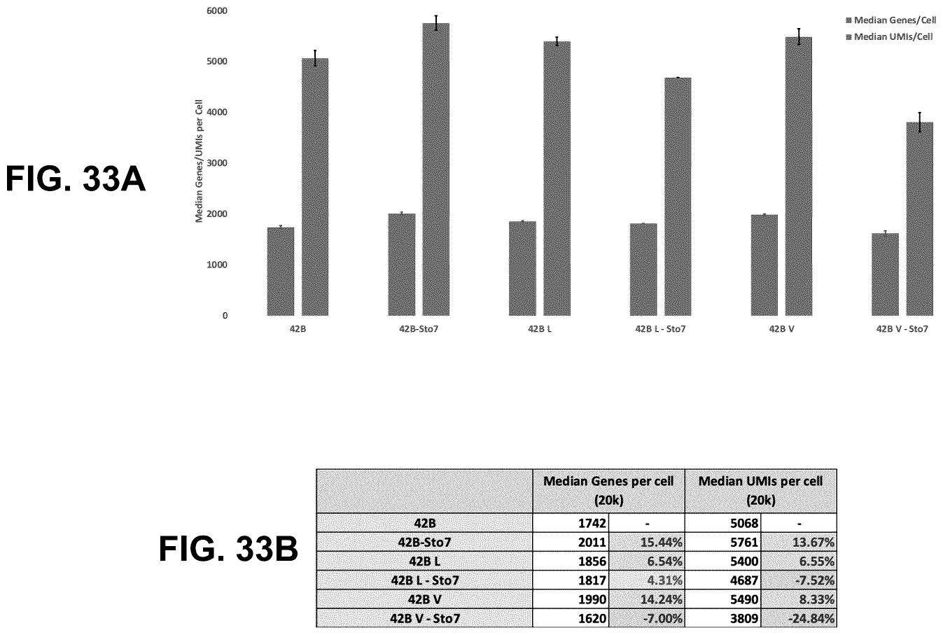

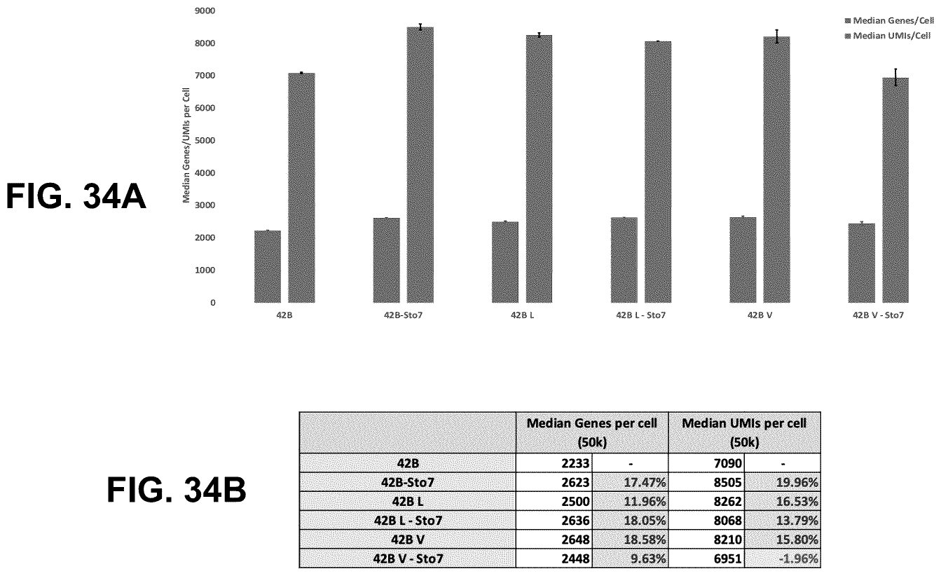

FIGS. 33A-B show median genes and UMIs/cell at 20 k raw-reads per cell comparing sensitivity of three reverse transcriptases with and without the Sto7 fusion domain; illustrating a clear benefit of the Sto7 fusion on 42B, but not on the mutants 42B L and 42B V. The difference is likely due to primer contamination issues in the final library and may be resolved with further assay optimization. FIGS. 34A-B show Median genes and UMIs/cell at 50 k raw-reads per cell comparing sensitivity of three reverse transcriptases with and without the Sto7 fusion domain; illustrating at that, at 50 k rrpc, some benefit could be seen with the Sto7 fusion on 42B L, but not 42B V. FIGS. 35A-B show median genes and UMIs/cell at maximum normalization depth for the sample set comparing sensitivity of three reverse transcriptases with and without the Sto7 fusion domain. The benefit of the Sto7 fusion on each RT variant backbone can clearly be seen at maximum normalized read depth (FIGS. 35A-B). Further optimization may allow this to be realized at lower read depths.

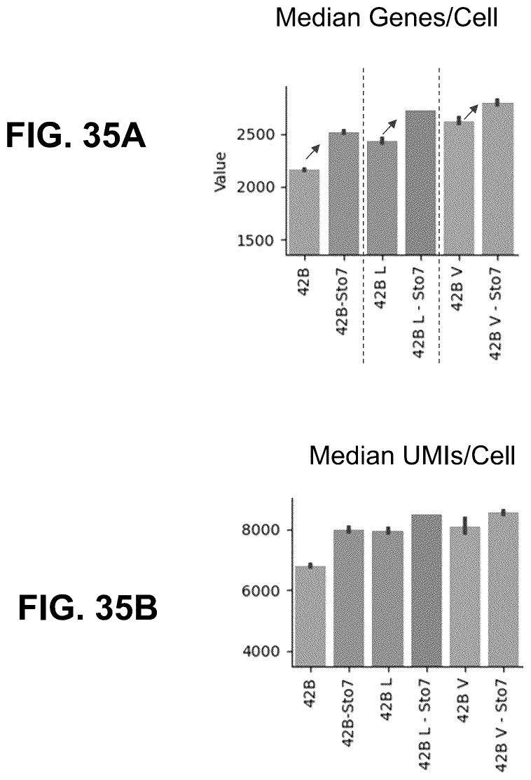

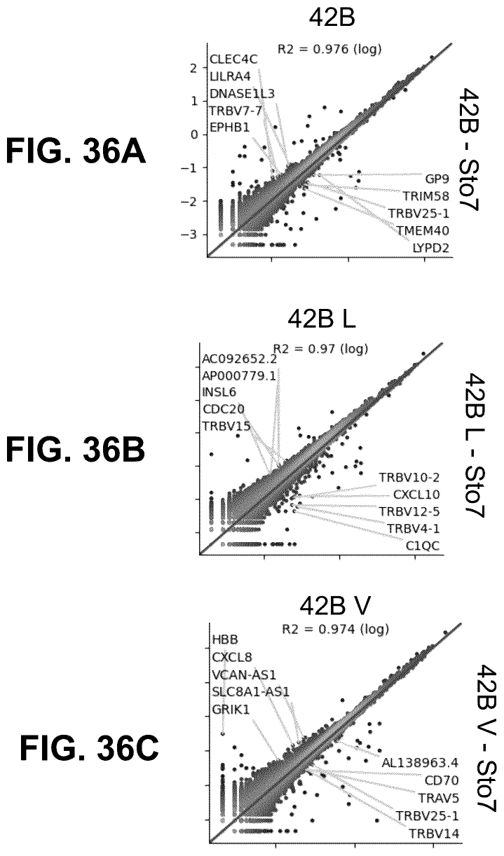

FIGS. 36A-B and FIGS. 37A-B show differential gene expression using the engineered RT fusion proteins comprising sto7. FIGS. 36A-B show scatter plots illustrating gene expression correlation of three reverse transcriptases with and without the Sto7 fusion domain and FIGS. 37A-B show volcano plots showing the number of differentially expressed genes between three reverse transcriptases with and without the Sto7 fusion domain. Together these figures illustrate that differential gene expression more pronounced with Sto7 fusions on 42B L and 42B V.

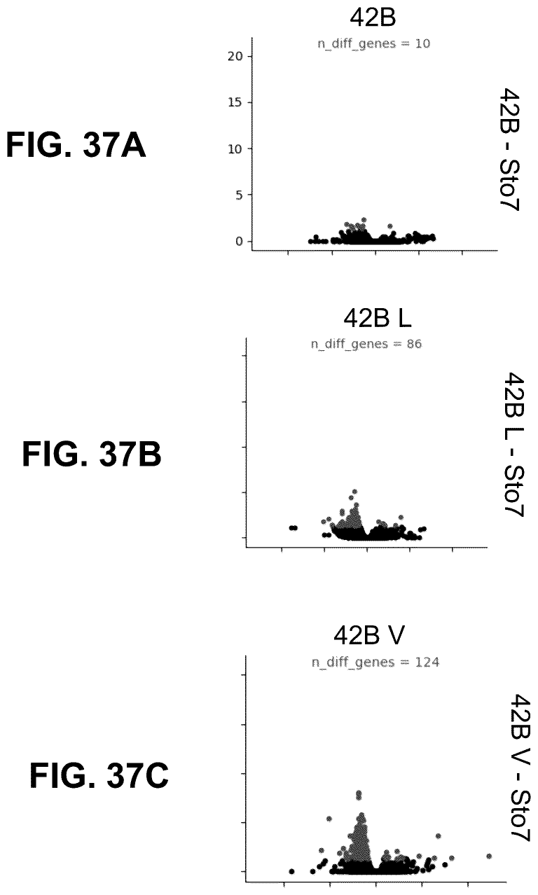

FIGS. 38A-C show graphs illustrating the performance comparison of the impact of the Sto7 fusion domain across three reverse transcriptase backbones based on median genes and UMIs/cell at maximum normalization depth (FIG. 38A), gene expression correlation (FIG. 38B), and differential gene expression (FIG. 38C). The aggregated metrics comparing the performance among 42B, 42B L, and 42B V backbones with and without the Sto7 fusion showed a clear performance benefit from including Sto7, such as e.g., enhanced sensitivity.

Surprisingly, the fusion of the DNA binding domain of sto7(K13L) to 42B+L further significantly enhanced the gain in sensitivity observed with the 42B+L variant (M66L) alone. Indeed, the 42B L Sto7 (SEQ ID NO: 20) fusion RT showed 46.16% (Median genes/cell) and 48.02% (median UMIs/cell) enhancement over 42B alone. This was an over 20% enhancement when compared to the 42B L variant alone. In addition, the 42B L Sto7 (SEQ ID NO: 20) fusion RT significantly enhanced the total number of genes detected in the single cell assay when compared to 42B, 42B+L (M66L), or 50A+G.

Therefore, the 42B L Sto7 (SEQ ID NO: 20) fusion RT significantly improve in single cell assay performance.

### Example 7: Assays for Analyses of RT Enzyme Variants

Any of the engineered RT enzymes of the invention, including without limitation any of the enzymes described in Table 4, Table 5, or Table 6 could be analyzed in any suitable assay, including without limitation the assays described herein. Assays include without limitation 5′ gene expression analyses, with or without VDJ analysis, 3′ gene expression analysis, epigenetic analysis, or multiomic analyses. In non-limiting embodiments, experiments are carried out as found in the manufacturer's instructions for the Chromium Single Cell 5′ Gene Expression Assay kit (10× Genomics); Chromium Single Cell 3′ Gene Expression Assay kit (10× Genomics), including any of mutliomic extensions or applications.

### Example 8: Single Cell 3′ and 5′ cDNA Yields

Various engineered reverse transcriptases were evaluated in single cell experiments with peripheral blood monocytes (PBMCs) at a cell load of 1,000, using the 3′ and 5′ configurations. Emulsion droplets contained gel beads with either barcoded poly-dT primer sequences (3′ configuration) or barcoded template switch oligo sequences (5′ configuration) that also include a UMI and Illumina Read 1 sequence. When cells are lysed within the droplet, the poly-dT primer hybridizes to the poly-A tail of the cellular mRNA, which is extended by the reverse transcriptase. Once the end of the template is reached, the reverse transcriptase will exhibit terminal transferase activity to add an overhang of three non-templated deoxycytidines (CCC) to the 3′ end of the synthesized cDNA. The CCC overhang will hybridize to the 3 riboguanosines (rGrGrG) present on the 3′ end of the template switch oligo, allowing the reverse transcriptase to “switch” templates and continue synthesis to the 5′ end of the template switch oligo. Depending on the which configuration of gel bead is used (3′ or 5′) the barcode and UMI will allow either the 3′ or 5′-end of the mRNA molecule to be identified in the final sequencing library. Following reverse transcription at 48° C. or 53° C. for 45 mins, and a 5 min heat-kill at 85° C., droplets were broken and the cDNA was purified with Dynabeads. The cDNA was then amplified via PCR, purified with a 0.6×SPRI, and quantified with an Agilent BioAnalyzer using the DNA High Sensitivity Kit. The cDNA yield (ng) was then obtained.

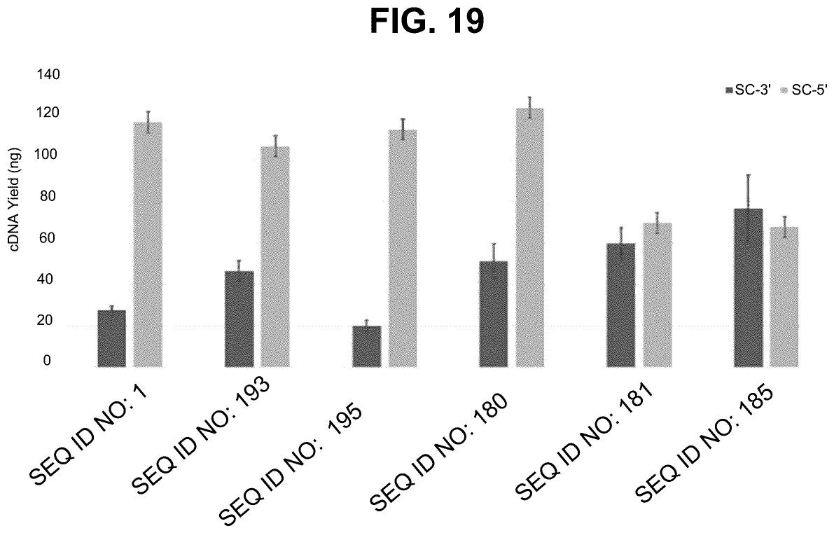

FIG. 19 shows a summary of the cDNA yield from a series of such experiments for the engineered reverse transcriptases having the amino acid sequence set forth in SEQ ID NOS: 1 or 179, 193, 195, 181, and 185. (n=2). The single cell experiments were performed in the 3′ and 5′ configurations. Results from the 3′ configuration are shown as the left bar for each enzyme, and results from the 5′ configuration are shown as the right bar for each enzyme. Yields for variants with a M66L mutation and/or the M39V mutation exceed the yield obtained from a reverse transcriptase having the amino acid sequence set forth in SEQ ID NO:1 or 179 in the 3′ experiments. These results are comparable to the results from tests of total product yield on the GAPDH template. Surprisingly, the yields for the single cell 5′ configuration differ from expectations based on the total product yield on the GAPDH template.

### Example 9: Single Cell 3′ Quality Metrics

Various engineered reverse transcriptases were evaluated in single cell experiments with peripheral blood monocytes (PBMCs) using the 3′ and 5′ reaction conditions. Either 10 μL of the amplified cDNA (3′ conditions) or 20 μL containing a maximum of 50 ng of amplified cDNA (5′ conditions) were then fragmented and A-tailed, cleaned with a double-sided SPRI (0.6×/0.8×), ligated to functional adaptors with an Illumina Read 2 sequence, cleaned with a 0.8×SPRI, and then further amplified with sample indexing primers that include the P5 and P7 priming sites and the i5 and i7 sample indexes. The amplification product was cleaned up with a double-side (0.6×/0.8×) SPRI, and the average size was determined with an Agilent BioAnalyzer using the DNA High Sensitivity Kit. The material was then quantified by qPCR and pooled for next generation sequencing on an Illumina Novaseq targeting a sequencing depth of at least 50,000 reads per cell and using the following run parameters (Read 1: 28 cycles, i7 Index: 10 cycles, i5 Index: 10 cycles, Read 2: 90 cycles). Data were collected, demultiplexed, and processed. Standard quality metrics were obtained.

Generally, the single cell 5′ reactions use less enzyme and TSO oligo than the single cell 3′ reactions. The 5′ TSO oligo is also twice the length of the 3′ TSO oligo with varied sequence context due to the presence of the UMI and the barcode. The single cell 5′ reaction conditions are generally considered a more stringent test of performance than the 3′ single cell reaction conditions.

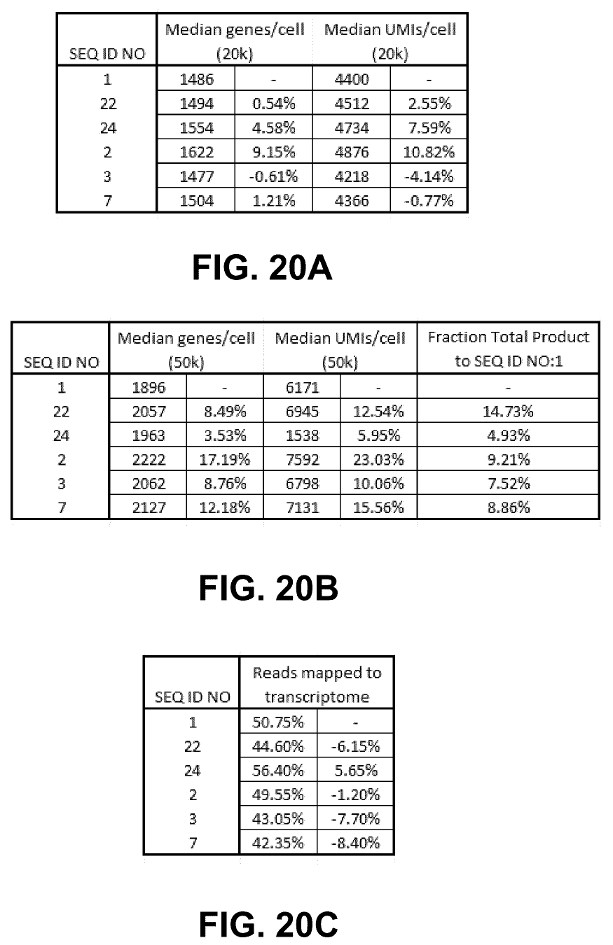

Results from one such series of experiments using 3′ reaction conditions are summarized in FIGS. 20A-C and 21A-B. FIGS. 20A-C show metrics of the 3′ single cell experiments; FIG. 20A provides 20 k read metrics, FIG. 20B provides 50K read metrics and FIG. 20C provides reads mapped to the transcriptome. The amino acid sequences of the indicated engineered reverse transcriptases are provided in the indicated SEQ ID NO. The percent indicates the percent change from the results obtained with an engineered reverse transcriptase having the amino acid sequence set forth in SEQ ID NO:1 or 179. All variants with a M66L mutation showed improved sensitivity at 50 kilo reads per cell (krpc) but the extent of the improvement depends on the context.

All variants with a M66L mutation showed improved sensitivity at 50 kilo reads per cell (krpc) but the extent of the improvement depends on the context. The trend correlated well with the capillary electrophoresis (CE) data with the engineered reverse transcriptase having the amino acid sequence set forth in SEQ ID NO: 195 underperforming relative to the other variants. Surprisingly, only the engineered reverse transcriptase having the amino acid sequence set forth in SEQ ID NO: 180 showed significant improvement at 20 krpc. The engineered reverse transcriptase having the amino acid sequence set forth in SEQ ID NO: 180 lacks the M39V mutation present in the amino acid sequences set forth in SEQ ID NO:181 and SEQ ID NO:185. Surprisingly, the M39V mutation improved template switching efficiency in vitro but in combination with M66L, the M39V mutation did not provide significant additional benefits. Further the engineered reverse transcriptase having the amino acid sequence set forth in SEQ ID NO: 180 lacked the P448A and D449G alterations present in SEQ ID NO:1 or 179, 193 and 185. Surprisingly, engineered reverse transcriptases having the amino acid sequences set forth in SEQ ID NO: 193 and 185 have similar sensitivities. The P448A and D449G alterations did not alter sensitivity in this context. Surprisingly, engineered reverse transcriptases with the M66L alteration, P448A, D449G and/or M39V suffered mapping loss. The exception is the engineered reverse transcriptase having the amino acid sequence set forth in SEQ ID NO: 180. Production of off-target products requires higher read depth to see improvements.

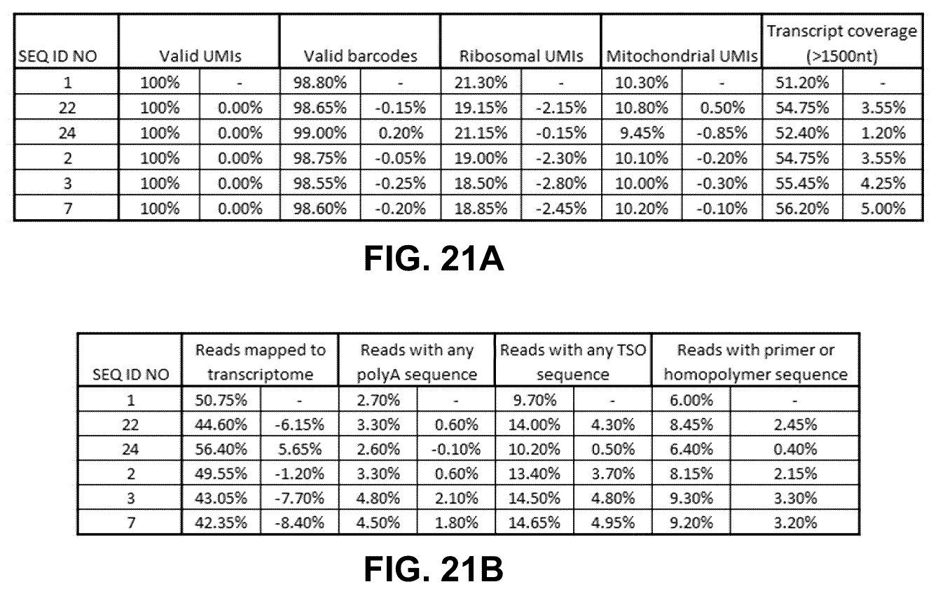

FIGS. 21A-B summarize additional metrics related to results obtained from the indicated engineered reverse transcriptase in single cell 3′ experiments. Most of the variants yielded metrics within parity for valid UMI's, valid barcodes, ribosomal UMI's, mitochondrial UMI's, transcript coverage (FIG. 21A), and reads with any poly A sequence, reads with any switch oligo sequence and reads with primer or homopolymer sequence (FIG. 21B). However, when the libraries produced by most variants with the M66L mutation in combination with either P448A, D449G and/or M39V were evaluated for reads mapped to the transcriptome, there was a decrease in reads mapped to the transcriptome. Surprisingly, the variant having the amino acid sequence set forth in SEQ ID NO: 180 which has the M66L alteration exhibited improved template switching efficiency and maintains levels of reads mapped to the transcriptome close to that obtained with the engineered reverse transcriptase having the amino acid sequence set forth in SEQ ID NO:1 or 179.

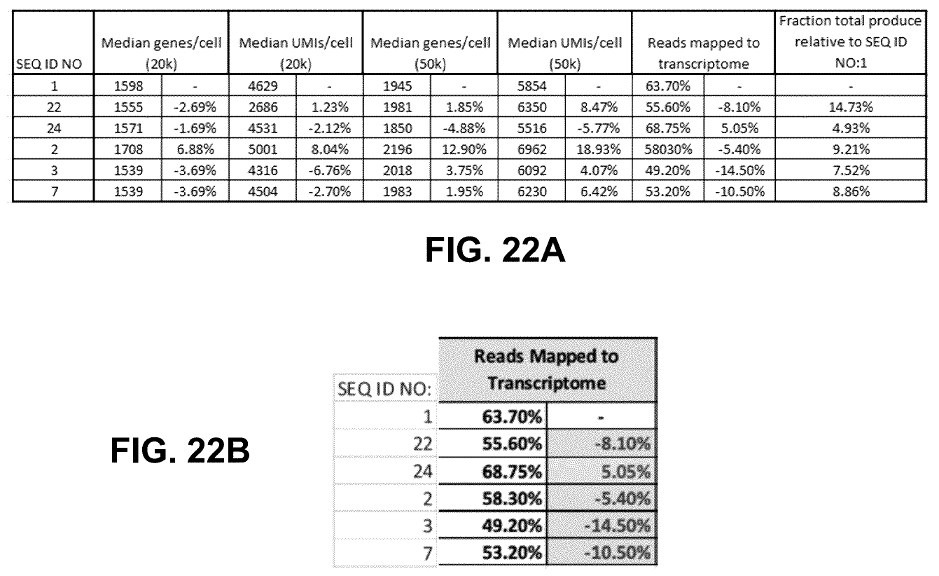

FIGS. 22A-B and 23A-B summarize results from a series of experiments using the 5′ reaction conditions. FIGS. 22A-B summarize metrics of the 5′ single cell experiments, including 20 k read metrics, 50K read metrics and reads mapped to the transcriptome. The engineered reverse transcriptase variants have the amino acid sequences provided in the indicated SEQ ID NO. The percent indicates the percent change from the results obtained with an engineered reverse transcriptase having the amino acid sequence set forth in SEQ ID NO:1 or 179. In particular, an engineered reverse transcriptase having the amino acid sequence set forth in SEQ ID NO:180 showed a significant improvement in sensitivity. Engineered reverse transcriptases with the M66L alteration, P448A, D449G and/or M39V suffered mapping loss.

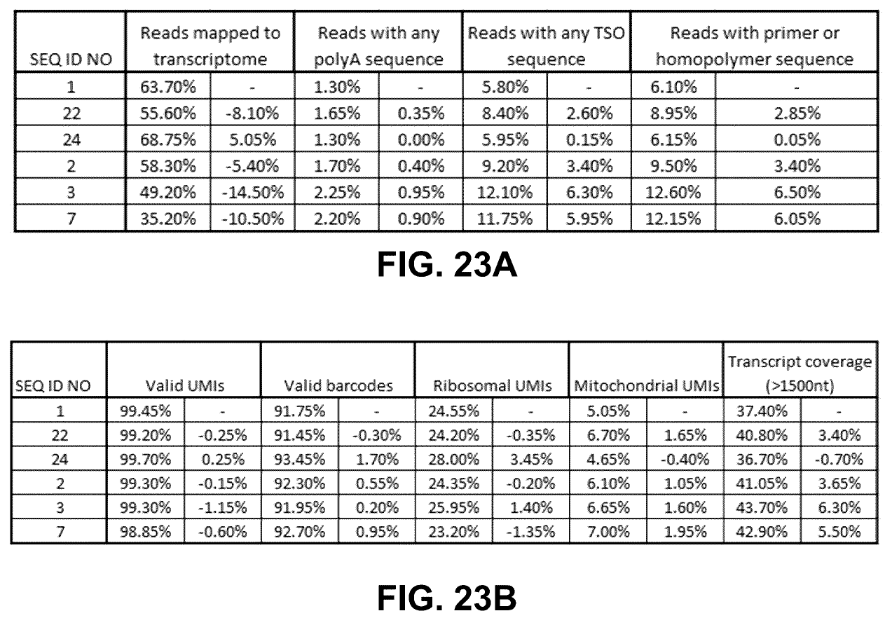

FIGS. 23A-B show additional metrics related to results obtained from the indicated engineered reverse transcriptase in single cell 5′ experiments. Most of the variants yielded metrics within parity for valid UMI's, valid barcodes, ribosomal UMI's, mitochondrial UMI's, transcript coverage (FIG. 23A), reads with any poly A sequence, reads with any switch oligo sequence and reads with primer or homopolymer sequence (FIG. 23B).

However, when the libraries produced by most variants with the M66L mutation in combination with either P448A, D449G and/or M39V were evaluated for reads mapped to the transcriptome, there was a decrease in reads mapped to the transcriptome. Surprisingly the variant having the amino acid sequence set forth in SEQ ID NO: 180, which has the M66L alteration exhibits improved template switching efficiency and the levels of reads mapped to the transcriptome is impacted less than when other engineered reverse transcriptases are used.

### Example 10: Single Cell Sensitivity and Mapping

Various engineered reverse transcriptases were evaluated in single cell experiments with human peripheral blood monocytes (PBMCs) and mouse peripheral blood monocyte cells (C57B/L6) using 3′ and 5′ reaction conditions as described above herein. Sensitivity and mapping were evaluated. Results from engineered reverse transcriptases were compared to results obtained from a commercially available engineered MMLV.

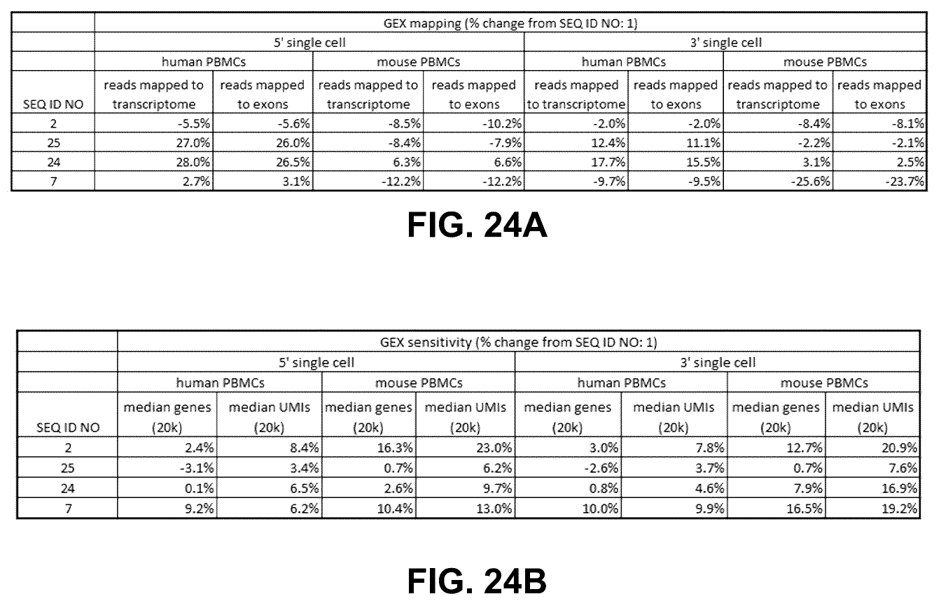

Results from one such series of experiments are summarized in FIGS. 24A-B. The percent change is as compared to a commercially available variant MMLV reverse transcriptase. The change in median genes and median UMI's queried at 20,000 reads per cell and the change in reads mapped to the transcriptome and reads mapped to exons are shown. A commercially available engineered reverse transcriptase was used as the control. The amino acid sequences of the engineered reverse transcriptases are set forth in SEQ ID NO: 180, SEQ ID NO: 185, SEQ ID NO: 195 and SEQ ID NO: 196. Improvements in both the 5′ and 3′ chemistries are more pronounced in the mouse PBMC's than in the human PBMCs. Note the significant improvements exhibited by the engineered reverse transcriptase having the amino acid sequence set forth in SEQ ID NO: 180. It is also noted that the reads mapped to the transcriptome or the exon obtained with an engineered reverse transcriptase having the amino acid sequence set forth in SEQ ID NO: 180 decreased as compared to a commercially available engineered reverse transcriptase.

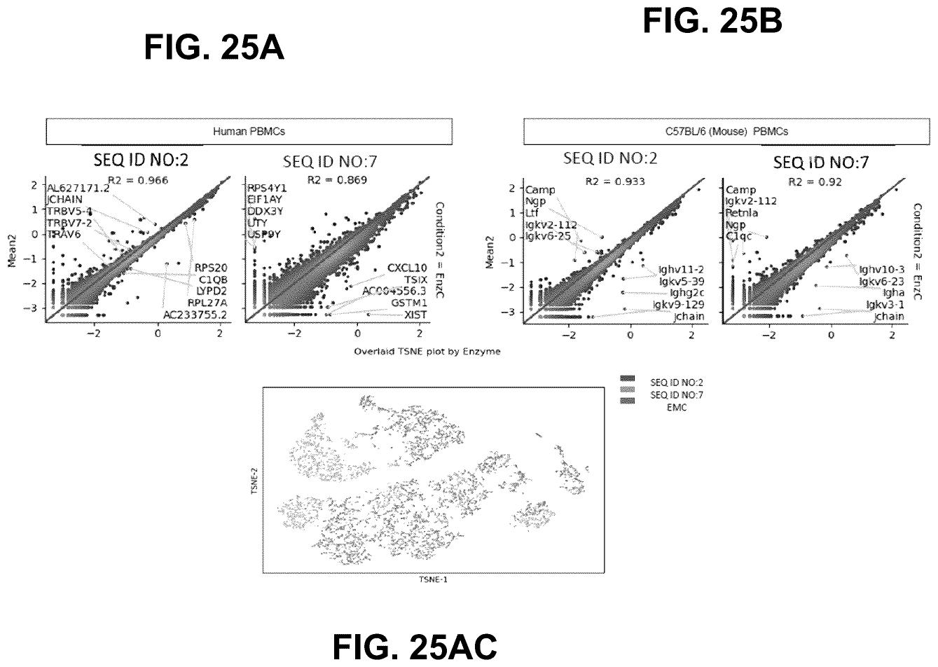

Further, t-Distributed Stochastic Neighbor Embedding (t-SNE) was used to evaluate the homogeneity of cell populations evaluated with engineered reverse transcriptase variants having the amino acid sequence set forth in SEQ ID NO:180 and SEQ ID NO:185 and a commercially available engineered reverse transcriptase. Results from a t-SNE analysis are shown in FIG. 25C. As shown therein, the correlation exhibited by an engineered reverse transcriptase having the amino acid sequence set forth in SEQ ID NO: 180 was tighter than the correlation exhibited by an engineered reverse transcriptase having the amino acid sequence set forth in SEQ ID NO:185 (FIGS. 25A-B). The engineered reverse transcriptase having the amino acid sequence set forth in SEQ ID NO:185 exhibited a tighter correlation in mouse cells than in human cells in 5′ and 3′ chemistries (3′ data not shown). FIG. 25C shows an overlaid t-SNE plot by enzyme. The engineered reverse transcriptase having the amino acid sequence set forth in SEQ ID NO: 180 and a commercially available engineered reverse transcriptase show homogeneity in cell populations compared to the engineered reverse transcriptase having the amino acid sequence set forth in SEQ ID NO:185.

### Example 11: Immunoprofiling and TCR Improvements

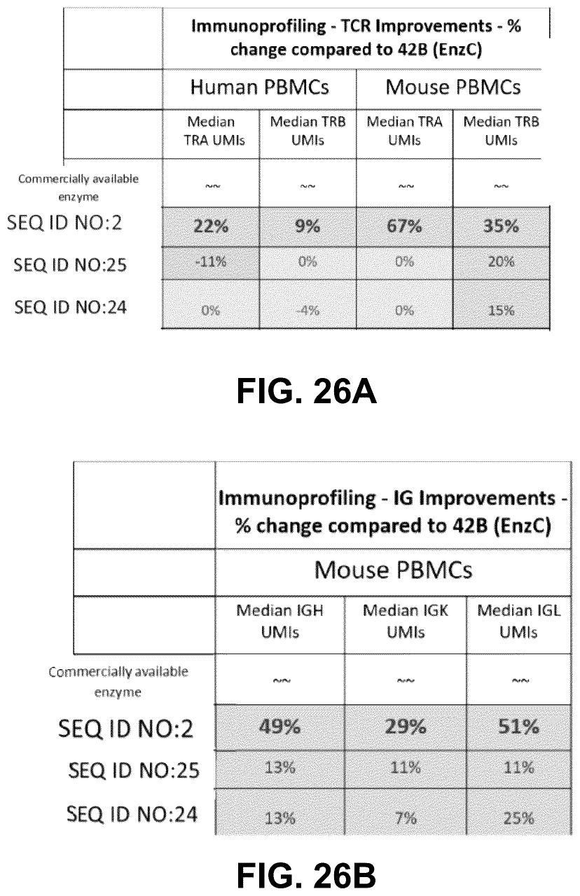

Immune profiling is an extension of the 5′ chemistry to profile genes specifically for T-cell and/or B-cell receptors in the mRNA pool. Methods of immune profiling are known in the art and generally include additional rounds of PCR on the cDNA with a pool of sequence specific primers to allow for targeted enrichment of T-cell and/or B-cell receptor genes. Immune profiling assays may also detect UMIs for B-cell receptor genes, namely IGH, IGK, and IGL (Immunoglobulin heavy chain (IGH), kappa (IGK), and light (IGL) chain). Immune profiling data is informative for immunology research and is an extension of standard gene expression evaluation. Methods of immune profiling include, but are not limited to Chromium Next Gen Single Cell™ kits (10× Genomics, Pleasanton CA). Amplified cDNA (2 μl) from the 5′ configuration of reverse transcription reactions were subjected to two additional rounds of PCR enrichment with TCR immune profiling, which included a double-sided (0.5×/0.8×) SPRI clean-up between the first and second round of thermal cycling reactions. The amplified products were then cleaned-up with a subsequent double-sided (0.5×/0.8×) SPRI, fragmented and A-tailed, ligated to functional adaptors with an Illumina Read 2 sequence, cleaned up with a 0.8×SPRI, and then further amplified with sample indexing primers that include the P5 and P7 priming sites and the i5 and i7 sample indexes. The amplification product was cleaned up with a 0.8×SPRI, and average size was determined with an Agilent BioAnalyzer using the DNA High Sensitivity Kit. The material was then quantified by qPCR and pooled for next generation sequencing on an Illumina Novaseq targeting a sequencing depth of at least 5,000 reads per cell and using the following run parameters (Read 1: 28 cycles, i7 Index: 10 cycles, i5 Index: 10 cycles, Read 2: 90 cycles). Data were collected, demultiplexed, and single-cell V(D)J analysis was performed. Results obtained from engineered reverse transcriptases were compared to results obtained from a commercially available enzyme (FIGS. 26A-B). The percent change in median TRA UMI's and median TRB UMI's from mouse and human PBMCs for each RT tested is shown in FIG. 26A. FIG. 26B shows the percent change in median IGH, IGK and IGL from mouse PBMC's.

The median TRA UMIs and median TRB UMIs obtained with an engineered reverse transcriptase having the amino acid sequence set forth in SEQ ID NO: 180 were greater than those obtained with a commercially available engineered reverse transcriptase in both human PBMCs and mouse PBMCs. Engineered reverse transcriptases previously shown to exhibit IG sensitivity exhibited a comparable or improved IG sensitivity as compared to previous results. In mouse PBMC's, the median IGH UMIs, median IGK UMIs and median IGL UMIs obtained with enzymes having the amino acid sequence set forth in SEQ ID NO: 180, SEQ ID NO: 196 or SEQ ID NO: 195 were greater than those obtained with a commercially available engineered reverse transcriptase (right chart). The results obtained with an engineered reverse transcriptase having the amino acid sequence set forth in SEQ ID NO: 180 were substantially higher than those obtained with engineered reverse transcriptases having the amino acid sequence set forth in SEQ ID NO: 195 or SEQ ID NO: 196. The improvement shown with mouse PBMCs were similar to the results observed with GEX in FIG. 24. Example 12: 3′ assay with SOLD25 and 34 enzymes.

SOLD 025 and SOLD 034 engineered reverse transcriptases were evaluated in single cell experiments with human peripheral blood monocytes (PBMCs) using 3′ reaction conditions as described above herein. Sensitivity and mapping were the evaluated. Results from engineered reverse transcriptases were compared to results obtained from a commercially available engineered MMLV.

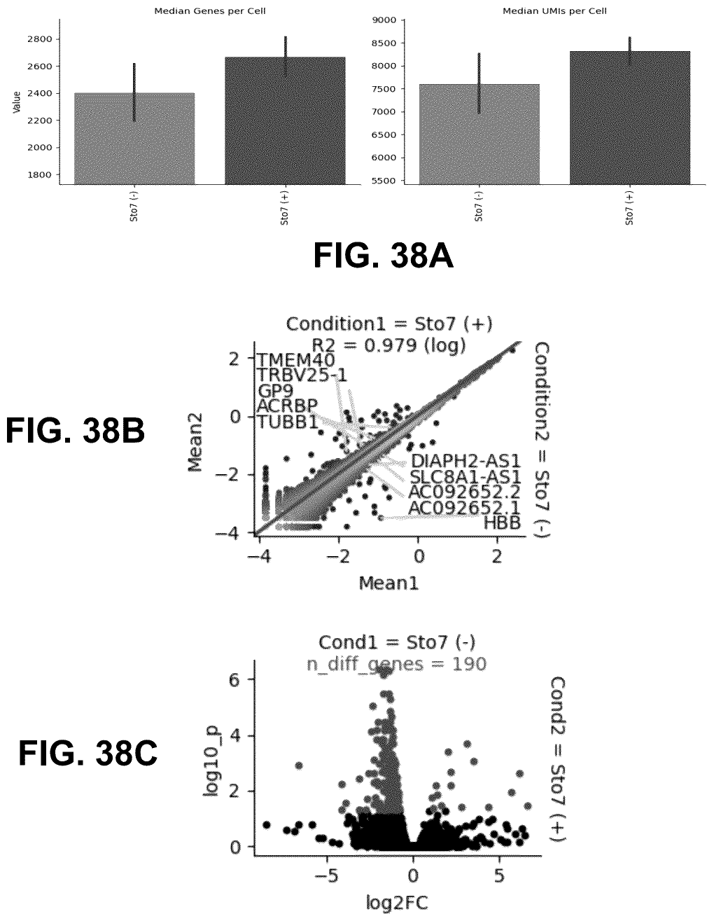

FIG. 39 also shows the performance of various RT variants in a Single Cell 3′ (SC-3′) gene expression assay at 50 k raw-reads per cell (rrpc). Median genes and UMIs/cell at 50 k rrpc show the enhanced performance, based on sensitivity gain, of the novel variants when compared to the control MMLV variant (enzyme mix B or enzyme mix C). SOLD 025 (SEQ ID NO: 111), and SOLD 034 (SEQ ID NO: 129). FIG. 39 further shows that SOLD enzymes show extensive (e.g. massive) complexity gains across the board.

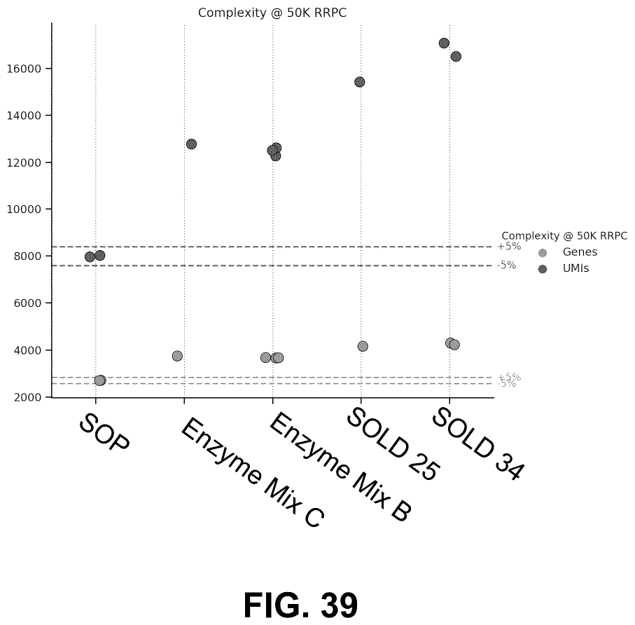

FIGS. 40A-D show the performance of various engineered RT variants (e.g., SOLD 025 (SEQ ID NO: 111), and SOLD 034 (SEQ ID NO: 129)) in a Single Cell 3′ (SC-3′) gene expression assay when compared to a control MMLV variants (enzyme mix B and enzyme mix C); and show the superiority of SOLD 25 and SOLD 34 as improved RTs for single cell assays. FIGS. 40A-B show saturation curves for the control MMLV and the engineered RT demonstrating the median genes (FIG. 40A) and counts/cell (FIG. 40B) as a function of read depth; and further demonstrating a clear benefit of using the engineered RT variants in the SC-3′assay as read depth increases. FIGS. 40C-D show bar graphs demonstrating the performance comparison summarizing median genes and UMIs per cell at maximum normalization depth; and illustrating the superior performance of the RT variants at maximum normalization depth. These data show Major complexity gains from 42B based assay mods, even more from SOLD enzymes.

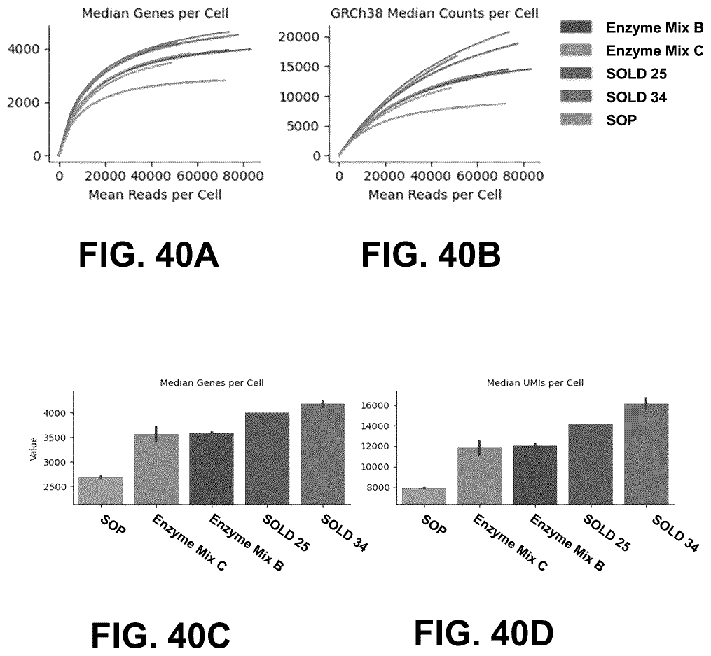

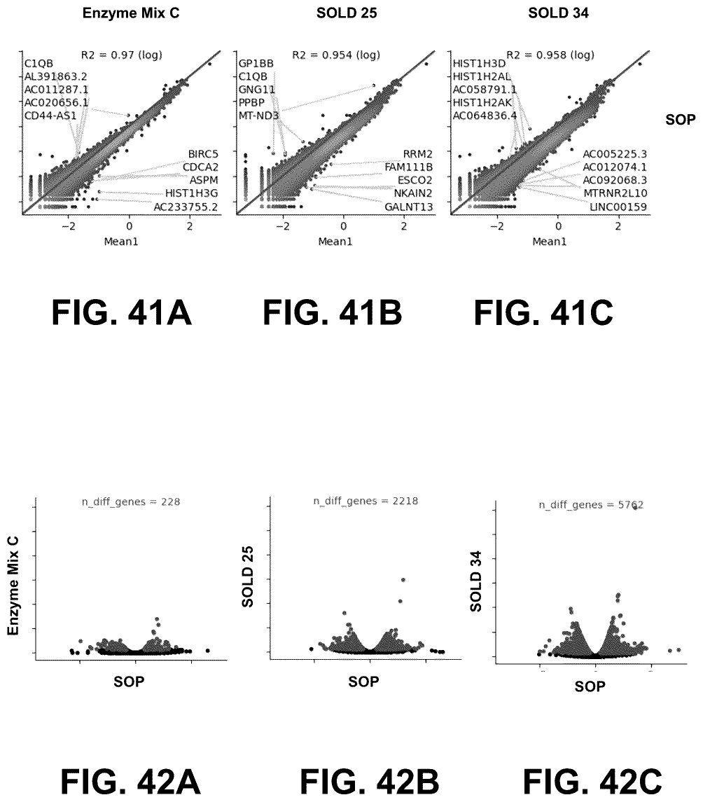

FIGS. 41A-C show scatter plots illustrating the differential gene expression of some top performing novel RT variants (SOLD 025 (SEQ ID NO: 111), and SOLD 034 (SEQ ID NO: 129)) based on a Single Cell 3′ (SC-3′) gene expression assay and gene expression correlation between a control RT (Enzyme Mix C), SOP and some top preforming novel RT variants. FIGS. 42A-C show volcano plots illustrating the number of differentially expressed genes between SOP, the control RT (Enzyme Mix C) and top preforming novel RT variants of FIGS. 41A-C. A good correlation in differential gene expression (e.g., gene calling) between of some top performing RT variants (SOLD 025 and SOLD 034) relative to the control MMLV (Enzyme Mix C) and was observed (FIGS. 41-42). In addition, significant levels of differential gene expression with SOLDs 34 and 25 were also observed.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

Selected sequences with annotation of the amino acid changes contemplated by the present disclosure are provided herein. “Mutation” shows the position within the amino acid sequence listed immediately below the annotation. “Label” is the name of the mutation as indexed to SEQ ID NO: 7.

| LOCUS: 42B 660 aa linear UNA (SEQ ID NO: 143) | |
|---|---|
| SOURCE: synthetic DNA construct (unknown) | |
| ORGANISM synthetic DNA construct | |
| synthetic DNA construct | |
| FEATURES | Location/Qualifiers |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| mutation | 501 |
| | /label = “D524N” |
| mutation | 580 |
| | /label = “L603W” |
| mutation | 584 |
| | /label = “E607K” |
| 1 TWLSDFPQAW AETGGMGLAV RQAPLIIPLK ATSTPVSIKQ YPMSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDILAEAH | |
| 481 GTRPDLTDQP LPDADHTWYT NGSSLLQEGQ RKAGAAVTTE TEVIWAKALP AGTSAQRAEL | |
| 541 IALTQALKMA EGKKLNVYTD SRYAFATAHI HGEIYRRRGW LTSKGKEIKN KDEILALLKA | |
| 601 LFLPKRLSII HCPGHQKGHS AEARGNRMAD QAARKAAITE TPDTSTLLIE NSSPNSRLIN | |
| LOCUS SOLD_025 660 aa linear UNA (SEQ ID NO: 111) | |
| SOURCE synthetic DNA construct (unknown) | |
| ORGANISM synthetic DNA construct | |
| synthetic DNA construct | |
| FEATURES | Location/Qualifiers |
| mutation | 16 |
| | /label = “M39V” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| mutation | 501 |
| | /label = “D524N” |
| mutation | 519 |
| | /label = “T542D” |
| mutation | 560 |
| | /label = “D583N” |
| mutation | 580 |
| | /label = “L603W” |
| mutation | 584 |
| | /note = “42B is E607K” |
| | /label = “E607G” |
| mutation | 621 |
| | /label = “A644V” |
| mutation | 630 |
| | /label = “D653H” |
| mutation | 635 |
| | /label = “K658R” |
| mutation | 648 |
| | /label = “L671P” |
| 1 TWLSDFPQAW AETGGVGLAV RQAPLIIPLK ATSTPVSIKQ YPMSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDILAEAH | |
| 481 GTRPDLTDQP LPDADHTWYT NGSSLLQEGQ RKAGAAVTDE TEVIWAKALP AGTSAQRAEL | |
| 541 IALTQALKMA EGKKLNVYTN SRYAFATAHI HGEIYRRRGW LTSGGKEIKN KDEILALLKA | |
| 601 LFLPKRLSII HCPGHQKGHS VEARGNRMAH QAARRAAITE TPDTSTLPIE NSSPNSRLIN | |
| LOCUS SOLD_034 660 aa linear UNA (SEQ ID NO: 129) | |
| SOURCE synthetic DNA construct (unknown) | |
| ORGANISM synthetic DNA construct | |
| synthetic DNA construct | |
| FEATURES | Location/Qualifiers |
| mutation | 16 |
| | /label = “M39V” |
| mutation | 43 |
| | /label = “M66I” |
| mutation | 46 |
| | /label = “E69K” |
| mutation | 68 |
| | /label = “Q91R” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 324 |
| | /label = “I347V” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| mutation | 501 |
| | /label = “D524N” |
| mutation | 571 |
| | /label = “H594Q” |
| mutation | 580 |
| | /label = “L603W” |
| mutation | 584 |
| | /label = “E607K” |
| 1 TWLSDFPQAW AETGGVGLAV RQAPLIIPLK ATSTPVSIKQ YPISQKARLG IKPHIQRLLD | |
| 61 QGILVPCRSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEVKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDILAEAH | |
| 481 GTRPDLTDQP LPDADHTWYT NGSSLLQEGQ RKAGAAVTTE TEVIWAKALP AGTSAQRAEL | |
| 541 IALTQALKMA EGKKLNVYTD SRYAFATAHI QGEIYRRRGW LTSKGKEIKN KDEILALLKA | |
| 601 LFLPKRLSII HCPGHQKGHS AEARGNRMAD QAARKAAITE TPDTSTLLIE NSSPNSRLIN | |
| LOCUS 50A+_G 660 aa linear UNA (SEQ ID NO: 147) | |
| SOURCE synthetic DNA construct (unknown) | |
| ORGANISM synthetic DNA construct | |
| synthetic DNA construct | |
| FEATURES | Location/Qualifiers |
| mutation | 43 |
| | /label = “M66L” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 431 |
| | /label = “N454K” |
| mutation | 480 |
| | /label = “H503V” |
| mutation | 501 |
| | /label = “D524N” |
| mutation | 580 |
| | /label = “L603W” |
| mutation | 584 |
| | /label = “E607K” |
| mutation | 611 |
| | /label = “H634Y” |
| 1 TWLSDFPQAW AETGGMGLAV RQAPLIIPLK ATSTPVSIKQ YPLSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPPDRWLS KARMTHYQAL LLDTDRVQFG PVVALNPATL LPLPEEGLQH NCLDILAEAV | |
| 481 GTRPDLTDQP LPDADHTWYT NGSSLLQEGQ RKAGAAVTTE TEVIWAKALP AGTSAQRAEL | |
| 541 IALTQALKMA EGKKLNVYTD SRYAFATAHI HGEIYRRRGW LTSKGKEIKN KDEILALLKA | |
| 601 LFLPKRLSII YCPGHQKGHS AEARGNRMAD QAARKAAITE TPDTSTLLIE NSSPNSRLIN | |
| LOCUS 42B_L_-_Sto7_K13L 728 aa linear UNA (SEQ ID NO: 155) | |
| SOURCE synthetic DNA construct (unknown) | |
| ORGANISM synthetic DNA construct | |
| synthetic DNA construct | |
| FEATURES | Location/Qualifiers |
| gene | 1 . . . 660 |
| | /label = “42B” |
| mutation | 43 |
| | /label = “M66L” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| mutation | 501 |
| | /label = “D524N” |
| mutation | 580 |
| | /label = “L603W” |
| mutation | 584 |
| | /label = “E607K” |
| misc_feature | 661 . . . 665 |
| | /feature_type = “Misc.” |
| | /label = “GGGGS Linker” |
| Region | 666 . . . 728 |
| | /region_type = “Domain” |
| | /label = “Sto7” |
| mutation | 677 |
| | /note = “RNAse silencing mutation on Sto7” |
| | /label = “K13L” |
| 1 TWLSDFPQAW AETGGMGLAV RQAPLIIPLK ATSTPVSIKQ YPLSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDILAEAH | |
| 481 GTRPDLTDQP LPDADHTWYT NGSSLLQEGQ RKAGAAVTTE TEVIWAKALP AGTSAQRAEL | |
| 541 IALTQALKMA EGKKLNVYTD SRYAFATAHI HGEIYRRRGW LTSKGKEIKN KDEILALLKA | |
| 601 LFLPKRLSII HCPGHQKGHS AEARGNRMAD QAARKAAITE TPDTSTLLIE NSSPNSRLIN | |
| 661 GGGGSVTVKF KYKGEELEVD ISKIKKVWRV GKMISFTYDD NGKTGRGAVS EKDAPKELLQ | |
| 721 MLEKSGKK | |
| LOCUS 42B_L_-_Sto7_Trunc_586 631 aa linear UNA (SEQ ID NO: 135) | |
| FEATURES | Location/Qualifiers |
| gene | 1 . . . 563 |
| | /label = “42B L Trunc 586” |
| mutation | 43 |
| | /label = “M66L” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| mutation | 501 |
| | /label = “D524N” |
| misc_feature | 564 . . . 568 |
| | /feature_type = “Misc.” |
| | /label = “Linker” |
| Region | 569 . . . 631 |
| | /region_type = “Domain” |
| | /label = “Sto7” |
| mutation | 580 |
| | /note = “RNAse silencing mutation on Sto7” |
| | /label = “K13L” |
| 1 TWLSDFPQAW AETGGMGLAV RQAPLIIPLK ATSTPVSIKQ YPLSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDILAEAH | |
| 481 GTRPDLTDQP LPDADHTWYT NGSSLLQEGQ RKAGAAVTTE TEVIWAKALP AGTSAQRAEL | |
| 541 IALTQALKMA EGKKLNVYTD SRYGGGGSVT VKFKYKGEEL EVDISKIKKV WRVGKMISFT | |
| 601 YDDNGKTGRG AVSEKDAPKE LLQMLEKSGK K | |
| LOCUS 42B_L_-_Sto7_Trunc_497 543 aa linear UNA (SEQ ID NO: 137) | |
| FEATURES | Location/Qualifiers |
| gene | 1 . . . 474 |
| | /label = “42B L Trunc 497” |
| mutation | 43 |
| | /label = “M66L” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| misc_feature | 475 . . . 480 |
| | /feature_type = “Misc.” |
| | /label = “Linker” |
| Region | 481 . . . 543 |
| | /region_type = “Domain” |
| | /label = “Sto7” |
| mutation | 492 |
| | /note = “RNAse silencing mutation on Sto7” |
| | /label = “K13L” |
| 1 TWLSDFPQAW AETGGMGLAV RQAPLIIPLK ATSTPVSIKQ YPLSQKARLG IKPHIQRLLD | |
| 61 QGILVPCQSP WNTPLLPVKK PGTNDYRPVQ DLREVNKRVE DIHPTVPNPY NLLSGPPPSH | |
| 121 QWYTVLDLKD AFFCLRLHPT SQPLFAFEWR DPEMGISGQL TWTRLPQGFK NSPTLFNEAL | |
| 181 HRDLADFRIQ HPDLILLQYV DDLLLAATSE LDCQQGTRAL LQTLGNLGYR ASAKKAQICQ | |
| 241 KQVKYLGYLL KEGQRWLTEA RKETVMGQPT PKTPRQLRRF LGKAGFCRLF IPGFAEMAAP | |
| 301 LYPLTKPGTL FNWGPDQQKA YQEIKQALLT APALGLPDLT KPFELFVDEK QGYAKGVLTQ | |
| 361 KLGPWRRPVA YLSKKLDPVA AGWPPCLRMV AAIAVLTKDA GKLTMGQPLV IGAPHAVEAL | |
| 421 VKQPAGRWLS KARMTHYQAL LLDTDRVQFG PVVALNPATL LPLPEEGLQH NCLDGTGGGG | |
| 481 VTVKFKYKGE ELEVDISKIK KVWRVGKMIS FTYDDNGKTG RGAVSEKDAP KELLQMLEKS | |
| 541 GKK | |
| LOCUS 42B_L_-_Sto7_Trunc_515 560 aa linear UNA (SEQ ID NO: 139) | |
| FEATURES | Location/Qualifiers |
| gene | 1 . . . 492 |
| | /label = “42B L Trunc 515” |
| mutation | 43 |
| | /label = “M66L” |
| mutation | 46 |
| | /label = “E69K” |
| mutation | 116 |
| | /label = “L139P” |
| mutation | 177 |
| | /label = “D200N” |
| mutation | 279 |
| | /label = “E302R” |
| mutation | 283 |
| | /label = “T306K” |
| mutation | 290 |
| | /label = “W313F” |
| mutation | 307 |
| | /label = “T330P” |
| mutation | 412 |
| | /label = “L435G” |
| mutation | 425 |
| | /label = “P448A” |
| mutation | 426 |
| | /label = “D449G” |
| mutation | 431 |
| | /label = “N454K” |
| misc_feature | 493 . . . 497 |
| | /feature_type = “Misc.” |
| | /label = “Linker” |
| Region | 498 . . . 560 |
| | /region_type = “Domain” |
| | /label = “Sto7” |
| mutation | 509 |
| | /note = “RNAse silencing muation on Sto7” |
| | /label = “K13L” |

```
    1 TWLSDFPQAW AETGGMGLAV RQAPLIIPLK ATSTPVSIKQ YPLSQKARLG IKPHIQRLLD
  61 QGILVPCQSP WNTPLLPVKK PGTNDYRPVQ DLREVNKRVE DIHPTVPNPY NLLSGPPPSH
121 QWYTVLDLKD AFFCLRLHPT SQPLFAFEWR DPEMGISGQL TWTRLPQGFK NSPTLFNEAL
181 HRDLADFRIQ HPDLILLQYV DDLLLAATSE LDCQQGTRAL LQTLGNLGYR ASAKKAQICQ
241 KQVKYLGYLL KEGQRWLTEA RKETVMGQPT PKTPRQLRRF LGKAGFCRLF IPGFAEMAAP
301 LYPLTKPGTL FNWGPDQQKA YQEIKQALLT APALGLPDLT KPFELFVDEK QGYAKGVLTQ
361 KLGPWRRPVA YLSKKLDPVA AGWPPCLRMV AAIAVLTKDA GKLTMGQPLV IGAPHAVEAL
421 VKQPAGRWLS KARMTHYQAL LLDTDRVQFG PVVALNPATL LPLPEEGLQH NCLDILAEAH
481 GTRPDLTDQP LPGGGGSVTV KFKYKGEELE VDISKIKKVW RVGKMISFTY DDNGKTGRGA
541 VSEKDAPKEL LQMLEKSGKK
```

Table 4 shows listing of non-limiting embodiments of RT enzymes of the present disclosure.

Tables 5 and 6 show additional listing of amino acid and nucleic acid sequences of non-limiting embodiments of the engineered RTs of the present disclosure.

TABLE 4
Non-limiting embodiments of RT variants of the invention

| SEQ | | |
|---|---|---|
| ID | | |
| NO | Description | Sequences |
| 1 | Engineered | MTWLSDFPQAWAETGGMGLAVRQAPLIIPLKATST |
| | reverse | PVSIKQYPMSQKARLGIKPHIQRLLDQGILVPCQS |
| | transcriptase | PWNTPLLPVKKPGTNDYRPVQDLREVNKRVEDIHP |
| | (MMLV RT | TVPNPYNLLSGPPPSHQWYTVLDLKDAFFCLRLHP |
| | Variant) | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLLIENSSPNSRLIN |
| 2 | Archeal | MXXXXXFKYKGXXXXVDXSKXKKVWXVGKMXSFTX |
| | consensus | DXXXGKTGRGAVSEKDAPKELXXXXXXXXXXXK |
| | sequence | |
| 3 | His-RT variant- | MGSSHHHHHHSSGLVPRGSTWLSDFPQAWAETGGM |
| | Sto7 Fusion | GLAVRQAPLIIPLKATSTPVSIKQYPMSQKARLGI |
| | protein | KPHIQRLLDQGILVPCQSPWNTPLLPVKKPGTNDY |
| | Recombinant | RPVQDLREVNKRVEDIHPTVPNPYNLLSGPPPSH |
| | MMLV-Sto7 | QWYTVLDLKDAFFCLRLHPTSQPLFAFEWRDPEMG |
| | | ISGQLTWTRLPQGFKNSPTLFNEALHRDLADFRIQ |
| | | HPDLILLQYVDDLLLAATSELDCQQGTRALLQTLG |
| | | NLGYRASAKKAQICQKQVKYLGYLLKEGQRWLTEA |
| | | RKETVMGQPTPKTPRQLRRFLGKAGFCRLFIPGFA |
| | | EMAAPLYPLTKPGTLFNWGPDQQKAYQEIKQALLT |
| | | APALGLPDLTKPFELFVDEKQGYAKGVLTQKLGPW |
| | | RRPVAYLSKKLDPVAAGWPPCLRMVAAIAVLTKDA |
| | | GKLTMGQPLVIGAPHAVEALVKQPAGRWLSKARMT |
| | | HYQALLLDTDRVQFGPVVALNPATLLPLPEEGLQH |
| | | NCLDILAEAHGTRPDLTDQPLPDADHTWYTNGSSL |
| | | LQEGQRKAGAAVTTETEVIWAKALPAGTSAQRAEL |
| | | IALTQALKMAEGKKLNVYTDSRYAFATAHIHGEIY |
| | | RRRGWLTSKGKEIKNKDEILALLKALFLPKRLSII |
| | | HCPGHQKGHSAEARGNRMADQAARKAAITETPDTS |
| | | TLLIENSSPNSRLINGGGSMVTVKFKYKGEELEVD |
| | | ISKIKKVWRVGKMISFTYDDNGKTGRGAVSEKDAP |
| | | KELLQMLEKSGKK |
| 4 | a poly-dT | TTTTTTTTTT |
| | sequence | |
| 5 | RT variant-Sto7 | MGSSHHHHHHSSGLVPRGSTWLSDFPQAWAETGGM |
| | Fusion protein | GLAVRQAPLIIPLKATSTPVSIKQYPMSQKARLGI |
| | Recombinant | KPHIQRLLDQGILVPCQSPWNTPLLPVKKPGTNDY |
| | | RPVQDLREVNKRVEDIHPTVPNPYNLLSGPPPSHQ |
| | | WYTVLDLKDAFFCLRLHPTSQPLFAFEWRDPEMGI |
| | | SGQLTWTRLPQGFKNSPTLFNEALHRDLADFRIQH |
| | | PDLILLQYVDDLLLAATSELDCQQGTRALLQTLGN |
| | | LGYRASAKKAQICQKQVKYLGYLLKEGQRWLTEAR |
| | | KETVMGQPTPKTPRQLRRFLGKAGFCRLFIPGFAE |
| | | MAAPLYPLTKPGTLFNWGPDQQKAYQEIKQALLTA |
| | | PALGLPDLTKPFELFVDEKQGYAKGVLTQKLGPWR |
| | | RPVAYLSKKLDPVAAGWPPCLRMVAAIAVLTKDAG |
| | | KLTMGQPLVIGAPHAVEALVKQPAGRWLSKARMTH |
| | | YQALLLDTDRVQFGPVVALNPATLLPLPEEGLOHN |
| | | CLDILAEAHGTRPDLTDQPLPDADHTWYTNGSSLL |
| | | QEGQRKAGAAVTTETEVIWAKALPAGTSAQRAELI |
| | | ALTQALKMAEGKKLNVYTDSRYAFATAHIHGEIYR |
| | | RRGWLTSKGKEIKNKDEILALLKALFLPKRLSIIH |
| | | CPGHQKGHSAEARGNRMADQAARKAAITETPDTST |
| | | LLIENSSPNSRLINGGGSMVTVKFKYKGEEKEVDI |
| | | SKIKKVWRVGKMISFTYDDNGKTGRGAVSEKDAPK |
| | | ELLQMLEKSGKK |
| 6 | RT variant- | MTWLSDFPQAWAETGGMGLAVRQAPLIIPLKATST |
| | Sso7d Fusion | PVSIKQYPMSQKARLGIKPHIQRLLDQGILVPCQS |
| | protein | PWNTPLLPVKKPGTNDYRPVQDLREVNKRVEDIHP |
| | Recombinant | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLLIENSSPNSRLINGMAT |
| | | VKFKYKGEEKEVDISKIKKVWRVGKMISFTYDEGG |
| | | GKTGRGAVSEKDAPKELLQMLEKQKK |
| 7 | Wild-type | TLNIEDEHRLHETSKEPDVSLGSTWLSDFPQAWAE |
| | MMLV | TGGMGLAVRQAPLIIPLKATSTPVSIKQYPMSQEA |
| | GenBank Seq ID | RLGIKPHIQRLLDQGILVPCQSPWNTPLLPVKKPG |
| | NP_955591.1 | TNDYRPVQDLREVNKRVEDIHPTVPNPYNLLSGL |
| | p80 RT | PPSHQWYTVLDLKDAFFCLRLHPTSQPLFAFEWRD |
| | | PEMGISGQLTWTRLPQGFKNSPTLFDEALHRDLAD |
| | | FRIQHPDLILLQYVDDLLLAATSELDCQQGTRALL |
| | | QTLGNLGYRASAKKAQICQKQVKYLGYLLKEGQRW |
| | | LTEARKETVMGQPTPKTPRQLREFLGTAGFCRLWI |
| | | PGFAEMAAPLYPLTKTGTLFNWGPDQQKAYQEIKQ |
| | | ALLTAPALGLPDLTKPFELFVDEKQGYAKGVLTQK |
| | | LGPWRRPVAYLSKKLDPVAAGWPPCLRMVAAIAVL |
| | | TKDAGKLTMGQPLVILAPHAVEALVKQPPDRWLSN |
| | | ARMTHYQALLLDTDRVQFGPVVALNPATLLPLPEE |
| | | GLQHNCLDILAEAHGTRPDLTDQPLPDADHTWYTD |
| | | GSSLLQEGQRKAGAAVTTETEVIWAKALPAGTSAQ |
| | | RAELIALTQALKMAEGKKLNVYTDSRYAFATAHIH |
| | | GEIYRRRGLLTSEGKEIKNKDEILALLKALFLPKR |
| | | LSIIHCPGHQKGHSAEARGNRMADQAARKAAITET |
| | | PDTSTLL |
| 8 | RT variant- | MATVKFKYKGEEKEVDISKIKKVWRVGKMISFTYD |
| | Sso7d (N- | EGGGKTGRGAVSEKDAPKELLQMLEKQKKGSTWLS |
| | terminal) Fusion | DFPQAWAETGGMGLAVRQAPLIIPLKATSTPVSIK |
| | protein | QYPMSQKARLGIKPHIQRLLDQGILVPCQSPWNTP |
| | Recombinant | LLPVKKPGTNDYRPVQDLREVNKRVEDIHPTVPNP |
| | | YNLLSGPPPSHQWYTVLDLKDAFFCLRLHPTSQPL |
| | | FAFEWRDPEMGISGQLTWTRLPQGFKNSPTLFNEA |
| | | LHRDLADFRIQHPDLILLQYVDDLLLAATSELDCQ |
| | | QGTRALLQTLGNLGYRASAKKAQICQKQVKYLGYL |
| | | LKEGQRWLTEARKETVMGQPTPKTPRQLRRFLGKA |
| | | GFCRLFIPGFAEMAAPLYPLTKPGTLFNWGPDQQK |
| | | AYQEIKQALLTAPALGLPDLTKPFELFVDEKQGYA |
| | | KGVLTQKLGPWRRPVAYLSKKLDPVAAGWPPCLRM |
| | | VAAIAVLTKDAGKLTMGQPLVIGAPHAVEALVKQP |
| | | AGRWLSKARMTHYQALLLDTDRVQFGPVVALNPA |
| | | TLLPLPEEGLQHNCLDILAEAHGTRPDLTDQPLPD |
| | | ADHTWYTNGSSLLQEGQRKAGAAVTTETEVIWAKA |
| | | LPAGTSAQRAELIALTQALKMAEGKKLNVYTDSRY |
| | | AFATAHIHGEIYRRRGWLTSKGKEIKNKDEILALL |
| | | KALFLPKRLSIIHCPGHQKGHSAEARGNRMADQAA |
| | | RKAAITETPDTSTLLIENSSPNSRLIN |
| 9 | | ACGACCGUCG UCAUGUAGCG UUUGUCGGAGACU |
| | | CCUAGAU CAGAUGUCCU CCUGGCUACUGCA |
| 10 | | CGACTCACTG ACACTCGC |
| 11 | TSO sequence | AAGCAGTGGT ATCAACGCAG AGTACATrGrGrG |
| 12 | Sto-7 amino acid | MVTVKFKYKGEEKEVDISKIKKVWRVGKMISFTYD |
| | sequence | DNGKTGRGAVSEKDAPKELLQMLEKSGKK |
| 13 | Sso7d amino | MATVKFKYKGEEKEVDISKIKKVWRVGKMISFTYD |
| | acid sequence | EGGGKTGRGAVSEKDAPKELLQMLEKQKK- |
| 14 | MMLV variant | MTWLSDFPQAWAETGGMGLAVRQAPLIIPLKATST |
| | | PVSIKQYPLSQEARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRE |
| | | FLGTAGFCRLWIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIKAPHAVEA |
| | | LVKQPPDRWLSNARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLLIENSSPNSRLIN |
| 15 | Affinity Tag | His His His His His |
| 16 | Sto-7 amino acid | VTVKFKYKGEEKEVDISKIKKVWRVGKMISFTYDD |
| | sequence (no | NGKTGRGAVSEKDAPKELLQMLEKSGKK |
| | start codon) | |
| 17 | Sso7d amino | ATVKFKYKGEEKEVDISKIKKVWRVGKMISFTYDE |
| | acid sequence | GGKTGRGAVSEKDAPKELLQMLEKQKK- |
| | (no start codon) | |
| 18 | Sto-7 (K13L) | VTVKFKYKGEELEVDISKIKKVWRVGKMISFTYDD |
| | amino acid | NGKTGRGAVSEKDAPKELLQMLEKSGKK |
| | sequence (no | |
| | start codon) | |
| 19 | Linker | GGGGS (one non-limiting embodiment) |
| 20 | RT varinat-Sto7 | TWLSDFPQAWAETGGMGLAVRQAPLIIPLKATST |
| | Fusion (42B_L_- | PVSIKQYPLSQKARLGIKPHIQRLLDQGILVPCQS |
| | Sto7_K13L) | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLLIENSSPNSRLINGGGG |
| | | SVTVKFKYKGEELEVDISKIKKVWRVGKMISFTYD |
| | | DNGKTGRGAVSEKDAPKELLQMLEKSGKK |
| 21 | Peptide fragment | IENSSPNSRLIN |
| | of a cloning | |
| | artefact | |
| 22 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKOPPDRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 23 | MMLV RT | MTWLSDFPQAWAETGGVGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIKAPHAVEA |
| | | LVKQPPDRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 24 | MMLV RT | MTWLSDFPQAWAETGGVGLAVRQAPLIIPLKATST |
| | Variant | PVSIKQYPMSQKARLGIKPHIQRLLDQGILVPCQ |
| | | SPWNTPLLPVKKPGTNDYRPVQDLREVNKRVEDIH |
| | | PTVPNPYNLLSGPPPSHQWYTVLDLKDAFFCLRLH |
| | | PTSQPLFAFEWRDPEMGISGQLTWTRLPQGFKNSP |
| | | TLFNEALHRDLADFRIQHPDLILLQYVDDLLLAAT |
| | | SELDCQQGTRALLQTLGNLGYRASAKKAQICQKQV |
| | | KYLGYLLKEGQRWLTEARKETVMGQPTPKTPRQLR |
| | | RFLGKAGFCRLFIPGFAEMAAPLYPLTKPGTLFNW |
| | | GPDQQKAYQEIKQALLTAPALGLPDLTKPFELFVD |
| | | EKQGYAKGVLTQKLGPWRRPVAYLSKKLDPVAAGW |
| | | PPCLRMVAAIAVLTKDAGKLTMGQPLVIKAPHAVE |
| | | ALVKQPPDRWLSKARMTHYQALLLDTDRVQFGPVV |
| | | ALNPATLLPLPEEGLQHNCLDILAEAVGTRPDLTD |
| | | QPLPDADHTWYTNGSSLLQEGQRKAGAAVTTETEV |
| | | IWAKALPAGTSAQRAELIALTQALKMAEGKKLNVY |
| | | TDSRYAFATAHIHGEIYRRRGWLTSKGKEIKNKDE |
| | | ILALLKALFLPKRLSIIYCPGHQKGHSAEARGNRM |
| | | ADQAARKAAITETPDTSTLL |
| 25 | MMLV RT | MTWLSDFPQAWAETGGMGLAVRQAPLIIPLKATST |
| | Variant | PVSIKQYPMSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIKAPHAVEA |
| | | LVKQPPDRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 26 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 27 | MMLV RT | MTWLSDFPQAWAETGGVGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLOHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 28 | MMLV RT | TLNIEDEHRLHETSKEPDVSLGSTWLSDFPQAWAE |
| | Variant | TGGVGLAVRQAPLIIPLKATSTPVSIKQYPMSQK |
| | | ARLGIKPHIQRLLDQGILVPCQSPWNTPLLPVKKP |
| | | GTNDYRPVQDLREVNKRVEDIHPTVPNPYNLLSGP |
| | | PPSHQWYTVLDLKDAFFCLRLHPTSQPLFAFEWRD |
| | | PEMGISGQLTWTRLPQGFKNSPTLFNEALHRDLAD |
| | | FRIQHPDLILLQYVDDLLLAATSELDCQQGTRALL |
| | | QTLGNLGYRASAKKAQICQKQVKYLGYLLKEGQRW |
| | | LTEARKETVMGQPTPKTPRQLRRFLGKAGFCRLFI |
| | | PGFAEMAAPLYPLTKPGTLFNWGPDQQKAYQEIKQ |
| | | ALLTAPALGLPDLTKPFELFVDEKQGYAKGVLTQK |
| | | LGPWRRPVAYLSKKLDPVAAGWPPCLRMVAAIAVL |
| | | TKDAGKLTMSQPLVIKAPHAVEALVKQPAGRWLSK |
| | | ARMTHYQALLLDTDRVQFGPVVALNPATLLPLPEE |
| | | GLQHNCLDILAEAHGTRPDLTDQPLPDADHTWYTN |
| | | GSSLLQEGQRKAGAAVTDETEVIWAKALPAGTSAQ |
| | | RAELIALTQALKMAEGKKLNVYTNSRYAFATAHIH |
| | | GEIYRRRGWLTSGGKEIKNKDEILALLKALFLSKR |
| | | LSIIHCPGHQKGHSVEARGNRMAHQAARRAAITET |
| | | PDTSTLP |
| 29 | MMLV RT | TLNIEDEHRLHETSKEPDVSLGSTWLSDFPQAWAE |
| | Variant | TGGVGLAVRQAPLIIPLKATSTPVSIKQYPMSQK |
| | | ARLGIKPHIQRLLDQGILVPCQSPWNTPLLPVKKP |
| | | GTNDYRPVQDLREVNKRVEDIHPTVPNPYNLLSGP |
| | | PPSHQWYTVLDLKDAFFCLRLHPTSQPLFAFEWRD |
| | | PEMGISGQLTWTRLPQGFKNSPTLFNEALHRDLAD |
| | | FRIQHPDLILLQYVDDLLLAATSELDCQQGTRALL |
| | | QTLGNLGYRASAKKAQICQKQVKYLGYLLKEGQRW |
| | | LTEARKETVMGQPTPKTPRQLRRFLGKAGFCRLFI |
| | | PGFAEMAAPLYPLTKPGTLFNWGPDQQKAYQEIKQ |
| | | ALLTAPALGLPDLTKPFELFVDEKQGYAKGVLTQK |
| | | LGPWRRPVAYLSKKLDPVAAGWPPCLRMVAAIAVL |
| | | TKDAGKLTMSQPLVIKAPHAVEALVKQPAERWLSK |
| | | ARMTHYQALLLDTDRVQFGPVVALNPATLLPLPEE |
| | | GLQHNCLDILAEAHGTRPDLTDQPLPDADHTWYTN |
| | | GSSLLQEGQRKAGAAVTDETEVIWAKALPAGTSAQ |
| | | RAELIALTQALKMAEGKKLNVYTDSRYAFATAHIH |
| | | GEIYRRRGWLTSGGKEIKNKDEILALLKALFLPKR |
| | | LSIIHCPGHQKGHSVEARGNHMAHQAARRAAITET |
| | | PDTSTLP |
| 30 | MMLV RT | TLNIEDEHRLHETSKEPDVSLGSTWLSDFPQAWAE |
| | Variant | TGGVGLAVRQAPLIIPLKATSTPVSIKQYPMSQKA |
| | | RLGIKPHIQRLLDQGILVPCQSPWNTPLLPVKKPG |
| | | TNDYRPVQDLREVNKRVEDIHPTVPNPYNLLSGPP |
| | | PSHQWYTVLDLKDAFFCLRLHPTSQPLFAFEWRDP |
| | | EMGISGQLTWTRLPQGFKNSPTLFNEALHRDLADF |
| | | RIQHPDLILLQYVDDLLLAATSELDCQQGTRALLQ |
| | | TLGNLGYRASAKKAQICQKQVKYLGYLLKEGQRWL |
| | | TEARKETVMGQPTPKTPRQLRRFLGKAGFCRLFIP |
| | | GFAEMAAPLYPLTKPGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMSQPLVIKAPHAVEALVKQPAGRWLSKA |
| | | RMTHYQALLLDTDRVQFGPVVALNPATLLPLPEEG |
| | | LQHNCLDILAEAHGTRPDLTDQPLPDADHTWYTDG |
| | | SSLLQEGQRKAGAAVTTETGVIWAKALPAGTSAQR |
| | | AELIALTQALKMAEGKKLNVYTNSRYAFATAHIQG |
| | | EIYRRRGFLTSKGKEIKNKDEILALLKALFLPKRL |
| | | SIIHCPGHQKGHSAEARGNRMADQAARKAAITETP |
| | | DTSTLP |
| 31 | MMLV RT | TLNIEDEHRLHETSKEPDVSLGSTWLSDFPQAWAE |
| | Variant | TGGVGLAVRQAPLIIPLKATSTPVSIKQYPMSQKA |
| | | RLGIKPHIQRLLDQGILVPCQSPWNTPLLPVKKPG |
| | | TNDYRPVQDLREVNKRVEDIHPTVPNPYNLLSGPP |
| | | PSHQWYTVLDLKDAFFCLRLHPTSQPLFAFEWRDP |
| | | EMGISGQLTWTRLPQGFKNSPTLFNEALHRDLADF |
| | | RIQHPDLILLQYVDDLLLAATSELDCQQGTRALLQ |
| | | TLGNLGYRASAKKAQICQKQVKYLGYLLKEGQRWL |
| | | TEARKETVMGQPTPKTPRQLRRFLGKAGFCRLFIP |
| | | GFAEMAAPLYPLTKPGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMSQPLVIKAPHAVEALVKQPAERWLSKA |
| | | RMTHYQALLLDTDRVQFGPVVALNPATLLPLPEEG |
| | | LQHNCLDILAEAHGTRPDLTDQPLPDADHTWYTNG |
| | | SSLLQEGQRKAGAAVTDETEVIWAKALPAGTSAQR |
| | | AELIALTQALKMAEGKKLNVYTDSRYAFATAHIHG |
| | | EIYRRRGWLTSGGKEIKNKDEILALLKALFLPKRL |
| | | SIIHCPGHQKGHSVEARGNRMAHQAARRAAITETP |
| | | DTSTLP |
| 32 | MMLV RT | TLNIEDEHRLHETSKEPDVSLGSTWLSDFPQAWAE |
| | Variant | TGGVGLAVRQAPLIIPLKATSTPVSIKQYPMSQKA |
| | | RLGIKPHIQRLLDQGILVPCQSPWNTPLLPVKKPG |
| | | TNDYRPVQDLREVNKRVEDIHPTVPNPYNLLSGPP |
| | | PSHQWYTVLDLKDAFFCLRLHPTSQPLFAFEWRDP |
| | | EMGISGQLTWTRLPQGFKNSPTLFEEALRRDLADF |
| | | RIQHPDLILLQYVDDLLLAATSELDCQQGTRALLQ |
| | | TLGNLGYRASAKKAQICQKQVKYLGYLLKEGQRWL |
| | | TEARKETVMGQPTPKTPRQLRRFLGKAGFCRLFIP |
| | | GFAEMAAPLYPLTKPGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMSQPLVIKAPHAVEALVKQPAGRWLSKA |
| | | RMTHYQALLLDTDRVQFGPVVALNPATLLPLPEEG |
| | | LQHNCLDILAEAHGTRPDLTDQPLPDADHTWYTNG |
| | | SSLLQEGQRKAGAAVTDETEVIWAKALPAGTSAQR |
| | | AELIALTQALKMAEGKKLNVYTNSRYAFATAHIHG |
| | | EIYRRRGWLTSGGKEIKNKDEILALLKALFLSKRL |
| | | SIIHCPGHQKGHSVEARGNRMAHQAARRAAITETP |
| | | DTSTLP |
| 33 | MMLV RT | TLNIEDEHRLHETSKEPDVSLGSTWLSDFPQAWAE |
| | Variant | TGGVGLAVRQAPLIIPLKATSTPVSIKQYPMSQKA |
| | | RLGIKPHIQRLLDQGILVPCQSPWNTPLLPVKKPG |
| | | TNDYRPVQDLREVNKRVEDIHPTVPNPYNLLSGP |
| | | PPSHQWYTVLDLKDAFFCLRLHPTSQPLFAFEWRD |
| | | PEMGISGQLTWTRLPQGFKNSPTLFEEALRRDLAD |
| | | FRIQHPDLILLQYVDDLLLAATSELDCQQGTRALL |
| | | QTLGNLGYRASAKKAQICQKQVKYLGYLLKEGQRW |
| | | LTEARKETVMGQPTPKTPRQLRRFLGKAGFCRLFI |
| | | PGFAEMAAPLYPLTKPGTLFNWGPDQQKAYQEIKQ |
| | | ALLTAPALGLPDLTKPFELFVDEKQGYAKGVLTQK |
| | | LGPWRRPVAYLSKKLDPVAAGWPPCLRMVAAIAVL |
| | | TKDAGKLTMSQPLVIKAPHAVEALVKQPAGRWLSK |
| | | ARMTHYQALLLDTDRVQFGPVVALNPATLLPLPEE |
| | | GLQHNCLDILAEAHGTRPDLTDQPLPDADHTWYTD |
| | | GSSLLQEGQRKAGAAVTTETGVIWAKALPAGTSAQ |
| | | RAELIALTQALKMAEGKKLNVYTNSRYAFATAHIQ |
| | | GEIYRRRGFLTSKGKEIKNKDEILALLKALFLPKR |
| | | LSIIHCPGHQKGHSAEARGNRMADQAARKAAITET |
| | | PDTSTLP |
| 34 | MMLV RT | TLNIEDEHRLHETSKEPDVSLGSTWLSDFPQAWAE |
| | Variant | TGGVGLAVRQALLIIPLKATSTPVSIKQYPMSQKA |
| | | RLGIKPHIQRLLDQGILVPCQSPWNTPLLPVKKPG |
| | | TNDYRPVQDLREVNKRVEDIHPTVPNPYNLLSGPP |
| | | PSHQWYTVLDLKDAFFCLRLHPTSQPLFAFEWRDP |
| | | EMGISGQLTWTRLPQGFKNSPTLFNEALHRDLADF |
| | | RIQHPDLILLQYVDDLLLAATSELDCQQGTRALLQ |
| | | TLGNLGYRASAKKAQICQKQVKYLGYLLKEGQRWL |
| | | TEARKETVMGQPTPKTPRQLRRFLGKAGFCRLFIP |
| | | GFAEMAAPLYPLTKPGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMSQPLVIKAPHAVEALVKQPAGRWLSKA |
| | | RMTHYQALLLDTDRVQFGPVVALNPATLLPLPEEG |
| | | LQHNCLDILAEAHGTRPDLTDQPLPDADHTWYTNG |
| | | SSLLQEGQRKAGAAVTDETEVIWAKALPAGTSAQR |
| | | AELIALTQALKMAEGKKLNVYTNSRYAFATAHIHG |
| | | EIYRRRGWLTSGGKEIKNKDEILALLKALFLSKRL |
| | | SIIHCPGHQKGHSVEARGNRMAHQAARRAAITETP |
| | | DTSTLP |
| 35 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLOHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 36 | MMLV RT | MTWLSDFPQAWAETGGVGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 37 | MMLV RT | MTWLSDFPQAWAETGGMGLAVRQAPLIIPLKATST |
| | Variant | PVSIKQYPMSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLOHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 38 | MMLV RT | MTWLSDFPQAWAETGGMGLAVRQAPLIIPLKATST |
| | Variant | PVSIKQYPMSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAYFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPAGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 39 | MMLV RT | MTWLSDFPQAWAETGGVGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKOPPDRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 40 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPADRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 41 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPGRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 42 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKOPPDRWLSKARMTHYQALLLDTDRVQFGPVVA |
| | | LNPATLLPLPEEGLQHNCLDILAEAVGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 43 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIYCPGHQKGHSAEARGNRMA |
| | | DQAARKAAITETPDTSTLL |
| 44 | MMLV RT | MTWLSDFPQAWAETGGMGLAVRQAPLIIPLKATST |
| | Variant | PVSIKQYPMSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 45 | MMLV RT | MTWLSDFPQAWAETGGMGLAVRQAPLIIPLKATST |
| | Variant | PVSIKQYPMSQKARLGIKPHIQRLLDQGILVPCQ |
| | | SPWNTPLLPVKKPGTNDYRPVQDLREVNKRVEDIH |
| | | PTVPNPYNLLSGPPPSHQWYTVLDLKDAYFCLRLH |
| | | PTSQPLFAFEWRDPEMGISGQLTWTRLPQGFKNSP |
| | | TLFNEALHRDLADFRIQHPDLILLQYVDDLLLAAT |
| | | SELDCQQGTRALLQTLGNLGYRASAKKAQICQKQV |
| | | KYLGYLLKEGQRWLTEARKETVMGQPTPKTPRQLR |
| | | RFLGKAGFCRLFIPGFAEMAAPLYPLTKPGTLFNW |
| | | GPDQQKAYQEIKQALLTAPALGLPDLTKPFELFVD |
| | | EKQGYAKGVLTQKLGPWRRPVAYLSKKLDPVAAGW |
| | | PPCLRMVAAIAVLTKDAGKLTMGQPLVIGAPHAVE |
| | | ALVKQPPDRWLSKARMTHYQALLLDTDRVQFGPVV |
| | | ALNPATLLPLPEEGLQHNCLDILAEAHGTRPDLTD |
| | | QPLPDADHTWYTNGSSLLQEGQRKAGAAVTTETEV |
| | | IWAKALPAGTSAQRAELIALTQALKMAEGKKLNVY |
| | | TDSRYAFATAHIHGEIYRRRGWLTSKGKEIKNKDE |
| | | ILALLKALFLPKRLSIIHCPGHQKGHSAEARGNRM |
| | | ADQAARKAAITETPDTSTLL |
| 46 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLQHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGGQKGHSAEARGNRMA |
| | | DQAARKAAITETPDTSTLL |
| 47 | MMLV RT | MTWLSDFPQAWAETGGMGLAVRQAPLIIPLKATST |
| | Variant | PVSIKQYPLSQKARLGIKPHIQRLLDQGILVPCQ |
| | | SPWNTPLLPVKKPGTNDYRPVQDLREVNKRVEDIH |
| | | PTVPNPYNLLSGPPPSHQWYTVLDLKDAYFCLRLH |
| | | PTSQPLFAFEWRDPEMGISGQLTWTRLPQGFKNSP |
| | | TLFNEALHRDLADFRIQHPDLILLQYVDDLLLAAT |
| | | SELDCQQGTRALLQTLGNLGYRASAKKAQICQKQV |
| | | KYLGYLLKEGQRWLTEARKETVMGQPTPKTPRQLR |
| | | RFLGKAGFCRLFIPGFAEMAAPLYPLTKPGTLFNW |
| | | GPDQQKAYQEIKQALLTAPALGLPDLTKPFELFVD |
| | | EKQGYAKGVLTQKLGPWRRPVAYLSKKLDPVAAGW |
| | | PPCLRMVAAIAVLTKDAGKLTMGQPLVIGAPHAVE |
| | | ALVKQPPDRWLSKARMTHYQALLLDTDRVQFGPVV |
| | | ALNPATLLPLPEEGLQHNCLDILAEAHGTRPDLTD |
| | | QPLPDADHTWYTNGSSLLQEGQRKAGAAVTTETEV |
| | | IWAKALPAGTSAQRAELIALTQALKMAEGKKLNVY |
| | | TDSRYAFATAHIHGEIYRRRGWLTSKGKEIKNKDE |
| | | ILALLKALFLPKRLSIIHCPGGQKGHSAEARGNRM |
| | | ADQAARKAAITETPDTSTLL |
| 48 | MMLV RT | MTWLSDFPQAWAETGGVGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLOHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 49 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAYFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLOHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 50 | MMLV RT | MTWLSDFPQAWAETGGMGLAVRQAPLIIPLKATST |
| | Variant | PVSIKQYPLSQKARLGIKPHIQRLLDQGILVPCQS |
| | | PWNTPLLPVKKPGTNDYRPVQDLREVNKRVEDIHP |
| | | TVPNPYNLLSGPPPSHQWYTVLDLKDAFFCLRLHP |
| | | TSQPLFAFEWRDPEMGISGQLTWTRLPQGFKNSPT |
| | | LFNEALHRDLADFRIQHPDLILLQYVDDLLLAATS |
| | | ELDCQQGTRALLQTLGNLGYRASAKKAQICQKQVK |
| | | YLGYLLKEGQRWLTEARKETVMGQPTPKTPRQLRR |
| | | FLGKAGFCRLFIPGFAEMAAPLYPLTKPGTLFNWG |
| | | PDQQKAYQEIKQALLTAPALGLPDLTKPFELFVDE |
| | | KQGYAKGVLTQKLGPWRRPVAYLSKKLDPVAAGWP |
| | | PCLRMVAAIAVLTKDAGKLTMGQPLVIGAPHAVEA |
| | | LVKQPPDRWLSKARMTHYQALLLDTDRVQFGPVVA |
| | | LNPATLLPLPEEGLOHNCLDILAEAHGTRPDLTDQ |
| | | PLPDADHTWYTNGSSLLQEGQRKAGAAVTTETEVI |
| | | WAKALPAGTSAQRAELIALTQALKMAEGKKLNVYT |
| | | DSRYAFATAHIHGEIYRRRGWLTSKGKEIKNKDEI |
| | | LALLKALFLPKRLSIIHCPGHQKGHSAEARGNRMA |
| | | DQAARKAAITETPDTSTLL |
| 51 | MMLV RT | MGSSHHHHHHSSGLVPRGSTWLSDFPQAWAETGGM |
| | Variant | GLAVRQAPLIIPLKATSTPVSIKQYPMSQKARLGI |
| | | KPHIQRLLDQGILVPCQSPWNTPLLPVKKPGTNDY |
| | | RPVQDLREVNKRVEDIHPTVPNPYNLLSGPPPSH |
| | | QWYTVLDLKDAFFCLRLHPTSQPLFAFEWRDPEMG |
| | | ISGQLTWTRLPQGFKNSPTLFNEALHRDLADFRIQ |
| | | HPDLILLQYVDDLLLAATSELDCQQGTRALLQTLG |
| | | NLGYRASAKKAQICQKQVKYLGYLLKEGQRWLTEA |
| | | RKETVMGQPTPKTPRQLRRFLGKAGFCRLFIPGFA |
| | | EMAAPLYPLTKPGTLFNWGPDQQKAYQEIKQALLT |
| | | APALGLPDLTKPFELFVDEKQGYAKGVLTQKLGPW |
| | | RRPVAYLSKKLDPVAAGWPPCLRMVAAIAVLTKDA |
| | | GKLTMGQPLVIGAPHAVEALVKQPAGRWLSKARMT |
| | | HYQALLLDTDRVQFGPVVALNPATLLPLPEEGLQH |
| | | NCLDILAEAHGTRPDLTDQPLPDADHTWYTNGSSL |
| | | LQEGQRKAGAAVTTETEVIWAKALPAGTSAQRAEL |
| | | IALTQALKMAEGKKLNVYTDSRYAFATAHIHGEIY |
| | | RRRGWLTSKGKEIKNKDEILALLKALFLPKRLSII |
| | | HCPGHQKGHSAEARGNRMADQAARKAAITETPDTS |
| | | TLLIENSSPNSRLIN |
| 52 | MMLV RT | MGSSHHHHHHSSGLVPRGSTWLSDFPQAWAETGGM |
| | Variant | GLAVRQAPLIIPLKATSTPVSIKQYPMSQKARLGI |
| | | KPHIQRLLDQGILVPCQSPWNTPLLPVKKPGTNDY |
| | | RPVQDLREVNKRVEDIHPTVPNPYNLLSGPPPSH |
| | | QWYTVLDLKDAFFCLRLHPTSQPLFAFEWRDPEMG |
| | | ISGQLTWTRLPQGFKNSPTLFNEALHRDLADFRIQ |
| | | HPDLILLQYVDDLLLAATSELDCQQGTRALLQTLG |
| | | NLGYRASAKKAQICQKQVKYLGYLLKEGQRWLTEA |
| | | RKETVMGQPTPKTPRQLRRFLGKAGFCRLFIPGFA |
| | | EMAAPLYPLTKPGTLFNWGPDQQKAYQEIKQALLT |
| | | APALGLPDLTKPFELFVDEKQGYAKGVLTQKLGPW |
| | | RRPVAYLSKKLDPVAAGWPPCLRMVAAIAVLTKDA |
| | | GKLTMGQPLVIGAPHAVEALVKQPAGRWLSKARMT |
| | | HYQALLLDTDRVQFGPVVALNPATLLPLPEEGLOH |
| | | NCLDILAEAHGTRPDLTDQPLPDADHTWYTNGSSL |
| | | LQEGQRKAGAAVTTETEVIWAKALPAGTSAQRAEL |
| | | IALTQALKMAEGKKLNVYTDSRYAFATAHIHGEIY |
| | | RRRGWLTSKGKEIKNKDEILALLKALFLPKRLSII |
| | | HCPGHQKGHSAEARGNRMADQAARKAAITETPDTS |
| | | TLLIENSSPNSRLIN |
| 53 | MMLV RT | MTWLSDFPQAWAETGGMGLAVRQAPLIIPLKATST |
| | Variant | PVSIKQYPMSQKARLGIKPHIQRLLDQGILVPCQ |
| | | SPWNTPLLPVKKPGTNDYRPVQDLREVNKRVEDIH |
| | | PTVPNPYNLLSGPPPSHQWYTVLDLKDAFFCLRLH |
| | | PTSQPLFAFEWRDPEMGISGQLTWTRLPQGFKNSP |
| | | TLFNEALHRDLADFRIQHPDLILLQYVDDLLLAAT |
| | | SELDCQQGTRALLQTLGNLGYRASAKKAQICQKQV |
| | | KYLGYLLKEGQRWLTEARKETVMGQPTPKTPRQLR |
| | | RFLGKAGFCRLFIPGFAEMAAPLYPLTKPGTLFNW |
| | | GPDQQKAYQEIKQALLTAPALGLPDLTKPFELFVD |
| | | EKQGYAKGVLTQKLGPWRRPVAYLSKKLDPVAAGW |
| | | PPCLRMVAAIAVLTKDAGKLTMGQPLVIGAPHAVE |
| | | ALVKQPAGRWLSKARMTHYQALLLDTDRVQFGPVV |
| | | ALNPATLLPLPEEGLQHNCLDILAEAHGTRPDLTD |
| | | QPLPDADHTWYTNGSSLLQEGQRKAGAAVTTETEV |
| | | IWAKALPAGTSAQRAELIALTQALKMAEGKKLNVY |
| | | TDSRYAFATAHIHGEIYRRRGWLTSKGKEIKNKDE |
| | | ILALLKALFLPKRLSIIHCPGHQKGHSAEARGNRM |
| | | ADQAARKAAITETPDTSTLLIENSSPNSRLIN |
| 54 | MMLV RT | GSTWLSDFPQAWAETGGMGLAVRQAPLIIPLKATS |
| | Variant | TPVSIKQYPMSQKARLGIKPHIQRLLDQGILVPCQ |
| | | SPWNTPLLPVKKPGTNDYRPVQDLREVNKRVEDI |
| | | HPTVPNPYNLLSGPPPSHQWYTVLDLKDAFFCLRL |
| | | HPTSQPLFAFEWRDPEMGISGQLTWTRLPQGFKNS |
| | | PTLFNEALHRDLADFRIQHPDLILLQYVDDLLLAA |
| | | TSELDCQQGTRALLQTLGNLGYRASAKKAQICQKQ |
| | | VKYLGYLLKEGQRWLTEARKETVMGQPTPKTPRQL |
| | | RRFLGKAGFCRLFIPGFAEMAAPLYPLTKPGTLFN |
| | | WGPDQQKAYQEIKQALLTAPALGLPDLTKPFELFV |
| | | DEKQGYAKGVLTQKLGPWRRPVAYLSKKLDPVAAG |
| | | WPPCLRMVAAIAVLTKDAGKLTMGQPLVIGAPHAV |
| | | EALVKQPAGRWLSKARMTHYQALLLDTDRVQFGPV |
| | | VALNPATLLPLPEEGLQHNCLDILAEAHGTRPDLT |
| | | DQPLPDADHTWYTNGSSLLQEGQRKAGAAVTTETE |
| | | VIWAKALPAGTSAQRAELIALTQALKMAEGKKLNV |
| | | YTDSRYAFATAHIHGEIYRRRGWLTSKGKEIKNKD |
| | | EILALLKALFLPKRLSIIHCPGHQKGHSAEARGNR |
| | | MADQAARKAAITETPDTSTLLIENSSPNSRLIN |
| 55 | MMLV RT | TWLSDFPQAWAETGGMGLAVRQAPLIIPLKATSTP |
| | Variant | VSIKQYPLSQKARLGIKPHIQRLLDQGILVPCQSP |
| | | WNTPLLPVKKPGTNDYRPVQDLREVNKRVEDIHPT |
| | | VPNPYNLLSGPPPSHQWYTVLDLKDAFFCLRLHPT |
| | | SQPLFAFEWRDPEMGISGQLTWTRLPQGFKNSPTL |
| | | FNEALHRDLADFRIQHPDLILLQYVDDLLLAATSE |
| | | LDCQQGTRALLQTLGNLGYRASAKKAQICQKQVKY |
| | | LGYLLKEGQRWLTEARKETVMGQPTPKTPRQLRRF |
| | | LGKAGFCRLFIPGFAEMAAPLYPLTKPGTLFNWGP |
| | | DQQKAYQEIKQALLTAPALGLPDLTKPFELFVDEK |
| | | QGYAKGVLTQKLGPWRRPVAYLSKKLDPVAAGWPP |
| | | CLRMVAAIAVLTKDAGKLTMGQPLVIGAPHAVEAL |
| | | VKQPAGRWLSKARMTHYQALLLDTDRVQFGPVVAL |
| | | NPATLLPLPEEGLQHNCLDILAEAHGTRPDLTDQP |
| | | LPDADHTWYTNGSSLLQEGQRKAGAAVTTETEVIW |
| | | AKALPAGTSAQRAELIALTQALKMAEGKKLNVYTD |
| | | SRYAFATAHIHGEIYRRRGWLTSKGKEIKNKDEIL |
| | | ALLKALFLPKRLSIIHCPGHQKGHSAEARGNRMAD |
| | | QAARKAAITETPDTSTLLIENSSPNSRLIN |
| 56 | a histidine | HHHHHH |
| | purification tag | |
| 57 | short peptide C- | SEEDEEKEEDG |
| | terminal tag | |
| 58 | Tobacco etch | ENLYFQ/G |
| | virus protease | |
| | (TEV) cleavage | |
| | site | |
| 59 | Enterokinase | DDDDK/ |
| | (EntK) cleavage | |
| | site | |
| 60 | Factor Xa (Xa) | IEGR/ |
| | cleavage site | |
| 61 | Thrombin (Thr) | LVPR/GS |
| | cleavage site | |
| 62 | Genetically | LEVLFQ/GP |
| | engineered | |
| | derivative of | |
| | human | |
| | rhinovirus 3C | |
| | protease | |
| | cleavage site | |

TABLE 5
Listing of amino acid and nucleic acid sequences of non-limiting embodiments of the
engineered RTs of the present disclosure

| SEQ | | | SEQ | |
|---|---|---|---|---|
| ID | | | ID | |
| NO: | Description | Amino Acid Sequence | NO: | Nucleic Acid Sequence |
| 63 | RTx | TWLSDFPQAWAETGGVGLAVRQAPLIIPLK | 64 | acttggctgtctgatttccctcaggcgtgg |
| | SOLD 016 | ATSTPVSIKQYPMSQKARLGIKPHIQRLLD | | gccgaaacgggtggcgtgggtctggcagtg |
| | N-Tag-His | QGILVPCQSPWNTPLLPVKKPGTNDYRPVQ | | cgtcaggcaccgctgattattccgctgaaa |
| | P_Code: | DLREVNKRVEDIHPTVPNPYNLLSGPPPSH | | gcgacgtcgaccccggtgagcatcaagcaa |
| | P001 | QWYTVLDLKDAFFCLRLHPTSQPLFAFEWR | | tatccgatgtcccaaaaggcgcgcttaggt |
| | | DPEMGISGQLTWTRLPQGFKNSPTLFEEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttgaagaagcgctg |
| | | IALTQALKMAEGKKLNVYTNSRYAFATAHI | | cgccgtgacctggcggatttccgtatccag |
| | | HGEIYRRRGWLTSGGKEIKNKDEILALLKA | | cacccggacctgatcttgctgcagtacgtt |
| | | LFLSKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgagcaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 65 | RTx | TWLSDFPQAWAETGGLGLAVRQAPLIIPLK | 66 | acttggctgtctgatttccctcaggcgtgg |
| | SOLD 001 | ATSTPVSIKQYPLSQKARLGIKPHIQRLLD | | gccgaaacgggtggcctgggtctggcagtg |
| | FIG. | QGILVPCQSPWNTPLLPVKKPGTNDYRPVQ | | cgtcaggcaccgctgattattccgctgaaa |
| | N-tag: His | DLREVNKRVEDIHPTVPNPYNLLSGPPPSH | | gcgacgtcgaccccggtgagcatcaagcaa |
| | P_code | QWYTVLDLKDAFFCLRLHPTSQPLFAFEWR | | tatccgctgtcccaaaaggcgcgcttaggt |
| | P002 | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 67 | RTx | TWLSDFPQAWAETGGLGLAVRQAPLIIPLK | 68 | acttggctgtctgatttccctcaggcgtgg |
| | SOLD 002 | ATSTPVSIKQYPLSQKARLGIKPHIQRLLD | | gccgaaacgggtggcctgggtctggcagtg |
| | N-Tag: his | QGILVPCQSPWNTPLLPVKKPGTSDYRPVQ | | cgtcaggcaccgctgattattccgctgaaa |
| | P_code | DLREVNKRVEDIHPTVPNPYNLLSGLPPSH | | gcgacgtcgaccccggtgagcatcaagcaa |
| | P003 | QWYTVLDLKDAFFCLRLHPTSQPLFAFEWR | | tatccgctgtcccaaaaggcgcgcttaggt |
| | | DPEMGISGQLTWTRLPQGFKNSPTLFVEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtctgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttgtggaagcgctg |
| | | IALTQALKMAEGKKLNVYTDSRYAFATAHI | | cgccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 69 | RTx | TWLSDFPQAWAETGGVGLAVRQAPLIIPLK | 70 | acttggctgtctgatttccctcaggcgtgg |
| | SOLD 003 | ATSTPVSIKQYPMSQKARLGIKPHIQRLLD | | gccgaaacgggtggcgtgggtctggcagtg |
| | N-tag: His | QGILVPCQSPWNTPLLPVKKPGTSDYRPVQ | | cgtcaggcaccgctgattattccgctgaaa |
| | P_code: | DLREVNKRVEDIHPTVPNPYNLLSGLPPSH | | gcgacgtcgaccccggtgagcatcaagcaa |
| | P004 | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtctgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | cgccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 71 | RTx | TWLSDFPQAWAETGGVGLAVRQAPLIIPLK | 72 | acttggctgtctgatttccctcaggcgtgg |
| | SOLD 004 | ATSTPVSIKQYPMSQKARLGIKPHIQRLLD | | gccgaaacgggtggcgtgggtctggcagtg |
| | N-tag-his | QGILVPCQSPWNTPLLPVKKPGTSDYRPVQ | | cgtcaggcaccgctgattattccgctgaaa |
| | P-code: | DLREVNKRVEDIHPTVPNPYNLLSGLPPSH | | gcgacgtcgaccccggtgagcatcaagcaa |
| | P005 | QWYTVLDLKDAFFCLRLHPTSQPLFAFEWR | | tatccgatgtcccaaaaggcgcgcttaggt |
| | | DPEMGISGQLTWTRLPQGFKNSPTLFVEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtctgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttgtggaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 73 | RTx | TWLSDFPQAWAETGGVGLAVRQAPLIIPLK | 74 | acttggctgtctgatttccctcaggcgtgg |
| | SOLD 005 | ATSTPVSIKQYPMSQKARLGIKPHIQRLLD | | gccgaaacgggtggcgtgggtctggcagtg |
| | N-Tag: His | QGILVPCQSPWNTPLLPVKKPGTSDYRPVQ | | cgtcaggcaccgctgattattccgctgaaa |
| | P-code: | DLREVNKRVEDIHPTVPNPYNLLSGLPPSH | | gcgacgtcgaccccggtgagcatcaagcaa |
| | P006 | QWYTVLDLKDAFFCLRLHPTSQPLFAFEWR | | tatccgatgtcccaaaaggcgcgcttaggt |
| | | DPEMGISGQLTWTRLPQGFKNSPTLFVEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtctgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttgtggaagcgctg |
| | | IALTQALKMAEGKKLNVYTDSRYAFATAHI | | cgccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 75 | RTx | TWLSDFPQAWAETGGVGLAVRQAPLIIPLK | 76 | acttggctgtctgatttccctcaggcgtgg |
| | SOLD 006 | ATSTPVSIKQYPMSQKARLGIKPHIQRLLD | | gccgaaacgggtggcgtgggtctggcagtg |
| | N-tag: His | QGILVPCQSPWNTPLLPVKKPGTNDYRPVQ | | cgtcaggcaccgctgattattccgctgaaa |
| | P007 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRKFLGRAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaaattc |
| | | | | ttgggccgcgcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 77 | RTx | TWLSDFPQAWAETGGVGLAVRQAPLIIPLK | 78 | acttggctgtctgatttccctcaggcgtgg |
| | SOLD 007 | ATSTPVSIKQYPMSQKARLGIKPHIQRLLD | | gccgaaacgggtggcgtgggtctggcagtg |
| | N tag-His | QGILVPCQSPWNTPLLPVKKPGTNDYRPVQ | | cgtcaggcaccgctgattattccgctgaaa |
| | P008 | DLREVNKRVEDIHPTVPNPYNLLSGLPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtctgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 79 | RTx | TWLSDFPQAWAETGGVGLAVRQAPLIIPLK | 80 | acttggctgtctgatttccctcaggcgtgg |
| | SOLD 008 | ATSTPVSIKQYPMSQKARLGIKPHIQRLLD | | gccgaaacgggtggcgtgggtctggcagtg |
| | P009 | QGILVPCQSPWNTPLLPVKKPGTSDYRPVQ | | cgtcaggcaccgctgattattccgctgaaa |
| | N-tag: His | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCRQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRKFLGRAGSCRLHIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccgccagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaaattc |
| | | | | ttgggccgcgcgggtagctgccgcctgcat |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 81 | RTx | TWLSDFPQAWAETGGVGLAVRQAPLIIPLK | 82 | acttggctgtctgatttccctcaggcgtgg |
| | SOLD 009 | ATSTPVSIKQYPMSQKARLGIKPHIQRLLD | | gccgaaacgggtggcgtgggtctggcagtg |
| | N-Tag: His | QGILVPCQSPWNTPLLPVKKPGTNDYRPVQ | | cgtcaggcaccgctgattattccgctgaaa |
| | P010 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLPKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 83 | RTx | TWLSDFPQAWAETWGVGLAVRQALLIIPLK | 84 | acttggctgtctgatttccctcaggcgtgg |
| | SOLD 010 | ATSTPVSIKQYPMSQKARLGIKPHIQRLLD | | gccgaaacgtggggcgtgggtctggcagtg |
| | N-tag: His | QGILVPCRSPWNTPLLPVKKPGTSDYRPVQ | | cgtcaggcactgctgattattccgctgaaa |
| | P011 | DLREVNKRVEDIRPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcgcagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcgcccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTNSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLSKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgagcaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 85 | RTx | TWLSDFPQAWAETGGVGLAVRQAPLIIPLK | 86 | acttggctgtctgatttccctcaggcgtgg |
| | SOLD 011 | ATSTPVSIKQYPMSQKARLGIKPHIQRLLD | | gccgaaacgggtggcgtgggtctggcagtg |
| | N-Tag: His | QGILVPCQSPWNTPLLPVKKPGTNDYRPVQ | | cgtcaggcaccgctgattattccgctgaaa |
| | P012 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFEEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTDGSSLLQEGQ | | acgtggacccgcctgccgcaaggttttaag |
| | | RKAGAAVTTETGVIWAKALPAGTSAQRAEL | | aattcccctacgctgtttgaagaagcgctg |
| | | IALTQALKMAEGKKLNVYTNSRYAFATAHI | | cgccgtgacctggcggatttccgtatccag |
| | | QGEIYRRRGFLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLPIENSSPNPRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | gatggtagcagcctgttgcaagagggtcag |
| | | | | cgtaaggccggtgccgcggtcaccaccgag |
| | | | | actggcgtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cagggcgagatctatcgccgtcgcggcttt |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgccgatcgag |
| | | | | aatagcagcccgaacccgcgtctgatcaat |
| 87 | RTx | TWLSDFPQAWAETGGVGLAVRQAPLIIPLK | 88 | acttggctgtctgatttccctcaggcgtgg |
| | SOLD 012 | ATSTPVSIKQYPMSQKARLGIKPHIQRLLD | | gccgaaacgggtggcgtgggtctggcagtg |
| | N-tag: His | QGILVPCQSPWNTPLLPVKKPGTNDYRPVQ | | cgtcaggcaccgctgattattccgctgaaa |
| | P013 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTNSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLSKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgagcaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 89 | RTx | TWLSDFPQAWAETWGVGLAVRQALLIIPLK | 90 | acttggctgtctgatttccctcaggcgtgg |
| | SOLD 013 | ATSTPVSIKQYPMSQKARLGIKPHIQRLLD | | gccgaaacgtggggcgtgggtctggcagtg |
| | N-tag: His | QGILVPCRSPWNTPLLPVKKPGTSDYRPVQ | | cgtcaggcactgctgattattccgctgaaa |
| | P014 | DLREVNKRVEDIRPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcgcagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcgcccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLPKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 91 | RTx | TWLSDFPQAWAETGGVGLAVRQAPLIIPLK | 92 | acttggctgtctgatttccctcaggcgtgg |
| | SOLD 014 | ATSTPVSIKQYPMSQKARLGIKPHIQRLLD | | gccgaaacgggtggcgtgggtctggcagtg |
| | N-Tag: His | QGILVPCQSPWNTPLLPVKKPGTNDYRPVQ | | cgtcaggcaccgctgattattccgctgaaa |
| | P015 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFEEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttgaagaagcgctg |
| | | IALTQALKMAEGKKLNVYTDSRYAFATAHI | | cgccgtgacctggcggatttccgtatccag |
| | | HGEIYRRRGWLTSGGKEIKNKDEILALLKA | | cacccggacctgatcttgctgcagtacgtt |
| | | LFLPKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 93 | RTx | TWLSDFPQAWAETGGVGLAVRQAPLIIPLK | 94 | acttggctgtctgatttccctcaggcgtgg |
| | SOLD 015 | ATSTPVSIKQYPMSQKARLGIKPHIQRLLD | | gccgaaacgggtggcgtgggtctggcagtg |
| | N-Tag: His | QGILVPCQSPWNTPLLPVKKPGTNDYRPVQ | | cgtcaggcaccgctgattattccgctgaaa |
| | P016 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTDGSSLLQEGQ | | acgtggacccgcctgccgcaaggttttaag |
| | | RKAGAAVTTETGVIWAKALPAGTSAQRAEL | | aattcccctacgctgtttaacgaagcgctg |
| | | IALTQALKMAEGKKLNVYTNSRYAFATAHI | | caccgtgacctggcggatttccgtatccag |
| | | QGEIYRRRGFLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLPIENSSPNPRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | gatggtagcagcctgttgcaagagggtcag |
| | | | | cgtaaggccggtgccgcggtcaccaccgag |
| | | | | actggcgtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cagggcgagatctatcgccgtcgcggcttt |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgccgatcgag |
| | | | | aatagcagcccgaacccgcgtctgatcaat |
| 95 | RTx | TWLSDFPQAWAETWGVGLAVRQALLIIPLK | 96 | acttggctgtctgatttccctcaggcgtgg |
| | SOLD 017 | ATSTPVSIKQYPMSQKARLGIKPHIQRLLD | | gccgaaacgtggggcgtgggtctggcagtg |
| | N-Tag: His | QGILVPCRSPWNTPLLPVKKPGTSDYRPVQ | | cgtcaggcactgctgattattccgctgaaa |
| | P017 | DLREVNKRVEDIRPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcgcagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcgcccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTDGSSLLQEGQ | | acgtggacccgcctgccgcaaggttttaag |
| | | RKAGAAVTTETGVIWAKALPAGTSAQRAEL | | aattcccctacgctgtttaacgaagcgctg |
| | | IALTQALKMAEGKKLNVYTNSRYAFATAHI | | caccgtgacctggcggatttccgtatccag |
| | | QGEIYRRRGFLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLPIENSSPNPRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | gatggtagcagcctgttgcaagagggtcag |
| | | | | cgtaaggccggtgccgcggtcaccaccgag |
| | | | | actggtgtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cagggcgagatctatcgccgtcgcggcttt |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgccgatcgag |
| | | | | aatagcagcccgaacccgcgtctgatcaat |
| 97 | RTx | TWLSDFPQAWAETGGVGLAVRQAPLIIPLK | 98 | acttggctgtctgatttccctcaggcgtgg |
| | SOLD 018 | ATSTPVSIKQYPMSQKARLGIKPHIQRLLD | | gccgaaacgggtggcgtgggtctggcagtg |
| | N-Tag: His | QGILVPCQSPWNTPLLPVKKPGTNDYRPVQ | | cgtcaggcaccgctgattattccgctgaaa |
| | P018 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTNSRYAFATAHI | | caccgtgacctggggatttccgtatccagc |
| | | HGEIYRRRGWLTSGGKEIKNKDEILALLKA | | acccggacctgatcttgctgcagtacgttg |
| | | LFLSKRLSIIHCPGHQKGHSVEARGNRMAH | | atgacctgttgctggcggcgacgagcgagc |
| | | QAARRAAITETPDTSTLPIENSSPNSRLIN | | tggattgccaacagggcacccgtgcgctgt |
| | | | | tgcagaccttgggtaacctgggttatcgcg |
| | | | | ctagcgcgaagaaagcgcagatttgccaaa |
| | | | | aacaagttaagtatctgggctacctgttaa |
| | | | | aggaaggccaacgttggctgaccgaagccc |
| | | | | gcaaagaaactgtcatgggtcagccgaccc |
| | | | | cgaaaacgccacgccaactgcgtaggttct |
| | | | | tgggcaaagcgggtttctgccgcctgttca |
| | | | | tcccgggctttgccgaaatggcagccccgc |
| | | | | tgtatccgttgaccaagccgggcaccctgt |
| | | | | tcaactggggtccggaccagcagaaagcgt |
| | | | | accaagaaattaaacaagcactgctgacgg |
| | | | | caccggcgctgggtctgccggacctgacca |
| | | | | agccgtttgagctgttcgtggatgagaagc |
| | | | | aaggttacgcgaagggcgtgttgacccaga |
| | | | | aattgggtccgtggcgtcgtccggttgcat |
| | | | | acctgtccaagaaactggacccggttgctg |
| | | | | ctggttggccgccttgcctgcgcatggttg |
| | | | | ccgctatcgcggtgctgactaaagacgcgg |
| | | | | gtaagctgacgatgagccaaccgctggtga |
| | | | | tcaaagcaccgcatgcagtcgaggcccttg |
| | | | | ttaagcaaccggcagaaagatggctgagca |
| | | | | aggcgcgtatgacgcattaccaggcactgc |
| | | | | tgttggacaccgatcgtgtgcagtttggcc |
| | | | | cggtcgttgcgctcaacccggcgaccctgc |
| | | | | tgccgctcccggaagaaggcttgcagcaca |
| | | | | actgtttggacatcctggcagaggcgcacg |
| | | | | gcactcgcccggatctgacggaccagccgc |
| | | | | tgccggacgccgatcatacctggtatacga |
| | | | | atggtagcagcctgttgcaagagggtcagc |
| | | | | gtaaggccggtgccgcggtcaccgatgaga |
| | | | | ctgaagtgatttgggctaaagcattgcctg |
| | | | | cgggtaccagcgcgcagcgtgccgagctga |
| | | | | tcgcactgacccaagcgctgaaaatggctg |
| | | | | agggtaagaaactgaatgtgtacacgaaca |
| | | | | gccgttatgcctttgcgaccgcccacattc |
| | | | | acggcgagatctatcgccgtcgcggctggc |
| | | | | tgacgtccggcggcaaagagatcaagaata |
| | | | | aagacgaaattctggcgctgctgaaagcgc |
| | | | | tgttcctgagcaaacgtctgtcgatcatcc |
| | | | | attgcccgggtcaccagaaaggccacagcg |
| | | | | tggaggcgcgtggtaatcgcatggctcatc |
| | | | | aggctgcgcgtcgcgccgcaattaccgaaa |
| | | | | ccccggacaccagcacgctgccgatcgaga |
| | | | | atagcagcccgaacagccgtctgatcaat |
| 99 | RTx | TWLSDFPQAWAETGGVGLAVRQAPLIIPLK | 100 | acttggctgtctgatttccctcaggcgtgg |
| | SOLD 019 | ATSTPVSIKQYPMSQKARLGIKPHIQRLLD | | gccgaaacgggtggcgtgggtctggcagtg |
| | N-tag: His | QGILVPCRSPWNTPLLPVKKPGTSDYRPVQ | | cgtcaggcaccgctgattattccgctgaaa |
| | P019 | DLREVNKRVEDIRPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcgcagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcgcccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTNSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLSKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgagcaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 101 | RTx | TWLSDFPQAWAETWGVGLAVRQALLIIPLK | 102 | acttggctgtctgatttccctcaggcgtgg |
| | SOLD 020 | ATSTPVSIKQYPMSQKARLGIKPHIQRLLD | | gccgaaacgtggggcgtgggtctggcagtg |
| | N-Tag: His | QGILVPCRSPWNTPLLPVKKPGTSDYRPVQ | | cgtcaggcactgctgattattccgctgaaa |
| | P020 | DLREVNKRVEDIRPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcgcagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgagcgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcgcccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSGGKEIKNKDEILALLKA | | cacccggacctgatcttgctgcagtacgtt |
| | | LFLPKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcaggcagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 103 | RTx | TWLSDFPQAWAETGGVGLAVRQAPLIIPLK | 104 | acttggctgtctgatttccctcaggcgtgg |
| | SOLD 021 | ATSTPVSIKQYPMSQKARLGIKPHIQRLLD | | gccgaaacgggtggcgtgggtctggcagtg |
| | N-tag: His | QGILVPCQSPWNTPLLPVKKPGTNDYRPVQ | | cgtcaggcaccgctgattattccgctgaaa |
| | P021 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFEEAL | | attaagccgcacattcagcgtctgctggat |
| | | RRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMSQPLVIKAPHAVEAL | | gctttcttctgtctgcgtctgcacccaacc |
| | | VKQPAERWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTDETEVIWAKALPAGTSAQRAEL | | aattcccctacgctgtttgaagaagcgctg |
| | | IALTQALKMAEGKKLNVYTNSRYAFATAHI | | cgccgtgacctggcggatttccgtatccag |
| | | HGEIYRRRGWLTSGGKEIKNKDEILALLKA | | cacccggacctgatcttgctgcagtacgtt |
| | | LFLSKRLSIIHCPGHQKGHSVEARGNRMAH | | gatgacctgttgctggcggcgacgagcgag |
| | | QAARRAAITETPDTSTLPIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgagccaaccgctggtg |
| | | | | atcaaagcaccgcatgcagtcgaggccctt |
| | | | | gttaagcaaccggcagaaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccgatgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacgaac |
| | | | | agccgttatgcctttgcgaccgcccacatt |
| | | | | cacggcgagatctatcgccgtcgcggctgg |
| | | | | ctgacgtccggcggcaaagagatcaagaat |
| | | | | aaagacgaaattctggcgctgctgaaagcg |
| | | | | ctgttcctgagcaaacgtctgtcgatcatc |
| | | | | cattgcccgggtcaccagaaaggccacagc |
| | | | | gtggaggcgcgtggtaatcgcatggctcat |
| | | | | caggctgcgcgtcgcgccgcaattaccgaa |
| | | | | accccggacaccagcacgctgccgatcgag |
| | | | | aatagcagcccgaacagccgtctgatcaat |
| 105 | RTx | TWLSDFPQAWAETGGVGLAVRQAPLIIPLK | 106 | acctggctgagcgattttccgcaggcatgg |
| | SOLD 022 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P022 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRKFLGAAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTDGSSLLQEGQ | | acctggacccgcctgccgcagggttttaaa |
| | | RKAGAAVTTETGVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | QGEIYRRRGFLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLPIENSSPNPRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgcaaattt |
| | | | | ctgggtgcagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | gatggtagcagcctgctgcaggaaggtcag |
| | | | | cgcaaagcaggtgcagcagtgaccaccgaa |
| | | | | accggtgtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | cagggtgaaatttatcgccgccgcggtttt |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgccgattgaa |
| | | | | aacagcagcccgaacccgcgcctgattaac |
| 107 | RTx | TWLSDFPQAWAETGGVGLAVRQAPLIIPLK | 108 | acctggctgagcgattttccgcaggcatgg |
| | SOLD 023 | ATSTPVSIKQYPMSQKARLGIKPHIQRLLD | | gcagaaaccggtggtgtgggtctggcagtg |
| | N-Tag: His | QGILVPCQSPWNTPLLPVKKPGTNDYRPVQ | | cgccaggcaccgctgattattccgctgaaa |
| | P023 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGYSAEARGNRMAV | | gatgatctgctgctggcagcaaccagcgaa |
| | | QAARKAAITETPDTSTLLIENSSPNPRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgt |
| | | | | gctgacccagaaactgggtccgtggcgccg |
| | | | | cccggtggcatatctgagcaaaaaactgga |
| | | | | tccggtggcagcaggttggccgccgtgcct |
| | | | | gcgcatggtggcagcaattgcagtgctgac |
| | | | | caaagatgcaggtaaactgaccatgggtca |
| | | | | gccgctggtgattggtgcaccgcatgcagt |
| | | | | ggaagcactggtgaaacagccggcaggtcg |
| | | | | ctggctgagcaaagcacgcatgacccatta |
| | | | | tcaggcactgctgctggataccgatcgcgt |
| | | | | gcagtttggtccggtggtggcactgaaccc |
| | | | | ggcaaccctgctgccgctgccggaagaagg |
| | | | | tctgcagcataactgcctggatattctggc |
| | | | | agaagcacatggtacccgcccggatctgac |
| | | | | cgatcagccgctgccggatgcagatcatac |
| | | | | ctggtataccaacggtagcagcctgctgca |
| | | | | ggaaggtcagcgcaaagcaggtgcagcagt |
| | | | | gaccaccgaaaccgaagtgatttgggcaaa |
| | | | | agcactgccggcaggtaccagcgcacagcg |
| | | | | cgcagaactgattgcactgacccaggcact |
| | | | | gaaaatggcagaaggtaaaaaactgaacgt |
| | | | | gtataccgatagccgctatgcatttgcaac |
| | | | | cgcacatattcatggtgaaatttatcgccg |
| | | | | ccgcggttggctgaccagcaaaggtaaaga |
| | | | | aattaaaaacaaagatgaaattctggcact |
| | | | | gctgaaagcactgtttctgccgaaacgcct |
| | | | | gagcattattcattgcccgggtcatcagaa |
| | | | | aggttatagcgcagaagcacgcggtaaccg |
| | | | | catggcagtgcaggcagcacgcaaagcagc |
| | | | | aattaccgaaaccccggataccagcaccct |
| | | | | gctgattgaaaacagcagcccgaacccgcg |
| | | | | cctgattaac |
| 109 | RTx | TWLSDFPQAWAETGGVGLAVRQAPLIIPLK | 110 | acctggctgagcgattttcc |
| | SOLD 024 | ATSTPVSIKQYPMSQRARLGIKPHIQRLLD | | gcaggcatgggcagaaaccggtggtgtggg |
| | N-tag: His | QGILVPCRSPWNTPLLPVKKPGTSDYRPVQ | | tctggcagtgcgccaggcaccgctgattat |
| | P024 | DLREVNKRVEDIRPTVPNPYNLLSGPPPSH | | tccgctgaaagcaaccagcaccccggtgag |
| | | QWYTVLDLKDAFFCLRLHPTSQPLFAFEWR | | cattaaacagtatccgatgagccagcgcgc |
| | | DPEMGISGQLTWTRLPQGFKNSPTLFEEAL | | acgcctgggtattaaaccgcatattcagcg |
| | | RRDLADFRIQHPDLILLQYVDDLLLAATSE | | cctgctggatcagggtattctggtgccgtg |
| | | LDCQQGTRALLQTLGNLGYRASAKKAQICQ | | ccgcagcccgtggaacaccccgctgctgcc |
| | | KQVKYLGYLLKEGQRWLTEARKETVMGQPT | | ggtgaaaaaaccgggtaccagcgattatcg |
| | | PKTPRQLRRFLGKAGFCRLFIPGFAEMAAP | | cccggtgcaggatctgcgcgaagtgaacaa |
| | | LYPLTKPGTLFNWGPDQQKAYQEIKQALLT | | acgcgtggaagatattcgcccgaccgtgcc |
| | | APALGLPDLTKPFELFVDEKQGYAKGVLTQ | | gaacccgtataacctgctgagcggtccgcc |
| | | KLGPWRRPVAYLSKKLDPVAAGWPPCLRMV | | gccgagccatcagtggtataccgtgctgga |
| | | AAIAVLTKDAGKLTMGQPLVIGAPHAVEAL | | tctgaaagatgcatttttttgcctgcgcct |
| | | VKQPAGRWLSKARMTHYQALLLDTDRVQFG | | gcatccgaccagccagccgctgtttgcatt |
| | | PVVALNPATLLPLPEEGLQHNCLDILAEAH | | tgaatggcgcgatccggaaatgggtattag |
| | | GTRPDLTDQPLPDADHTWYTNGSSLLQEGQ | | cggtcagctgacctggacccgcctgccgca |
| | | RKAGAAVTTETEVIWAKALPAGTSAQRAEL | | gggttttaaaaacagcccgaccctgtttga |
| | | IALTQALKMAEGKKLNVYTDSRYAFATAHI | | agaagcactgcgccgcgatctggcagattt |
| | | HGEIYRRRGWLTSKGKEIKNKDEILALLKA | | tcgcattcagcatccggatctgattctgct |
| | | LFLPKRLSIIHCPGHQKGHSAEARGNRMAD | | gcagtatgtggatgatctgctgctggcagc |
| | | QAARKAAITETPDTSTLLIENSSPNSRLIN | | aaccagcgaactggattgccagcagggtac |
| | | | | ccgcgcactgctgcagaccctgggtaacct |
| | | | | gggttatcgcgcaagcgcaaaaaaagcaca |
| | | | | gatttgccagaaacaggtgaaatatctggg |
| | | | | ttatctgctgaaagaaggtcagcgctggct |
| | | | | gaccgaagcacgcaaagaaaccgtgatggg |
| | | | | tcagccgaccccgaaaaccccgcgccagct |
| | | | | gcgccgctttctgggtaaagcaggtttttg |
| | | | | ccgcctgtttattccgggttttgcagaaat |
| | | | | ggcagcaccgctgtatccgctgaccaaacc |
| | | | | gggtaccctgtttaactggggtccggatca |
| | | | | gcagaaagcatatcaggaaattaaacaggc |
| | | | | actgctgaccgcaccggcactgggtctgcc |
| | | | | ggatctgaccaaaccgtttgaactgtttgt |
| | | | | ggatgaaaaacagggttatgcaaaaggtgt |
| | | | | gctgacccagaaactgggtccgtggcgccg |
| | | | | cccggtggcatatctgagcaaaaaactgga |
| | | | | tccggtggcagcaggttggccgccgtgcct |
| | | | | gcgcatggtggcagcaattgcagtgctgac |
| | | | | caaagatgcaggtaaactgaccatgggtca |
| | | | | gccgctggtgattggtgcaccgcatgcagt |
| | | | | ggaagcactggtgaaacagccggcaggtcg |
| | | | | ctggctgagcaaagcacgcatgacccatta |
| | | | | tcaggcactgctgctggataccgatcgcgt |
| | | | | gcagtttggtccggtggtggcactgaaccc |
| | | | | ggcaaccctgctgccgctgccggaagaagg |
| | | | | tctgcagcataactgcctggatattctggc |
| | | | | agaagcacatggtacccgcccggatctgac |
| | | | | cgatcagccgctgccggatgcagatcatac |
| | | | | ctggtataccaacggtagcagcctgctgca |
| | | | | ggaaggtcagcgcaaagcaggtgcagcagt |
| | | | | gaccaccgaaaccgaagtgatttgggcaaa |
| | | | | agcactgccggcaggtaccagcgcacagcg |
| | | | | cgcagaactgattgcactgacccaggcact |
| | | | | gaaaatggcagaaggtaaaaaactgaacgt |
| | | | | gtataccgatagccgctatgcatttgcaac |
| | | | | cgcacatattcatggtgaaatttatcgccg |
| | | | | ccgcggttggctgaccagcaaaggtaaaga |
| | | | | aattaaaaacaaagatgaaattctggcact |
| | | | | gctgaaagcactgtttctgccgaaacgcct |
| | | | | gagcattattcattgcccgggtcatcagaa |
| | | | | aggtcatagcgcagaagcacgcggtaaccg |
| | | | | catggcagatcaggcagcacgcaaagcagc |
| | | | | aattaccgaaaccccggataccagcaccct |
| | | | | gctgattgaaaacagcagcccgaacagccg |
| | | | | cctgattaac |
| 111 | RTx | TWLSDFPQAWAETGGVGLAVRQAPLIIPLK | 112 | acctggctgagcgattttccgcaggcatgg |
| | SOLD 025 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P025 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTDETEVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | HGEIYRRRGWLTSGGKEIKNKDEILALLKA | | catccggatctgattctgctgcagtatgtg |
| | | LFLPKRLSIIHCPGHQKGHSVEARGNRMAH | | gatgatctgctgctggcagcaaccagcgaa |
| | | QAARRAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccgatgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | catggtgaaatttatcgccgccgcggttgg |
| | | | | ctgaccagcggtggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcggtaaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| | | | | gaagcacgcggtaaccgcatggcagatcag |
| | | | | gcagcacgcaaagcagcaattaccgaaacc |
| | | | | ccggataccagcaccctgccgattgaaaac |
| | | | | agcagcccgaacccgcgcctgattaac |
| 113 | RTx | TWLSDFPQAWAETGGVGLAVRQAPLIIPLK | 114 | acctggctgagcgattttccgcaggcatgg |
| | SOLD 026 | ATSTPVSIKQYPMSQKARLGIKPHIQRLLD | | gcagaaaccggtggtgtgggtctggcagtg |
| | N-Tag: His | QGILVPCQSPWNTPLLPVKKPGTNDYRPVQ | | cgccaggcaccgctgattattccgctgaaa |
| | P026 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTDGSSLLQEGQ | | acctggacccgcctgccgcagggttttaaa |
| | | RKAGAAVTTETGVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | QGEIYRRRGFLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLPIENSSPNPRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | gatggtagcagcctgctgcaggaaggtcag |
| | | | | cgcaaagcaggtgcagcagtgaccaccgaa |
| | | | | accggtgtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | cagggtgaaatttatcgccgccgcggtttt |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgccgattgaa |
| | | | | aacagcagcccgaacccgcgcctgattaac |
| 115 | RTx | TWLSDFPQAWAETGGVGLAVRQAPLIIPLK | 116 | acctggctgagcgattttccgcaggcatggg |
| | SOLD 027 | ATSTPVSIKQYPMSQRARLGIKPHIQRLLD | | cagaaaccggtggtgtgggtctggcagtgc |
| | N-tag: His | QGILVPCRSPWNTPLLPVKKPGTSDYRPVQ | | gccaggcaccgctgattattccgctgaaag |
| | P027 | DLREVNKRVEDIRPTVPNPYNLLSGPPPSH | | caaccagcaccccggtgagcattaaacagt |
| | | QWYTVLDLKDAFFCLRLHPTSQPLFAFEWR | | atccgatgagccagcgcgcacgcctgggta |
| | | DPEMGISGQLTWTRLPQGFKNSPTLFNEAL | | ttaaaccgcatattcagcgcctgctggatc |
| | | HRDLADFRIQHPDLILLQYVDDLLLAATSE | | agggtattctggtgccgtgccgcagcccgt |
| | | LDCQQGTRALLQTLGNLGYRASAKKAQICQ | | ggaacaccccgctgctgccggtgaaaaaac |
| | | KQVKYLGYLLKEGQRWLTEARKETVMGQPT | | cgggtaccagcgattatcgcccggtgcagg |
| | | PKTPRQLRRFLGKAGFCRLFIPGFAEMAAP | | atctgcgcgaagtgaacaaacgcgtggaag |
| | | LYPLTKPGTLFNWGPDQQKAYQEIKQALLT | | atattcgcccgaccgtgccgaacccgtata |
| | | APALGLPDLTKPFELFVDEKQGYAKGVLTQ | | acctgctgagcggtccgccgccgagccatc |
| | | KLGPWRRPVAYLSKKLDPVAAGWPPCLRMV | | agtggtataccgtgctggatctgaaagatg |
| | | AAIAVLTKDAGKLTMGQPLVIGAPHAVEAL | | catttttttgcctgcgcctgcatccgacca |
| | | VKQPAGRWLSKARMTHYQALLLDTDRVQFG | | gccagccgctgtttgcatttgaatggcgcg |
| | | PVVALNPATLLPLPEEGLQHNCLDILAEAH | | atccggaaatgggtattagcggtcagctga |
| | | GTRPDLTDQPLPDADHTWYTNGSSLLQEGQ | | cctggacccgcctgccgcagggttttaaaa |
| | | RKAGAAVTTETEVIWAKALPAGTSAQRAEL | | acagcccgaccctgtttaacgaagcactgc |
| | | IALTQALKMAEGKKLNVYTDSRYAFATAHI | | atcgcgatctggcagattttcgcattcagc |
| | | HGEIYRRRGWLTSKGKEIKNKDEILALLKA | | atccggatctgattctgctgcagtatgtgg |
| | | LFLPKRLSIIHCPGHQKGHSAEARGNRMAD | | atgatctgctgctggcagcaaccagcgaac |
| | | QAARKAAITETPDTSTLLIENSSPNSRLIN | | tggattgccagcagggtacccgcgcactgc |
| | | | | tgcagaccctgggtaacctgggttatcgcg |
| | | | | caagcgcaaaaaaagcacagatttgccaga |
| | | | | aacaggtgaaatatctgggttatctgctga |
| | | | | aagaaggtcagcgctggctgaccgaagcac |
| | | | | gcaaagaaaccgtgatgggtcagccgaccc |
| | | | | cgaaaaccccgcgccagctgcgccgctttc |
| | | | | tgggtaaagcaggtttttgccgcctgttta |
| | | | | ttccgggttttgcagaaatggcagcaccgc |
| | | | | tgtatccgctgaccaaaccgggtaccctgt |
| | | | | ttaactggggtccggatcagcagaaagcat |
| | | | | atcaggaaattaaacaggcactgctgaccg |
| | | | | caccggcactgggtctgccggatctgacca |
| | | | | aaccgtttgaactgtttgtggatgaaaaac |
| | | | | agggttatgcaaaaggtgtgctgacccaga |
| | | | | aactgggtccgtggcgccgcccggtggcat |
| | | | | atctgagcaaaaaactggatccggtggcag |
| | | | | caggttggccgccgtgcctgcgcatggtgg |
| | | | | cagcaattgcagtgctgaccaaagatgcag |
| | | | | gtaaactgaccatgggtcagccgctggtga |
| | | | | ttggtgcaccgcatgcagtggaagcactgg |
| | | | | tgaaacagccggcaggtcgctggctgagca |
| | | | | aagcacgcatgacccattatcaggcactgc |
| | | | | tgctggataccgatcgcgtgcagtttggtc |
| | | | | cggtggtggcactgaacccggcaaccctgc |
| | | | | tgccgctgccggaagaaggtctgcagcata |
| | | | | actgcctggatattctggcagaagcacatg |
| | | | | gtacccgcccggatctgaccgatcagccgc |
| | | | | tgccggatgcagatcatacctggtatacca |
| | | | | acggtagcagcctgctgcaggaaggtcagc |
| | | | | gcaaagcaggtgcagcagtgaccaccgaaa |
| | | | | ccgaagtgatttgggcaaaagcactgccgg |
| | | | | caggtaccagcgcacagcgcgcagaactga |
| | | | | ttgcactgacccaggcactgaaaatggcag |
| | | | | aaggtaaaaaactgaacgtgtataccgata |
| | | | | gccgctatgcatttgcaaccgcacatattc |
| | | | | atggtgaaatttatcgccgccgcggttggc |
| | | | | tgaccagcaaaggtaaagaaattaaaaaca |
| | | | | aagatgaaattctggcactgctgaaagcac |
| | | | | tgtttctgccgaaacgcctgagcattattc |
| | | | | attgcccgggtcatcagaaaggtcatagcg |
| | | | | cagaagcacgcggtaaccgcatggcagatc |
| | | | | aggcagcacgcaaagcagcaattaccgaaa |
| | | | | ccccggataccagcaccctgctgattgaaa |
| | | | | acagcagcccgaacagccgcctgattaac |
| 117 | RTx | TWLSDFPQAWAETGGVGLAVRQAPLIIPLK | 118 | acctggctgagcgattttccgcaggcatgg |
| | SOLD 028 | ATSTPVSIKQYPMSQRARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P028 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagcgcgcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | catggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| 119 | RTx | TWLSDFPQAWAETGGVGLAVRQAPLIIPLK | 120 | acctggctgagcgattttccgcaggcatgg |
| | SOLD 029 | ATSTPVSIKQYPMSQRARLGIKPHIQRLLD | | gcagaaaccggtggtgtgggtctggcagtg |
| | N-Tag: His | QGILVPCRSPWNTPLLPVKKPGTSDYRPVQ | | cgccaggcaccgctgattattccgctgaaa |
| | P029 | DLREVNKRVEDIRPTVPNPYNLLSGPPPSH | | gcaaccagcaccccggtgagcattaaacag |
| | | QWYTVLDLKDAFFCLRLHPTSQPLFAFEWR | | tatccgatgagccagcgcgcacgcctgggt |
| | | DPEMGISGQLTWTRLPQGFKNSPTLFEEAL | | attaaaccgcatattcagcgcctgctggat |
| | | RRDLADFRIQHPDLILLQYVDDLLLAATSE | | cagggtattctggtgccgtgccgcagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccagcgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcgcccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMSQPLVIKAPHAVEAL | | gcatttttttgcctgcgcctgcatccgacc |
| | | VKQPAERWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTDETEVIWAKALPAGTSAQRAEL | | aacagcccgaccctgtttgaagaagcactg |
| | | IALTQALKMAEGKKLNVYTNSRYAFATAHI | | cgccgcgatctggcagattttcgcattcag |
| | | HGEIYRRRGWLTSGGKEIKNKDEILALLKA | | catccggatctgattctgctgcagtatgtg |
| | | LFLPKRLSIIHCPGHQKGHSVEARGNRMAH | | gatgatctgctgctggcagcaaccagcgaa |
| | | QAARRAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgagccagccgctggtg |
| | | | | attaaagcaccgcatgcagtggaagcactg |
| | | | | gtgaaacagccggcagaacgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccgatgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | catggtgaaatttatcgccgccgcggttgg |
| | | | | ctgaccagcggtggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcggtaaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| 121 | RTx | TWLSDFPQAWAETGGVGLAVRQAPLIIPLK | 122 | acctggctgagcgattttccgcaggcatgg |
| | SOLD 030 | ATSTPVSIKQYPMSQRARLGIKPHIQRLLD | | gcagaaaccggtggtgtgggtctggcagtg |
| | N-tag: His | QGILVPCRSPWNTPLLPVKKPGTNDYRPVQ | | cgccaggcaccgctgattattccgctgaaa |
| | P030 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagcgcgcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccgcagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | catggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| 123 | RTx | TWLSDFPQAWAETGGVGLAVRQAPLIIPLK | 124 | acctggctgagcgattttccgcaggcatgg |
| | SOLD 031 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P031 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGHSVEARGNRMAH | | gatgatctgctgctggcagcaaccagcgaa |
| | | QAARRAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | catggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcggtaaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| 125 | RTx | TWLSDFPQAWAETGGVGLAVRQAPLIIPLK | 126 | acctggctgagcgattttccgcaagcatgg |
| | SOLD 032 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P032 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFEEAL | | attaaaccgcatattcagcgcctgctggat |
| | | RRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttgaagaagcactg |
| | | IALTQALKMAEGKKLNVYTNSRYAFATAHI | | cgccgcgatctggcagattttcgcattcag |
| | | QGEIYRRRGFLTSKGKEIKNKDEILALLKA | | catccggatctgattctgctgcagtatgtg |
| | | LFLPKRLSIIHCPGHQKGYSAEARVNRMAD | | gatgatctgctgctggcagcaaccagcgaa |
| | | QAARKAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgtta |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtactctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccttttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtt |
| | | | | gcagcaattgcagtgctgaccaaagatgca |
| | | | | ggtaaactgaccatgggtcagcctctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ttactggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgactgatcagccg |
| | | | | ctgcctgatgcagatcatacctggtatacc |
| | | | | aacggtagcagcctgttacaggaaggtcag |
| | | | | cgtaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | cagggtgaaatctatcgccgccgcggtttc |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggttatagc |
| | | | | gcagaagcacgcgtgaaccgcatggcagat |
| | | | | caggcagcacgcaaagcagcaattaccgaa |
| | | | | accccggataccagcaccctgccgattgaa |
| | | | | aacagcagcccgaacagccgcctgattaac |
| 127 | RTx | TWLSDFPQAWAETGGFGLAVRQAPLIIPLK | 128 | acctggctgagcgattttccgcaggcatgg |
| | SOLD 033 | ATSTPVSIKQYPISQKARLGIKPHIQRLLD | | gcagaaaccggtggttttggtctggcagtg |
| | N-tag: His | QGILVPCRSPWNTPLLPVKKPGTNDYRPVQ | | cgccaggcaccgctgattattccgctgaaa |
| | P033 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgattagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccgcagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | HGEIYRRRGWLTSGGKEIKNKDEILALLKA | | catccggatctgattctgctgcagtatgtg |
| | | LFLPKRLSIIHCPGHQKGHSVEARDNRMAH | | gatgatctgctgctggcagcaaccagcgaa |
| | | QAARRAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | catggtgaaatttatcgccgccgcggttgg |
| | | | | ctgaccagcggtggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcgataaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| 129 | RTx | TWLSDFPQAWAETGGVGLAVRQAPLIIPLK | 130 | acctggctgagcgattttccgcaggcatgg |
| | SOLD 034 | ATSTPVSIKQYPISQKARLGIKPHIQRLLD | | gcagaaaccggtggtgtgggtctggcagtg |
| | N-Tag: his | QGILVPCRSPWNTPLLPVKKPGTNDYRPVQ | | cgccaggcaccgctgattattccgctgaaa |
| | P034 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgattagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccgcagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEVKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | QGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaagtgaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | cagggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| 131 | RTx | TWLSDFPQAWAETGGVGLAVRQAPLIIPLK | 132 | acctggctgagcgattttccgcaggcatgg |
| | SOLD 035 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P035 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMSQPLVIKAPHAVEAL | | gcatttttttgcctgcgcctgcatccgacc |
| | | VKQPAERWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTDETEVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | HGEIYRRRGWLTSGGKEIKNKDEILALLKA | | catccggatctgattctgctgcagtatgtg |
| | | LFLPKRLSIIHCPGHQKGHSVEARGNRMAH | | gatgatctgctgctggcagcaaccagcgaa |
| | | QAARRAAITETPDTSTLPIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgagccagccgctggtg |
| | | | | attaaagcaccgcatgcagtggaagcactg |
| | | | | gtgaaacagccggcagaacgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccgatgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | catggtgaaatttatcgccgccgcggttgg |
| | | | | ctgaccagcggtggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcggtaaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| 133 | RTx | TWLSDFPQAWAETGGVGLAVRQAPLIIPLK | 134 | acctggctgagcgattttccgcaggcatgg |
| | SOLD 036 | ATSTPVSIKQYPMSQKARLGIKPHIQRLLD | | gcagaaaccggtggtgtgggtctggcagtg |
| | N-tag: His | QGILVPCQSPWNTPLLPVKKPGTNDYRPVQ | | cgccaggcaccgctgattattccgctgaaa |
| | P036 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMSQPLVIKAPHAVEAL | | gcatttttttgcctgcgcctgcatccgacc |
| | | VKQPAERWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgagccagccgctggtg |
| | | | | attaaagcaccgcatgcagtggaagcactg |
| | | | | gtgaaacagccggcagaacgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | catggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| 135 | RTx | TWLSDFPQAWAETGGMGLAVRQAPLIIPLK | 136 | acctggctgtctgactttccgcaggcttgg |
| | 42B L- | ATSTPVSIKQYPLSQKARLGIKPHIQRLLD | | gcagagaccggggggatgggtttagcggta |
| | Sto7 Trunc | QGILVPCQSPWNTPLLPVKKPGTNDYRPVQ | | cggcaggcaccactgattataccgctgaaa |
| | 586 | DLREVNKRVEDIHPTVPNPYNLLSGPPPSH | | gcaacatcgactcctgtctcgataaagcaa |
| | N-Tag: His | QWYTVLDLKDAFFCLRLHPTSQPLFAFEWR | | taccctctgtcccaaaaggctcgcttaggc |
| | C-Tag: Sto7 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRAEL | | aactccccgactctgtttaatgaagcgctg |
| | | IALTQALKMAEGKKLNVYTDSRYGGGGSVT | | caccgcgacctggccgactttagaatacag |
| | | VKFKYKGEELEVDISKIKKVWRVGKMISFT | | cacccagatcttatccttctgcagtacgtc |
| | | YDDNGKTGRGAVSEKDAPKELLQMLEKSGK | | gatgatcttttgctggccgcaacaagcgag |
| | | K | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagagccgagtta |
| | | | | attgcgttaacacaggcgctgaagatggct |
| | | | | gaagggaaaaaactgaacgtctataccgac |
| | | | | agccgctatggtggaggtgggtcagtaaca |
| | | | | gtaaaatttaaatataaaggtgaagagttg |
| | | | | gaggttgatatttcaaagataaaaaaagtg |
| | | | | tggagagtcggtaaaatgataagttttacg |
| | | | | tatgatgacaacgggaaaaccggtcggggc |
| | | | | gccgtgtccgaaaaagacgctccgaaggaa |
| | | | | ctactccaaatgcttgagaaaagtggaaaa |
| | | | | aaa |
| 137 | RTx | TWLSDFPQAWAETGGMGLAVRQAPLIIPLK | 138 | acctggctgtctgactttccgcaggcttgg |
| | 42B L- | ATSTPVSIKQYPLSQKARLGIKPHIQRLLD | | gcagagaccggggggatgggtttagcggta |
| | Sto7 Trunc | QGILVPCQSPWNTPLLPVKKPGTNDYRPVQ | | cggcaggcaccactgattataccgctgaaa |
| | 497 | DLREVNKRVEDIHPTVPNPYNLLSGPPPSH | | gcaacatcgactcctgtctcgataaagcaa |
| | N-Tag: His | QWYTVLDLKDAFFCLRLHPTSQPLFAFEWR | | taccctctgtcccaaaaggctcgcttaggc |
| | C-Tag: Sto7 | DPEMGISGQLTWTRLPQGFKNSPTLFNEAL | | attaagccacatatccagcgtctgctagat |
| | P038 | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDGTGGGG | | gacccggaaatgggaattagcggtcagctc |
| | | VTVKFKYKGEELEVDISKIKKVWRVGKMIS | | acctggacaagactcccgcaaggattcaag |
| | | FTYDDNGKTGRGAVSEKDAPKELLQMLEKS | | aactccccgactctgtttaatgaagcgctg |
| | | GKK | | caccgcgacctggccgactttagaatacag |
| | | | | cacccagatcttatccttctgcagtacgtc |
| | | | | gatgatcttttgctggccgcaacaagcgag |
| | | | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatgggacgggaggaggcgga |
| | | | | gtaacagtaaaatttaaatataaaggtgaa |
| | | | | gagttggaggttgatatttcaaagataaaa |
| | | | | aaagtgtggagagtcggtaaaatgataagt |
| | | | | tttacgtatgatgacaacgggaaaaccggt |
| | | | | cggggcgccgtgtccgaaaaagacgctccg |
| | | | | aaggaactactccaaatgcttgagaaaagt |
| | | | | ggaaaaaaa |
| 139 | RTx | TWLSDFPQAWAETGGMGLAVRQAPLIIPLK | 140 | acctggctgtctgactttccgcaggcttgg |
| | 42B L- | ATSTPVSIKQYPLSQKARLGIKPHIQRLLD | | gcagagaccggggggatgggtttagcggta |
| | Sto7 Trunc | QGILVPCQSPWNTPLLPVKKPGTNDYRPVQ | | cggcaggcaccactgattataccgctgaaa |
| | 515 | DLREVNKRVEDIHPTVPNPYNLLSGPPPSH | | gcaacatcgactcctgtctcgataaagcaa |
| | N-tag: His | QWYTVLDLKDAFFCLRLHPTSQPLFAFEWR | | taccctctgtcccaaaaggctcgcttaggc |
| | C-tag: Sto7 | DPEMGISGQLTWTRLPQGFKNSPTLFNEAL | | attaagccacatatccagcgtctgctagat |
| | P039 | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPGGGGSVTVKFKYKGEELE | | acctggacaagactcccgcaaggattcaag |
| | | VDISKIKKVWRVGKMISFTYDDNGKTGRGA | | aactccccgactctgtttaatgaagcgctg |
| | | VSEKDAPKELLQMLEKSGKK | | caccgcgacctggccgactttagaatacag |
| | | | | cacccagatcttatccttctgcagtacgtc |
| | | | | gatgatcttttgctggccgcaacaagcgag |
| | | | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccggtggaggtgggtcagtaacagta |
| | | | | aaatttaaatataaaggtgaagagttggag |
| | | | | gttgatatttcaaagataaaaaaagtgtgg |
| | | | | agagtcggtaaaatgataagttttacgtat |
| | | | | gatgacaacgggaaaaccggtcggggcgcc |
| | | | | gtgtccgaaaaagacgctccgaaggaacta |
| | | | | ctccaaatgcttgagaaaagtggaaaaaaa |
| 141 | RTx | TWLSDFPQAWAETGGVGLAVRQAPLIIPLK | 142 | acctggctgagcgattttccgcaagcatgg |
| | SOLD 037 | ATSTPVSIKQYPMSQKARLGIKPHIQRLLD | | gcagaaaccggtggtgtgggtctggcagtg |
| | N-tag: His | QGILVPCQSPWNTPLLPVKKPGTSDYRPVQ | | cgccaggcaccgctgattattccgctgaaa |
| | P041 | DLREVNKRVEDIHPTVPNPYNLLSGPPPSH | | gcaaccagcaccccggtgagcattaaacag |
| | | QWYTVLDLKDAFFCLRLHPTSQPLFAFEWR | | tatccgatgagccagaaagcacgcctgggt |
| | | DPEMGISGQLTWTRLPQGFKNSPTLFVEAL | | attaaaccgcatattcagcgcctgctggat |
| | | RRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccagcgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttgtggaagcactg |
| | | IALTQALKMAEGKKLNVYTDSRYAFATAHI | | cgccgcgatctggcagattttcgcattcag |
| | | HGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | | | ctgcagaccctgggtaacctgggttatcgc |
| | | | | gcaagcgcaaaaaaagcacagatttgccag |
| | | | | aaacaggtgaaatatctgggttatctgtta |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtactctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccttttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtt |
| | | | | gcagcaattgcagtgctgaccaaagatgca |
| | | | | ggtaaactgaccatgggtcagcctctggtg |
| | | | | attggtgcaccgcatgcagtggaagcatta |
| | | | | gtgaaacagccggcaggtcgctggctgagc |
| | | | | aaagcacgcatgacccattatcaggcactg |
| | | | | ttactggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgactgatcagccg |
| | | | | ctgcctgatgcagatcatacctggtatacc |
| | | | | aacggtagcagcctgttacaggaaggtcag |
| | | | | cgtaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | catggtgaaatctatcgccgccgcggttgg |
| | | | | ctgaccagcaaaggtaaagaaattaaaaac |
| | | | | aaagatgagatcctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| 143 | RTx | TWLSDFPQAWAETGGMGLAVRQAPLIIPLK | 144 | acttggctgtctgatttccctcaggcgtgg |
| | 42B | ATSTPVSIKQYPMSQKARLGIKPHIQRLLD | | gccgaaacgggtggcatgggtctggcagtg |
| | N-tag: His | QGILVPCQSPWNTPLLPVKKPGTNDYRPVQ | | cgtcaggcaccgctgattattccgctgaaa |
| | P043 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgatgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 145 | RTx | TWLSDFPQAWAETGGMGLAVRQAPLIIPLK | 146 | acttggctgtctgatttccctcaggcgtgg |
| | 42B L | ATSTPVSIKQYPLSQKARLGIKPHIQRLLD | | gccgaaacgggtggcatgggtctggcagtg |
| | N-tag: His | QGILVPCQSPWNTPLLPVKKPGTNDYRPVQ | | cgtcaggcaccgctgattattccgctgaaa |
| | P061 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgctgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPAGRWLSKARMTHYQALLLDTDRVQFG | | agccagcctctgtttgcatttgagtggcgt |
| | | PVVALNPATLLPLPEEGLQHNCLDILAEAH | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIHCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggcgcaccgcatgcagtcgaggccctt |
| | | | | gttaagcaaccggcaggaagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcgcac |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | cattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 147 | RTx | TWLSDFPQAWAETGGMGLAVRQAPLIIPLK | 148 | acttggctgtctgatttccctcaggcgtgg |
| | 50A+G | ATSTPVSIKQYPLSQKARLGIKPHIQRLLD | | gccgaaacgggtggcatgggtctggcagtg |
| | N-tag: His | QGILVPCQSPWNTPLLPVKKPGTNDYRPVQ | | cgtcaggcaccgctgattattccgctgaaa |
| | P072 | DLREVNKRVEDIHPTVPNPYNLLSGPPPSH | | gcgacgtcgaccccggtgagcatcaagcaa |
| | | QWYTVLDLKDAFFCLRLHPTSQPLFAFEWR | | tatccgctgtcccaaaaggcgcgcttaggt |
| | | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPPDRWLSKARMTHYQALLLDTDRVQFG | | agccagcctctgtttgcatttgagtggcgt |
| | | PVVALNPATLLPLPEEGLQHNCLDILAEAV | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIYCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | | | ttgcagaccttgggtaacctgggttatcgc |
| | | | | gctagcgcgaagaaagcgcagatttgccaa |
| | | | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggtgcaccgcatgcagtcgaggccctt |
| | | | | gttaagcaaccgccggatagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggcggtg |
| | | | | ggcactcgcccggatctgacggaccagccg |
| | | | | ctgccggacgccgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | tattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| 149 | RTx | TWLSDFPQAWAETGGVGLAVRQAPLIIPLK | 150 | acctggctgagcgattttccgcaggcatgg |
| | SOLD 34- | ATSTPVSIKQYPISQKARLGIKPHIQRLLD | | gcagaaaccggtggtgtgggtctggcagtg |
| | Sto7 (K13L) | QGILVPCRSPWNTPLLPVKKPGTNDYRPVQ | | cgccaggcaccgctgattattccgctgaaa |
| | N-tag: His | DLREVNKRVEDIHPTVPNPYNLLSGPPPSH | | gcaaccagcaccccggtgagcattaaacag |
| | C-Tag: Sto7 | QWYTVLDLKDAFFCLRLHPTSQPLFAFEWR | | tatccgattagccagaaagcacgcctgggt |
| | P140 | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccgcagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEVKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTTETEVIWAKALPAGTSAQRAEL | | aacagcccgaccctgtttaacgaagcactg |
| | | IALTQALKMAEGKKLNVYTDSRYAFATAHI | | catcgcgatctggcagattttcgcattcag |
| | | QGEIYRRRGWLTSKGKEIKNKDEILALLKA | | catccggatctgattctgctgcagtatgtg |
| | | LFLPKRLSIIHCPGHQKGHSAEARGNRMAD | | gatgatctgctgctggcagcaaccagcgaa |
| | | QAARKAAITETPDTSTLLIENSSPNSRLIN | | ctggattgccagcagggtacccgcgcactg |
| | | GGGGSVTVKFKYKGEELEVDISKIKKVWRV | | ctgcagaccctgggtaacctgggttatcgc |
| | | GKMISFTYDDNGKTGRGAVSEKDAPKELLQ | | gcaagcgcaaaaaaagcacagatttgccag |
| | | MLEKSGKK | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaagtgaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccaccgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccgat |
| | | | | agccgctatgcatttgcaaccgcacatatt |
| | | | | cagggtgaaatttatcgccgccgcggttgg |
| | | | | ctgaccagcaaaggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gcagaagcacgcggtaaccgcatggcagat |
| | | | | caggcagcacgcaaagcagcaattaccgaa |
| | | | | accccggataccagcaccctgctgattgaa |
| | | | | aacagcagcccgaacagccgcctgattaac |
| | | | | ggtggaggtgggtcagtaacagtaaaattt |
| | | | | aaatataaaggtgaagagttggaggttgat |
| | | | | atttcaaagataaaaaaagtgtggagagtc |
| | | | | ggtaaaatgataagttttacgtatgatgac |
| | | | | aacgggaaaaccggtcggggcgccgtgtcc |
| | | | | gaaaaagacgctccgaaggaactactccaa |
| | | | | atgcttgagaaaagtggaaaaaaa |
| 151 | RTx | TWLSDFPQAWAETGGVGLAVRQAPLIIPLK | 152 | acctggctgagcgattttccgcaggcatgg |
| | SOLD 025- | ATSTPVSIKQYPMSQKARLGIKPHIQRLLD | | gcagaaaccggtggtgtgggtctggcagtg |
| | Sto7 (K13L) | QGILVPCQSPWNTPLLPVKKPGTNDYRPVQ | | cgccaggcaccgctgattattccgctgaaa |
| | N-tag: his | DLREVNKRVEDIHPTVPNPYNLLSGPPPSH | | gcaaccagcaccccggtgagcattaaacag |
| | C-tag: Sto7 | QWYTVLDLKDAFFCLRLHPTSQPLFAFEWR | | tatccgatgagccagaaagcacgcctgggt |
| | P142 | DPEMGISGQLTWTRLPQGFKNSPTLFNEAL | | attaaaccgcatattcagcgcctgctggat |
| | | HRDLADFRIQHPDLILLQYVDDLLLAATSE | | cagggtattctggtgccgtgccagagcccg |
| | | LDCQQGTRALLQTLGNLGYRASAKKAQICQ | | tggaacaccccgctgctgccggtgaaaaaa |
| | | KQVKYLGYLLKEGQRWLTEARKETVMGQPT | | ccgggtaccaacgattatcgcccggtgcag |
| | | PKTPRQLRRFLGKAGFCRLFIPGFAEMAAP | | gatctgcgcgaagtgaacaaacgcgtggaa |
| | | LYPLTKPGTLFNWGPDQQKAYQEIKQALLT | | gatattcatccgaccgtgccgaacccgtat |
| | | APALGLPDLTKPFELFVDEKQGYAKGVLTQ | | aacctgctgagcggtccgccgccgagccat |
| | | KLGPWRRPVAYLSKKLDPVAAGWPPCLRMV | | cagtggtataccgtgctggatctgaaagat |
| | | AAIAVLTKDAGKLTMGQPLVIGAPHAVEAL | | gcatttttttgcctgcgcctgcatccgacc |
| | | VKQPAGRWLSKARMTHYQALLLDTDRVQFG | | agccagccgctgtttgcatttgaatggcgc |
| | | PVVALNPATLLPLPEEGLQHNCLDILAEAH | | gatccggaaatgggtattagcggtcagctg |
| | | GTRPDLTDQPLPDADHTWYTNGSSLLQEGQ | | acctggacccgcctgccgcagggttttaaa |
| | | RKAGAAVTDETEVIWAKALPAGTSAQRAEL | | aacagcccgaccctgtttaacgaagcactg |
| | | IALTQALKMAEGKKLNVYTNSRYAFATAHI | | catcgcgatctggcagattttcgcattcag |
| | | HGEIYRRRGWLTSGGKEIKNKDEILALLKA | | catccggatctgattctgctgcagtatgtg |
| | | LFLPKRLSIIHCPGHQKGHSVEARGNRMAH | | gatgatctgctgctggcagcaaccagcgaa |
| | | QAARRAAITETPDTSTLPIENSSPNSRLIN | | ctggattgccagcagggtacccgcgcactg |
| | | GGGGSVTVKFKYKGEELEVDISKIKKVWRV | | ctgcagaccctgggtaacctgggttatcgc |
| | | GKMISFTYDDNGKTGRGAVSEKDAPKELLQ | | gcaagcgcaaaaaaagcacagatttgccag |
| | | MLEKSGKK | | aaacaggtgaaatatctgggttatctgctg |
| | | | | aaagaaggtcagcgctggctgaccgaagca |
| | | | | cgcaaagaaaccgtgatgggtcagccgacc |
| | | | | ccgaaaaccccgcgccagctgcgccgcttt |
| | | | | ctgggtaaagcaggtttttgccgcctgttt |
| | | | | attccgggttttgcagaaatggcagcaccg |
| | | | | ctgtatccgctgaccaaaccgggtaccctg |
| | | | | tttaactggggtccggatcagcagaaagca |
| | | | | tatcaggaaattaaacaggcactgctgacc |
| | | | | gcaccggcactgggtctgccggatctgacc |
| | | | | aaaccgtttgaactgtttgtggatgaaaaa |
| | | | | cagggttatgcaaaaggtgtgctgacccag |
| | | | | aaactgggtccgtggcgccgcccggtggca |
| | | | | tatctgagcaaaaaactggatccggtggca |
| | | | | gcaggttggccgccgtgcctgcgcatggtg |
| | | | | gcagcaattgcagtgctgaccaaagatgca |
| | | | | ggtaaactgaccatgggtcagccgctggtg |
| | | | | attggtgcaccgcatgcagtggaagcactg |
| | | | | gtgaaacagccggcaggtcgctggctgagc |
| | | | | aaagcacgcatgacccattatcaggcactg |
| | | | | ctgctggataccgatcgcgtgcagtttggt |
| | | | | ccggtggtggcactgaacccggcaaccctg |
| | | | | ctgccgctgccggaagaaggtctgcagcat |
| | | | | aactgcctggatattctggcagaagcacat |
| | | | | ggtacccgcccggatctgaccgatcagccg |
| | | | | ctgccggatgcagatcatacctggtatacc |
| | | | | aacggtagcagcctgctgcaggaaggtcag |
| | | | | cgcaaagcaggtgcagcagtgaccgatgaa |
| | | | | accgaagtgatttgggcaaaagcactgccg |
| | | | | gcaggtaccagcgcacagcgcgcagaactg |
| | | | | attgcactgacccaggcactgaaaatggca |
| | | | | gaaggtaaaaaactgaacgtgtataccaac |
| | | | | agccgctatgcatttgcaaccgcacatatt |
| | | | | catggtgaaatttatcgccgccgcggttgg |
| | | | | ctgaccagcggtggtaaagaaattaaaaac |
| | | | | aaagatgaaattctggcactgctgaaagca |
| | | | | ctgtttctgccgaaacgcctgagcattatt |
| | | | | cattgcccgggtcatcagaaaggtcatagc |
| | | | | gtggaagcacgcggtaaccgcatggcacat |
| | | | | caggcagcacgccgcgcagcaattaccgaa |
| | | | | accccggataccagcaccctgccgattgaa |
| | | | | aacagcagcccgaacagccgcctgattaac |
| | | | | ggtggaggtgggtcagtaacagtaaaattt |
| | | | | aaatataaaggtgaagagttggaggttgat |
| | | | | atttcaaagataaaaaaagtgtggagagtc |
| | | | | ggtaaaatgataagttttacgtatgatgac |
| | | | | aacgggaaaaccggtcggggcgccgtgtcc |
| | | | | gaaaaagacgctccgaaggaactactccaa |
| | | | | atgcttgagaaaagtggaaaaaaa |
| 153 | RTx | TWLSDFPQAWAETGGMGLAVRQAPLIIPLK | 154 | acctggctgtctgactttccgcaggcttgg |
| | 42B-Sto7 | ATSTPVSIKQYPMSQKARLGIKPHIQRLLD | | gcagagaccggggggatgggtttagcggta |
| | K13L | QGILVPCQSPWNTPLLPVKKPGTNDYRPVQ | | cggcaggcaccactgattataccgctgaaa |
| | N-tag: His | DLREVNKRVEDIHPTVPNPYNLLSGPPPSH | | gcaacatcgactcctgtctcgataaagcaa |
| | C-Tag: Sto7 | QWYTVLDLKDAFFCLRLHPTSQPLFAFEWR | | taccctatgtcccaaaaggctcgcttaggc |
| | P143 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRAEL | | aactccccgactctgtttaatgaagcgctg |
| | | IALTQALKMAEGKKLNVYTDSRYAFATAHI | | caccgcgacctggccgactttagaatacag |
| | | HGEIYRRRGWLTSKGKEIKNKDEILALLKA | | cacccagatcttatccttctgcagtacgtc |
| | | LFLPKRLSIIHCPGHQKGHSAEARGNRMAD | | gatgatcttttgctggccgcaacaagcgag |
| | | QAARKAAITETPDTSTLLIENSSPNSRLIN | | ctggattgtcaacaggggacgcgtgctctt |
| | | GGGGSVTVKFKYKGEELEVDISKIKKVWRV | | ttacagaccctgggtaatctcggttaccgc |
| | | GKMISFTYDDNGKTGRGAVSEKDAPKELLQ | | gcgagcgcaaaaaaagcccaaatatgtcaa |
| | | MLEKSGKK | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagagccgagtta |
| | | | | attgcgttaacacaggcgctgaagatggct |
| | | | | gaagggaaaaaactgaacgtctataccgac |
| | | | | agccgctatgcatttgctacagcccatatt |
| | | | | catggagaaatttatcgccggcgaggttgg |
| | | | | ctcacgtcgaaaggtaaagaaaaaaaaata |
| | | | | aggatgaaattctggcacttctgaaagcgt |
| | | | | tatttctgcctaaacgcttaagtatcatac |
| | | | | actgtcctggtcaccaaaaaggtcatagtg |
| | | | | ctgaagcgcgcgggaaccgcatggccgatc |
| | | | | aggccgcccgtaaagctgcaattaccgaaa |
| | | | | cccccgatacatctacgctgctgattgaaa |
| | | | | actctagcccaaatagcagactgattaatg |
| | | | | gtggaggtgggtcagtaacagtaaaattta |
| | | | | aatataaaggtgaagagttggaggttgata |
| | | | | tttcaaagataaaaaaagtgtggagagtcg |
| | | | | gtaaaatgataagttttacgtatgatgaca |
| | | | | acgggaaaaccggtcggggcgccgtgtccg |
| | | | | aaaaagacgctccgaaggaactactccaaa |
| | | | | tgcttgagaaaagtggaaaaaaa |
| 155 | RTx | TWLSDFPQAWAETGGMGLAVRQAPLIIPLK | 156 | acctggctgtctgactttccgcaggcttgg |
| | 42B L- | ATSTPVSIKQYPLSQKARLGIKPHIQRLLD | | gcagagaccggggggatgggtttagcggta |
| | Sto7 K13L | QGILVPCQSPWNTPLLPVKKPGTNDYRPVQ | | cggcaggcaccactgattataccgctgaaa |
| | N-tag: His | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctctgtcccaaaaggctcgcttaggc |
| | | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPV | | cagtggtacacagtcctggacttaaaagat |
| | | AAGWPPCLRMVAAIAVLTKDAGKLTMGQPL | | gcattcttctgcctccgtctgcatccaact |
| | | VIGAPHAVEALVKQPAGRWLSKARMTHYQA | | agccaacctctgttcgcgtttgaatggcgg |
| | | LLLDTDRVQFGPVVALNPATLLPLPEEGLQ | | gacccggaaatgggaattagcggtcagctc |
| | | HNCLDILAEAHGTRPDLTDQPLPDADHTWY | | acctggacaagactcccgcaaggattcaag |
| | | TNGSSLLQEGQRKAGAAVTTETEVIWAKAL | | aactccccgactctgtttaatgaagcgctg |
| | | PAGTSAQRAELIALTQALKMAEGKKLNVYT | | caccgcgacctggccgactttagaatacag |
| | | DSRYAFATAHIHGEIYRRRGWLTSKGKEIK | | cacccagatcttatccttctgcagtacgtc |
| | | NKDEILALLKALFLPKRLSIIHCPGHQKGH | | gatgatcttttgctggccgcaacaagcgag |
| | | SAEARGNRMADQAARKAAITETPDTSTLLI | | ctggattgtcaacaggggacgcgtgctctt |
| | | ENSSPNSRLINGGGGSVTVKFKYKGEELEV | | ttacagaccctgggtaatctcggttaccgc |
| | | DISKIKKVWRVGKMISFTYDDNGKTGRGAV | | gcgagcgcaaaaaaagcccaaatatgtcaa |
| | | SEKDAPKELLQMLEKSGKK | | aaacaggtaaagtatctgggatacctgctt |
| | | | | aaagagggtcagcgatggctcactgaggca |
| | | | | cgtaaagagaccgttatgggacagccgaca |
| | | | | c |
| | | | | caaaaacaccgcgacaactgcggcggtttc |
| | | | | tgggaaaagcgggtttttgtcgattattta |
| | | | | tccctgggtttgccgaaatggcggcacctt |
| | | | | tatatccactcactaagccaggcacactgt |
| | | | | ttaattggggaccggatcaacagaaggcgt |
| | | | | accaggagataaaacaagcgctgttaaccg |
| | | | | cccccgctttaggtctgcctgacctgacta |
| | | | | agccgttcgaactgtttgtagatgaaaaac |
| | | | | aaggttacgcgaaaggtgttttaactcaga |
| | | | | aacttgggccatggcgtcgtcctgtcgcgt |
| | | | | atctctctaaaaaacttgacccggttgcgg |
| | | | | ctggctggcctccatgcctgagaatggtgg |
| | | | | cagctatagcagtcctgactaaagatgccg |
| | | | | gtaaattgaccatgggtcagccacttgtga |
| | | | | tcggtgctccccacgcggtggaggcgttgg |
| | | | | ttaaacagcctgccgggcgttggttgtcga |
| | | | | aagctcgcatgacacattatcaggccttat |
| | | | | tacttgatacggacagagttcagtttggac |
| | | | | cggtagtcgccctgaacccggccacgttac |
| | | | | ttccgctgccagaagaaggtttacagcata |
| | | | | attgtctcgatatccttgccgaggctcatg |
| | | | | gaacccgcccagaccttactgaccaaccac |
| | | | | ttcccgatgctgaccacacctggtatacca |
| | | | | atggttcatctctgctacaggagggtcaac |
| | | | | gcaaagcgggtgcagcagttaccactgaaa |
| | | | | ctgaggttatttgggcgaaagcgctgccag |
| | | | | caggaacgagcgctcagagagccgagttaa |
| | | | | ttgcgttaacacaggcgctgaagatggctg |
| | | | | aagggaaaaaactgaacgtctataccgaca |
| | | | | gccgctatgcatttgctacagcccatattc |
| | | | | atggagaaatttatcgccggcgaggttggc |
| | | | | tcacgtcgaaaggtaaagaaataaaaaata |
| | | | | aggatgaaattctggcacttctgaaagcgt |
| | | | | tatttctgcctaaacgcttaagtatcatac |
| | | | | actgtcctggtcaccaaaaaggtcatagtg |
| | | | | ctgaagcgcgcgggaaccgcatggccgatc |
| | | | | aggccgcccgtaaagctgcaattaccgaaa |
| | | | | cccccgatacatctacgctgctgattgaaa |
| | | | | actctagcccaaatagcagactgattaatg |
| | | | | gtggaggtgggtcagtaacagtaaaattta |
| | | | | aatataaaggtgaagagttggaggttgata |
| | | | | tttcaaagataaaaaaagtgtggagagtcg |
| | | | | gtaaaatgataagttttacgtatgatgaca |
| | | | | acgggaaaaccggtcggggcgccgtgtccg |
| | | | | aaaaagacgctccgaaggaactactccaaa |
| | | | | tgcttgagaaaagtggaaaaaaa |
| 157 | RTx | TWLSDFPQAWAETGGVGLAVRQAPLIIPLK | 158 | acctggctgtctgactttccgcaggcttgg |
| | 42B V- | ATSTPVSIKQYPMSQKARLGIKPHIQRLLD | | gcagagaccgggggggtgggtttagcggta |
| | Sto7 K13L | QGILVPCQSPWNTPLLPVKKPGTNDYRPVQ | | cggcaggcaccactgattataccgctgaaa |
| | N-Tag: His | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctatgtcccaaaaggctcgcttaggc |
| | P145 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRAEL | | aactccccgactctgtttaatgaagcgctg |
| | | IALTQALKMAEGKKLNVYTDSRYAFATAHI | | caccgcgacctggccgactttagaatacag |
| | | HGEIYRRRGWLTSKGKEIKNKDEILALLKA | | cacccagatcttatccttctgcagtacgtc |
| | | LFLPKRLSIIHCPGHQKGHSAEARGNRMAD | | gatgatcttttgctggccgcaacaagcgag |
| | | QAARKAAITETPDTSTLLIENSSPNSRLIN | | ctggattgtcaacaggggacgcgtgctctt |
| | | GGGGSVTVKFKYKGEELEVDISKIKKVWRV | | ttacagaccctgggtaatctcggttaccgc |
| | | GKMISFTYDDNGKTGRGAVSEKDAPKELLQ | | gcgagcgcaaaaaaagcccaaatatgtcaa |
| | | MLEKSGKK | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagagccgagtta |
| | | | | attgcgttaacacaggcgctgaagatggct |
| | | | | gaagggaaaaaactgaacgtctataccgac |
| | | | | agccgctatgcatttgctacagcccatatt |
| | | | | catggagaaatttatcgccggcgaggttgg |
| | | | | ctcacgtcgaaaggtaaagaaataaaaaat |
| | | | | aaggatgaaattctggcacttctgaaagcg |
| | | | | ttatttctgcctaaacgcttaagtatcata |
| | | | | cactgtcctggtcaccaaaaaggtcatagt |
| | | | | gctgaagcgcgcgggaaccgcatggccgat |
| | | | | caggccgcccgtaaagctgcaattaccgaa |
| | | | | acccccgatacatctacgctgctgattgaa |
| | | | | aactctagcccaaatagcagactgattaat |
| | | | | ggtggaggtgggtcagtaacagtaaaattt |
| | | | | aaatataaaggtgaagagttggaggttgat |
| | | | | atttcaaagataaaaaaagtgtggagagtc |
| | | | | ggtaaaatgataagttttacgtatgatgac |
| | | | | aacgggaaaaccggtcggggcgccgtgtcc |
| | | | | gaaaaagacgctccgaaggaactactccaa |
| | | | | atgcttgagaaaagtggaaaaaaa |
| 159 | RTx | TWLSDFPQAWAETGGMGLAVRQAPLIIPLK | 160 | acctggctgtctgactttccgcaggcttgg |
| | N-tag: His | ATSTPVSIKQYPLSQKARLGIKPHIQRLLD | | gcagagaccggggggatgggtttagcggta |
| | C-tag: Sto7 | QGILVPCQSPWNTPLLPVKKPGTNDYRPVQ | | cggcaggcaccactgattataccgctgaaa |
| | 42B L-Sto7 | DLREVNKRVEDIHPTVPNPYNLLSGPPPSH | | gcaacatcgactcctgtctcgataaagcaa |
| | Trunc 529 | QWYTVLDLKDAFFCLRLHPTSQPLFAFEWR | | taccctctgtcccaaaaggctcgcttaggc |
| | P146 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggggg |
| | | PVVALNPATLLPLPEEGLQHNCLDILAEAH | | acccggaaatgggaattagcggtcagctca |
| | | GTRPDLTDQPLPDADHTWYTNGSSLLGGGG | | cctggacaagactcccgcaaggattcaaga |
| | | SVTVKFKYKGEELEVDISKIKKVWRVGKMI | | actccccgactctgtttaatgaagcgctgc |
| | | SFTYDDNGKTGRGAVSEKDAPKELLQMLEK | | accgcgacctggccgactttagaatacagc |
| | | SGKK | | acccagatcttatccttctgcagtacgtcg |
| | | | | atgatcttttgctggccgcaacaagcgagc |
| | | | | tggattgtcaacaggggacgcgtgctcttt |
| | | | | tacagaccctgggtaatctcggttaccgcg |
| | | | | cgagcgcaaaaaaagcccaaatatgtcaaa |
| | | | | aacaggtaaagtatctgggatacctgctta |
| | | | | aagagggtcagcgatggctcactgaggcac |
| | | | | gtaaagagaccgttatgggacagccgacac |
| | | | | caaaaacaccgcgacaactgcggcggtttc |
| | | | | tgggaaaagcgggtttttgtcgattattta |
| | | | | tccctgggtttgccgaaatggcggcacctt |
| | | | | tatatccactcactaagccaggcacactgt |
| | | | | ttaattggggaccggatcaacagaaggcgt |
| | | | | accaggagataaaacaagcgctgttaaccg |
| | | | | cccccgctttaggtctgcctgacctgacta |
| | | | | agccgttcgaactgtttgtagatgaaaaac |
| | | | | aaggttacgcgaaaggtgttttaactcaga |
| | | | | aacttgggccatggcgtcgtcctgtcgcgt |
| | | | | atctctctaaaaaacttgacccggttgcgg |
| | | | | ctggctggcctccatgcctgagaatggtgg |
| | | | | cagctatagcagtcctgactaaagatgccg |
| | | | | gtaaattgaccatgggtcagccacttgtga |
| | | | | tcggtgctccccacgcggtggaggcgttgg |
| | | | | ttaaacagcctgccgggcgttggttgtcga |
| | | | | aagctcgcatgacacattatcaggccttat |
| | | | | tacttgatacggacagagttcagtttggac |
| | | | | cggtagtcgccctgaacccggccacgttac |
| | | | | ttccgctgccagaagaaggtttacagcata |
| | | | | attgtctcgatatccttgccgaggctcatg |
| | | | | gaacccgcccagaccttactgaccaaccac |
| | | | | ttcccgatgctgaccacacctggtatacca |
| | | | | atggttcatctctgctaggtggaggtgggt |
| | | | | cagtaacagtaaaatttaaatataaaggtg |
| | | | | aagagttggaggttgatatttcaaagataa |
| | | | | aaaaagtgtggagagtcggtaaaatgataa |
| | | | | gttttacgtatgatgacaacgggaaaaccg |
| | | | | gtcggggcgccgtgtccgaaaaagacgctc |
| | | | | cgaaggaactactccaaatgcttgagaaaa |
| | | | | gtggaaaaaaa |
| 161 | RTx | TWLSDFPQAWAETGGMGLAVRQAPLIIPLK | 162 | acctggctgtctgactttccgcaggcttgg |
| | 42B L-Sto7 | ATSTPVSIKQYPLSQKARLGIKPHIQRLLD | | gcagagaccggggggatgggtttagcggta |
| | Trunc 534 | QGILVPCQSPWNTPLLPVKKPGTNDYRPVQ | | cggcaggcaccactgattataccgctgaaa |
| | N-tag: His | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctctgtcccaaaaggctcgcttaggc |
| | P147 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggggg |
| | | PVVALNPATLLPLPEEGLQHNCLDILAEAH | | acccggaaatgggaattagcggtcagctca |
| | | GTRPDLTDQPLPDADHTWYTNGSSLLQEGQ | | cctggacaagactcccgcaaggattcaaga |
| | | RGGGGSVTVKFKYKGEELEVDISKIKKVWR | | actccccgactctgtttaatgaagcgctgc |
| | | VGKMISFTYDDNGKTGRGAVSEKDAPKELL | | accgcgacctggccgactttagaatacagc |
| | | QMLEKSGKK | | acccagatcttatccttctgcagtacgtcg |
| | | | | atgatcttttgctggccgcaacaagcgagc |
| | | | | tggattgtcaacaggggacgcgtgctcttt |
| | | | | tacagaccctgggtaatctcggttaccgcg |
| | | | | cgagcgcaaaaaaagcccaaatatgtcaaa |
| | | | | aacaggtaaagtatctgggatacctgctta |
| | | | | aagagggtcagcgatggctcactgaggcac |
| | | | | gtaaagagaccgttatgggacagccgacac |
| | | | | caaaaacaccgcgacaactgcggcggtttc |
| | | | | tgggaaaagcgggtttttgtcgattattta |
| | | | | tccctgggtttgccgaaatggcggcacctt |
| | | | | tatatccactcactaagccaggcacactgt |
| | | | | ttaattggggaccggatcaacagaaggcgt |
| | | | | accaggagataaaacaagcgctgttaaccg |
| | | | | cccccgctttaggtctgcctgacctgacta |
| | | | | agccgttcgaactgtttgtagatgaaaaac |
| | | | | aaggttacgcgaaaggtgttttaactcaga |
| | | | | aacttgggccatggcgtcgtcctgtcgcgt |
| | | | | atctctctaaaaaacttgacccggttgcgg |
| | | | | ctggctggcctccatgcctgagaatggtgg |
| | | | | cagctatagcagtcctgactaaagatgccg |
| | | | | gtaaattgaccatgggtcagccacttgtga |
| | | | | tcggtgctccccacgcggtggaggcgttgg |
| | | | | ttaaacagcctgccgggcgttggttgtcga |
| | | | | aagctcgcatgacacattatcaggccttat |
| | | | | tacttgatacggacagagttcagtttggac |
| | | | | cggtagtcgccctgaacccggccacgttac |
| | | | | ttccgctgccagaagaaggtttacagcata |
| | | | | attgtctcgatatccttgccgaggctcatg |
| | | | | gaacccgcccagaccttactgaccaaccac |
| | | | | ttcccgatgctgaccacacctggtatacca |
| | | | | atggttcatctctgctacaggagggtcaac |
| | | | | gcggtggaggtgggtcagtaacagtaaaat |
| | | | | ttaaatataaaggtgaagagttggaggttg |
| | | | | atatttcaaagataaaaaaagtgtggagag |
| | | | | tcggtaaaatgataagttttacgtatgatg |
| | | | | acaacgggaaaaccggtcggggcgccgtgt |
| | | | | ccgaaaaagacgctccgaaggaactactcc |
| | | | | aaatgcttgagaaaagtggaaaaaaa |
| 163 | RTx | TWLSDFPQAWAETGGMGLAVRQAPLIIPLK | 164 | acctggctgtctgactttccgcaggcttgg |
| | 42B L-Sto7 | ATSTPVSIKQYPLSQKARLGIKPHIQRLLD | | gcagagaccggggggatgggtttagcggta |
| | Trunc 560 | QGILVPCQSPWNTPLLPVKKPGTNDYRPVQ | | cggcaggcaccactgattataccgctgaaa |
| | N-tag: his | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctctgtcccaaaaggctcgcttaggc |
| | P148 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRGGG | | aactccccgactctgtttaatgaagcgctg |
| | | GSVTVKFKYKGEELEVDISKIKKVWRVGKM | | caccgcgacctggccgactttagaatacag |
| | | ISFTYDDNGKTGRGAVSEKDAPKELLQMLE | | cacccagatcttatccttctgcagtacgtc |
| | | KSGKK | | gatgatcttttgctggccgcaacaagcgag |
| | | | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagaggtggaggt |
| | | | | gggtcagtaacagtaaaatttaaatataaa |
| | | | | ggtgaagagttggaggttgatatttcaaag |
| | | | | ataaaaaaagtgtggagagtcggtaaaatg |
| | | | | ataagttttacgtatgatgacaacgggaaa |
| | | | | accggtcggggcgccgtgtccgaaaaagac |
| | | | | gctccgaaggaactactccaaatgcttgag |
| | | | | aaaagtggaaaaaaa |
| 166 | RTx | TWLSDFPQAWAETGGMGLAVRQAPLIIPLK | 167 | acctggctgtctgactttccgcaggcttgg |
| | 42B L-Sto7 | ATSTPVSIKQYPLSQKARLGIKPHIQRLLD | | gcagagaccggggggatgggtttagcggta |
| | Trunc 613 | QGILVPCQSPWNTPLLPVKKPGTNDYRPVQ | | cggcaggcaccactgattataccgctgaaa |
| | N-tag: his | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctctgtcccaaaaggctcgcttaggc |
| | P149 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRAEL | | aactccccgactctgtttaatgaagcgctg |
| | | IALTQALKMAEGKKLNVYTDSRYAFATAHI | | caccgcgacctggccgactttagaatacag |
| | | HGEIYRRRGWLTSKGKEIKNGGGGSVTVKF | | cacccagatcttatccttctgcagtacgtc |
| | | KYKGEELEVDISKIKKVWRVGKMISFTYDD | | gatgatcttttgctggccgcaacaagcgag |
| | | NGKTGRGAVSEKDAPKELLQMLEKSGKK | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagagccgagtta |
| | | | | attgcgttaacacaggcgctgaagatggct |
| | | | | gaagggaaaaaactgaacgtctataccgac |
| | | | | agccgctatgcatttgctacagcccatatt |
| | | | | catggagaaatttatcgccggcgaggttgg |
| | | | | ctcacgtcgaaaggtaaagaaataaaaaat |
| | | | | ggtggaggtgggtcagtaacagtaaaattt |
| | | | | aaatataaaggtgaagagttggaggttgat |
| | | | | atttcaaagataaaaaaagtgtggagagtc |
| | | | | ggtaaaatgataagttttacgtatgatgac |
| | | | | aacgggaaaaccggtcggggcgccgtgtcc |
| | | | | gaaaaagacgctccgaaggaactactccaa |
| | | | | atgcttgagaaaagtggaaaaaaa |
| 168 | RTx | TWLSDFPQAWAETGGMGLAVRQAPLIIPLK | 169 | acctggctgtctgactttccgcaggcttgg |
| | 42B L-Sto7 | ATSTPVSIKQYPLSQKARLGIKPHIQRLLD | | gcagagaccggggggatgggtttagcggta |
| | Trunc 609 | QGILVPCQSPWNTPLLPVKKPGTNDYRPVQ | | cggcaggcaccactgattataccgctgaaa |
| | N-tag: His | DLREVNKRVEDIHPTVPNPYNLLSGPPPSH | | gcaacatcgactcctgtctcgataaagcaa |
| | C-tag: Sto7 | QWYTVLDLKDAFFCLRLHPTSQPLFAFEWR | | taccctctgtcccaaaaggctcgcttaggc |
| | P150 | DPEMGISGQLTWTRLPQGFKNSPTLFNEAL | | attaagccacatatccagcgtctgctagat |
| | | HRDLADFRIQHPDLILLQYVDDLLLAATSE | | caaggtattctcgttccttgtcagtctccg |
| | | LDCQQGTRALLQTLGNLGYRASAKKAQICQ | | tggaataccccgttgctgccagttaagaaa |
| | | KQVKYLGYLLKEGQRWLTEARKETVMGQPT | | ccgggcacaaacgactatcgcccggttcag |
| | | PKTPRQLRRFLGKAGFCRLFIPGFAEMAAP | | gacctgcgagaggtaaacaaacgtgtggaa |
| | | LYPLTKPGTLFNWGPDQQKAYQEIKQALLT | | gatatccatccgacagtgcccaacccctac |
| | | APALGLPDLTKPFELFVDEKQGYAKGVLTQ | | aatctgttgtctggtcctcctccaagccac |
| | | KLGPWRRPVAYLSKKLDPVAAGWPPCLRMV | | cagtggtacacagtcctggacttaaaagat |
| | | AAIAVLTKDAGKLTMGQPLVIGAPHAVEAL | | gcattcttctgcctccgtctgcatccaact |
| | | VKQPAGRWLSKARMTHYQALLLDTDRVQFG | | agccaacctctgttcgcgtttgaatggcgg |
| | | PVVALNPATLLPLPEEGLQHNCLDILAEAH | | gacccggaaatgggaattagcggtcagctc |
| | | GTRPDLTDQPLPDADHTWYTNGSSLLQEGQ | | acctggacaagactcccgcaaggattcaag |
| | | RKAGAAVTTETEVIWAKALPAGTSAQRAEL | | aactccccgactctgtttaatgaagcgctg |
| | | IALTQALKMAEGKKLNVYTDSRYAFATAHI | | caccgcgacctggccgactttagaatacag |
| | | HGEIYRRRGWLTSKGKGGGGSVTVKFKYKG | | cacccagatcttatccttctgcagtacgtc |
| | | EELEVDISKIKKVWRVGKMISFTYDDNGKT | | gatgatcttttgctggccgcaacaagcgag |
| | | GRGAVSEKDAPKELLQMLEKSGKK | | ctggattgtcaacaggggacgcgtgctctt |
| | | | | ttacagaccctgggtaatctcggttaccgc |
| | | | | gcgagcgcaaaaaaagcccaaatatgtcaa |
| | | | | aaacaggtaaagtatctgggatacctgctt |
| | | | | aaagagggtcagcgatggctcactgaggca |
| | | | | cgtaaagagaccgttatgggacagccgaca |
| | | | | ccaaaaacaccgcgacaactgcggcggttt |
| | | | | ctgggaaaagcgggtttttgtcgattattt |
| | | | | atccctgggtttgccgaaatggcggcacct |
| | | | | ttatatccactcactaagccaggcacactg |
| | | | | tttaattggggaccggatcaacagaaggcg |
| | | | | taccaggagataaaacaagcgctgttaacc |
| | | | | gcccccgctttaggtctgcctgacctgact |
| | | | | aagccgttcgaactgtttgtagatgaaaaa |
| | | | | caaggttacgcgaaaggtgttttaactcag |
| | | | | aaacttgggccatggcgtcgtcctgtcgcg |
| | | | | tatctctctaaaaaacttgacccggttgcg |
| | | | | gctggctggcctccatgcctgagaatggtg |
| | | | | gcagctatagcagtcctgactaaagatgcc |
| | | | | ggtaaattgaccatgggtcagccacttgtg |
| | | | | atcggtgctccccacgcggtggaggcgttg |
| | | | | gttaaacagcctgccgggcgttggttgtcg |
| | | | | aaagctcgcatgacacattatcaggcctta |
| | | | | ttacttgatacggacagagttcagtttgga |
| | | | | ccggtagtcgccctgaacccggccacgtta |
| | | | | cttccgctgccagaagaaggtttacagcat |
| | | | | aattgtctcgatatccttgccgaggctcat |
| | | | | ggaacccgcccagaccttactgaccaacca |
| | | | | cttcccgatgctgaccacacctggtatacc |
| | | | | aatggttcatctctgctacaggagggtcaa |
| | | | | cgcaaagcgggtgcagcagttaccactgaa |
| | | | | actgaggttatttgggcgaaagcgctgcca |
| | | | | gcaggaacgagcgctcagagagccgagtta |
| | | | | attgcgttaacacaggcgctgaagatggct |
| | | | | gaagggaaaaaactgaacgtctataccgac |
| | | | | agccgctatgcatttgctacagcccatatt |
| | | | | catggagaaatttatcgccggcgaggttgg |
| | | | | ctcacgtcgaaaggtaaaggtggaggtggg |
| | | | | tcagtaacagtaaaatttaaatataaaggt |
| | | | | gaagagttggaggttgatatttcaaagata |
| | | | | aaaaaagtgtggagagtcggtaaaatgata |
| | | | | agttttacgtatgatgacaacgggaaaacc |
| | | | | ggtcggggcgccgtgtccgaaaaagacgct |
| | | | | ccgaaggaactactccaaatgcttgagaaa |
| | | | | agtggaaaaaaa |
| 170 | RTx | TWLSDFPQAWAETGGMGLAVRQAPLIIPLK | 171 | acttggctgtctgatttccctcaggcgtgg |
| | 50A+G- | ATSTPVSIKQYPLSQKARLGIKPHIQRLLD | | gccgaaacgggtggcatgggtctggcagtg |
| | Sto7 K13L | QGILVPCQSPWNTPLLPVKKPGTNDYRPVQ | | cgtcaggcaccgctgattattccgctgaaa |
| | N-tag: his | DLREVNKRVEDIHPTVPNPYNLLSGPPPSH | | gcgacgtcgaccccggtgagcatcaagcaa |
| | C-Tag: Sto7 | QWYTVLDLKDAFFCLRLHPTSQPLFAFEWR | | tatccgctgtcccaaaaggcgcgcttaggt |
| | P151 | DPEMGISGQLTWTRLPQGFKNSPTLFNEAL | | attaagccgcacattcagcgtctgctggat |
| | | HRDLADFRIQHPDLILLQYVDDLLLAATSE | | caaggtattctggttccgtgtcagagcccg |
| | | LDCQQGTRALLQTLGNLGYRASAKKAQICQ | | tggaataccccgcttctcccggtgaagaaa |
| | | KQVKYLGYLLKEGQRWLTEARKETVMGQPT | | ccgggcacgaacgattaccgtccagtccaa |
| | | PKTPRQLRRFLGKAGFCRLFIPGFAEMAAP | | gacttgcgcgaagttaacaagcgcgttgaa |
| | | LYPLTKPGTLFNWGPDQQKAYQEIKQALLT | | gatattcacccgaccgtcccgaacccgtac |
| | | APALGLPDLTKPFELFVDEKQGYAKGVLTQ | | aatctgctgagcggtccgccgccaagccac |
| | | KLGPWRRPVAYLSKKLDPVAAGWPPCLRMV | | caatggtacaccgtgctggatctgaaagat |
| | | AAIAVLTKDAGKLTMGQPLVIGAPHAVEAL | | gctttcttctgtctgcgtctgcacccaacc |
| | | VKQPPDRWLSKARMTHYQALLLDTDRVQFG | | agccagcctctgtttgcatttgagtggcgt |
| | | PVVALNPATLLPLPEEGLQHNCLDILAEAV | | gaccctgagatgggtattagcggccagctg |
| | | GTRPDLTDQPLPDADHTWYTNGSSLLQEGQ | | acgtggacccgcctgccgcaaggttttaag |
| | | RKAGAAVTTETEVIWAKALPAGTSAQRAEL | | aattcccctacgctgtttaacgaagcgctg |
| | | IALTQALKMAEGKKLNVYTDSRYAFATAHI | | caccgtgacctggcggatttccgtatccag |
| | | HGEIYRRRGWLTSKGKEIKNKDEILALLKA | | cacccggacctgatcttgctgcagtacgtt |
| | | LFLPKRLSIIYCPGHQKGHSAEARGNRMAD | | gatgacctgttgctggcggcgacgagcgag |
| | | QAARKAAITETPDTSTLLIENSSPNSRLIN | | ctggattgccaacagggcacccgtgcgctg |
| | | GGGGSVTVKFKYKGEELEVDISKIKKVWRV | | ttgcagaccttgggtaacctgggttatcgc |
| | | GKMISFTYDDNGKTGRGAVSEKDAPKELLQ | | gctagcgcgaagaaagcgcagatttgccaa |
| | | MLEKSGKK | | aaacaagttaagtatctgggctacctgtta |
| | | | | aaggaaggccaacgttggctgaccgaagcc |
| | | | | cgcaaagaaactgtcatgggtcagccgacc |
| | | | | ccgaaaacgccacgccaactgcgtaggttc |
| | | | | ttgggcaaagcgggtttctgccgcctgttc |
| | | | | atcccgggctttgccgaaatggcagccccg |
| | | | | ctgtatccgttgaccaagccgggcaccctg |
| | | | | ttcaactggggtccggaccagcagaaagcg |
| | | | | taccaagaaattaaacaagcactgctgacg |
| | | | | gcaccggcgctgggtctgccggacctgacc |
| | | | | aagccgtttgagctgttcgtggatgagaag |
| | | | | caaggttacgcgaagggcgtgttgacccag |
| | | | | aaattgggtccgtggcgtcgtccggttgca |
| | | | | tacctgtccaagaaactggacccggttgct |
| | | | | gctggttggccgccttgcctgcgcatggtt |
| | | | | gccgctatcgcggtgctgactaaagacgcg |
| | | | | ggtaagctgacgatgggtcaaccgctggtg |
| | | | | atcggtgcaccgcatgcagtcgaggccctt |
| | | | | gttaagcaaccgccggatagatggctgagc |
| | | | | aaggcgcgtatgacgcattaccaggcactg |
| | | | | ctgttggacaccgatcgtgtgcagtttggc |
| | | | | ccggtcgttgcgctcaacccggcgaccctg |
| | | | | ctgccgctcccggaagaaggcttgcagcac |
| | | | | aactgtttggacatcctggcagaggctgtg |
| | | | | ggaacccgcccagaccttactgaccaacca |
| | | | | cttcccgatgctgatcatacctggtatacg |
| | | | | aatggtagcagcctgttgcaagagggtcag |
| | | | | cgtaaggccggtgccgcggtcaccaccgag |
| | | | | actgaagtgatttgggctaaagcattgcct |
| | | | | gcgggtaccagcgcgcagcgtgccgagctg |
| | | | | atcgcactgacccaagcgctgaaaatggct |
| | | | | gagggtaagaaactgaatgtgtacacggat |
| | | | | agccgttatgcctttgcgaccgcccacatt |
| | | | | cacggcgagatctatcgccgtcgcggctgg |
| | | | | ctgacgtccaaaggcaaagagatcaagaat |
| | | | | aaagacgaaattctggcgctgctgaaagcg |
| | | | | ctgttcctgccgaaacgtctgtcgatcatc |
| | | | | tattgcccgggtcaccagaaaggccacagc |
| | | | | gcagaggcgcgtggtaatcgcatggctgac |
| | | | | caggctgcgcgtaaagccgcaattaccgaa |
| | | | | accccggacaccagcacgctgctgatcgag |
| | | | | aatagcagcccgaacagccgtctgatcaat |
| | | | | ggtggaggtgggtcagtaacagtaaaattt |
| | | | | aaatataaaggtgaagagttggaggttgat |
| | | | | atttcaaagataaaaaaagtgtggagagtc |
| | | | | ggtaaaatgataagttttacgtatgatgac |
| | | | | aacgggaaaaccggtcggggcgccgtgtcc |
| | | | | gaaaaagacgctccgaaggaactactccaa |
| | | | | atgcttgagaaaagtggaaaaaaa |
| 172 | RTx | TLNIEDEHRLHETSKEPDVSLGSTWLSDFP | | |
| | MMLV-WT | QAWAETGGMGLAVRQAPLIIPLKATSTPVS | | |
| | | IKQYPMSQEARLGIKPHIQRLLDQGILVPC | | |
| | | QSPWNTPLLPVKKPGTNDYRPVQDLREVNK | | |
| | | RVEDIHPTVPNPYNLLSGLPPSHQWYTVLD | | |
| | | LKDAFFCLRLHPTSQPLFAFEWRDPEMGIS | | |
| | | GQLTWTRLPQGFKNSPTLFDEALHRDLADF | | |
| | | RIQHPDLILLQYVDDLLLAATSELDCQQGT | | |
| | | RALLQTLGNLGYRASAKKAQICQKQVKYLG | | |
| | | YLLKEGQRWLTEARKETVMGQPTPKTPRQL | | |
| | | REFLGTAGFCRLWIPGFAEMAAPLYPLTKT | | |
| | | GTLFNWGPDQQKAYQEIKQALLTAPALGLP | | |
| | | DLTKPFELFVDEKQGYAKGVLTQKLGPWRR | | |
| | | PVAYLSKKLDPVAAGWPPCLRMVAAIAVLT | | |
| | | KDAGKLTMGQPLVILAPHAVEALVKQPPDR | | |
| | | WLSNARMTHYQALLLDTDRVQFGPVVALNP | | |
| | | ATLLPLPEEGLQHNCLDILAEAHGTRPDLT | | |
| | | DQPLPDADHTWYTDGSSLLQEGQRKAGAAV | | |
| | | TTETEVIWAKALPAGTSAQRAELIALTQAL | | |
| | | KMAEGKKLNVYTDSRYAFATAHIHGEIYRR | | |
| | | RGLLTSEGKEIKNKDEILALLKALFLPKRL | | |
| | | SIIHCPGHQKGHSAEARGNRMADQAARKAA | | |
| | | ITETPDTSTLL | | |

TABLE 6
Non-limiting embodiments of RT variants of the invention

| SEQ | | |
|---|---|---|
| ID | | |
| NO: | Description | Sequences |
| 173 | GAPDH | aga ggg aaa gca gcc cgc agc cuc ccg cuu cgc ucu cug cuc cuc cug uuc gac |
| | (RNA) | agu cag ccg cau cuu cuu uug cgu cgc cag ccg agc cac auc gcu cag aca cca |
| | Template | ugg gga agg uga agg ucg gag uca acg gau uug guc gua uug ggc gcc ugg uca |
| | | cca ggg cug cuu uua acu cug gua aag ugg aua uug uug cca uca aug acc ccu |
| | | uca uug acc uca acu aca ugg uuu aca ugu ucc aau aug auu cca ccc aug gca |
| | | aau ucc aug gca ccg uca agg cug aga acg gga agc uug uca uca aug gaa auc |
| | | cca uca cca ucu ucc agg agc gag auc ccu cca aaa uca agu ggg gcg aug cug |
| | | gcg cug agu acg ucg ugg agu cca cug gcg ucu uca cca cca ugg aga agg cug |
| | | ggg cuc auu ugc agg ggg gag cca aaa ggg uca uca ucu cug ccc ccu cug cug |
| | | aug ccc cca ugu ucg uca ugg gug uga acc aug aga agu aug aca aca gcc uca |
| | | aga uca uca gca aug ccu ccu gca cca cca acu gcu uag cac ccc ugg cca agg uca |
| | | ucc aug aca acu uug gua ucg ugg aag gac uca uga cca cag ucc aug cca uca |
| | | cug cca ccc aga aga cug ugg aug gcc ccu ccg gga aac ugu ggc gug aug gcc |
| | | gcg ggg cuc ucc aga aca uca ucc cug ccu cua cug gcg cug cca agg cug ugg |
| | | gca agg uca ucc cug agc uga acg gga agc uca cug gca ugg ccu ucc gug ucc |
| | | cca cug cca acg ugu cag ugg ugg acc uga ccu gcc guc uag aaa aac cug cca |
| | | aau aug aug aca uca aga agg ugg uga agc agg cgu cgg agg gcc ccc uca agg |
| | | gca ucc ugg gcu aca cug agc acc agg ugg ucu ccu cug acu uca aca gcg aca |
| | | ccc acu ccu cca ccu uug acg cug ggg cug gca uug ccc uca acg acc acu uug |
| | | uca agc uca uuu ccu ggu aug aca acg aau uug gcu aca gca aca ggg ugg ugg |
| | | acc uca ugg ccc aca ugg ccu cca agg agu aag acc ccu gga cca cca gcc cca gca |
| | | aga gca caa gag gaa gag aga gac ccu cac ugc ugg gga guc ccu gcc aca cuc |
| | | agu ccc cca cca cac uga auc ucc ccu ccu cac agu ugc cau gua gac ccc uug aag |
| | | agg gga ggg gcc uag gga gcc gca ccu ugu cau gua cca uca aua aag uac ccu |
| | | gug cuc aac c |
| 174 | GAPDH | /56-FAM/tgg ttg agc aca ggg tac ttt att gat gg |
| | Primer-56 | |
| | FAM | |
| 175 | TSO1 | aag cag tgg tat caa cgc aga gta cat rgrgrg |
| | Oligo | |
| 176 | GAPDH | TGG TTG AGC ACA GGG TAC TTT ATT GAT GG |
| | Primer | |
| 177 | 5x His tag | His His His His His |
| | amino | |
| | acid | |
| 178 | Wild Type | TLNIEDEHRLHETSKEPDVSLGSTWLSDFPQAWAETGGMGLAVRQAPLIIPL |
| | MMLV | KATSTPVSIKQYPMSQEARLGIKPHIQRLLDQGILVPCQSPWNTPLLPVKKP |
| | Identical | GTNDYRPVQDLREVNKRVEDIHPTVPNPYNLLSGLPPSHQWYTVLDLKDAF |
| | to SEQ ID | FCLRLHPTSQPLFAFEWRDPEMGISGQLTWTRLPQGFKNSPTLFDEALHRDL |
| | NO: 7 | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLREFLGTAG |
| | | FCRLWIPGFAEMAAPLYPLTKTGTLFNWGPDQQKAYQEIKQALLTAPALGL |
| | | PDLTKPFELFVDEKQGYAKGVLTQKLGPWRRPVAYLSKKLDPVAAGWPPC |
| | | LRMVAAIAVLTKDAGKLTMGQPLVILAPHAVEALVKQPPDRWLSNARMT |
| | | HYQALLLDTDRVQFGPVVALNPATLLPLPEEGLQHNCLDILAEAHGTRPDL |
| | | TDQPLPDADHTWYTDGSSLLQEGQRKAGAAVTTETEVIWAKALPAGTSAQ |
| | | RAELIALTQALKMAEGKKLNVYTDSRYAFATAHIHGEIYRRRGLLTSEGKEI |
| | | KNKDEILALLKALFLPKRLSIIHCPGHQKGHSAEARGNRMADQAARKAAIT |
| | | ETPDTSTLL |
| 179 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | (42B) | MGISGQLTWTRLPQGFKNSPTLFNEALHRDLADFRIQHPDLILLQYVDDLLL |
| | Identical | AATSELDCQQGTRALLQTLGNLGYRASAKKAQICQKQVKYLGYLLKEGQR |
| | to SEQ ID | WLTEARKETVMGQPTPKTPRQLRRFLGKAGFCRLFIPGFAEMAAPLYPLTK |
| | NO: 1 | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIHCPGHQKGHSAEARGNRMADQAARKAAITETPDTSTLLIENSSPNSRLIN |
| 180 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN |
| 181 | MMLV | MTWLSDFPQAWAETGGVGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIKAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN |
| 182 | MMLV | MTWLSDFPQAWAETGGVGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIKAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN* |
| 183 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIKAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN* |
| 184 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAVGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIYCPGHQKGHSAEARGNRMADQAARKAAITETPDTSTLLIENSSPNSRLIN |
| | | * |
| 185 | MMLV | MTWLSDFPQAWAETGGVGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAVGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIYCPGHQKGHSAEARGNRMADQAARKAAITETPDTSTLLIENSSPNSRLIN |
| | | * |
| 186 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAGRWLSKARMTH |
| | | YQALLLDTDRVQFGPVVALNPATLLPLPEEGLQHNCLDILAEAHGTRPDLT |
| | | DQPLPDADHTWYTNGSSLLQEGQRKAGAAVTDETEVIWAKALPAGTSAQR |
| | | AELIALTQALKMAEGKKLNVYTNSRYAFATAHIHGEIYRRRGWLTSGGKEI |
| | | KNKDEILALLKALFLSKRLSIIHCPGHQKGHSVEARGNRMAHQAARRAAIT |
| | | ETPDTSTLP |
| 187 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAERWLSKARMTH |
| | | YQALLLDTDRVQFGPVVALNPATLLPLPEEGLQHNCLDILAEAHGTRPDLT |
| | | DQPLPDADHTWYTNGSSLLQEGQRKAGAAVTDETEVIWAKALPAGTSAQR |
| | | AELIALTQALKMAEGKKLNVYTDSRYAFATAHIHGEIYRRRGWLTSGGKEI |
| | | KNKDEILALLKALFLPKRLSIIHCPGHQKGHSVEARGNHMAHQAARRAAIT |
| | | ETPDTSTLP |
| 188 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAGRWLSKARMTH |
| | | YQALLLDTDRVQFGPVVALNPATLLPLPEEGLQHNCLDILAEAHGTRPDLT |
| | | DQPLPDADHTWYTDGSSLLQEGQRKAGAAVTTETGVIWAKALPAGTSAQR |
| | | AELIALTQALKMAEGKKLNVYTNSRYAFATAHIQGEIYRRRGFLTSKGKEI |
| | | KNKDEILALLKALFLPKRLSIIHCPGHQKGHSAEARGNRMADQAARKAAIT |
| | | ETPDTSTLP |
| 189 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAERWLSKARMTH |
| | | YQALLLDTDRVQFGPVVALNPATLLPLPEEGLQHNCLDILAEAHGTRPDLT |
| | | DQPLPDADHTWYTNGSSLLQEGQRKAGAAVTDETEVIWAKALPAGTSAQR |
| | | AELIALTQALKMAEGKKLNVYTDSRYAFATAHIHGEIYRRRGWLTSGGKEI |
| | | KNKDEILALLKALFLPKRLSIIHCPGHQKGHSVEARGNRMAHQAARRAAIT |
| | | ETPDTSTLP |
| 190 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFEEALRRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAGRWLSKARMTH |
| | | YQALLLDTDRVQFGPVVALNPATLLPLPEEGLQHNCLDILAEAHGTRPDLT |
| | | DQPLPDADHTWYTNGSSLLQEGQRKAGAAVTDETEVIWAKALPAGTSAQR |
| | | AELIALTQALKMAEGKKLNVYTNSRYAFATAHIHGEIYRRRGWLTSGGKEI |
| | | KNKDEILALLKALFLSKRLSIIHCPGHQKGHSVEARGNRMAHQAARRAAIT |
| | | ETPDTSTLP |
| 191 | MMLV | TLNIEDEHRLHETSKEPDVSLGSTWLSDFPQAWAETGGVGLAVRQAPLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFEEALRRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAGRWLSKARMTH |
| | | YQALLLDTDRVQFGPVVALNPATLLPLPEEGLQHNCLDILAEAHGTRPDLT |
| | | DQPLPDADHTWYTDGSSLLQEGQRKAGAAVTTETGVIWAKALPAGTSAQR |
| | | AELIALTQALKMAEGKKLNVYTNSRYAFATAHIQGEIYRRRGFLTSKGKEI |
| | | KNKDEILALLKALFLPKRLSIIHCPGHQKGHSAEARGNRMADQAARKAAIT |
| | | ETPDTSTLP |
| 192 | MMLV | TLNIEDEHRLHETSKEPDVSLGSTWLSDFPQAWAETGGVGLAVRQALLIIPL |
| | RT | KATSTPVSIKQYPMSQKARLGIKPHIQRLLDQGILVPCQSPWNTPLLPVKKP |
| | Variant | GTNDYRPVQDLREVNKRVEDIHPTVPNPYNLLSGPPPSHQWYTVLDLKDAF |
| | | FCLRLHPTSQPLFAFEWRDPEMGISGQLTWTRLPQGFKNSPTLFNEALHRDL |
| | | ADFRIQHPDLILLQYVDDLLLAATSELDCQQGTRALLQTLGNLGYRASAKK |
| | | AQICQKQVKYLGYLLKEGQRWLTEARKETVMGQPTPKTPRQLRRFLGKAG |
| | | FCRLFIPGFAEMAAPLYPLTKPGTLFNWGPDQQKAYQEIKQALLTAPALGLP |
| | | DLTKPFELFVDEKQGYAKGVLTQKLGPWRRPVAYLSKKLDPVAAGWPPCL |
| | | RMVAAIAVLTKDAGKLTMSQPLVIKAPHAVEALVKQPAGRWLSKARMTH |
| | | YQALLLDTDRVQFGPVVALNPATLLPLPEEGLQHNCLDILAEAHGTRPDLT |
| | | DQPLPDADHTWYTNGSSLLQEGQRKAGAAVTDETEVIWAKALPAGTSAQR |
| | | AELIALTQALKMAEGKKLNVYTNSRYAFATAHIHGEIYRRRGWLTSGGKEI |
| | | KNKDEILALLKALFLSKRLSIIHCPGHQKGHSVEARGNRMAHQAARRAAIT |
| | | ETPDTSTLP |
| 193 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIHCPGHQKGHSAEARGNRMADQAARKAAITETPDTSTLLIENSSPNSRLIN |
| 194 | MMLV | MTWLSDFPQAWAETGGVGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIHCPGHQKGHSAEARGNRMADQAARKAAITETPDTSTLLIENSSPNSRLIN |
| 195 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN |
| 196 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAYFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPAGRWLSKARMTHYQALLLDTDRVQFGPVVA |
| | | LNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIHCPGHQKGHSAEARGNRMADQAARKAAITETPDTSTLLIENSSPNSRLIN |
| 197 | MMLV | MTWLSDFPQAWAETGGVGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN |
| 198 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPADRWLSKARMTHYQALLLDTDRVQFGPVVA |
| | | LNPATLLPLPEEGLQHNCLDILAEAHGTRPDLTDQPLPDADHTWYTNGSSL |
| | | LQEGQRKAGAAVTTETEVIWAKALPAGTSAQRAELIALTQALKMAEGKKL |
| | | NVYTDSRYAFATAHIHGEIYRRRGWLTSKGKEIKNKDEILALLKALFLPKRL |
| | | SIIHCPGHQKGHSAEARGNRMADQAARKAAITETPDTSTLLIENSSPNSRLIN |
| 199 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPGRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 200 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAVGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 201 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIYCPGHQKGHSAEARGNRMADQAARKAAITETPDTSTLLIENSSPNSRLIN |
| 202 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 203 | MMLV | MTWLSDFPQAWAETGGMGLAVRQAPLIIPLKATSTPVSIKQYPMSQKARLG |
| | RT | IKPHIQRLLDQGILVPCQSPWNTPLLPVKKPGTNDYRPVQDLREVNKRVEDI |
| | Variant | HPTVPNPYNLLSGPPPSHQWYTVLDLKDAYFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 204 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGGQKGHSAEARGNRMADQAARKAAITETPDTSTLLIENSSPNSRLIN |
| 205 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAYFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGGQKGHSAEARGNRMADQAARKAAITETPDTSTLLIENSSPNSRLIN |
| 206 | MMLV | MTWLSDFPQAWAETGGVGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 207 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAYFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |
| 208 | MMLV | MTWLSDFPQAWAETGGMGLAVRQAPLIIPLKATSTPVSIKQYPLSQKARLGI |
| | RT | KPHIQRLLDQGILVPCQSPWNTPLLPVKKPGTNDYRPVQDLREVNKRVEDI |
| | Variant | HPTVPNPYNLLSGPPPSHQWYTVLDLKDAFFCLRLHPTSQPLFAFEWRDPE |
| | | MGISGQLTWTRLPQGFKNSPTLFNEALHRDLADFRIQHPDLILLQYVDDLLL |
| | | AATSELDCQQGTRALLQTLGNLGYRASAKKAQICQKQVKYLGYLLKEGQR |
| | | WLTEARKETVMGQPTPKTPRQLRRFLGKAGFCRLFIPGFAEMAAPLYPLTK |
| | | PGTLFNWGPDQQKAYQEIKQALLTAPALGLPDLTKPFELFVDEKQGYAKG |
| | | VLTQKLGPWRRPVAYLSKKLDPVAAGWPPCLRMVAAIAVLTKDAGKLTM |
| | | GQPLVIGAPHAVEALVKQPPDRWLSKARMTHYQALLLDTDRVQFGPVVAL |
| | | NPATLLPLPEEGLQHNCLDILAEAHGTRPDLTDQPLPDADHTWYTNGSSLL |
| | | QEGQRKAGAAVTTETEVIWAKALPAGTSAQRAELIALTQALKMAEGKKLN |
| | | VYTDSRYAFATAHIHGEIYRRRGWLTSKGKEIKNKDEILALLKALFLPKRLS |
| | | IIHCPGHQKGHSAEARGNRMADQAARKAAITETPDTSTLLIENSSPNSRLIN |

1.-11. (canceled)

12. An engineered fusion reverse transcriptase (RT) comprising:
(a) at least one DNA binding domain selected from a DNA binding domain from an archaeal DNA binding protein, wherein the archaeal DNA binding protein is selected from the group consisting of Sto7d, Sso7d, Sis7b, Sis7a, Ssh7b, Sto7, Aho7C, Aho7B, Aho7A, Mcu7, Mse7, Sac7e, and Sac7d; and
(b) an engineered reverse transcriptase, wherein the engineered reverse transcriptase comprises the combination of the following amino acid substitutions in SEQ ID NO: 7:
(i) E69K, L139P, E302R, T306K, W313F, T330P, and N454K; and one or more of M39V, P47L, M66L, F155Y, D200N, D200E, H204R, G429S, L435G, L435K, P448A, D449G, H503V, D524N, T542D, E545G, D583N, H5940, L603W, L603F, E607K, E607G, P627S, H634Y, H638G, A644V, D653H, K658R and L671P: or
(ii) E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H5940, P627S, H634Y, H638G, A644V, D653H, K658R and L671P.

13. The engineered fusion reverse transcriptase of claim 12, wherein the engineered reverse transcriptase comprises:
(a) M39V, M66I, Q91R, I347V, H594Q, or a combination thereof, and optionally M39V, M66I, Q91R, I347V, H594Q, or a combination thereof, substituted in the RT sequence of SEQ ID NO: 143;
(b) M39V, T542D, D583N, E607G, A644V, D653H, K658R, L671P, or a combination thereof, substituted in the RT sequence of SEQ ID NO: 143 or SEQ ID NO: 7;
(c) M39V, T542D, D583N, E607G, A644V, D653H, K658R, L671P, or a combination thereof, and optionally M39V, T542D, D583N, E607G, A644V, D653H, K658R, L671P, or a combination thereof, substituted in the RT sequence of SEQ ID NO: 143 or SEQ ID NO: 7;
(d) M39V, M66I, Q91R, I347V, or H594Q, or a combination thereof in the RT backbone of SEQ ID NO: 143 or SEQ ID NO: 7; or
(e) at least one mutation selected from an M39V mutation or an M66L mutation, and wherein the mutation is indexed to an amino acid sequence set forth in SEQ ID NO:7.

14.-20. (canceled)

21. The engineered fusion reverse transcriptase of claim 12, wherein the engineered reverse transcriptase comprises an amino acid sequence that is at least about 90% identical to an amino acid sequence selected from:
(a) SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55;
(b) SEQ ID NOs: 180-208;
(c) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172; or
(d) SEQ ID NO: 143, SEQ ID NO: 145, SEQ ID NO: 147, SEQ ID NO: 105, SEQ ID NO: 107, SEQ ID NO: 111, SEQ ID NO: 123, SEQ ID NO: 127, SEQ ID NO: 129, SEQ ID NO: 131 or SEQ ID NO: 172.

22. The engineered fusion reverse transcriptase of claim 12, wherein the amino acid sequence of the engineered reverse transcriptase comprises E69K, L139P, D200N, E302R, T306K, W313F, T330P, N454K, H503V, D524N, L603W, E607K, and H634Y.

23. The engineered fusion reverse transcriptase of claim 22, wherein the amino acid sequence of the engineered reverse transcriptase comprises a combination of mutations selected from:
(a) M66L and L435G;
(b) M39V, M66L, and L435K;
(c) M39V and L435K;
(d) M66L, L435G, P448A and D449G;
(e) M39V, M66L, L435G, P448A and D449G; or
(f) M66L.

24. The engineered fusion reverse transcriptase of claim 12, wherein the amino acid sequence of the engineered reverse transcriptase comprises E69K, L139P, D200N, E302R, T306K, W313F, T330P, L435G, P448A, D449G, N454K, D524N, L603W, and E607K; and further comprises a combination of mutations selected from
(a) M66L;
(b) M66L and H503V;
(c) M66L and H634Y; or
(d) M66L, H503V, and H634Y.

25. The engineered fusion reverse transcriptase of claim 12, wherein the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P, and further comprises a second combination of mutations selected from:
(a) D524N, T542D, P627S, A644V, D653H, K658R mutation, and wherein the D200 mutation is a D200N mutation, the D449 mutation is a D449G mutation, the L603 mutation is an L603W mutation, and the E607 mutation is an E607G mutation;
(b) D524N, T542D, A644V, D653H, an R650H and K658R, and wherein the D200 mutation is a D200N mutation, the D449 mutation is a D449E mutation, the L603 mutation is an L603W mutation, and the E607 mutation is an E607G mutation;
(c) E545G, D583N, and H594Q, and wherein the D200 mutation is a D200N mutation, the D449 mutation is a D449G mutation, the L603 mutation is an L603F mutation, and the E607 mutation is an E607K mutation;
(d) D524N, T542D, A644V, D653H, and K658R, wherein the D200 mutation is a D200N mutation, the D449 mutation is a D449E mutation, the L603 mutation is an L603W mutation, the E607 mutation is an E607G mutation;
(e) H204R, D524N, T542D, P627S, D583N, A644V, D653H and K658R, wherein the D200 mutation is a D200E mutation, the D449 mutation is a D449G mutation, the L603 mutation is an L603W mutation, the E607 mutation is an E607G mutation;
(f) H204R, E545G, D583N, and H594Q, wherein the D200 mutation is a D200E mutation, the D449 mutation is a D449G mutation, the L603 mutation is an L603F mutation, the E607 mutation is an E607K mutation; or
(g) P47L, D524N, T542D, D583N, P627S, A644V, D653H, and K658R, wherein the D200 mutation is a D200N mutation, the D449 mutation is a D449G mutation, the L603 mutation is an L603W mutation, the E607 mutation is an E607G mutation.

26. The engineered fusion reverse transcriptase of claim 12, wherein the at least one DNA binding domain:
(a) is located at the C-terminus or at the N-terminus of the engineered fusion reverse transcriptase; or
(b) comprises a Sto7 DNA binding domain at the C-terminus or N-terminus of the engineered fusion reverse transcriptase; or
(c) comprises Stod7d DNA binding domain.

27.-28. (canceled)

29. The engineered fusion reverse transcriptase of claim 12, wherein the DNA binding domain comprises:
(a) an amino acid sequence selected from SEQ ID NO: 12, 13, 16, 17, or 18; or
(b) an amino acid sequence having at least about 90%, at least about 91%, at least about 92%, at least about 93%, at least about 94%, at least about 95%, at least about 96%, at least about 97%, at least about 98%, or at least about 99% sequence identity to any one of SEQ ID NO: 12, 13, 16, 17, or 18;
(c) a DNA binding domain consensus motif set forth in SEQ ID NO: 2; or
(d) a mutation selected from a K13 mutation, a D36 mutation, an N37 mutation, a V2 mutation, an insertion, or a combination thereof;
(e) a K13L mutation, a D36L mutation, or a combination thereof; or
(f) the amino acid sequence of SEQ ID NO: 18.

30.-35. (canceled)

36. The engineered fusion reverse transcriptase of claim 12, wherein:
(a) the DNA binding domain comprises an amino acid sequence selected from SEQ ID NO: 12, 13, 16, 17, or 18;
(b) the engineered reverse transcriptase comprises:
(i) an amino acid sequence selected from: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, or SEQ ID NO: 55; or
(ii) SEQ ID NOs: 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 141, 143, 145, 147, 149, 151, 157, 159, or 172;
(c) the DNA binding domain is located at the C-terminus or N-terminus of the engineered fusion reverse transcriptase.

37. The engineered fusion reverse transcriptase of claim 12, wherein the amino acid sequence of the engineered fusion reverse transcriptase comprises:
(a) an amino acid sequence selected from SEQ ID NO: 3, SEQ ID NO: 5, SEQ ID NO: 6, SEQ ID NO: 8, SEQ ID NO: 20, SEQ ID NO: 111, SEQ ID NO: 129, SEQ ID NO: 135, SEQ ID NO: 137, SEQ ID NO: 139, SEQ ID NO: 153, SEQ ID NO: 155, SEQ ID NO: 157, SEQ ID NO: 159, SEQ ID NO: 161, SEQ ID NO: 163, SEQ ID NO: 166, SEQ ID NO: 168, or SEQ ID NO: 170; or
(b) an amino acid sequence having at least about 90%, at least about 92%, at least about 95%, at least about 97%, at least about 98%, or at least about 99% sequence identity to SEQ ID NO: 3, SEQ ID NO: 5, SEQ ID NO: 6, SEQ ID NO: 8, SEQ ID NO: 20, SEQ ID NO: 111, SEQ ID NO: 129, SEQ ID NO: 135, SEQ ID NO: 137, SEQ ID NO: 139, SEQ ID NO: 153, SEQ ID NO: 155, SEQ ID NO: 157, SEQ ID NO: 159, SEQ ID NO: 161, SEQ ID NO: 163, SEQ ID NO: 166, SEQ ID NO: 168, or SEQ ID NO: 170; or
c) SEQ ID NO: 111, SEQ ID NO: 129, or SEQ ID NO: 20.

38. (canceled)

39. The engineered fusion reverse transcriptase of claim 12, wherein the engineered fusion reverse transcriptase comprises:
(a) at least two DNA binding domains,
(b) at least two DNA binding domains and at least one DNA binding domain is located at the N-terminus of the engineered fusion reverse transcriptase and at least one DNA binding domain is located at the C-terminus of the engineered fusion reverse transcriptase; or
(c) at least two DNA binding domains and the at least two DNA binding domains are both located at the C-terminus or N-terminus of the engineered fusion reverse transcriptase.

40.-41. (canceled)

42. The engineered fusion reverse transcriptase of claim 39, wherein at least one DNA binding domain is located at the N-terminus of the engineered fusion reverse transcriptase and at least one DNA binding domain is located at the C-terminus of the engineered fusion reverse transcriptase and:
(a) the DNA binding fusion domain located at the N-terminus is Sso7d DNA binding domain and the DNA binding domain located at the C-terminus of is Sso7d DNA binding domain; or
(b) the DNA binding domain located at the N-terminus is Sto7 DNA binding domain and the DNA binding domain located at the C-terminus is Sto7 DNA binding domain;
(c) the DNA binding domain located at the N-terminus is Ss07d DNA binding domain and the DNA binding domain located at the C-terminus is Sto7 DNA binding domain; or
(d) the DNA binding domain located at the N-terminus is Sto7 DNA binding domain and the DNA binding domain located at the C-terminus is Ss07d DNA binding domain.

43.-44. (canceled)

45. The engineered fusion reverse transcriptase of claim 12, wherein the engineered reverse transcriptase is at least about 95% identical to SEQ ID NO: 1, and wherein the amino acid sequence of the engineered reverse transcriptase comprises a second combination of mutations indexed to SEQ ID NO: 7 selected from:
(a) an E69K mutation, an E302R mutation, a T306K mutation, a W313F mutation, an L435G mutation, and an N454K mutation, and comprising at least one mutation selected from an M39V mutation, an M66L mutation, an L139P mutation, an F155Y mutation, a D200N mutation, an E201Q mutation, a T287A mutation, a T330P mutation, an R411F mutation, a P448A mutation, a D449G mutation, an H503V mutation, an H594K mutation, L603W mutation, an E607K mutation, an H634Y mutation, a G637R mutation or an H638G mutation;
(b) an L139P mutation, a D200N mutation, a T330P mutation, an L603W mutation, and an E607K mutation, and comprising at least one mutation selected from: an M39V mutation, an M66L mutation, an E69K mutation, an F155Y mutation, an E201Q mutation, a T287A mutation, an E302R mutation, a T306K mutation, a W313F mutation, an R411F mutation, an L435G mutation, a P448A mutation, a D449G mutation, an N454K mutation, an H503V mutation, an H594K mutation, an H634Y mutation, a G637R mutation or an H638G mutation;
(c) an A32V mutation, an L72R mutation, a D200C mutation, a G248C mutation, an E286R mutation, an E302R mutation, a W388R mutation, and an L435G mutation; or
(d) a Y344L mutation and an I347L mutation.

46. (canceled)

47. An isolated nucleic acid molecule encoding the engineered fusion reverse transcriptase of claim 12.

48. (canceled)

49. A host cell transfected with the isolated nucleic acid of claim 47.

50.-66. (canceled)

67. A method of using the engineered fusion reverse transcriptase of claim 12, the method comprising contacting the engineered fusion reverse transcriptase with a nucleic acid template under suitable conditions to produce a polymerized nucleic acid product, wherein the nucleic acid template is an RNA, a DNA, or a nucleic acid comprising an unnatural nucleotide.

68. A nucleic acid extension method comprising:
(a) contacting a target nucleic acid molecule with an engineered fusion reverse transcriptase and a plurality of nucleic acid barcoded molecules comprising a barcode sequence, and
(b) incubating the target nucleic acid, the engineered fusion reverse transcriptase and barcoded molecules under conditions in which the barcoded molecules are extended by the engineered fusion reverse transcriptase, wherein the engineered fusion reverse transcriptase comprises the amino acid sequence of an engineered transcriptase of claim 12.

69.-78. (canceled)

79. The isolated nucleic acid molecule of claim 47, wherein the isolated nucleic acid molecule comprises a sequence selected from SEQ ID NO: 136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:167, SEQ ID NO:169, or SEQ ID NO: 171.

80. An expression vector comprising the isolated nucleic acid of claim 79.

81.-85. (canceled)
